(19) United States
(12) Patent Application Publication
(10) Pub. No.: US 2025/0312434 A1
(43) Pub. Date: Oct. 9, 2025

(54) LIPOPOLYSACCHARIDE (LPS) DEFICIENT ACINETOBACTER BAUMANNII MULTIVALENT VACCINE

(71) Applicants: VAXDYN S.L., Dos Hermanas (Sevilla) (ES); INSTITUTO DE SALUD CARLOS III, Madrid (ES)

(72) Inventors: Juan José INFANTE VIÑOLO, Dos Hermanas (Sevilla) (ES); Michael James MCCONNELL, Madrid (ES); Ana Isabel RODRÍGUEZ ROSADO, Dos Hermanas (Sevilla) (ES); María del Mar CORDERO ALBA, Dos Hermanas (Sevilla) (ES); Astrid PÉREZ GÓMEZ, Dos Hermanas (Sevilla) (ES); Andrés CORRAL LUGO, Madrid (ES)

(21) Appl. No.: 18/702,767

(22) PCT Filed: Oct. 20, 2022
(86) PCT No.: PCT/EP22/79329

§ 371 Date: Apr. 18, 2024

(30) Foreign Application Priority Data
Oct. 20, 2021 (EP) 21382943.5

(51) Int. Cl. A61K 39/104; A61K 39/00; A61K 39/39; A61P 37/04

(52) U.S. Cl. CPC A61K 39/104; A61K 39/39; A61P 37/04; A61K 2039/523; A61K 2039/55505

(57) ABSTRACT

The invention refers to a composition comprising inactivated cells deficient in LPS from the genus Acinetobacter and/or outer membrane vesicles form the same and their use for the manufacture of a medicament, preferably a vaccine, for the prevention of diseases caused by K. pneumoniae, P. aeruginosa, E. coli, and/or A. pleuropneumoniae, and optionally A. baumannii.

### TECHNICAL FIELD

The invention refers to a composition comprising inactivated cells from the genus Acinetobacter unable to synthesize Lipid A, the core component of the LPS and able to express heterologous outer-membrane proteins from other bacteria, and/or outer membrane vesicles derived from the same cells, and their use for the manufacture of a medicament, preferably a vaccine, for the prevention of diseases caused by the bacteria from which the heterologous outer-membrane proteins originally derived from and optionally Acinetobacter baumannii.

### BACKGROUND ART

The management of carbapenem-resistant infections is often based on antibiotics, including polymyxins, tigecycline, aminoglycosides and their combinations. However, in recent reviews, we have found that Gram-negative bacteria (GNB) co-resistant to carbapenems, aminoglycosides, polymyxins and tigecycline (CAPT-resistant) are increasingly being reported worldwide. There is thus a need for further treatment options in combination with rapid diagnostic methods. The most comprehensive study about the impact of Anti-Microbial Resistance (AMR) showed that only in 2019, five million people died in the world from a cause related to an infection produced by an AMR pathogen. Seven bacterial species account for 73% of the global deaths: the Gram-negative bacteria Klebsiella pneumoniae, Pseudomonas aeruginosa, Acinetobacter baumannii, and Escherichia coli, and the Gram-positive bacteria Staphylococcus aureus and Streptococcus pneumoniae. There are only vaccines available against one out of these six global killers, Streptococcus pneumoniae. In this invention, we specifically focus on a technology able to deliver drug substances for use as vaccines against bacterial infections, and specifically against Gram-negative bacteria, including Klebsiella pneumoniae, Pseudomonas aeruginosa, Acinetobacter baumannii, and Escherichia co/i. The technology can be applied to any Gram-negative bacterium, for instance to Actinobacillus pleuropneumoniae, the causative agent of porcine pleuropneumonia. The invention is based on the fact that, as shown before, the use of inactivated cells derived from Lipid-A mutants of Acinetobacter baumannii (A. baumannii) unable to synthesize LPS as vaccines can prevent infections produced by A. baumannii, and not only by certain specific clones of A. baumannii but from diverse clones, since immunity raised by the Lipid-A (LPS) null inactivated cells is focused against multiple highly conserved epitopes from outer-membrane proteins (OMPs) of the external membrane of A. baumannii. The invention also relies on the fact that OMPs are immunogenic bacterial components leading to the natural immune response in humans and animals to prevent opportunistic infections. And it relies on the fact that by focusing immunity on outer-membrane protein (OMP) antigens, which are highly conserved proteins, the vaccines could lead to universal immunity against all circulating clones or variants of a bacterial pathogen, overcoming a typical problem of bacterial vaccines whose active principles are antigens from the LPS or capsular polysaccharides. The antigens from the LPS or capsular polysaccharides are very variable between clones or variants of the same bacterial species, and therefore, vaccines, including conjugate vaccines, whose active principle is based on immunogenic antigens derived from the LPS or the capsular polysaccharides, can only cover a limited number of variants. Moreover, inactivated Lipid-A null mutant cells of A. baumannii used as vaccines raise immunity against OMP proteins conformed in their native conformation in the external membrane of the cell, differentiating the immune response from that obtained from using recombinant OMP proteins as vaccines. And finally, the antigenic presentation of multiple OMPs in their native conformation as part of a Lipid-A null mutant cell of A. baumannii is enhanced over presenting particular proteins, in the form of recombinant proteins, or peptides derived from the OMPs as vaccines. With all this background, the technologies that led to this invention aimed to deliver a method of producing Lipid-A null mutant cells of A. baumannii able to present in their membrane not only immunogenic OMPs from A. baumannii but also immunogenic OMPs from other Gram-negative bacterial pathogens. By this way, the advantages mentioned above of using inactivated Lipid-A null mutants of A. baumannii as vaccines against A. baumannii can be applied to the fight against other Gram-negative bacterial pathogens.

### BRIEF DESCRIPTION OF THE DRAWINGS

FIG. 1. This figure shows that AcinetoVax, a vaccine based on inactivated whole-cells of an LPS-deficient A. baumannii strain characterized by the complete inactivation of lpxC as taught in EP2942389A1 plus the adjuvant aluminum hydroxide, protects against infection by different A. baumannii clinical isolates. Mice were infected with the indicated strains of A. baumannii (ATCC19606, Ab-154, and Ab-113-16) 7 days after the second immunization with AcinetoVax (day 21) and survival was monitored for 7 days. ATCC19606 is a urine infection clinical isolate used as reference strain in sepsis and pneumonia models of infection in mice. Ab-154 is a clinical isolate sensitive to carbapenems, from an outbreak at the Hospital Virgen del Rocio of Seville in 2002. Ab-113-16 is a panresistant clinical isolate from a patient who died as a consequence of the 2002 outbreak.

FIG. 2. Humoral response raised by VXD001 (AcinetoVax) (A) only 7 days after the first dose. Analysis of IgG subtypes has shown both IgG1 and IgG2 subtypes, with a partial bias to IgG1. In B we see the release of the Thl-cytokine IFN-gamma (linked to CD8+ T-cells) of splenocytes from mice vaccinated with a low-dose (yellow bars) or a high-dose (grey bars) of VXD001 and stimulated ex-vivo with the vaccine antigen. We see that there is also a significant response after the first dose, which faints only to be boosted with the 2nd shot at day 14. We observed the same pattern by analysis of release of IL-4 (Thl) and IL-17 (Thl7) cytokines.

FIG. 3. Analysis of LPS-null derivatives from cells of the drug-substance KapaVax. The figure at the left shows the results of PCR amplification of a genomic region comprising the LPS synthesis gene lpxC. The expected wild-type product is 1.5 Kb-long. Clones 4 and 5 are colistin-resistant mutants selected after plating cells of KapaVax2 on plates supplemented with colistin. The lpxC genomic region was later sequenced and the insertion of a 1Kb ISAbal transposable element at lpxC was found responsible for inactivation of lpxC and the loss of LPS. The figure at the right shows the results of quantification of the LPS (endotoxin) by a chromogenic LAL assay (Pierce™ Chromogenic Endotoxin Quant Kit, Thermo Fisher Scientific) in cultures of the parental strain A. baumannii Ab283, an LPS-null derivative obtained earlier from Ab283, and the 2 LPS-null clones of KapaVax2. KapaVax or KapaVax2 are cells of A. baumannii LPS-null strain Ab283 expressing the heterologous OMPs from K. pneumoniae OmpA and OmpK36 and the heterologous OMPs from P. aeruginosa OprF and fusion protein OprI::PcrV.

FIG. 4. Analysis of LPS-null derivatives from cells of the drug-substances K-Vax and P-Vax. The figure at the upper left side shows the results of PCR amplification of a genomic region comprising the LPS synthesis gene lpxC. The expected wild-type product is 1.5 Kb-long. The figure shows PCR products obtained from DNA from several colistin-resistant derivatives of 3 different clones (clone 4, 20, 42) of K-Vax cells, i.e. A. baumannii Ab283 cells expressing the OMP proteins OmpA and OmpK36 from K. pneumoniae in the external membrane. The mutants showing the products marked with an arrow were sequenced and ISAbAl insertions at the lpxC gene were found. The insertions made lpxC inactivated and therefore the cells could not synthesize LPS. At the upper right side there are plates with cells from four of the detected LPS-null mutants, with growth shown on Muller-Hilton media and no growth shown on agar-MacConkey media. Loss of LPS is liked to inability to grow on agar-MacConkey media. The figure at the lower left side shows the results of PCR amplification of genomic regions comprising the LPS synthesis genes lpxC and lpxD of P-Vax cells, i.e. A. baumannii Ab283 cells expressing the OMP proteins OprF and fusion OprI::PcrV from P. aeruginosa in the external membrane. The expected wild-type product is 1.5 Kb-long. The figure shows PCR products obtained from DNA from several colistin-resistant derivatives of P-Vax cells, i.e. A. baumannii Ab283 cells expressing the OMP proteins OprF and fusion OprI::PcrV from P. aeruginosa in the external membrane. The mutants showing the products marked with an arrow were sequenced and ISAbAl insertions at the lpxC (c48) and lpxD (d69) genes were found. The insertions made lpxC or lpxD inactivated and therefore these P-Vax cells could not synthesize LPS. At the lower right side, a plate is showing growth of the parental strain Ab283 LPS+ but no growth of an LPS-null Ab283 derivative or the LPS-null mutants of P-Vax cells c48 and d69 on agar-MacConkey media. Loss of LPS is liked to inability to grow on agar-MacConkey media. In addition, the results of quantification of the LPS (endotoxin) by a chromogenic LAL assay (Pierce™ Chromogenic Endotoxin Quant Kit, Thermo Fisher Scientific) in cultures of several strains are shown. The only strain showing endotoxin levels above the detection level of the kit was the parental strain A. baumannii Ab283 LPS+. The results showed absence of endotoxin (LPS) of an LPS-null Ab283 derivative or the LPS-null mutants of K-Vax cells (clones 15 and 25) and P-Vax cells c48 and d69.

FIG. 5. Examples of confirmatory PCRs of the integration after a 2^{nd} recombination event of the expression constructs into the A. baumannii chromosome. A: at the left side there are PCR products obtained by using specific primers for amplification of the genomic region shown above the gel. The genomic region shown is the final expression construct after the second recombination event in A. baumannii Ab283 cells transformed with the plasmid pVXD50::K-Vax, integrated at the cysI locus. The integration led to the construction of K-Vax cells, i.e. A. baumannii Ab283 cells expressing the OMP proteins OmpA and OmpK36 from K. pneumoniae. As shown, the PCR products marked with arrows showed the right size expected for the final integration of the expression construct in several clones (clone 20, 4, 42, and 44). In the gel shown at the right side the results of several confirmatory PCR products by amplification of internal sequences in the expression construct are shown, made in cells of K-Vax clones 4, 42, 44, and 20, respectively; B: PCR products obtained by using specific primers for amplification of the genomic region shown above the gel. The genomic region shown is the final expression construct after the second recombination event in A. baumannii Ab283 cells transformed with the plasmid pVXD50::Eco1-Vax, integrated at the cysI locus. The integration led to the construction of Eco1-Vax cells, i.e. A. baumannii Ab283 cells expressing the OMP proteins OmpA and OmpX from E. coli. The PCR products of 4 clones of Eco1-Vax cells showed the right size expected for the final integration of the expression construct, confirming the construction of Eco1-Vax cells.

FIG. 6. Example of confirmation of expression and location of the heterologous OMP antigens in the drug-substance K-Vax. Upper figure: Surface exposure of Kp-OmpK36 and Kp-OmpA. Western-Blot with membrane samples obtained from DS3 (K-Vax) or Ab283 LPS-vaccine batches. DS3 (K-Vax) cells are A. baumannii LPS-null Ab283 cells expressing the OMPs from K. pneumoniae OmpA and OmpK36. Murine polyclonal sera against either Kp-OmpK36 or Kp-OmpA was used as primary antibody. Treatment with Proteinase K is indicated by +K (treated) or −K (untreated) in each sample). Protocol described in the examples. Lower figure: Expression analysis in DS3 (K-Vax) by ELISA in vaccine batches. ELISA plates were coated with either inactivated DS3 cells or carrier cells Ab-283 LPS-, after treatment with Proteinase K (+) or untreated (−). Detection with primary antibody was done with polyclonal sera raised against recombinant versions of Kp-OmpA, Kp-OmpK36, or Ab-Omp22. Average signals (error bars CI95%) from 3 technical replicates are shown.

FIG. 7. Example of confirmation of expression and location of the heterologous OMP antigens in the drug-substance P-Vax. Upper, left: Surface exposure of Pa-OprF. Western-Blot with membrane samples obtained from DS4 (P-Vax), Ab283 LPS-vaccine batches or from wild-type P. aeruginosa strain PA14 cells. DS4 (P-Vax) cells are A. baumannii LPS-null Ab283 cells expressing the OMPs from P. aeruginosa OprF and fusion protein OprI::PcrV. Cultures were washed after growth, resuspended in PBS and treated with 0.5 mg/ml of Proteinase K for 1 h at 37° C. Western-Blot with the monoclonal antibody Hy221-n5 against Pa-OprF. Treatment with Proteinase K is indicated by +K (treated) or −K (untreated) in each sample). Protocol described in the examples; Upper, right: Surface exposure of Pa-OprI::PcrV fusion. Western-Blot with membrane samples obtained from DS4 (P-Vax), Ab283 LPS-vaccine batches or from wild-type P. aeruginosa strain PA14 cells. Cultures were washed after growth, resuspended in PBS and treated with 0.5 mg/ml of Proteinase K for 1 h at 37° C. Western-Blot with the monoclonal antibody Hy243-n20 against Pa-OprI. Treatment with Proteinase K is indicated by +K (treated) or −K (untreated) in each sample). Protocol described in the examples; Lower part: Detection of Pseudomonas aeruginosa OprF, OprI and PcrV in 2 clones of DS4 (P-Vax) measured by ELISA using polyclonal sera against recombinant versions of Pa-OprF, Pa-OprI, and Pa-PcrV, respectively. Bars represents the mean of 3 replicates, error bars show the s.d. The ELISA plates were coated with either wild-type cells of P. aeruginosa PA14 strain, the carrier cells Ab283 LPS-, and clones c48 and d69 of DS4opt (P-Vax). Statistical differences compared to Ab283 LPS—by one-way ANOVA multiple comparisons indicated with asterisks. (*p<0.05;** p<0.01; ***p<0.001; ****p<0.0001) FIG. 8. Example of confirmation of expression and location of the heterologous OMP antigens in the drug-substance KapaVax. Surface exposure of heterologous antigens (Upper and Mid figures). Western-Blot with membrane samples obtained from KapaVax or Ab283 LPS-vaccine batches. KapaVax cells are A. baumannii LPS-null Ab283 cells expressing the OMPs from K. pneumoniae OmpA and OmpK36 and P. aeruginosa OprF and fusion protein OprI::PcrV. Cultures were washed after growth, resuspended in PBS and treated with 0.5 mg/ml of Proteinase K for 1 h at 37° C. Western-Blot with polyclonal anti-sera against recombinant proteins Kp-OmpA, Kp-OmpK36, Pa-OprF, Pa-OprI and Pa-PcrV. Note that both anti-Pa-OprI and anti-Pa-PcrV recognize the same fusion protein OprI::PcrV expressed in KapaVax2. Treatment with Proteinase K is indicated by +(treated) or −(untreated) in each sample). Mb: Outer-Membrane extract; S: extract supernatant. Protocol described in the examples. Lower figure: Detection of heterologous OMP antigens measured by ELISA using polyclonal anti-sera against recombinant proteins of each antigen. Bars represents the mean of 3 replicates, error bars show the standard deviation. Significant differences in pairwise comparisons 2-tailed T test indicated with asterisks. The anti-PaOprI and anti-PcrV sera target the same OprI::PcrV fusion protein. The ELISA plates were coated with either KapaVax2@cysI vaccine cells (light grey bars) or the carrier cells Ab283 LPS- (dark grey bars).

FIG. 9. Examples of expression of heterologous antigens from E. coli and A. pleuropneumioniae in LPS-null A. baumannii cells. Upper panel: examples of differential expression of OMPs between LPS-null A. baumannii carrier cells and cells expressing E. coli antigens. The upper arrows indicate a band that is present in the drug substance Eco2-Vax mentioned in Example 1. The band correspond to the antigen FyuA with expected molecular weight of 73.3 KDa (native protein). Lower arrows indicate a band present in the drug substance Eco1-Vax mentioned in Example 1, but not present in the carrier cell LPS-null A. baumannii LPS- (Ab LPS-) or in the candidate Eco2-Vax. The band corresponds with the expected sized of E. coli OmpA (38 KDa, native protein). Lower panel: example of differential expression of OMPs between the LPS-null A. baumannii carrier cell and cells expressing OMP antigens from A. pleuropneumoniae (APP). The arrow indicates a band that is present in the drug substance Appe3-Vax mentioned in Example 1, expressing the APP OmpA (APP OmpA). The band corresponds to the antigen OmpA with expected a molecular weight of 37.5 KDa (native protein). This band is not present in the control lane of A. baumannii carrier cell LPS-(Ab LPS-).

FIG. 10. shows a survival plot of murine sepsis model by K. pneumoniae infection. Animals were immunized with 2 doses (10⁹ inactivated cells/dose) in alternate weeks with KapaVax2 (N=18), the carrier Ab283 LPS-cells (N=8) or vehicle alone (N=25). IP challenge with the strain Klebsiella pneumoniae ATCC43816 (day 7 after 2nd immunization). The plot merged data from 3 independent experiments, with challenge doses 8.5×10³, 6.7×10³, and 6.7×10³ cfu, respectively, corresponding with the Minimal Lethal Dose (MLD). Survival was monitored for 7-12 days. Statistical differences were measured by Log-rank (Mantel-Cox) test (ns stands for non-significant). K. pneumoniae ATCC43816 is a hypervirulent, hypercapsulated clinical isolate, producing acute pneumonia and systemic spread in humans and mice.

FIG. 11 shows a survival plot of sepsis model by P. aeruginosa infection. Animals were immunized with 2 doses (10⁹ inactivated cells/dose) in alternate weeks of the vaccine candidate KapaVax2, or vehicle alone. IP challenge with the strain P. aeruginosa PA14 (4.4×10⁶ cfu; equivalent to the MLD) was carried out 7 days after second immunization. Survival was monitored for 7 days. N/group=8. Statistical differences of the survival curves are measured by Log-rank (Mantel-Cox) test. P. aeruginosa PA14 is a virulent wound-infection clinical isolate

FIG. 12. shows a survival plot of murine sepsis model by K. pneumoniae infection. Animals were immunized with 2 doses in alternate weeks with DS3 (K-Vax) (N=10; dose of K-Vax 108 inactivated cells/dose), the carrier Ab283 LPS-cells (N=8; dose 10⁹ inactivated cells/dose) or vehicle alone (N=25). IP challenge with the strain Klebsiella pneumoniae ATCC43816 (day 7 after 2nd immunization). The challenge doses was equivalent to 5 times the Minimal Lethal Dose (5×MLD). Survival was monitored for 7-12 days. Statistical differences were measured by Log-rank (Mantel-Cox) test. K. pneumoniae ATCC43816 is a hypervirulent, hypercapsulated clinical isolate, producing acute pneumonia and systemic spread in humans and mice.

FIG. 13 shows a survival plot of sepsis model by P. aeruginosa infection. Animals were immunized with 2 doses (10⁹ inactivated cells/dose) in alternate weeks of the vaccine candidate DS4 (P-Vax), the carrier Ab283 LPS-cells, or vehicle alone. IP challenge with the strain P. aeruginosa PA14 (1.3×10⁶ cfu; 1×MLD) was carried out 7 days after second immunization. Survival was monitored for 7 days. N/group=10. Statistical differences of the survival curves are measured by Log-rank (Mantel-Cox) test. P. aeruginosa PA14 is a virulent wound-infection clinical isolate.

FIG. 14. Survival plot of sepsis model by A. baumannii. Animals (N=8 per group) were immunized with 2 doses in alternate weeks of KapaVax2 (10⁹ inactivated cells/dose formulated with 0.83 mg Aluminum Hydroxide per dose as adjuvant), AcinetoVax (10⁹ inactivated cells/dose formulated with 0.83 mg Aluminum Hydroxide per dose as adjuvant) or vehicle (only adjuvant). IP challenge at day 7 after 2nd immunization, with 4.8×10⁶ cells (lx MLD) of A. baumannii ATCC19606. Survival was monitored for 7 days. A. baumannii ATCC19606 is a urine infection clinical isolate used as reference strain in sepsis and pneumonia models of infection in mice.

FIG. 15 shows IgG levels against K. pneumoniae cells. ELISA recognition of K. pneumoniae ATCC43816 cells by antisera from animals immunized with 2 doses of KapaVax (N=10) or vehicle alone (N=10), sampled at day 21. Statistical analysis by 2-tailed Mann-Whitney test (ns, p>0,05; *p<0.05; ** p<0.005; ***p<0.001). Red dashed line indicates detection limit.

FIG. 16 shows IgG levels against K. pneumoniae antigens. ELISA recognition of recombinant proteins Kp-OmpA and Kp-OmpK36 by antisera from animals immunized with KapaVax (N=4) or vehicle alone (N=4), sampled at day 21 (7 days after 2nd immunization). Statistical analysis by Unpaired 2-tailed T-test (ns p>0,05; *p<0.05; ** p<0.005; ***p<0.001). Red dashed line indicates detection limit.

FIG. 17 shows IgG levels against P. aeruginosa cells. ELISA recognition of P. aeruginosa PA14 cells by antisera from animals immunized with KapaVax (N=5) or vehicle alone (N=5), sampled at day 21 (7 days after 2nd immunization). Statistical significance: ns p>0,05; *p<0.05; ** p<0.005; ***p<0.001. Red dashed line indicates detection limit.

FIG. 18 shows IgG levels against P. aeruginosa antigens. ELISA recognition of recombinant proteins by antisera from animals immunized with 2 doses of KapaVax (N=4) or vehicle alone (N=4), sampled at day 21 (7 days after 2nd immunization). Statistical significance: ns p>0,05; *p<0.05; ** p<0.005; ***p<0.001. Red dashed line indicates detection limit.

FIG. 19 shows IgG levels against A. baumannii cells. ELISA recognition of A. baumannii ATCC19606 cells by antisera from animals immunized with 2 doses of KapaVax2 (N=9) or vehicle alone (N=9), sampled at day 21 (7 days after 2nd immunization). Boxes show IQR, horizontal line and cross show median and mean, respectively. Error bars extend to the CI95%. Statistical analysis by Unpaired 2-tailed Mann-Whitney test (ns p>0,05; *p<0.05; ** p<0.005; ***p<0.001). Red dashed line indicates detection limit.

FIG. 20 shows ELISA IgG titers (1:dilution) of detection of 15 clinical isolates strains of K. pneumoniae (9/15 from Low and Middle Income Countries, LMICs), P. aeruginosa (8/15 LMICs) by antisera from KapaVax2 (LPS-deficient A. baumannii cells edited for expression of K. pneumoniae OmpA and OpmK36 and P. aeruginosa OprF and fusion protein OprI::PcrV) and AcinetoVax (LPS-deficient A. baumannii cells).

FIG. 21. shows ELISA IgG titers (right panel; 1:dilution) of detection of 15 clinical isolates strains of A. baumannii (10/15 from Low and Middle Income Countries, LMICs) by antisera from KapaVax2 (LPS-deficient A. baumannii cells edited for expression of K. pneumoniae OmpA and OpmK36 and P. aeruginosa OprF and fusion protein OprI::PcrV) and AcinetoVax (LPS-deficient A. baumannii cells). The left panel shows information about the collection of A. baumannii clinical isolates used, specifically the country of origin of the clinical isolate and the international clone type of A. baumannii to which they belong to.

FIG. 22 shows Expression of A. baumannii antigens in KapaVax and AcinetoVax vaccine batches: total lysate preparations from vaccine batches (2×10¹⁰ cells/ml). Samples were run on gels with 4-16% acrylamide gradient. Panels show the signal obtained with monoclonal antibodies raised against OmpA (left) and Omp22 (right).

FIG. 23 shows Surface exposure of A. baumannii Omp22 at the OM of KapaVax and the carrier cell Ab283 LPS-. Cultures were washed after growth, resuspended in PBS and treated with 0.5 mg/ml of Proteinase K for 1 h at 37° C. Western-Blot with a monoclonal antibody raised against Ab-Omp22. Treatment with Proteinase K is indicated by +(treated) or −(untreated) in each sample). Mb: Outer-Membrane extract; S: extract supernatant. Protocol described in the examples.

FIG. 24. Upper panel: Coomassie staining of A. baumannii OMVs purified fractions (left) and Western Blot for detection of A. baumannii Omp22 in OMVs purified fractions (right). Omp22 is detected with a monoclonal antibody in LPS-null A. baumannii Ab283 carrier cells (up) and K-Vax (DS3) cells (LPS-null A. baumannii Ab283 expressing the OMP antigens OmpA and OmpK36 from K. pneumoniae; down). Lower panel: Each sample from every fraction of the purification protocol for OMVs (F1-F10; see Example) are analyzed by WB, either from A. baumannii LPS-carrier cell (−) or DS3 candidate (D) expressing K. pneumoniae heterologous antigens OmpA and OmpK36. Polyclonal antisera obtained by immunizing C57BL/6J females with recombinant proteins OmpA or OmpK36 are used as primary antibody.

### DESCRIPTION OF EMBODIMENTS

The following detailed description discloses specific and/or preferred variants of the individual features of the invention. The present invention also contemplates as particularly preferred embodiments those embodiments, which are generated by combining two or more of the specific and/or preferred variants described for two or more of the features of the present invention. Unless expressly specified otherwise, the term “comprising” is used in the context of the present document to indicate that further members may optionally be present in addition to the members of the list introduced by “comprising”.

The present invention is directed to:
a. an A. baumannii strain deficient in lipopolysaccharide (LPS) characterized by the partial or complete inactivation of one or various cellular nucleic acid molecules that encode endogenous LPS biosynthesis genes; wherein the A. baumannii strain deficient in lipopolysaccharide (LPS) is characterized by the partial or complete inactivation of the genes selected from the list consisting of lpxA, lpxB, lpxC, lpxD, lpxK, lpxL and/or lpxM; wherein the A. baumannii strain deficient in lipopolysaccharide (LPS) expresses one or more heterologous, preferably outer-membrane protein (OMP), antigens with targeted location at the Outer Membrane, so that there is a surface exposure of such heterologous antigens; or
b. an outer membrane vesicle (OMV) derived from an A. baumannii strain deficient in lipopolysaccharide (LPS) as defined in the paragraph above.

Inactivated LPS-null A. baumannii cells can be used efficiently as vaccines to prevent infections by A. baumannii. The efficient antigenic presentation of this immunogen is shown by the rapid humoral response and significant T-cell mediated response raised in mice. Because the most variable antigenic component of the bacterial cell wall, i.e. the LPS, is not present in the immunogen, the immune response is directed against conserved outer-membrane proteins, which are presented in multiple copies and, importantly, in their native conformation. This leads to a very universal immune response, able to neutralize infections by diverse variants of A. baumannii with the same vaccine product. The properties of the immune response raised by the inactivated LPS-null whole cells of A. baumannii might presumably overcome the classic challenges of vaccination against bacterial pathogens, like the lack of immunogenicity or too much specificity of vaccines based on recombinant proteins or defined sugar antigens from the LPS or the capsular polysaccharide.

The aim of the present invention is to take advantage of the immunogenic properties of the LPS-null cells of A. baumannii and extend their applicability to the fight against other bacterial pathogens, and specifically against Gram-negative bacteria, including but not limiting to Klebsiella pneumoniae, Pseudomonas aeruginosa, Acinetobacter baumannii, and Escherichia coli. The technology can be applied to any Gram-negative bacterium, for instance to Actinobacillus pleuropneumoniae, the causative agent of porcine pleuropneumonia.

The present invention teaches methods for construction of A. baumannii LPS-null cells expressing on the outer-membrane OMPs from A. baumannii and from other heterologous bacterial species in the same cell. The authors of the present invention demonstrate that expression of OMP proteins from other bacterial cells in A. baumannii can be achieved. The nature of these OMP proteins is well known in the art (Koebnik et al., 2000; Smithers et al. 2021). The outer membrane protects Gram-negative bacteria against a harsh environment. At the same time, the embedded proteins fulfil several tasks that are crucial to the bacterial cell, such as solute and protein translocation, as well as signal transduction. One of the types of OMP considered in this invention are integral OMP proteins (Koebnik et al. 2000). Unlike membrane proteins from all other sources, integral OMP proteins do not consist of transmembrane alpha-helices, but instead fold into antiparallel beta-barrels. They include the OmpA membrane domain, the OmpX protein, phospholipase A, general porins, substrate-specific porins, and iron siderophore transporters (Koebnik et al. 2000). The second type of OMP proteins considered in this invention are bacterial lipoproteins (Juncker et al. 2003, Smithers et al. 2021). Bacterial lipoproteins are a class of lipid-post/translationally modified, outer-membrane anchored proteins that perform a variety of often essential functions (Smithers et al. 2021). The characteristic feature of all lipoproteins is a signal sequence in the N-terminal end, followed by a cysteine. So far, a few hundred putative lipoproteins in Gram-negative bacteria have been annotated (Juncker et al. 2003). The presence of a transmembrane domain for anchoring to the outer-membrane of Gram-negative bacteria makes the transmembrane subunits of fimbria also a type of protein considered in the present invention (Zhang et al. 2000; Antenuci et al. 2020).

Since expression of the heterologous OMP proteins of any family, including the families described in Koebnik et al., 2000 (integral OMPs) or in Smithers et al. 2021 (bacterial lipoproteins), is intended to be done in A. baumannii cells and the fate of the expressed OMP proteins is intended to be the outer-membrane of A. baumannii, in the present invention the heterologous OMP proteins include a signal sequence from an OMP protein that can be processed by A. baumannii cells to promote the location of the expressed protein in the outer-membrane of A. baumannii. Preferably, the signal sequence derives from any OMP of A. baumannii, and most preferably, from the OmpA protein of A. baumannii. It is noted that the OmpA protein of A. baumannii consists of SEQ ID NO 1.

The present invention also encompasses any signal sequence that has at least 90%, 91%, 92%, 93%, 94%, 95%, 96%, 97%, 98%, or 99% identity with the full/length sequence of SEQ ID NO 1 and/or known signal sequences of OMP proteins from A. baumannii with the proviso that these sequences can be processed by A. baumannii cells to promote the location of the expressed protein in the outer-membrane of A. baumannii.

The terms “sequence identity” or “percent identity” in the context of antigens, peptides or proteins indicated through-out the present invention, refers to two or more sequences or subsequences that are the same (“identical”) or have a specified percentage of amino acid residues that are identical (“percent identity”) when compared and aligned for maximum correspondence with a second molecule, as measured using a sequence comparison algorithm (e.g., by a BLAST alignment, or any other algorithm known to persons of skill), or alternatively, by visual inspection.

Therefore, the expression of the heterologous antigens in the outer membrane of the A. baumannii strain deficient in lipopolysaccharide (LPS) of the present invention comprises three intrinsic properties. First, the post-translational processing of the heterologous OMP proteins must be directed by a signal sequence derived from an OMP protein as defined above, which could be processed by A. baumannii to direct the location of the expressed protein to the external membrane of A. baumannii. Second, the heterologous OMP proteins must include OMP transmembrane domains, including the typical transmembrane domains of integral OMP proteins as described in Koebnik et al. 2000 or from bacterial lipoproteins as described in Juncker et al. 2003, which will allow insertion of the expressed protein at the external membrane of A. baumannii. And third, the heterologous OMP proteins must include immunogenic domains. It is noted that the sera from humans infected by bacterial pathogens recognize predominantly OMP proteins on a crude protein extract from the bacterial cells, indicating the presence of a significant titer of serum antibodies raised against the bacterial OMP proteins.

It is noted that the immunogenic domains of the heterologous OMP proteins expressed in A. baumannii are those included in the wild type OMPs from the heterologous pathogen. In the examples of the present specification the construction of different drug-substances based on A. baumannii inactivated cells is shown. In particular, the examples show expression of the full-length proteins—i.e. the native full-length OMP sequence including the native transmembrane domains and immunogenic domains—from integral OMPs from K. pneumoniae (OmpA and OmpK36), P. aeruginosa (OprF), E. coli (OmpA, OmpX, FuyA, HmA, IutA) or A. pleuropneumoniae (OmpA, OmpW, TbpA, and ApfA).

It is further noted that the immunogenic domains of the heterologous OMP proteins expressed in A. baumannii can be engineered to remove unwanted sequences or to add extra immunogenic domains either from OMP proteins or any other protein from a pathogen, if such removal or addition does not preclude the correct post-translational processing and integration of the expressed OMP protein into the external membrane of A. baumannii. In the examples of the present specification, the immunogenic domains of the Type 3 secretion system protein PcrV from P. aeruginosa were fused to the lipoprotein of the outer-membrane OprI from P. aeruginosa.

It is noted that the OMP proteins expressed in A. baumannii shown in the examples belong not only to different bacterial pathogens but also to different OMP types, including several integral OMP families as described in Koebnik et al. 2000, including proteins from the small beta-barrel membrane anchors families OmpA (OmpA from either K. pneumoniae, E. coli or A. pleuropneumoniae) or OmpX (OmpX from E. coli), classical trimeric porins like OmpK36 from K. pneumoniae, non-specific porins like OprF from P. aeruginosa, or substrate-specific porins like TbpA from A. pleuropneumoniae, or TonB-dependent iron receptors like FuyA, Hma and IutA from E. coli. It is also noted that the OMP proteins that could be directed to the outer-membrane of the A. baumannii cells by the methods of the present invention could be lipoproteins of the outer-membrane like OprI from P. aeruginosa, or the membrane-anchored Type IV fimbria subunit ApfA from A. pleuropneumomiae, as shown in the examples.

In addition, the authors of the present invention demonstrate that, preferably inactivated, whole cells of A. baumannii deficient in LPS expressing one or multiple copies of antigenic outer-membrane heterologous proteins from one or more microorganisms, upon being inoculated in a subject, preferably a human subject, in need thereof, produce immunization not only against A. baumannii infection but also against infections caused by any microorganisms originally expressing the one or multiple copies of the antigenic outer-membrane proteins, which demonstrates the utility of these cells as prophylactic multivalent vaccines.

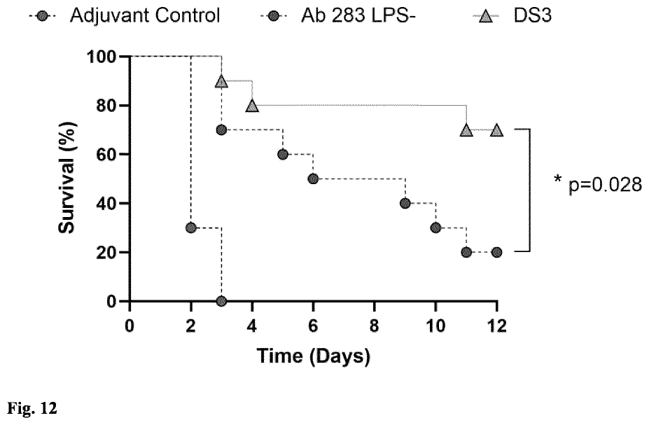

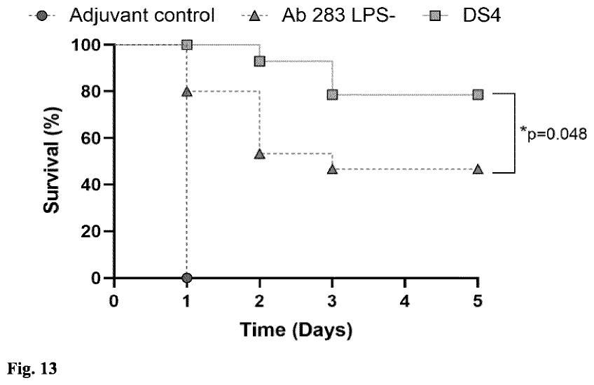

It is noted that the immunodominant proteins of the vaccine consisting of inactivated LPS-null cells of A. baumannii are the OMP proteins from A. baumannii OmpA and Omp22, which are the most abundant integral OMP proteins at the external membrane of these cells. It is also noted and shown in the examples that these proteins are still present as the most abundant proteins detected in the external membrane of the A. baumannii LPS-null cells expressing on the external membrane OMP proteins from other bacterial pathogens. Importantly, it is noted and shown in the examples that the presence of the A. baumannii antigens in LPS-null A. baumannii cells expressing OMP proteins from a different heterologous bacterial pathogen not only raises protective immunity against an infection produced by A. baumannii but that also raises partial cross-protective immunity against an infection produced by the heterologous pathogen. The authors showed, for instance, that a drug-substance consisting of inactivated LPS-null cells of A. baumannii expressing the OMP proteins OmpA and OmpK36 from K. pneumoniae used as vaccine is able to neutralize an infection produced by a hypervirulent, hypercapsulated strain K. pneumoniae ATCC43816 (see FIGS. 10 and 12); and removal of the heterologous OMP proteins OmpA and OmpK36 from K. pneumoniae decreases significantly the protection against the infection produced by K. pneumoniae ATCC43816 although there is still partial cross-protective protection raised by the A. baumannii antigens (see FIGS. 10 and 12). It is also shown in the examples that a drug-substance consisting of inactivated LPS-null cells of A. baumannii expressing the OMP proteins OprF and the fusion OprI::PcrV from P. aeruginosa used as vaccine is able to neutralize an infection produced by a hypervirulent strain P. aeruginosa PA14 (see FIG. 13); and that removal of the heterologous OMP proteins OprF and the fusion OprI::PcrV from P. aeruginosa decreases significantly the protection against the infection produced by P. aeruginosa PA14 although there is still partial cross-protective protection raised by the A. baumannii antigens (see FIG. 13).

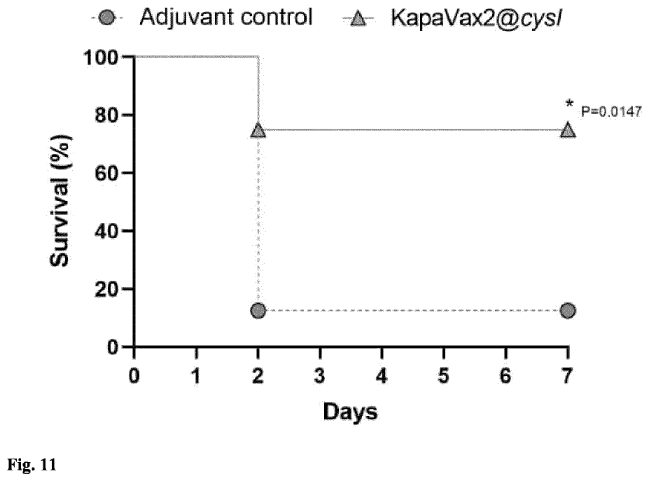

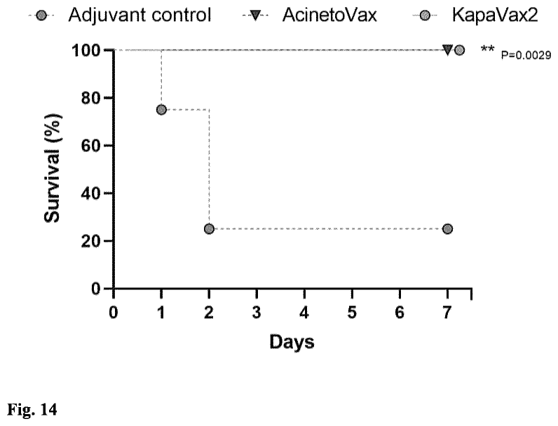

The ability of the technology for delivering multi-pathogen vaccine candidates is another aspect of the present invention. The authors showed, for instance, that a drug-substance consisting of inactivated LPS-null cells of A. baumannii expressing the OMP proteins OmpA and OmpK36 from K. pneumoniae and the OMP proteins OprF and the fusion OprI::PcrV from P. aeruginosa in the same cells can be constructed. When used as vaccine, the immunity raised by such drug-substance is able to neutralize an infection produced by a hypervirulent, hypercapsulated strain K. pneumoniae ATCC43816 (see FIG. 10) and also the infection produced by a hypervirulent strain P. aeruginosa PA14 (see FIG. 11). In addition, the immunity raised by the drug-substance, still containing the antigens from A. baumannii, as all drug-substances of the present invention do, led to protection against lethal sepsis produced by A. baumannii ATCC19606 (see FIG. 14).

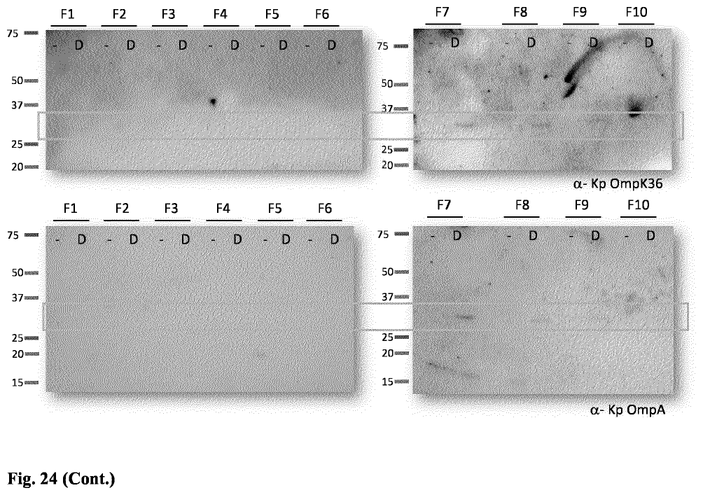

It is also noted and shown in the examples that expression of the heterologous OMP antigens in the LPS-null A. baumannii cells does not preclude the formation of the typical outer-membrane vesicles derived from Gram-negative bacteria (see FIG. 24).

Consequently, a first aspect of the present invention refers to:
a. an A. baumannii strain deficient in lipopolysaccharide (LPS) characterized by the partial or complete inactivation of one or various cellular nucleic acid molecules that encode endogenous LPS biosynthesis genes; wherein the A. baumannii strain deficient in lipopolysaccharide (LPS) is characterized by the partial or complete inactivation of the genes selected from the list consisting of lpxA, lpxB, lpxC, lpxD, lpxK, lpxL and/or lpxM; and wherein the A. baumannii strain deficient in lipopolysaccharide (LPS) expresses one or multiple copies of antigenic outer-membrane heterologous proteins from one or more microorganisms with targeted location at the Outer Membrane; and/or
b. an outer membrane vesicle (OMV) derived from an A. baumannii strain deficient in lipopolysaccharide (LPS) as defined in the paragraph above.

It is understood that “lipopolysaccharide (LPS) or lipooligosaccharide” is a component that is found on the external membrane of Gram-negative bacteria. The term LPS is used often and interchangeably with “endotoxin”, due to its history of discovery. LPS consists of a polysaccharide chain and the rest is lipid, known as lipid A, which is responsible for the endotoxin activity. The polysaccharide chain is variable between different bacteria and determines the serotype. Endotoxin is of approximately 10 kDa in size but can form large aggregates of up to 1000 kDa. Humans are able to produce antibodies against LPS, but in general these antibodies can only protect against bacteria of a specific serotype. Endotoxin is responsible for many of the clinical manifestations of infections caused by Gram-negative bacteria such as Neisseria meningitidis and A. baumannii.

It is understood that “cells of the A. baumannii” in the present invention are those cells pertaining to the domain Bacteria, phylum Proteobacteria, class Gammaproteobacteria, order Pseudomonadales, family Moraxellaceae, genus Acinetobacer, species Acinetobacter baumannii. The species of Acinetobacter are strictly aerobic non-fermenting and non-motile bacilli that are oxidase negative and appear in pairs by microscopy. They are distributed widely in nature and are important in soil and contribute to its mineralization.

It is understood that the A. baumannii strains deficient in lipopolysaccharide (LPS) of the present invention are preferably whole inactivated cells, whole “inactivated cells” in the present invention are cells that do not have the ability to replicate but conserve their immunogenic capacity. The cells of the present invention are inactivated prior to their inoculation to prevent their replication in the host, and therefore prevent infection produced by their administration. The inactivation of the cells of the invention can be performed using diverse methods known in the state of the art, for example, although not limited to, adsorption, heat, ultraviolet light, ionizing radiation, ultrasound, phenol, formol, formaldehyde, crystal violet, glyceraldehyde, ethylene oxide, propiolactone, ethylenamina, bromoethyleneamina or formalin. In a preferred form the cells of the invention are inactivated with heat. In another preferred form the cells of the invention are from the species Acinetobacter baumannii and they are inactivated with heat.

In a preferred embodiment of this aspect of the invention, the deficiency in LPS, as taught in EP2942389A1, can be achieved by partial or complete inactivation of one or various cellular molecules of nucleic acids that encode the endogenous genes for the LPS subunits, particularly lpxA, lpxB and/or lpxC of LPS, that leads to complete LPS-loss. The sequences of lpxA, lpxB and/or lpxC from Acinetobacter species, particularly A. baumannii. In a preferred embodiment of the invention, the endogenous genes of LPS are selected from lpxA, lpxB and/or lpxC, or any combination of these genes.

Preferably, the one or more mutations in the endogenous LPS biosynthesis genes of the A. baumannii strain deficient in lipopolysaccharide (LPS) of the first aspect of the invention, are obtained by selecting colistin-resistant mutants on plates supplemented with the antibiotic colistin, and screening of selected colistin-resistant mutants for mutations in the LPS synthesis genes. In this sense, the authors of this invention show in the examples that multiple diverse strains of A. baumannii can become colistin-resistant by different mutations in the LPS-synthesis genes leading to LPS-loss (see Table 1).

TABLE 1

| | | Colistin | EU/ |
|---|---|---|---|
| | | MIC | 10⁶ |
| Strain | Mutation/Description | (mg/l) | cells^{a} |
| ATCC 19606 | Antibiotic susceptible reference | ≤0.25 | 1060 ± |
| | strain (parental) | | 814 |
| IB002 (derived | ISAba11 insertion at nucleotide | >128 | <1 |
| from ATCC 19606) | 372 of lpxC gene | | |
| IB003 (derived | ISAba11 insertion at nucleotide | >128 | <1 |
| from ATCC 19606) | 394 of lpxC gene | | |
| IB004 (derived | Deletion of nucleotide 461 of | >128 | <1 |
| from ATCC 19606) | lpxA gene generating frameshift | | |
| | after S153 | | |
| AB001 | Antibiotic susceptible reference | ≤0.25 | 1180 ± |
| | strain (parental) | | 687 |
| IB006 (AB001- | ISAba11 insertion at nucleotide | >128 | <1 |
| VXD, derived from | 393 of lpxC gene | | |
| AB001) | | | |
| IB010, derived | 462 nucleotide deletion in lpxD | >128 | <1 |
| from AB001) | gene (nucleotides 104-565) | | |
| Ab283 | Antibiotic susceptible reference | ≤0.25 | 1198 ± |
| | strain (parental) | | 776 |
| K1 (derived from | Deletion at lpxA gene | >128 | <1 |
| Ab283 by site- | | | |
| directed | | | |
| mutagenesis) | | | |
| DS1 (Ab283 LPS- | ISAba11 insertion at lpxC gene | >128 | <1 |
| null, derived from | | | |
| Ab283) | | | |
| DS2 (Kapa Vax, | ISAba11 insertion at lpxC gene | >128 | <1 |
| derived from | | | |
| Ab283) | | | |
| DS3 (K-Vax, | ISAba11 insertion at lpxC gene | >128 | <1 |
| derived from | | | |
| Ab283)) | | | |
| DS4 (P-Vax, | ISAba11 insertion at lpxD gene | >128 | <1 |
| derived from | | | |
| Ab283)) | | | |
| DS5 (Eco-Vax, | ISAba11 insertion at lpxC gene | >128 | <1 |
| derived from | | | |
| Ab283)) | | | |
| DS6 (Appe-Vax, | ISAba11 insertion at lpxC gene | >128 | <1 |
| derived from | | | |
| Ab283)) | | | |

^{a}Data represent the mean ± the standard error of the mean of three independent assays.
EU; Endotoxin Units

Table 1. Examples of LPS-null derivatives from several parental A. baumannii strains obtained by the authors. The table shows examples of LPS-null mutants obtained by the authors from three different unrelated clinical isolates of A. baumannii, specifically an old clinical isolate now available at the ATCC as strain ATCC19606 and used as reference A. baumannii strain in the experimentation on animal models elsewhere, and clinical isolates from an outbreak in the Hospital Virgen del Rocio of Seville in 2002, named IB001 and Ab283.

All LPS-null mutants were constructed by selection of colistin-resistant mutants on plates supplemented with colistin, excepting K1, which was obtained by direct mutagenesis of the lpxA gene. The table shows the description of the mutation found or produced at an LPS-synthesis gene, the minimal inhibitory concentration of colistin and the amount of endotoxin (LPS) measured in a cell culture of each strain by an Chromogenic LAL Assay for quantification of bacterial endotoxin.

In a preferred embodiment of the first aspect of the invention, the sequence of the LPS-synthesis gene is inactivated e.g by construction of a suicide vector that contains the gene lpxA, lpxB, lpxC, lpxD, lpxK, lpxL and/or lpxM or any of their combination, or interrupting with a marker gene for selection, transforming the target cells with the vector and screening for positive cells that are negative for LPS expression. In yet another preferred embodiment of the invention, the cell of Acinetobacter deficient in LPS is obtained by deletions and/or insertions of one or various nucleotides in nucleic acid sequences encoding the gene involved in the biosynthesis of LPS and/or the sequences that control their expression. The deletions and/or insertions can be generated by homologous recombination, insertion of transposons, or other adequate methods known in the state of the art.

In a preferred embodiment of the first aspect of the invention, although the cell of the invention is preferably an A. baumannii cell, such cell can be potentially replaced by other Acinetobacter species Acinetobacters such as those selected from the list consisting of; Acinetobacter baylyi, A. beijerinckii, A. bereziniae, A. boissieri, A. bouvetii, A. brisouii, A. calcoaceticus, A.gerneri, A. guillouiae, A. grimontii, A. gyllenbergii, A. haemolyticus, A. indicus, A. johnsonii, A. junii, A.Iwoffii, A. nectaris, A. nosocomialis, A. parvus, A. pittii, A. puyangensis, A. radioresistens, A. rudis, A. schindleri, A. soli, A. tandoii, A. tjernbergiae, A. towneri, A. ursingii or A. venetianus. In this invention, it is understood that Acinetobacter refers to the kingdom Bacteria, phylum Proteobacteria, class Gammaproteobacteria, order Pseudomonadales, family Moraxellaceae.

On the other hand, and as already indicated earlier in the specification, three intrinsic properties for the expression and exposure of the heterologous antigens in the outer membrane of A. baumannii cells of the first aspect of the invention are needed. First, the post-translational processing of the heterologous OMP proteins must be directed by a signal sequence derived from an OMP protein, which could be processed by A. baumannii to direct the location of the expressed protein to the external membrane of A. baumannii. Second, the heterologous OMP proteins must include OMP transmembrane domains typical of integral OMP proteins as described in Koebnik et al. 2000 or the typical signal sequence of bacterial lipoproteins as described in Juncker et al. 2003, which will allow insertion of the expressed protein at the external membrane of A. baumannii. And third, the heterologous OMP proteins must include immunogenic domains.

Hence, the one or more heterologous antigens (or heterologous OMP proteins) are characterized by comprising in the N-terminus, a signal sequence derived from an OMP protein, which is processed by A. baumannii to direct the location of the expressed protein to the external membrane of A. baumannii. In addition, the one or more heterologous antigens are characterized by comprising OMP transmembrane domains, including the typical transmembrane domains from integral OMP proteins as described in Koebnik et al. 2000 or from bacterial lipoproteins as described in Juncker et al. 2003, which will allow insertion of the expressed protein at the external membrane of A. baumannii. And third, the one or more heterologous antigens are characterized by comprising immunogenic domains.

Therefore, in another preferred embodiment of the first aspect of the invention, the one or more heterologous antigens (or heterologous OMP proteins) with targeted location at the Outer Membrane are characterized by comprising:
1. at the N-terminus of the heterologous antigens (or heterologous OMP protein), a signal sequence derived from an OMP protein which is processed by A. baumannii to direct the location of the expressed protein to the external membrane of A. baumannii;
2. OMP transmembrane domains typical of integral OMP proteins or from bacterial lipoproteins which allow insertion of the expressed protein at the external membrane of A. baumannii; and
3. immunogenic domains.

Preferably, the heterologous antigen (or heterologous OMP protein), is comprised by the signal sequence at the N-terminus of the protein bound directly, or optionally via a linker, to the N-terminal amino acid of the part of the protein comprising the transmembrane domains and immunogenic domains.

Preferably, as stated above, in the present invention the heterologous OMP proteins include a signal sequence from an OMP protein that can be processed by A. baumannii cells to promote the location of the expressed protein in the outer-membrane of A. baumannii. Preferably, the signal sequence derives from any OMP of A. baumannii, and most preferably, from the OmpA protein of A. baumannii. The present invention also encompasses any signal sequence that has at least 90%, 91%, 92%, 93%, 94%, 95%, 96%, 97%, 98%, or 99% identity with the full/length sequence of SEQ ID No. 1 from the OmpA protein of A. baumannii, with the proviso that the resulting sequences can be processed by A. baumannii cells to promote the location of the expressed protein in the outer-membrane of A. baumannii.

Preferably, the immunogenic domains of the heterologous OMP proteins expressed in A. baumannii are those included in the wild type OMPs from the heterologous pathogen. In the examples of the present specification the construction of different drug-substances based on A. baumannii inactivated cells is shown. In particular, the examples show expression of the full-length proteins—i.e. the native full-length OMP sequence including the native transmembrane domains and immunogenic domains—from integral OMPs from K. pneumoniae (OmpA and OmpK36), P. aeruginosa (OprF), E. coli (OmpA, OmpX, FuyA, HmA, IutA) or A. pleuropneumoniae (OmpA, OmpW, TbpA, and ApfA). All of these immunogenic domains are explicitly included in the present invention. Also preferably, it is further noted that the immunogenic domains of the heterologous OMP proteins expressed in A. baumannii can be engineered to remove unwanted sequences or to add extra immunogenic domains either from OMP proteins or any other protein from a pathogen, if such removal or addition does not preclude the correct post-translational processing and integration of the expressed OMP protein into the external membrane of A. baumannii. In the examples of the present specification, the immunogenic domains of the Type 3 secretion system protein PcrV from P. aeruginosa were fused to the lipoprotein of the outer-membrane OprI from P. aeruginosa.

In another preferred embodiment of the first aspect of the invention, the heterologous OMP proteins expressed in A. baumannii shown in the examples belong not only to different bacterial pathogens but also to different OMP types, including several integral OMP families as described in Koebnik et al. 2000, including proteins from the small beta-barrel membrane anchors families OmpA (OmpA from either K. pneumoniae, E. coli or A. pleuropneumoniae) or OmpX (OmpX from E. coli), classical trimeric porins like OmpK36 from K. pneumoniae, non-specific porins like OprF from P. aeruginosa, or substrate-specific porins like TbpA from A. pleuropneumoniae, TonB-dependent iron receptors like FuyA, Hma and IutA from E. coli. It is also noted that the heterologous OMP proteins that could be directed to the outer-membrane of the A. baumannii cells by the methods of the present invention could be lipoproteins of the outer-membrane like OprI from P. aeruginosa or the membrane-anchored Type IV fimbria subunit ApfA from A. pleuropneumomiae, as shown in the examples.

In addition, and as already indicated, the authors of the present invention demonstrate that, preferably inactivated, whole cells of A. baumannii deficient in LPS expressing the one or multiple copies of antigenic outer-membrane heterologous proteins, as defined above, from one or more microorganisms, upon being inoculated in a subject, preferably a human subject, in need thereof, produce immunization not only against A. baumannii infection but also against infections caused by any microorganisms originally expressing the one or multiple copies of the antigenic outer-membrane proteins, which demonstrates the utility of these cells as prophylactic, and even therapeutic, multivalent vaccines.

On the other hand, and as reflected above, the A. baumannii strain deficient in lipopolysaccharide (LPS) must express one or multiple copies of antigenic outer-membrane heterologous proteins from one or more microorganisms, with targeted location at its Outer Membrane. Such expression shall be carried out so that there is a surface exposure of such heterologous antigens. For that purpose, vaccine candidates of the present invention can be constructed by genome editing of A. baumannii using any allelic exchange technology for recombinant strain production that involves one or more recombination steps for insertion of an expression construct at a particular locus into the A. baumannii genome. The expression construct to be inserted could be carried into the cell by a linear piece of DNA or a plasmid. In particular, the construct coding for the one or multiple copies of the antigenic outer-membrane heterologous proteins can be inserted in any suitable locus of an A. baumannii strain previously, simultaneously or subsequently to the cell becoming deficient in LPS, as for example taught in EP2942389A1. A suitable locus is any locus on the A. baumannii chromosome that can incorporate an insert by recombination, such as: cysI, trpE, lpxA, lpxC, lpxD, lpxB, lpxK, lpxL, lpxM, and/or Tn5/Tn7 sites. It is noted that, Acinetobacter baumannii cells can be preferably transformed with a vector, preferably a suicide vector, comprising sequences for promoting recombination at the aimed target locus into the A. baumannii genome, being such locus any locus of the A. baumannii genome comprising sequences suitable to undergo recombination as detailed above.

In this sense, in a preferred embodiment of the invention, the strain is modified to express the one or more heterologous antigens (or heterologous OMP proteins) with targeted location at the Outer Membrane by insertion of an expression construct coding for the one or more heterologous antigens. More preferably, Acinetobacter baumannii cells can be preferably transformed with a vector, wherein the vector is characterized by comprising sequences for promoting recombination flanking the expression construct which in turn comprises at least one or more transcription promoter sequences, one or more ORFs encoding proteins heterologous for A. baumannii, and one or more transcription termination sequences. The promoter sequence might be any known sequence in the state of the art able to promote transcription in an A. baumannii cell. These promoter sequences are routinely used in bacteriology research. In a preferred embodiment of this invention, the promoter sequence used is a promoter sequence located upstream of the A. baumannii ORF encoding the outer-membrane protein OmpA. The expression construct might comprise several ORFs in tandem, each one flanked by a promoter and a termination, transcriptional termination, sequence upstream and downstream of the ORF, respectively, therefore built for allowing independent expression of each ORF, controlled by a specific promoter. Alternatively, the expression construct might have one or several ORFs within an operon-like structure with polycistronic expression controlled by a common promoter. The ORFs of the expression vector encode outer-membrane proteins or peptides selected by their immunogenic properties, derived from known humans or animal pathogens distinct from A. baumannii and whose expression in the A. baumannii cell is intended for raising an immune response against pathogens distinct from A. baumannii in a human or animal vaccinated with the A. baumannii cell. Since expression of the heterologous outer-membrane proteins, including proteins from any family described in Koebnik et al., 2000 (integral OMPs) or in Smithers et al. 2021 (bacterial lipoproteins), is intended to be done in A. baumannii cells and the fate of the expressed OMP proteins is intended to be the outer-membrane of A. baumannii, in the present invention the heterologous OMP proteins include a signal sequence from an OMP protein that can be processed by A. baumannii cells to promote the location of the expressed protein in the outer-membrane of A. baumannii. Preferably, the signal sequence derives from any OMP of A. baumannii, and most preferably, from the OmpA protein of A. baumannii, including any signal sequence that has at least 90%, 91%, 92%, 93%, 94%, 95%, 96%, 97%, 98%, or 99% identity with the full/length sequence of SEQ ID No. 1 from the OmpA protein of A. baumannii, and that can be processed by A. baumannii cells to promote the location of the expressed protein in the outer-membrane of A. baumannii. Preferably, ORFs of the expression vector encode heterologous antigens (or heterologous OMP proteins) characterized, as defined above, by comprising:
1. At the N-terminus of the heterologous antigens (or heterologous OMP proteins), a signal sequence derived from an OMP protein which is processed by A. baumannii to direct the location of the expressed protein to the external membrane of A. baumannii;
2. OMP transmembrane domains typical of integral OMP proteins or of bacterial lipoproteins which allow insertion of the expressed protein at the external membrane of A. baumannii; and
3. immunogenic domains.

Preferably, the signal sequence derives from any OMP of A. baumannii, and most preferably, from the OmpA protein of A. baumannii, including any signal sequence that has at least 90%, 91%, 92%, 93%, 94%, 95%, 96%, 97%, 98%, or 99% identity with the full/length sequence of SEQ ID No. 1 from the OmpA protein of A. baumannii, and that can be processed by A. baumannii cells to promote the location of the expressed protein in the outer-membrane of A. baumannii.

Preferably, the immunogenic domains of the heterologous OMP proteins expressed in A. baumannii are those included in the wild type OMPs from the heterologous pathogen. In the examples of the present specification the construction of different drug-substances based on A. baumannii inactivated cells is shown. In particular, the examples show expression of the full-length proteins—i.e. the native full-length OMP sequence including the native transmembrane domains and immunogenic domains—from integral OMPs from K. pneumoniae (OmpA and OmpK36), P. aeruginosa (OprF), E. coli (OmpA, OmpX, FuyA, Hma, IutA) or A. pleuropneumoniae (OmpA, OmpW, TbpA, and ApfA). All of these immunogenic domains are explicitly included in the present invention as potentially encoded by the ORFs of the expression construct. Also preferably, it is further noted that the immunogenic domains of the heterologous OMP proteins encoded by the ORFs of the expression construct can be engineered to remove unwanted sequences or to add extra immunogenic domains either from OMP proteins or any other protein from a pathogen, if such removal or addition does not preclude the correct post-translational processing and integration of the expressed OMP protein into the external membrane of A. baumannii. In the examples of the present specification, the immunogenic domains of the Type 3 secretion system protein PcrV from P. aeruginosa were fused to the lipoprotein of the outer-membrane OprI from P. aeruginosa.

In another preferred embodiment of the first aspect of the invention, the OMP proteins encoded by the ORFs of the expression construct shown in the examples belong not only to different bacterial pathogens but also to different OMP types, including several integral OMP families as described in Koebnik et al. 2000, including proteins from the small beta-barrel membrane anchors families OmpA (OmpA from either K. pneumoniae, E. coli or A. pleuropneumoniae) or OmpX (OmpX from E. coli), classical trimeric porins like OmpK36 from K. pneumoniae, non-specific porins like OprF from P. aeruginosa, or substrate-specific porins like TbpA from A. pleuropneumoniae, or TonB-dependent iron receptors like FuyA, Hma and IutA from E. coli. It is also noted that the heterologous OMP proteins encoded by the ORFs of the expression construct and directed to the outer-membrane of the A. baumannii cells could be lipoproteins of the outer-membrane like OprI from P. aeruginosa or the membrane-anchored Type IV fimbria subunit ApfA from A. pleuropneumomiae, as shown in the examples.

Furthermore, and as explained below in the specification, once the A. baumannii cells are transformed with the vector, preferably a suicide vector, recombination between the sequences of the target loci in the host and the sequences in the vector leads to the production of recombinant cells where the sequences of the vector have integrated into the targeted loci on the A. baumannii chromosome. This recombination event can be assisted by strategies well-known in the art, like inducing double strand breaks at the insertion site with genome-editing tools, like those based on the CRISPR methodology. In a preferred embodiment of this invention, a first recombination event leads to the insertion of the vector sequences comprising the selectable markers and the expression constructs. The selectable markers are used for selection of cells where such first recombination event has occurred. A second recombination event might lead to recombinant cells where only the expression construct but not the sequences encoding the selectable markers remain integrated at the insertion site. By using as a selection tool the lack of the selectable markes, A. baumannii cells where such second recombination event has occurred can be selected. Once selected, common techniques like PCR and DNA sequencing are used to check whether the integration of the vector sequences into the targeted loci has occurred as expected.

On the other hand, prior to, simultaneously to or subsequently to, the expression of the heterologous proteins analyzed in the recombinant A. baumannii cells is considered satisfactory, loss of the LPS is induced in the A. baumannii cells by alternative methods like site-directed mutagenesis of a LPS-synthesis gene leading to partial or complete inactivation of LPS-synthesis genes, including the genes selected from the list consisting lpxA, lpxB, lpxC, lpxD, lpxK, lpxL and/or lpxM; or by selecting A. baumannii cells resistant to the antibiotic colistin and screening the cells by common genomic methods like PCR and/or DNA sequencing for mutations leading to partial or complete inactivation of LPS-synthesis genes, including the genes selected from the list consisting of lpxA, lpxB, lpxC, lpxD, lpxK, lpxL and/or lpxM.

In a particular embodiment of this invention, when the targeted locus for insertion of the recombination events described above is a locus comprising an ORF encoding an LPS-synthesis gene, including genes selected from the list consisting of lpxA, lpxB, lpxC, lpxD, lpxK, lpxL and/or lpxM, and the recombination leads to partial or complete inactivation of such ORF, the resulting recombinant A. baumannii cells are LPS-negative cells due to partial or complete inactivation of LPS-synthesis genes, including the genes selected from the list consisting lpxA, lpxB, lpxC, lpxD, lpxK, lpxL and/or lpxM.

After having inserted the constructs at the suitable locus, such as at the cysI locus, proof of expression and localization of the heterologous antigens at the outer-membrane can be performed (as taught in the examples), then proof of selection of an LPS-negative derivative can be carried out for example by plating in colistin and selection of an LPS-negative mutant. Expression and location at the outer membrane of each antigen can be confirmed by Western Blot and ELISA. Western blot analysis of whole cell lysates and outer membrane extracts with antigen-specific antibodies can be thus used to confirm expression and localization of the heterologous antigens in the A. baumannii cell. Recognition of recombinant antigens on the surface of whole cells can also be verified using antigen-specific antibodies in ELISA experiments.

In a further particular embodiment of the first aspect of the invention, the heterologous antigens expressed at the Outer Membrane of the A. baumannii strain deficient in lipopolysaccharide (LPS) are at least derived from K. pneumoniae, P. aeruginosa, E. coli, and/or A. pleuropneumoniae.

In particular, the heterologous antigens expressed at the Outer Membrane of the A. baumannii strain deficient in lipopolysaccharide (LPS) are at least derived from K. pneumoniae and are selected from the list consisting of Kp-OmpA (SEQ ID No, 31 or 17) and Kp-Ompk36 (SEQ ID No. 32 or 18), including any sequence that has at least 90%, 91%, 92%, 93%, 94%, 95%, 96%, 97%, 98%, or 99% identity with the full/length sequence of any of SEQ ID No. 31, 32, 17 or 18 that when expressed or exposed in one or multiple copies in the outer-membrane of A. baumannii as taught in the present invention by using a signal sequence that can be processed by A. baumannii cells to promote the location of the expressed protein in the outer-membrane of A. baumannii, and upon being inoculated in a subject, preferably a human subject, in need thereof, produces immunization, preferably a protective immune response, not only against A. baumannii infection but also against infections caused by K. pneumoniae. SEQ ID: 2 or 3 as identified in the present invention retains the native signal peptide from K. pneumoniae. However, as taught in this invention, the native peptide is substituted by a signal sequence from an OMP protein that can be processed by A. baumannii cells to promote the location of the expressed protein in the outer-membrane of A. baumannii. Preferably, the signal sequence derives from any OMP of A. baumannii, and most preferably, from the OmpA protein of A. baumannii (SEQ ID No. 1). SEQ ID: 17 and 18 shows the final sequence of Kp-OmpA and Kp-OmpK36 with the signal peptide from A. baumannii OmpA. SEQ ID: 31 and 32 shows the final sequence of Kp-OmpA and Kp-OmpK36 without the signal peptide, i.e. the transmembrane and antigenic domains.

-Kp-OmpA. OmpA is a highly conserved membrane porin among the Enterobacteriaceae. It is not only the homologous version of one of the 2 major antigens identified as responsible for protection exerted by our LPS-null A. baumannii cells in animal models, but it has been described as the protein antigen most recognized by antisera from patients with acute infections produced by K. pneumoniae (Kurupati et al. 2006). Moreover, its use as a DNA vaccine (Kurupati et al. 2011) led to protective immune responses in murine models of sepsis. Briefly, results published by Kurupati et al. (2011) showed that intramuscular immunization of BALB/c females with 4 doses of 50 ul of ompA DNA preparations increases survival at 8 days up to 60% against a challenge with a lethal dose of K. pneumoniae clinical isolate.

-Kp-OmpK36. OmpK36, also a highly conserved membrane porin in K. pneumoniae, has been reported as the second (after OmpA) most recognized protein by antisera from infected patients, with the highest coverage with sera from different clinical infections (Kurupati et al. 2006). Its use as a DNA vaccine in a murine sepsis model described above (Kurupati et al. 2011), showed even higher levels of protection than those obtained by vaccination with OmpA (Kurupati et al. 2011), specifically survival rate at 8 days increases up to 75%, versus 60% obtained with a vaccine expressing only OmpA. In addition, when used as a single subunit vaccine, immunization with 3 doses of 25 μg of full-length recombinant OmpK36 in combination with incomplete Freund's adjuvant was able to protect mice from intraperitoneal challenge (survival rate of 60%), and immune sera were able to neutralize diverse strains of K. pneumoniae (Babu et al. 2017, Hussein et al. 2018).

In another particular embodiment of the first aspect of the invention, the heterologous antigens expressed at the Outer Membrane of the Acinetobacter baumannii strain deficient in lipopolysaccharide (LPS) are at least derived from P. aeruginosa and are selected from the list consisting of Pa-OprF (SEQ ID NO. 33 or 19), Pa-OprI (SEQ ID NO. 35 or 21), Pa-PcrV (SEQ ID NO 45), or Pa-OprI:PcrV (SEQ ID NO 34 or 20), or any sequence that has at least 90%, 91%, 92%, 93%, 94%, 95%, 96%, 97%, 98%, or 99% identity with the full/length sequence of any of SEQ ID No. 33 to 35, 19 to 21 or 45 that when expressed or exposed in one or multiple copies in the outer-membrane of A. baumannii as taught in the present invention by using a signal sequence that can be processed by A. baumannii cells to promote the location of the expressed protein in the outer-membrane of A. baumannii, and upon being inoculated in a subject, preferably a human subject, in need thereof, produces immunization, preferably a protective immune response, not only against A. baumannii infection but also against infections caused by P. aeruginosa. SEQ ID No. 4 to 7 retains the native signal peptide from P. aeruginosa. As taught in this invention, the native peptide is substituted by a signal sequence from an OMP protein that can be processed by A. baumannii cells to promote the location of the expressed protein in the outer-membrane of A. baumannii. Preferably, the signal sequence derives from any OMP of A. baumannii, and most preferably, from the OmpA protein of A. baumannii (SEQ ID No. 1). SEQ ID: 19 to 21 show the final sequence of Pa-OprF, Pa-OprF::PcrV, and Pa-OprI, respectively, with the signal peptide from A. baumannii OmpA. SEQ ID No: 33 to 35 show the final sequence of Pa-OprF, Pa-OprF::PcrV, and Pa-OprI, respectively, without the signal peptide, i.e. the transmembrane and antigenic domains. SEQ ID No. 45 shows the final sequence of Pa-PcrV, without the signal i.e. the antigenic domains.

-Pa-OprI. OprI is a highy conserved membrane lipoprotein that has been demonstrated to exert immunostimulatory activities via activation of Toll-like Receptors (TLRs) 2 and 4, an important step in the induction of Th1 type T cells, when used as adjuvant in a protein based vaccine against Mycobacterium tuberculosis consisting in a fusion protein N-terminal OprI-Ag85 of M. tuberculosis (Gartner et al. 2007). This Th1 inducing property of OprI was previously demonstrated in a mouse model of cutaneous leishmaniasis (Cote-Sierra et al. 2002) and in a model of classical swine fever (Rau et al. 2006). The protective effect of OprI against P. aeruginosa infection was demonstrated in a murine model of sepsis using immunocompromised Balb/c females, where the LD50 was up to 35-fold higher in animals immunized intraperitoneally with 4 doses of 50 μg of purified OprI, versus animals not immunized (Finke et al. 1990 & 1991). Importantly, immunization with OprI was shown to be safe and immunogenic in healthy volunteers (von Specht et al. 1996).

-Pa-PcrV. PcrV is the major antigen of the needles formed by the P. aeruginosa Type 3 secretion system apparatus. It has been shown that anti-PcrV antibodies contribute significantly to protection against virulent P. aeruginosa infections (Sawa et al. 1999 & 2014; Moriyama et al. 2009; Milla et al. 2014), and that active vaccination with PcrV can induce protective immunity against P. aeruginosa infection (Meynet et al. 2018; Aguilera-Herce et al. 2019). Results obtained by Meynet et al. (2018) showed that subcutaneal immunization of C57Bl/6J females with 3 doses at 2 week intervals containing 1-2×10⁸ CFU of killed but metabolic active P. aeruginosa overexpressing PcrV results in survival at 7 days of 58.3% compared to unvaccinated animals against in an acute lung infection model. In addition, Hamaoka et al. (2017) tested the effect of immunization with recombinant PcrV (rPcrV) in combination with three adjuvants in a murine model of acute pneumonia. Results of intraperitoneal immunization with 3 doses of 10 μg of rPcrV each, in combination with AlOH₃ increased survival to 73% compared to the control group immunized with equivalent doses of adjuvant. Importantly, two monoclonal antibody-based therapies targeting PcrV are currently in clinical phases of development, with one study demonstrating a significant decrease in P. aeruginosa infection rates (pneumonia) compared to placebo (Francois et al. 2012; Ali et al. 2019).

-Pa-OprF. OprF is a highly-conserved OmpA homolog, and a major outer membrane porin in P. aeruginosa. Previous immunization studies in experimental models have shown that OprF protects against P. aeruginosa infections. Gilleland et al. (1984) demonstrated that intraperitoneal immunization with 2 doses of 10 ug purified OprF from P. aeruginosa PAO1 protects up to 67% in a murine model of sepsis using CD-1 mice. Moreover, Hassan et al. 2018 have recently reported immunization of mice with recombinant full-length OprF from P. aeruginosa obtained by heterologous expression in Escherichia coli under the control of an inducible promoter. The full-length OprF used as a subunit vaccine in a murine model of acute pneumonia was able to protect Balb/c female mice from infection by non-mucoid and mucoid P. aeruginosa strains PAO1 and PAK, respectively. While 100% of non-immunized animals died 48-72 after intranasal inoculation with P. aeruginosa, survival at 7 days of animals immunized subcutaneously with 4 doses recombinant OprF (rOprF), containing 50ug of recombinant protein, was 50 and 60% for PaO1 and PAK respectively. Nevertheless, in the same study, survival against PAO1 challenge is improved to 100% when the rOprF is combined with recombinant OprI.

In another particular embodiment of the first aspect of the invention, the heterologous antigens expressed at the Outer Membrane of the Acinetobacter baumannii strain deficient in lipopolysaccharide (LPS) are at least derived from E. coli and are selected from the list consisting of Ec-OmpA (SEQ ID NO. 36 or 22), Ec-OmpX (SEQ ID NO. 37 or 23), Ec-FuyA (SEQ ID NO 38 or 24), Ec-Hma (SEQ ID NO 39 or 25) or Ec-IutA (SEQ ID NO 40 or 26), including any sequence that has at least 90%, 91%, 92%, 93%, 94%, 95%, 96%, 97%, 98%, or 99% identity with the full/length sequence of any of SEQ ID No: 22 to 26 or 36 to 40 that when expressed or exposed in one or multiple copies in the outer-membrane of A. baumannii as taught in the present invention by using a signal sequence that can be processed by A. baumannii cells to promote the location of the expressed protein in the outer-membrane of A. baumannii, and upon being inoculated in a subject, preferably a human subject, in need thereof, produces immunization, preferably a protective immune response, not only against A. baumannii infection but also against infections caused by E. coli. SEQ ID No. 8 to 12 retains the native signal peptide from E. coli. As taught in this invention, the native peptide is substituted by a signal sequence from an OMP protein that can be processed by A. baumannii cells to promote the location of the expressed protein in the outer-membrane of A. baumannii. Preferably, the signal sequence derives from any OMP of A. baumannii, and most preferably, from the OmpA protein of A. baumannii (SEQ ID No. 1). SEQ ID: 22 to 26 show the final sequence of Ec-OmpA, Ec-OmpX, Ec-FuyA, Ec-Hma, and Ec-IutA, respectively, with the signal peptide from A. baumannii OmpA. SEQ ID: 36 to 40 show the final sequence of Ec-OmpA, Ec-OmpX, Ec-FuyA, Ec-Hma, and Ec-IutA, respectively, without the signal peptide, i.e. the transmembrane and antigenic domains.

-Ec-OmpA. Outer membrane protein A (OmpA) is a major protein in the Escherichia coli outer membrane. OmpA is composed of three functional domains including a hydrophilic extracellular mass, a beta-barrel transmembrane structure, and a peptidoglycan binding domain (Wang, 2002). Gu et al. 2018 showed that vaccination of mice with a vaccine based on OmpA induced Thl, Th2, and Thl7 immune responses and conferred effective protection. In addition, OmpAVac-specific antibodies were able to mediate opsonophagocytosis and inhibit bacterial invasion, thereby conferring prophylactic protection in E. coli K1-challenged adult mice and neonatal mice. These results suggest that OmpA-based vaccines could good vaccine candidates for the control of E. coli infection.

-Ec-OmpX. The integral outer membrane protein X (OmpX) from Escherichia coli belongs to a family of highly conserved bacterial proteins that promote bacterial adhesion to and entry into mammalian cells. Moreover, these proteins have a role in the resistance against attack by the human complement system (Vogt and Schulz, 1999). Maisnier-Patin et al. (2003), using recombinant OmpX from E. coli reported that EcOmpX binds to and is internalized by human antigen-presenting cells.

-Ec-FuyA. Yersiniabactin is a siderophore found in the pathogenic bacteria Yersinia pestis, Yersinia pseudotuberculosis, and Yersinia enterocolitica, as well as several strains of enterobacteria including enteropathogenic Escherichia coli and Salmonella enterica. Urinary tract infectious E. coli have highly efficient mechanisms of iron acquisition, one of which is the yersiniabactin system. The fyuA gene, encoding the yersiniabactin receptor, was found to be highly important for biofilm formation in iron-poor environments such as human urine (Hancock et al. 2008). In a mice model, Habibi et al. (2017) showed that vaccination with FyuA induced humoral responses of both IgG1 (Th2) and IgG2a (Thl) types and reduced uropathogenic E. coli colonization in the bladder and kidney.

-Ec-Hma. Hma is a 79 kDa heme receptor of the outer-membrane, heme acquisition protein, which functions independently of ChuA to mediate hemin uptake by uropathogenic E. coli strains (Hagan and Mobley, 2009). Intranasal immunization of mice with outer membrane iron receptors FyuA, Hma, IreA, and IutA, conjugated to cholera toxin, provided protection in the bladder or kidneys under conditions of challenge with uropathogenic E. coli strains in mice (Forsyth et al. 2020).

-Ec-IutA. IutA is a 75-kDa ferric aerobactin receptor of the outer-membrane of E. coli. Active immunization of mice with recombinant antigens EcpA, EcpD, IutA or IroN elicited high levels of total IgG antibody of IgG1/IgG2a isotypes, and were determined to be highly protective against E. coli infection in lethal and non-lethal sepsis challenges (Mellata et al. 2016).

In another particular embodiment of the first aspect of the invention, the heterologous antigens expressed at the Outer Membrane of the Acinetobacter baumannii strain deficient in lipopolysaccharide (LPS) are at least derived from A. pleuropneumoniae and are selected from the list consisting of Ap-OmpA (SEQ ID NO. 27 or 41), Ap-OmpW (SEQ ID NO. 28 or 42), Ap-TbpA (SEQ ID NO 29 or 43), or Ap-ApfA (SEQ ID NO 30 or 44), including any sequence that has at least 90%, 91%, 92%, 93%, ₉₄%, 95%, 96%, 97%, 98%, or 99% identity with the full/length sequence of any of SEQ ID: 27 to 30 or 41 to 44 that when expressed or exposed in one or multiple copies in the outer-membrane of A. baumannii as taught in the present invention by using a signal sequence that can be processed by A. baumannii cells to promote the location of the expressed protein in the outer-membrane of A. baumannii, and upon being inoculated in a subject, preferably a human subject, in need thereof, produces immunization, preferably a protective immune response, not only against A. baumannii infection but also against infections caused by A. pleuropneumoniae. SEQ ID No. 13 to 16 retains the native signal peptide from A. pleuropneumoniae. As taught in this invention, the native peptide is substituted by a signal sequence from an OMP protein that can be processed by A. baumannii cells to promote the location of the expressed protein in the outer-membrane of A. baumannii. Preferably, the signal sequence derives from any OMP of A. baumannii, and most preferably, from the OmpA protein of A. baumannii (SEQ ID No. 1). SEQ ID: 27 to 30 shows the final sequence of Ap-OmpA, Ap-OmpW, Ap-TbpA, and Ap-ApfA, respectively, with the signal peptide from A. baumannii OmpA. SEQ ID: 41 to 44 show the final sequence of Ap-OmpA, Ap-OmpW, Ap-TbpA, and Ap-ApfA, respectively, without the signal peptide, i.e. the transmembrane and antigenic domains.

-Ap-OmpA. It is the A. pleuropneumoniae homolog of major protein in the outer membrane of other Gram-negative bacteria like E. coli. OmpA is composed of three functional domains including a hydrophilic extracellular mass, a beta-barrel transmembrane structure, and a peptidoglycan binding domain (Wang, 2002).

-Ap-OmpW. It is another integral OMP protein in A. pleuropneumoniae. The ompW gene is found in many bacteria, such as Escherichia coli, Aeromonas hydrophila, and Vibrio harveyi. Immune response to the OmpW protein has been shown to provide provides immunity to Aeromonas challenge. In A. pleuropneumoniae OmpW seems to regulate the phenotype during infections (Chen et al. 2022).

-Ap-TbpA. TbpA (transferrin-biding protein A) is an 100 KDa outer-membrane P-barrel that forms the import channel for transferrin, which has been shown as a molecular determinant of virulence and pathogenesis in A. pleuropneumoniae (Nahar et al. 2021).

-Ap-ApfA. ApfA is the outer-membrane protein anchoring the Type IV fimbria of A. pleuropneumoniae. Type 4 fimbriae have been shown to increase bacteria-bacteria interactions and to promote bacterial adherence and colonization, thereby facilitating the progress of bacterial infection (Zhang et al. 2000). In A. pleuropneumoniae, a conserved general secretion pathway (GSP) domain in the N-terminal part of the outer membrane protein ApfA, adjacent to the transmembrane domain has been identified. ApfAs has been used as an outer membrane anchor, to which potential immunogens can be attached (Antenuci et al. 2020).

A. baumannii OmpA signal peptide
Sequence ID Number 1
MKLSRIALATMLVAAPLAAANA.
Nucleotide sequence from Sequence ID Number 1
Atgaaattgagtcgtattgcacttgctactatgcttgttgctgctccattagctgctgctaatgc
t
K. pneumoniae OmpA
Sequence ID Number 2
MKAIFVLNAAPKDNTWYAGGKLGWSQYHDTGFYGNGFQNNNGPTRNDQLGAGAFGGYQVNPYLGF
EMGYDWLGRMAYKGSVDNGAFKAQGVQLTAKLGYPITDDLDIYTRLGGMVWRADSKGNYASTGVS
RSEHDTGVSPVFAGGVEWAVTRDIATRLEYQWVNNIGDAGTVGTRPDNGMLSLGVSYRFGQEDAA
PVVAPAPAPAPEVATKHFTLKSDVLFNFNKATLKPEGQQALDQLYTQLSNMDPKDGSAVVLGYTD
RIGSEAYNQQLSEKRAQSVVDYLVAKGIPAGKISARGMGESNPVTGNTCDNVKARAALIDCLAPD
RRVEIEVKGYKEVVTQPQA.
*In bold K. pneumoniae native signal peptide
Nucleotide sequence from Sequence ID Number 2
atgaaagcaattttcgtactgaatgcggctccgaaagataacacctggtatgcaggtggtaaact
gggttggtcccagtatcacgacaccggtttctacggtaacggtttccagaacaacaacggtccga
cccgtaacgatcagcttggtgctggtgcgttcggtggttaccaggttaacccgtacctcggtttc
gaaatgggttatgactggctgggccgtatggcatataaaggcagcgttgacaacggtgctttcaa
agctcagggcgttcagctgaccgctaaactgggttacccgatcactgacgatctggacatctaca
cccgtctgggcggcatggtttggcgcgctgactccaaaggcaactacgcttctaccggcgtttcc
cgtagcgaacacgacactggcgtttccccagtatttgctggcggcgtagagtgggctgttactcg
tgacatcgctacccgtctggaataccagtgggttaacaacatcggcgacgcgggcactgtgggta
cccgtcctgataacggcatgctgagcctgggcgtttcctaccgcttcggtcaggaagatgctgca
ccggttgttgctccggctccggctccggctccggaagtggctaccaagcacttcaccctgaagtc
tgacgttctgttcaacttcaacaaagctaccctgaaaccggaaggtcagcaggctctggatcagc
tgtacactcagctgagcaacatggaCccgaaagacggttccgctgttgttctgggctacaccgac
cgcatcggttccgaagcttacaaccagcagctgtctgagaaacgtgctcagtccgttgttgacta
cctggttgctaaaggcatcccggctggcaaaatctccgctcgcggcatgggtgaatccaacccgg
ttactggcaacacctgtgacaacgtgaaagctcgcgctgccctgatcgattgcctggctccggat
cgtcgtgtagagatcgaagttaaaggctacaaagaagttgtaactcagccgcaggcttaa
K. pneumoniae OmpK36
Sequence ID Number 3
MKVKVLSLLVPALLVAGAANAAEIYNKDGNKLDLYGKIDGLHYFSDDKSVDGDQTYMRVGVKGET
QINDQLTGYGQWEYNVQANNTESSSDQAWTRLAFAGLKFGDAGSFDYGRNYGVVYDVTSWTDVLP
EFGGDTYGSDNFLQSRANGVATYRNSDFFGLVDGLNFALQYQGKNGSVSGEGALSPTNNGRTALK
QNGDGYGTSLTYDIYDGISAGFAYSNSKRLGDQNSKLALGRGDNAETYTGGLKYDANNIYLATQY
TQTYNATRAGSLGFANKAQNFEVVAQYQFDFGLRPSVAYLQSKGKDLEGYGDQDILKYVDVGATY
YFNKNMSTYVDYKINLLDDNSFTHNAGISTDDVVALGLVYQF.
*In bold K. pneumoniae native signal peptide
Nucleotide sequence from Sequence ID Number 3
atgaaagttaaagtactgtccctcctggtaccggctctgctggtagcaggcgcagcaaatgcggc
tgaaatttataacaaagacggcaacaaattagacctgtacggtaaaattgacggtctgcactact
tctctgacgacaagagcgtcgacggcgaccagacctacatgcgtgtaggcgtgaaaggcgaaacc
cagatcaacgaccagctgaccggttacggccagtgggaatacaacgttcaggcgaacaacactga
aagctccagcgatcaggcatggactcgtctggcattcgcaggcctgaaatttggcgacgcgggct
ctttcgactacggtcgtaactacggcgtagtatacgacgtaacgtcctggaccgacgttctgccg
gaattcggcggcgacacctacggttctgacaacttcctgcagtcccgtgctaacggcgttgcaac
ctaccgtaactctgatttcttcggtctggttgacggcctgaactttgctctgcagtatcagggta
aaaacggcagcgtcagcggcgaaggcgctctgtctcctaccaacaacggtcgtaccgccttgaaa
cagaacggcgacggttacggtacttctctgacctatgacatctatgatggcatcagcgctggttt
cgcatactctaactccaaacgtcttggcgaccagaacagcaagctggcactgggtcgtggcgaca
acgctgaaacctacaccggcggtctgaaatacgacgcgaacaacatctacctggccactcagtac
acccagacctacaacgcgacccgcgccggttccctgggctttgctaacaaagcgcagaacttcga
agtggttgctcagtaccagttcgacttcggtctgcgtccgtccgtggcttacctgcagtctaaag
gtaaggatctggaaggctacggcgaccaggacatcctgaaatatgttgacgttggcgcgacctac
tacttcaacaaaaacatgtccacctatgttgactacaaaatcaacctgctggacgacaatagctt
cacccacaacgccggtatctctaccgacgacgtggttgcactgggcctggtttaccagttctaa
P. aeruginosa OprF
Sequence ID Number 4
MKLKNTLGVVIGSLVAASAMNAFAQGQNSVEIEAFGKRYFTDSVRNMKNADLYGGSIGYFLTDDV
ELALSYGEYHDVRGTYETGNKKVHGNLTSLDAIYHFGTPGVGLRPYVSAGLAHQNITNINSDSQG
RQQMTMANIGAGLKYYFTENFFAKASLDGQYGLEKRDNGHQGEWMAGLGVGFNFGGSKAAPAPEP
VADVCSDSDNDGVCDNVDKCPDTPANVTVDANGCPAVAEVVRVQLDVKFDFDKSKVKENSYADIK
NLADFMKQYPSTSTTVEGHTDSVGTDAYNQKLSERRANAVRDVLVNEYGVEGGRVNAVGYGESRP
VADNATAEGRAINRRVEAEVEAEAK.
*In bold P. aeruginosa native signal peptide
Nucleotide sequence from Sequence ID Number 4
atgaaactgaagaacaccttaggcgttgtcatcggctcgctggttgccgcttcggcaatgaacgc
ctttgcccagggccagaactcggtagagatcgaagccttcggcaagcgctacttcaccgacagcg
ttcgcaacatgaagaacgcggacctgtacggcggctcgatcggttacttcctgaccgacgacgtc
gagctggcgctgtcctacggtgagtaccatgacgttcgtggcacctacgaaaccggcaacaagaa
ggtccacggcaacctgacctccctggacgccatctaccacttcggtaccccgggcgtaggtctgc
gtccgtacgtgtcggctggtctggctcaccagaacatcaccaacatcaacagcgacagccaaggc
cgtcagcagatgaccatggccaacatcggcgctggtctgaagtactacttcaccgagaacttctt
cgccaaggccagcctcgacggccagtacggtctggagaagcgtgacaacggtcaccagggcgagt
ggatggctggcctgggcgtcggcttcaacttcggtggttcgaaagccgctccggctccggaaccg
gttgccgacgtttgctccgactccgacaacgacggcgtttgcgacaacgtcgacaagtgcccgga
taccccggccaacgtcaccgttgacgccaacggctgcccggctgtcgccgaagtcgtacgcgtac
agctggacgtgaagttcgacttcgacaagtccaaggtcaaagagaacagctacgctgacatcaag
aacctggctgacttcatgaagcagtacccgtccacttccaccaccgttgaaggtcacaccgactc
cgtcggcaccgacgcttacaaccagaagctgtccgagcgtcgtgccaacgccgttcgtgacgtac
tggtcaacgagtacggtgtagaaggtggtcgcgtgaacgctgttggttacggcgagtcccgcccg
gttgccgacaacgccaccgctgaaggccgcgctatcaaccgtcgcgttgaagccgaagtagaagc
tgaagccaagtaa
P. aeruginosa OprI::PcrV fusion protein
Sequence ID Number 5
MNNVLKFSALALAAVLATGCSSHSKETEARLTATEDAAARAQARADEAYRKADEALGAAQKAQQT
ADEANERALRMLEKASRKGGGGSGGGGSGGGGSSAAPASAEQEELLALLRSERIVLAHAGQPLSE
AQVLKALAWLLAANPSAPPGQGLEVLREVLQARRQPGAQWDLREFLVSAYFSLHGRLDEDVIGVY
KDVLQTQDGKRKALLDELKALTAELKVYSVIQSQINAALSAKQGIRIDAGGIDLVDPTLYGYAVG
DPRWKDSPEYALLSNLDTFSGKLSIKDFLSGSPKQSGELKGLSDEYPFEKDNNPVGNFATTVSDR
SRPLNDKVNEKTTLLNDTSSRYNSAVEALNRFIQKYDSVLRDILSAI.
*In bold P. aeruginosa native signal peptide
*linker of the fusion protein underlined
Nucleotide sequence from Sequence ID Number 5
atgaacaacgttctgaaattctctgctctggctctggctgctgttctggccaccggttgcagcag
ccactccaaagaaaccgaagctcgtctgaccgctaccgaagacgcagctgctcgtgctcaggctc
gcgctgacgaagcctatcgcaaggctgacgaagctctgggcgctgctcagaaagctcagcagact
gctgacgaggctaacgagcgtgccctgcgcatgctggaaaaagccagccgcaagggcggcggcgg
cagcggcggcggcggcagcggcggcggcggcagctcggcggcgcctgccagtgccgagcaggagg
aactgctggccctgttgcgcagcgagcggatcgtgctggcccacgccggccagccgctgagcgag
gcgcaagtgctcaaggcgctcgcctggttgctcgcggccaatccgtccgcgcctccggggcaggg
cctcgaggtactccgcgaagtcctgcaggcacgtcggcagcccggtgcgcagtgggatctgcgcg
agttcctggtgtcggcctatttcagcctgcacgggcgtctcgacgaggatgtcatcggtgtctac
aaggatgtcctgcagacccaggacggcaagcgcaaggcgctgctcgacgagctgaaggcgctgac
cgcggagttgaaggtctacagcgtgatccagtcgcagatcaacgccgcgctgtcggccaagcagg
gcatcaggatcgacgctggcggtatcgatctggtcgaccccacgctatatggctatgccgtcggc
gatcccaggtggaaggacagccccgagtatgcgctgctgagcaatctggataccttcagcggcaa
gctgtcgatcaaggattttctcagcggctcgccgaagcagagcggggaactcaagggcctcagcg
atgagtaccccttcgagaaggacaacaacccggtcggcaatttcgccaccacggtgagcgaccgc
tcgcgtccgctgaacgacaaggtcaacgagaagaccaccctgctcaacgacaccagctcccgcta
caactcggcggtcgaggcgctcaaccgcttcatccagaaatacgacagcgtcctgcgcgacattc
tcagcgcgatctag
P. aeruginosa OprI
Sequence ID Number 6
MNNVLKFSALALAAVLATGCSSHSKETEARLTATEDAAARAQARADEAYRKADEALGAAQKAQQT
ADEANERALRMLEKASRK.
*In bold P. aeruginosa native signal peptide
Nucleotide sequence from Sequence ID Number 6
atgaacaacgttctgaaattctctgctctggctctggctgctgttctggccaccggttgcagcag
ccactccaaagaaaccgaagctcgtctgaccgctaccgaagacgcagctgctcgtgctcaggctc
gcgctgacgaagcctatcgcaaggctgacgaagctctgggcgctgctcagaaagctcagcagact
gctgacgaggctaacgagcgtgccctgcgcatgctggaaaaagccagccgcaatag
P. aeruginosa PcrV
Sequence ID Number 7
MEVRNLNAARELFLDELLAASAAPASAEQEELLALLRSERIVLAHAGQPLSEAQVLKALAWLLAA
NPSAPPGQGLEVLREVLQARRQPGAQWDLREFLVSAYFSLHGRLDEDVIGVYKDVLQTQDGKRKA
LLDELKALTAELKVYSVIQSQINAALSAKQGIRIDAGGIDLVDPTLYGYAVGDPRWKDSPEYALL
SNLDTFSGKLSIKDFLSGSPKQSGELKGLSDEYPFEKDNNPVGNFATTVSDRSRPLNDKVNEKTT
LLNDTSSRYNSAVEALNRFIQKYDSVLRDILSAI.
*In bold P. aeruginosa native signal peptide
Nucleotide sequence from Sequence ID Number 7
atggaagtcagaaaccttaatgccgctcgcgagctgttcctggacgagctcctggccgcgtcggc
ggcgcctgccagtgccgagcaggaggaactgctggccctgttgcgcagcgagcggatcgtgctgg
cccacgccggccagccgctgagcgaggcgcaagtgctcaaggcgctcgcctggttgctcgcggcc
aatccgtccgcgcctccggggcagggcctcgaggtactccgcgaagtcctgcaggcacgtcggca
gcccggtgcgcagtgggatctgcgcgagttcctggtgtcggcctatttcagcctgcacgggcgtc
tcgacgaggatgtcatcggtgtctacaaggatgtcctgcagacccaggacggcaagcgcaaggcg
ctgctcgacgagctcaaggcgctgaccgcggagttgaaggtctacagcgtgatccagtcgcagat
caacgccgcgctgtcggccaagcagggcatcaggatcgacgctggcggtatcgatctggtcgacc
ccacgctatatggctatgccgtcggcgatcccaggtggaaggacagccccgagtatgcgctgctg
agcaatctggataccttcagcggcaagctgtcgatcaaggattttctcagcggctcgccgaagca
gagcggggaactcaagggcctcagcgatgagtaccccttcgagaaggacaacaacccggtcggca
atttcgccaccacggtgagcgaccgctcgcgtccgctgaacgacaaggtcaacgagaagaccacc
ctgctcaacgacaccagctcccgctacaactcggcggtcgaggcgctcaaccgcttcatccagaa
atacgacagcgtcctgcgcgacattctcagcgcgatctag
E. coli OmpA
Sequence ID Number 8
MKKTAIAIAVALAGFATVAQAAPKDNTWYTGAKLGWSQYHDTGFIPNNGPTHENQLGAGAFGGYQ
VNPYVGFEMGYDWLGRMPYKGDNINGAYKAQGVQLTAKLGYPITDDLDIYTRLGGMVWRADTKAN
VPGGASFKDHDTGVSPVFAGGVEYAITPEIATRLEYQWTNNIGDAHTIGTRPDNGMLSLGVSYRF
GQGEVAPVVAPAPAPAPEVQTKHFTLKSDVLFTFNKATLKPEGQAALDQLYSQLSNLDPKDGSVV
VLGYTDRIGSDAYNQALSERRAQSVVDYLISKGIPADKISARGMGESNPVTGNTCDNVKQRAALI
DCLAPDRRVEIEVKGIKDVVTQPQA.
*In bold E. coli native signal peptide
Nucleotide sequence from Sequence ID Number 8
atgaaaaagacagctatcgcgattgcagtggcactggctggtttcgctaccgtagcgcaggccgc
tccgaaagataacacctggtacactggtgctaaactgggctggtcccagtaccatgacactggtt
ttattcctaacaatggtccgacccacgaaaaccaactgggtgcaggtgcttttggtggttaccag
gttaacccgtatgttggctttgaaatgggttacgactggttaggtcgtatgccgtacaaaggcga
caacatcaacggcgcatacaaagctcagggcgttcagctgaccgctaaactgggttacccaatca
ctgacgatctggacatctacactcgtctgggtggtatggtatggcgtgcagacaccaaggctaac
gtacctggtggcgcatcctttaaagaccacgacaccggcgtttctccggtcttcgctggcggtgt
tgagtatgcgatcactcctgaaatcgctacccgtctggaataccagtggaccaacaacatcggtg
acgcacacaccatcggcactcgtccggacaacggcatgctgagcctgggtgtttcctaccgtttc
ggtcagggcgaagtagctccagtagttgctccggctccagctccggcaccggaagtacagaccaa
gcacttcactctgaagtctgacgttctgttcaccttcaacaaagcaaccctgaaaccggaaggtc
aggctgctctggatcagctgtacagccagctgagcaacctggatccgaaagacggttccgtagtt
gttctgggttacactgaccgcatcggttctgacgcttataaccaggctctgtccgagcgtcgtgc
tcagtccgttgttgattacctgatctctaaaggtatcccggcagacaaaatctccgcacgtggta
tgggcgaatccaacccggttactggcaacacctgtgacaacgtgaaacagcgtgctgcactgatc
gattgcctggctccggatcgtcgcgtagagatcgaagttaaaggcatcaaagacgttgtaactca
gccgcaggcttaa
E. coli OmpX
Sequence ID Number 9
MKKIACLSALAAVLAFTAGTSVAATSTVTGGYAQSDAQGQMNKMGGFNLKYRYEEDNSPLGVIGS
FTYTEKSRTASSGDYNKNQYYGITAGPAYRINDWASIYGVVGVGYGKFQTTEYPTYKHDTSDYGF
SYGAGLQFNPMENVALDFSYEQSRIRSVDVGTWIAGVGYRF.
*In bold E. coli native signal peptide
Nucleotide sequence from Sequence ID Number 9
atgaaaaaaattgcatgtctttcagcactggccgcagttctggctttcaccgcaggtacttccgt
agctgcgacttctaccgtaactggcggttacgcacaaagcgacgctcagggccaaatgaacaaaa
tgggggtttcaacctgaaataccgctatgaagaagacaacagcccgctgggtgtgatcggttctt
tcacttacaccgagaaaagccgtactgcaagctctggtgactacaacaaaaaccagtactacggc
atcactgctggtccggcttaccgcattaacgactgggcaagcatctacggtgtagtgggtgtggg
ttatggtaaattccagaccactgaatacccgacctacaaacacgacaccagcgactacggtttct
cctacggtgctggtttgcagttcaacccgatggaaaacgttgctctggacttctcttacgagcag
agccgtattcgtagcgttgacgtaggcacctggattgccggtgttggttaccgcttctaa
E. coli FyuA
Sequence ID Number 10
MKMTRLYPLALGGLLLPAIANAQTSQQDESTLVVTASKQSSRSASANNVSSTVVSAPELSDAGVT
ASDKLPRVLPGLNIENSGNMLFSTISLRGVSSAQDFYNPAVTLYVDGVPQLSTNTIQALTDVQSV
ELLRGPQGTLYGKSAQGGIINIVTQQPDSTPRGYIEGGVSSRDSYRSKFNLSGPIQDGLLYGSVT
LLRQVDDGDMINPATGSDDLGGTRASIGNVKLRLAPDDQPWEMGFAASRECTRATQDAYVGWNDI
KGRKLSISDGSPDPYMRRCTDSQTLSGKYTTDDWVFNLISAWQQQHYSRTFPSGSLIVNMPQRWN
QDVQELRAATLGDARTVDMVFGLYRQNTREKLNSAYDMPTMPYLSSTGYTTAETLAAYSDLTWHL
TDRFDIGGGVRFSHDKSSTQYHGSMLGNPFGDQGKSNDDQVLGQLSAGYMLTDDWRVYTRVAQGY
KPSGYNIVPTAGLDAKPFVAEKSINYELGTRYETADVTLQAATFYTHTKDMQLYSGPVGMQTLSN
AGKADATGVELEAKWRFAPGWSWDINGNVIRSEFTNDSELYHGNRVPFVPRYGAGSSVNGVIDTR
YGALMPRLAVNLVGPHYFDGDNQLRQGTYATLDSSLGWQATERMNISVYVDNLFDRRYRTYGYMN
GSSAVAQVNMGRTVGINTRIDFF.
*In bold E. coli native signal peptide
Nucleotide sequence from Sequence ID Number 10
atgaaaatgacacggctttatcctctggccttggggggattattgctccccgccattgctaatgc
ccagacttcacagcaagacgaaagcacgctggtggttaccgccagtaaacaatcttcccgctcgg
catcagccaacaacgtctcatctactgttgtcagcgcgccggaattaagcgacgccggcgtcacc
gccagcgacaaactccccagagtcttgcccgggctcaatattgaaaatagcggcaacatgctttt
ttcgacgatctcgctacgcggcgtctcttcagcgcaggacttctataaccccgccgtcaccctgt
atgtcgatggcgtccctcagctttccaccaacaccatccaggcgcttaccgatgtgcaaagcgtg
gagttgctgcgaggcccacagggaacgttatatggcaaaagcgctcagggcgggatcatcaacat
cgtcacccagcagccggacagcacgccgcgcggctatattgaaggcggcgtcagtagccgcgaca
gttatcgaagtaagttcaacctgagcggccccattcaggatggcctgctgtacggcagcgtcacc
ctgttacgccaggttgatgacggcgacatgattaaccccgcgacgggaagcgatgacttaggcgg
cacccgcgccagcatagggaatgtgaaactgcgtctggcgccggacgatcagccctgggaaatgg
gctttgccgcctcacgcgaatgtacccgcgccacccaggatgcctatgtgggatggaatgatatt
aagggccgtaagctgtcgatcagcgatggttcaccagacccgtacatgcggcgctgcactgacag
ccagaccctgagtgggaaatacaccaccgatgactgggttttcaacctgatcagcgcctggcagc
agcagcattattcgcgcaccttcccttccggttcgttaatcgtcaatatgcctcagcgctggaat
caggatgtgcaggagctgcgcgccgcaaccctgggcgatgcgcgtaccgttgatatggtgtttgg
gctgtaccggcagaacacccgcgagaagttaaattcagcctacgacatgccgacaatgccttatt
taagcagtaccggctataccaccgctgaaacgctggccgcatacagtgacctgacctggcattta
accgatcgttttgatatcggcggcggcgtgcgcttctcgcatgataaatccagtacacaatatca
cggcagcatgctcggcaacccgtttggcgaccagggtaagagcaatgacgatcaggtgctcgggc
agctatccgcaggctatatgctgaccgatgactggagagtgtatacccgtgtagcccagggatat
aaaccttccgggtacaacatcgtgcctactgcgggtcttgatgccaaaccgttcgtcgccgagaa
atccatcaactatgaacttggcacccgctacgaaaccgctgacgtcacgctgcaagccgcgacgt
tttatacccacaccaaagacatgcagctttactctggcccggtcgggatgcagacattaagcaat
gcgggtaaagccgacgccaccggcgttgagcttgaagcgaagtggcggtttgcgccaggctggtc
atgggatatcaatggcaacgtgatccgttccgaattcaccaatgacagtgagttgtatcacggta
accgggtgccgttcgtaccacgttatggcgcgggaagcagcgtgaacggcgtgattgatacgcgc
tatggcgcactgatgccccgactggcggttaatctggtcgggccgcattatttcgatggcgacaa
ccagttgcggcaaggcacctatgccaccctggacagcagcctgggctggcaggcgactgaacgga
tgaacatttccgtctatgtcgataacctgttcgaccgtcgttaccgtacctatggctacatgaac
ggcagcagcgccgtcgcgcaggtcaatatgggtcgcaccgtcggtatcaatacgcgaattgattt
cttctga
E. coli Hma
Sequence ID Number 11
MLYNIPCRIYILSTLSLCISGIVSTATATSSETKISNEETLVVTTNRSASNLWESPATIQVIDQQ
TLQNSTNASIADNLQDIPGVEITDNSLAGRKQIRIRGEASSRVLILIDGQEVTYQRAGDNYGVGL
LIDESALERVEVVKGPYSVLYGSQAIGGIVNFITKKGGDKLASGVVKAVYNSATAGWEESIAVQG
SIGGFDYRINGSYSDQGNRDTPDGRLPNTNYRNNSQGVWLGYNSGNHRFGLSLDRYRLATQTYYE
DPDGSYEAFSVKIPKLEREKVGVFYDTDVDGDYLKKIHFDAYEQTIQRQFANEVKTTQPVPSPMI
QALTVHNKTDTHDKQYTQAVTLQSHFSLPANNELVTGAQYKQDRVSQRSGGMTSSKSLTGFINKE
TRTRSYYESEQSTVSLFAQNDWRFADHWTWTMGVRQYWLSSKLTRGDGVSYTAGIISDTSLARES
ASDHEMVISTSLRYSGFDNLELRAAFAQGYVFPTLSQLFMQTSAGGSVTYGNPDLKAEHSNNFEL
GARYNGNQWLIDSAVYYSEAKDYIASLICDGSIVCNGNTNSSRSSYYYYDNIDRAKTWGLEISAE
YNGWVFSPYISGNLIRRQYETSTLKTTNTGEPAINGRIGLKHTLVMGQANIISDVFIRAASSAKD
DSNGTETNVPGWATLNFAVNTEFGNEDQYRINLALNNLTDKRYRTAHETIPAAGFNAAIGFVWN
F.
*In bold E. coli native signal peptide
Nucleotide sequence from Sequence ID Number 11
atgttatataatataccttgtcgaatttatatcctttccactctgtcattatgcatttctgggat
agtttctactgcaaccgcaacttcttcagaaacaaaaatcagcaacgaagagacgctcgtcgtga
ccacgaatcgttcggcaagcaacctttgggaaagcccggcgactatacaggttattgaccaacaa
acattgcagaactccaccaatgcctccatagccgataatttgcaggacatccccggagtagagat
aacagacaactccttggcaggccgtaaacaaatccgcattcgtggcgaagcatcctcccgtgttt
taattctcattgatggtcaggaggtaacttatcagcgcgccggagataattatggtgtgggactg
ttgatagatgagtctgcgctggagcgtgttgaggtagtgaaaggtccatattccgtactgtacgg
ttcacaggcaattggcggtattgttaacttcataaccaaaaagggaggtgacaaacttgcatctg
gagttgtgaaagctgtttataattccgcaacagcaggctgggaagaatcaatcgcggtccagggg
agcatcggtggatttgattatcgcatcaacggtagttattctgatcagggcaatcgtgatacgcc
ggatggacgtctgccgaataccaactatcgtaacaatagtcagggtgtatggttgggttataact
ccggaaaccatcgttttggcctctcgcttgatcgctacagactcgcgacgcaaacttactatgag
gatccagacggaagctatgaggcatttagtgtcaaaatacctaaacttgaacgagagaaagttgg
ggtattctatgacacagacgtggacggtgactatctaaaaaaaattcatttcgacgcgtatgagc
agaccatccagcgccaatttgccaacgaagtaaaaacgacacagcctgttcccagtccgatgatt
caggctctgaccgttcataacaagactgacacccatgataagcaatacactcaggcggtcacatt
gcagagtcacttttcgctgcctgctaataatgaacttgttaccggtgcacagtacaaacaagaca
gggtcagccaaaggtccggtggcatgacctcaagcaaatctctgaccggcttcattaataaggaa
acacgaactcgctcctattatgagtcagagcaaagtacagtctcactattcgcacaaaatgactg
gcgattcgccgatcactggacatggacaatgggagttcgccaatactggctttcttcaaagttga
cgcgtggtgacggagtatcatataccgcaggcattataagcgatacctctcttgccagagagtct
gcgagtgatcacgaaatggtaacatctacaagcctgcgctattcaggtttcgataacttggagtt
acgcgctgcgttcgcgcaaggctacgtatttcccacactctcccagctttttatgcagacatctg
cgggcggcagtgtcacatacggaaatcctgatcttaaggctgaacactccaataactttgaatta
ggtgcacgatataatggtaatcagtggctgattgacagcgcagtttactactcagaagctaaaga
ttatattgcaagtctgatctgtgatggcagtatagtttgcaatggtaacaccaactcctcccgta
gtagctactattattatgacaatattgatcgggcaaaaacatggggactggaaataagcgcggaa
tataatggctgggttttctcgccatatatcagtggcaatttaattcgtcggcaatatgaaacttc
aacattaaaaacaactaatacaggagaaccagcgataaacggacgtatagggctgaaacatactc
ttgtgatgggtcaggccaacataatctctgatgtttttattcgtgctgcctctagtgcaaaagat
gacagtaacggtaccgaaacaaatgttccgggctgggccactctcaactttgcagtaaatacaga
attcggtaacgaggatcagtaccggattaacctagcactcaataacctgacagacaaacgctacc
gtacagcacatgaaactattcctgcagcaggttttaatgcagctataggttttgtatggaatttc
tga
E. coli IutA
Sequence ID Number 12
MPRALGPLLLVVLSPAVAQQNDDNEIIVSASRSNRTVAEMAQTTWVIENAELEQQIQGGKELKDA
LAQLIPGLDVSSQSRTNYGMNMRGRPLVVLIDGVRLNSSRSDSRQLDSVDPFNIDHIEVISGATA
LYGGGSTGGLINIVTKKGQPETMMEFEAGTKSGFNSSKDHDERIAGAVSGGNDHISGRLSVAYQK
FGGWFDGNGDATLLDNTQTGLQHSNRLDIMGTGTLNIDESRQLQLITQYYKSQGDDNYGLNLGKG
FSAISGSSTPYVSKGLNSDRIPGTERHLISLQYSDSDFLRQELVGQVYYRDESLRFYPFPTVNAN
KQATAFSSSQQDTDQYGMKLTLNSQLMDGWQITWGLDAEHERFTSNQMFFDLAQASASGGLNNHK
IYTTGRYPSYDITNLAAFLQSSYDINDIFTVSGGVRYQYTENRVDDFIDYTQQQKIAAGKAISAD
AIPGGSVDYDNFLFNAGLLMHITERQQAWFNFSQGVALPDPGKYYGRGIYGAAVNGHLPLTKSVN
VSDSKLEGVKVDSYELGWRFTGDNLRTQIAAYYSLSNKSVERNKDLTISVKDDRRRIYGVEGAVD
YLIPDTDWSTGVNFNVLKTESKVNGQWQKYDVKESSPSKATAYINWAPEPWSLRVQSTTSFDVSD
AEGNDINGYTTVDFISSWQLPVGTLSFSVENLFDRDYTTVWGQRAPLYYSPGYGPASLYDYKGRG
RTFGLNYSVLF.
*In bold E. coli native signal peptide
Nucleotide sequence from Sequence ID Number 12
atgccccgggctcttggtccgctgcttcttgtcgtgctgtcaccagctgtcgcccagcaaaacga
tgataatgagatcatagtgtctgccagccgcagcaatcgaactgtagcggagatggcgcaaacca
cctgggttatcgaaaatgccgaactggagcagcagattcagggcggtaaagagctgaaagacgca
ctggctcagttaatccccggccttgatgtcagcagccagagccgaaccaactacggtatgaacat
gcgtggccgcccgctggttgtcctgattgacggtgtgcgcctcaactcttcacgttccgacagcc
gacaactggactctgtcgatccttttaatatcgaccatattgaagtgatctccggcgcgacggcc
ctgtacggtggcgggagtaccggagggttgatcaacatcgtgaccaaaaaaggccagccggaaac
catgatggagtttgaggctggcacaaaaagtggctttaacagcagtaaagatcacgatgagcgca
ttgccggtgctgtctccggcggaaatgaccatatctccggacgtctttccgtggcatatcagaaa
tttggcggctggtttgacggtaacggcgatgccaccctgcttgataacacccagaccggcctgca
gcactccaatcggctggacatcatgggaaccggtacgctgaacatcgatgaatcccggcagcttc
aactgataacgcagtactataaaagtcagggggacgacaattacgggcttaatctcgggaaaggc
ttttccgccatcagcgggagcagcacaccatacgtcagtaaggggctgaattctgaccgcattcc
cggcactgagcggcatttgatcagcctgcagtactctgacagtgatttcctgagacaggaactgg
tcggtcaggtttactaccgcgatgagtcgttgcggttctacccgttcccgacggtaaatgcgaat
aaacaggcgacggctttctcctcgtcacagcaggataccgaccagtacggcatgaaactgactct
gaacagccaacttatggacggctggcaaatcacctgggggctggatgctgagcatgagcgcttta
cctccaaccagatgttcttcgatctggctcaggcaagtgcttccggagggctgaacaaccataag
atttacaccaccgggcgctatccgtcatatgacatcaccaatctggcggccttcctgcaatccag
ctatgacattaatgatatttttaccgttagcggtggcgtacgctatcagtatactgagaacaggg
tagatgatttcatcgactacacgcagcaacagaagattgctgccgggaaggcgatatctgccgac
gccattcctggtggttcggtagattacgataactttctgttcaatgctggtctgctgatgcacat
caccgaacgtcagcaggcatggttcaatttttcccagggggtggcattgccggatccggggaaat
attatggtcgcggcatctatggtgcagcagtgaacggccatcttcccctgacaaagagcgtgaac
gtcagcgacagtaagctggaaggcgtgaaagtcgattcttatgaactgggctggcgctttaccgg
tgacaacctgcggactcaaatcgcggcatattactcgctttccaataagagcgtggaaaggaata
aagatctgaccatcagtgtgaaggacgacaggcgccgtatttacggcgtggaaggtgcggtggac
tacctgatcccggatactgactggagtaccggtgtgaacttcaatgtgctgaaaaccgagtcgaa
agtgaacggtcaatggcaaaaatatgacgtgaaggaatcaagtccatcgaaagcgacagcttaca
ttaactgggcgccggaaccgtggagtctgcgtgtacagagcaccacttctttcgacgtaagcgat
gcagagggtaacgatattaatggttacactaccgtcgattttatcagtagttggcagcttccggt
gggaacactcagcttcagcgttgagaacctcttcgaccgtgactataccactgtctggggacagc
gtgcacctctgtactacagcccgggttacggccctgcttcactgtacgactacaaaggccggggc
cgaacctttggtctgaactactcagtgctgttctga
A. pleuropneumoniae OmpA
Sequence ID Number 13
MKKSLVALTVLSAAAVAQAAPQQNTFYAGAKAGWASFHDGIEQLDSAKNTDRGTKYGINRNSVTY
GVFGGYQILNQDKLGLAAELGYDYFGRVRGSEKPNGKADKKTFRHAAHGATIALKPSYEVLPDLD
VYGKVGIALVNNTYKTFNAAQEKVKTRRFQSSLILGAGVEYAILPELAARVEYQWLNNAGKASYS
TLNRMGATDYRSDISSVSAGLSYRFGQGAVPVAAPAVETKNFAFSSDVLFAFGKSNLKPAAATAL
DAMQTEINNAGLSNAAIQVNGYTDRIGKEASNLKLSQRRAETVANYIVSKGALAANVTAVGYGEA
NPVTGATCDKVKGRKALIACLAPDRRVEVQVQGTKEVTM.
*In bold A.pleuropneumoniae native signal peptide
Nucleotide sequence from Sequence ID Number 13
atgaaaaaatcattagttgctttaacagtattatcggctgcagcggtagctcaagcagcgccaca
acaaaatactttctacgcaggtgcgaaagcaggttgggcgtcattccatgatggtatcgaacaat
tagattcagctaaaaacacagatcgcggtacaaaatacggtatcaaccgtaattcagtaacttac
ggcgtattcggcggttaccaaattttaaaccaagacaaattaggtttagcggctgaattaggtta
tgactatttcggtcgtgtgcgcggttctgaaaaaccaaacggtaaagcggacaagaaaactttcc
gtcacgctgcacacggtgcgacaatcgcattaaaacctagctacgaagtattacctgacttagac
gtttacggtaaagtaggtatcgcattagtaaacaatacatataaaacattcaatgcagcacaaga
gaaagtgaaaactcgtcgtttccaaagttctttaattttaggtgcgggtgttgagtacgcaattc
ttcctgaattagcggcacgtgttgaataccaatggttaaacaacgcaggtaaagcaagctactct
actttaaatcgtatgggtgcaactgactaccgttcggatatcagttccgtatctgcaggtttaag
ctaccgtttcggtcaaggtgcggtaccggttgcagctccggcagttgaaactaaaaacttcgcat
tcagctctgacgtattattcgcattcggtaaatcaaacttaaaaccggctgcggcaacagcatta
gatgcaatgcaaaccgaaatcaataacgcaggtttatcaaatgctgcgatccaagtaaacggtta
cacggaccgtatcggtaaagaagcttcaaacttaaaactttcacaacgtcgtgcggaaacagtag
ctaactacatcgtttctaaaggtgctctggcagctaacgtaactgcagtaggttacggtgaagca
aaccctgtaaccggcgcaacatgtgacaaagttaaaggtcgtaaagcattaatcgcttgcttagc
accggatcgtcgtgttgaagttcaagttcaaggtactaaagaagtaactatgtaa
A. pleuropneumoniae OmpW
Sequence ID Number 14
MKKAVLAAVLGGALLADSAMAHQAGDVIFRAGAIGVIANSSSDYQTGADVNLDVNNNIQLGLTGT
YMLSDNLGLELLAATPFSHKITGKLGATDLGEVAKVKHLPPSLYLQYYFFDSNATVRPYVGAGLN
YTRFFSAESLKPQLVQNLRVKKHSVAPIANLGVDVKLTDNLSFNAAAWYTRIKTTADYDVPGLGH
VSTPITLDPVVLFSGISYKF.
*In bold A. pleuropneumoniae native signal peptide
Nucleotide sequence from Sequence ID Number 14
atgaaaaaagcagtattagcggcagtattaggcggtgcgttattagcggattcggcaatggcaca
tcaagcgggcgatgtgattttccgtgccggtgcgatcggtgtgattgcaaattcaagttcggatt
atcaaaccggggcggacgtaaacttagatgtaaataataatattcagcttggtttaaccggtacc
tatatgttaagtgataatttaggtcttgaattattagcggcaacaccgttttctcacaaaatcac
cggtaagttaggtgcaacagatttaggcgaagtggcaaaagtaaaacatcttccgccgagccttt
acttacaatattatttctttgattctaatgcgacagttcgtccatacgttggtgccggtttaaac
tatactcgctttttcagtgctgaaagtttaaaaccgcaattagtacaaaacttacgtgttaaaaa
acattccgtcgcaccgattgcgaatttaggtgttgatgtgaaattaacggataatctatcattca
atgcggcagcttggtacacacgtattaaaactactgccgattatgatgttccgggattaggtcat
gtaagtacaccgattactttagatcctgttgtattattctcaggtattagctacaaattctaa
A. pleuropneumoniae TbpA
Sequence ID Number 15
MKNKLNLISLALLSLFAVQSYAEQAVQLNDVYVTGTKKKAHKKENEVTGLGKVVKTPDTLSKEQV
LGIRDLTRYDPGISVVEQGRGATTGYSIRGVDRNRVGLALDGLPQIQSYVSQYSRSSSGAINEIE
YENLRSIQISKGASSSEFGSGSLGGSVQFRTKEVSDIIKPGQSWGLDTKSAYSSKNQQWLNSLAF
AGTHNGFDALVIYTHRDGKETKAHKNAESRSQNITRVGVETNELDTSNRYTATTNNQHTYGWFLI
KDECPTLDCTPKQMARVTKDTPSFRSYPEYTPEEKQAYENQKHITERLNAQDYTGEYRALPDPLK
YKSDSWLVKLGYTFSPKHYVAGTYEHSKQRYDTRDMTYTAYWQPSDLLRTGRNWYPMNNAKGLYR
DNALDGVAIDYFTEDGVKSSKGLRWAKARFIDEWHTRDRLGALYRYTNQDGNRLIDRLSLSFDQQ
KINLSTRLRENNCSEYPTIDKNCRATLDKLWSSTKNEQSSYEEKHDTIQLSLDKTVQTGLGKHQL
NMLLGSDRFNSTLKRHEILSEFSVGSWGLVRDIGYRNGSYNNPYVYELKDQAIYSKNECDYSGTI
AGRADCATSKIKGHNHYIALRDNFAITKYLDIGLGYRFDKHKFRSTHRWANQGDYKNSAWNIGIV
AKPTSFLSLSYRASSGFRVPSFQELFGLRYDGAMKGSSDAYQKTEKLSPEKSLNQEVAATFKGDF
GVVEVSYFKNDYKQLIAPAERMHQTQSMINYFNVQDIKLDGINLIGKLDWNGVFDKIPEGIYTTL
AYSKMRVKEVKNYQGYMNIRSPLLDTIQPARYVVGVGYDQPDEKWGVNLTMTHSSGKNPNELRGN
EQVGFANYERTATKKRTRSWHTFDLTGYITPWKHTTVRAGVYNLMNYRYTTWESVRQSSLNAIHQ
HTNVKDYARYAAPGRNYVVSFEMKF.
*In bold A. pleuropneumoniae native signal peptide
Nucleotide sequence from Sequence ID Number 15
atgaaaaataaattaaatctgattagccttgctctgcttagcctctttgccgtacaaagctatgc
agaacaagcggtgcaattgaacgatgtttatgtcacaggaacaaaaaagaaagcacataaaaaag
agaacgaagtcacaggcttagggaaagtagttaaaacaccagatactcttagtaaggagcaagtg
ttaggaatacgagatctgactcgttacgaccccggtatttctgtcgtagaacaagggagaggtgc
gactacaggctactcaattcgtggggtagatcgtaaccgtgttggcttggcattagatggtttac
cacagattcagtcctatgtaagccagtattcacgttcctcaagcggtgccattaatgaaatagaa
tacgaaaatctgcgttcgatccaaattagtaaaggggctagttcttctgagtttggtagtggctc
actaggcggttcggtgcaattccgtaccaaagaggtaagcgacattattaagccagggcaatctt
ggggattagataccaaaagtgcctacagtagcaaaaatcaacaatggttaaactcacttgctttt
gctggtactcacaatggctttgatgctcttgtgatttacactcaccgtgatggtaaggaaacgaa
agctcataaaaatgcagagagtcgttctcagaatatcacccgagtaggagtggaaaccaacgagc
ttgatacctcaaatagatatactgcgacgacgaataatcaacatacttatggctggtttttgatt
aaagatgaatgtccaacgttagattgtacgccgaaacagatggctagggtgacaaaagatacgcc
atctttccgttcttaccctgaatatactcctgaggaaaaacaggcttatgagaaccaaaaacata
ttacagagcgtctaaatgctcaggattacactggtgaatatagagctttacctgatccgcttaaa
tataaatctgattcttggctggttaaattaggatacacattctctccgaaacattatgtcgctgg
tacttatgaacatagcaaacagcgttacgacacccgagatatgacctataccgcttattggcaac
catcggatttacttagaactggtagaaattggtatccaatgaataatgctaaaggattatatcgt
gataatgctttagatggtgttgctattgactactttacggaagatggtgtgaaatcctcaaaagg
tttacgttgggcaaaagctcgttttattgacgagtggcacactcgtgatcgcttaggtgctttat
atcgttatactaatcaagatggaaatcgtctgattgatagactatccttgagtttcgatcagcaa
aaaattaatttatctacccgcttgagagaaaacaactgttcggaatatccaaccatagataagaa
ttgccgtgcaactcttgataaactttggtcttctactaaaaatgagcaaagttcttatgaagaaa
aacacgacactattcagctctcgttagataaaaccgtacaaacgggattgggtaaacatcaatta
aatatgttattaggttcagaccgtttcaattccaccttaaaacgccatgaaattttgagtgaatt
ttctgtagggagttgggggcttgttagagacattggctatcgaaatggatcttacaataatcctt
atgtgtatgagctaaaagatcaggcaatttatagtaaaaatgaatgtgattatagtggcactatt
gcaggtagggctgattgtgctacaagtaaaatcaaagggcataatcactacatcgctctgagaga
taattttgccataaccaagtatttggatattggtttgggttaccgtttcgataagcataaattcc
gtagcactcatcgctgggcaaatcaaggcgattataaaaatagtgcgtggaatattggcatagtc
gcaaaaccaacgtcattcctatcgctctcttatcgagcatcatctggctttagagtgccaagttt
ccaagagctatttggcttacgttatgatggtgcaatgaaaggctccagcgatgcttaccaaaaaa
cagagaagttatctcctgaaaaatccttaaaccaagaggttgctgcgactttcaaaggtgatttt
ggtgtcgttgaagtcagttatttcaaaaatgactataagcagttaattgctccagcagaaagaat
gcaccaaactcaatcaatgattaactattttaatgtgcaagatattaaattggacggcattaatc
ttattggtaagctagattggaatggggtatttgataaaattcctgagggcatttacacaacattg
gcttatagcaaaatgcgagtaaaagaggtgaaaaactatcaagggtatatgaatattcgttctcc
attgttagataccattcagcctgctcgctatgttgtaggagtggggtacgatcagccagatgaaa
aatggggcgtgaatctaacaatgacacactccagtggaaaaaatccaaatgagttaagaggtaat
gaacaagtcggttttgccaattatgagcgaactgccacgaagaaaagaacacgttcttggcatac
ctttgacttaacgggatatatcaccccttggaaacatacaacggtacgagctggcgtatataacc
tgatgaattatcgttacaccacttgggaatccgtacgtcaatcttcgcttaatgcaattcatcag
catactaacgtaaaagactatgcaaggtatgcagcgcccggtagaaattatgttgtttcattcga
aatgaaattctaa
A. pleuropneumoniae ApfA
Sequence ID Number 16
MQKLSLIRPLTNAFTLIELMIVIAIIAILATVAIPSYNSYTQKAALSELLAASASYKTDVEICIY
NTGDSKNCSGGQNGVRKMTELRQAKYLNAITVEGGTITVTGKGNLQEYGYTMTPIHNGSTISWET
KCKGEDLSLFPANFCAIN.
*In bold A. pleuropneumoniae native signal peptide
Nucleotide sequence from Sequence ID Number 16
atgcaaaaactaagtcttattcgaccgcttactaacgcgtttactttaattgaattgatgatcgt
gattgcgattattgccattttagctacggttgcaattccgtcatataacagttatacccaaaaag
cggcgctttcggagctattggcggcatcggcttcttataaaacggatgtcgagatctgcatatat
aacaccggagattctaaaaactgtagcggcggtcaaaacggtgtcagaaaaatgacggagcttag
acaggctaaatatttaaatgccattacggtggaaggcggaacgattacggtaacggggaagggga
atctgcaggaatacggttatacgatgacaccgattcataacggtagcactatttcttgggaaacg
aaatgtaaaggggaggacttaagtttatttccggcaaatttctgtgcgataaattag
K. pneumoniae OmpA
Sequence ID Number 17
KLSRIALATMLVAAPLAAANAGGKLGWSQYHDTGFYGNGFQNNNGPTRNDQLGAGAFGGYQVNPY
LGFEMGYDWLGRMAYKGSVDNGAFKAQGVQLTAKLGYPITDDLDIYTRLGGMVWRADSKGNYAST
GVSRSEHDTGVSPVFAGGVEWAVTRDIATRLEYQWVNNIGDAGTVGTRPDNGMLSLGVSYRFGQE
DAAPVVAPAPAPAPEVATKHFTLKSDVLFNFNKATLKPEGQQALDQLYTQLSNMDPKDGSAVVLG
YTDRIGSEAYNQQLSEKRAQSVVDYLVAKGIPAGKISARGMGESNPVTGNTCDNVKARAALIDCL
APDRRVEIEVKGYKEVVTQPQA.
*In bold A. baumannii signal peptide
Nucleotide sequence from Sequence ID Number 17
atgaaattgagtcgtattgcacttgctactatgcttgttgctgctccattagctgctgctaatgc
tggtggtaaactgggttggtcccagtatcacgacaccggtttctacggtaacggtttccagaaca
acaacggtccgacccgtaacgatcagcttggtgctggtgcgttcggtggttaccaggttaacccg
tacctcggtttcgaaatgggttatgactggctgggccgtatggcatataaaggcagcgttgacaa
cggtgctttcaaagctcagggcgttcagctgaccgctaaactgggttacccgatcactgacgatc
tggacatctacacccgtctgggcggcatggtttggcgcgctgactccaaaggcaactacgcttct
accggcgtttcccgtagcgaacacgacactggcgtttccccagtatttgctggcggcgtagagtg
ggctgttactcgtgacatcgctacccgtctggaataccagtgggttaacaacatcggcgacgcgg
gcactgtgggtacccgtcctgataacggcatgctgagcctgggcgtttcctaccgcttcggtcag
gaagatgctgcaccggttgttgctccggctccggctccggctccggaagtggctaccaagcactt
caccctgaagtctgacgttctgttcaacttcaacaaagctaccctgaaaccggaaggtcagcagg
ctctggatcagctgtacactcagctgagcaacatggaCccgaaagacggttccgctgttgttctg
ggctacaccgaccgcatcggttccgaagcttacaaccagcagctgtctgagaaacgtgctcagtc
cgttgttgactacctggttgctaaaggcatcccggctggcaaaatctccgctcgcggcatgggtg
aatccaacccggttactggcaacacctgtgacaacgtgaaagctcgcgctgccctgatcgattgc
ctggctccggatcgtcgtgtagagatcgaagttaaaggctacaaagaagttgtaactcagccgca
ggcttaa
K. pneumoniae OmpK36
Sequence ID Number 18
MKLSRIALATMLVAAPLAAANAAEIYNKDGNKLDLYGKIDGLHYFSDDKSVDGDQTYMRVGVKGE
TQINDQLTGYGQWEYNVQANNTESSSDQAWTRLAFAGLKFGDAGSFDYGRNYGVVYDVTSWTDVL
PEFGGDTYGSDNFLQSRANGVATYRNSDFFGLVDGLNFALQYQGKNGSVSGEGALSPTNNGRTAL
KQNGDGYGTSLTYDIYDGISAGFAYSNSKRLGDQNSKLALGRGDNAETYTGGLKYDANNIYLATQ
YTQTYNATRAGSLGFANKAQNFEVVAQYQFDFGLRPSVAYLQSKGKDLEGYGDQDILKYVDVGAT
YYFNKNMSTYVDYKINLLDDNSFTHNAGISTDDVVALGLVYQF.
*In bold A. baumannii signal peptide
Nucleotide sequence from Sequence ID Number 18
atgaaattgagtcgtattgcacttgctactatgcttgttgctgctccattagctgctgctaatgc
tgctgaaatttataacaaagacggcaacaaattagacctgtacggtaaaattgacggtctgcact
acttctctgacgacaagagcgtcgacggcgaccagacctacatgcgtgtaggcgtgaaaggcgaa
acccagatcaacgaccagctgaccggttacggccagtgggaatacaacgttcaggcgaacaacac
tgaaagctccagcgatcaggcatggactcgtctggcattcgcaggcctgaaatttggcgacgcgg
gctctttcgactacggtcgtaactacggcgtagtatacgacgtaacgtcctggaccgacgttctg
ccggaattcggcggcgacacctacggttctgacaacttcctgcagtcccgtgctaacggcgttgc
aacctaccgtaactctgatttcttcggtctggttgacggcctgaactttgctctgcagtatcagg
gtaaaaacggcagcgtcagcggcgaaggcgctctgtctcctaccaacaacggtcgtaccgccttg
aaacagaacggcgacggttacggtacttctctgacctatgacatctatgatggcatcagcgctgg
tttcgcatactctaactccaaacgtcttggcgaccagaacagcaagctggcactgggtcgtggcg
acaacgctgaaacctacaccggcggtctgaaatacgacgcgaacaacatctacctggccactcag
tacacccagacctacaacgcgacccgcgccggttccctgggctttgctaacaaagcgcagaactt
cgaagtggttgctcagtaccagttcgacttcggtctgcgtccgtccgtggcttacctgcagtcta
aaggtaaggatctggaaggctacggcgaccaggacatcctgaaatatgttgacgttggcgcgacc
tactacttcaacaaaaacatgtccacctatgttgactacaaaatcaacctgctggacgacaatag
cttcacccacaacgccggtatctctaccgacgacgtggttgcactgggcctggtttaccagttct
aa
P. aeruginosa OprF
Sequence ID Number 19
MKLSRIALATMLVAAPLAAANAQGQNSVEIEAFGKRYFTDSVRNMKNADLYGGSIGYFLTDDVEL
ALSYGEYHDVRGTYETGNKKVHGNLTSLDAIYHFGTPGVGLRPYVSAGLAHQNITNINSDSQGRQ
QMTMANIGAGLKYYFTENFFAKASLDGQYGLEKRDNGHQGEWMAGLGVGFNFGGSKAAPAPEPVA
DVCSDSDNDGVCDNVDKCPDTPANVTVDANGCPAVAEVVRVQLDVKFDFDKSKVKENSYADIKNL
ADFMKQYPSTSTTVEGHTDSVGTDAYNQKLSERRANAVRDVLVNEYGVEGGRVNAVGYGESRPVA
DNATAEGRAINRRVEAEVEAEAK.
*In bold A. baumannii signal peptide
Nucleotide sequence from Sequence ID Number 19
atgaaattgagtcgtattgcacttgctactatgcttgttgctgctccattagctgctgctaatgc
tcagggccagaactcggtagagatcgaagccttcggcaagcgctacttcaccgacagcgttcgca
acatgaagaacgcggacctgtacggcggctcgatcggttacttcctgaccgacgacgtcgagctg
gcgctgtcctacggtgagtaccatgacgttcgtggcacctacgaaaccggcaacaagaaggtcca
cggcaacctgacctccctggacgccatctaccacttcggtaccccgggcgtaggtctgcgtccgt
acgtgtcggctggtctggctcaccagaacatcaccaacatcaacagcgacagccaaggccgtcag
cagatgaccatggccaacatcggcgctggtctgaagtactacttcaccgagaacttcttcgccaa
ggccagcctcgacggccagtacggtctggagaagcgtgacaacggtcaccagggcgagtggatgg
ctggcctgggcgtcggcttcaacttcggtggttcgaaagccgctccggctccggaaccggttgcc
gacgtttgctccgactccgacaacgacggcgtttgcgacaacgtcgacaagtgcccggatacccc
ggccaacgtcaccgttgacgccaacggctgcccggctgtcgccgaagtcgtacgcgtacagctgg
acgtgaagttcgacttcgacaagtccaaggtcaaagagaacagctacgctgacatcaagaacctg
gctgacttcatgaagcagtacccgtccacttccaccaccgttgaaggtcacaccgactccgtcgg
caccgacgcttacaaccagaagctgtccgagcgtcgtgccaacgccgttcgtgacgtactggtca
acgagtacggtgtagaaggtggtcgcgtgaacgctgttggttacggcgagtcccgcccggttgcc
gacaacgccaccgctgaaggccgcgctatcaaccgtcgcgttgaagccgaagtagaagctgaagc
caagtaa
P. aeruginosa OprI::PcrV fusion protein
Sequence ID Number 20
MKLSRIALATMLVAAPLAAANASHSKETEARLTATEDAAARAQARADEAYRKADEALGAAQKAQQ
TADEANERALRMLEKASRKGGGGSGGGGSGGGGSSAAPASAEQEELLALLRSERIVLAHAGQPLS
EAQVLKALAWLLAANPSAPPGQGLEVLREVLQARRQPGAQWDLREFLVSAYFSLHGRLDEDVIGV
YKDVLQTQDGKRKALLDELKALTAELKVYSVIQSQINAALSAKQGIRIDAGGIDLVDPTLYGYAV
GDPRWKDSPEYALLSNLDTFSGKLSIKDFLSGSPKQSGELKGLSDEYPFEKDNNPVGNFATTVSD
RSRPLNDKVNEKTTLLNDTSSRYNSAVEALNRFIQKYDSVLRDILSAI.
*In bold A. baumannii signal peptide
*linker underlined
Nucleotide sequence from Sequence ID Number 20
atgaaattgagtcgtattgcacttgctactatgcttgttgctgctccattagctgctgctaatgc
tagccactccaaagaaaccgaagctcgtctgaccgctaccgaagacgcagctgctcgtgctcagg
ctcgcgctgacgaagcctatcgcaaggctgacgaagctctgggcgctgctcagaaagctcagcag
actgctgacgaggctaacgagcgtgccctgcgcatgctggaaaaagccagccgcaagggcggcgg
cggcagcggcggcggcggcagcggcggcggcggcagctcggcggcgcctgccagtgccgagcagg
aggaactgctggccctgttgcgcagcgagcggatcgtgctggcccacgccggccagccgctgagc
gaggcgcaagtgctcaaggcgctcgcctggttgctcgcggccaatccgtccgcgcctccggggca
gggcctcgaggtactccgcgaagtcctgcaggcacgtcggcagcccggtgcgcagtgggatctgc
gcgagttcctggtgtcggcctatttcagcctgcacgggcgtctcgacgaggatgtcatcggtgtc
tacaaggatgtcctgcagacccaggacggcaagcgcaaggcgctgctcgacgagctgaaggcgct
gaccgcggagttgaaggtctacagcgtgatccagtcgcagatcaacgccgcgctgtcggccaagc
agggcatcaggatcgacgctggcggtatcgatctggtcgaccccacgctatatggctatgccgtc
ggcgatcccaggtggaaggacagccccgagtatgcgctgctgagcaatctggataccttcagcgg
caagctgtcgatcaaggattttctcagcggctcgccgaagcagagcggggaactcaagggcctca
gcgatgagtaccccttcgagaaggacaacaacccggtcggcaatttcgccaccacggtgagcgac
cgctcgcgtccgctgaacgacaaggtcaacgagaagaccaccctgctcaacgacaccagctcccg
ctacaactcggcggtcgaggcgctcaaccgcttcatccagaaatacgacagcgtcctgcgcgaca
ttctcagcgcgatctag
P. aeruginosa OprI
Sequence ID Number 21
MKLSRIALATMLVAAPLAAANASHSKETEARLTATEDAAARAQARADEAYRKADEALGAAQKAQQ
TADEANERALRMLEKASRK.
*In bold A. baumannii signal peptide
Nucleotide sequence from Sequence ID Number 21
atgaaattgagtcgtattgcacttgctactatgcttgttgctgctccattagctgctgctaatgc
tagccactccaaagaaaccgaagctcgtctgaccgctaccgaagacgcagctgctcgtgctcagg
ctcgcgctgacgaagcctatcgcaaggctgacgaagctctgggcgctgctcagaaagctcagcag
actgctgacgaggctaacgagcgtgccctgcgcatgctggaaaaagccagccgcaatag
E. coli OmpA
Sequence ID Number 22
MKLSRIALATMLVAAPLAAANAAPKDNTWYTGAKLGWSQYHDTGFIPNNGPTHENQLGAGAFGGY
QVNPYVGFEMGYDWLGRMPYKGDNINGAYKAQGVQLTAKLGYPITDDLDIYTRLGGMVWRADTKA
NVPGGASFKDHDTGVSPVFAGGVEYAITPEIATRLEYQWTNNIGDAHTIGTRPDNGMLSLGVSYR
FGQGEVAPVVAPAPAPAPEVQTKHFTLKSDVLFTFNKATLKPEGQAALDQLYSQLSNLDPKDGSV
VVLGYTDRIGSDAYNQALSERRAQSVVDYLISKGIPADKISARGMGESNPVTGNTCDNVKQRAAL
IDCLAPDRRVEIEVKGIKDVVTQPQA.
*In bold A. baumannii signal peptide
Nucleotide sequence from Sequence ID Number 22
atgaaattgagtcgtattgcacttgctactatgcttgttgctgctccattagctgctgctaatgc
tgctccgaaagataacacctggtacactggtgctaaactgggctggtcccagtaccatgacactg
gttttattcctaacaatggtccgacccacgaaaaccaactgggtgcaggtgcttttggtggttac
caggttaacccgtatgttggctttgaaatgggttacgactggttaggtcgtatgccgtacaaagg
cgacaacatcaacggcgcatacaaagctcagggcgttcagctgaccgctaaactgggttacccaa
tcactgacgatctggacatctacactcgtctgggtggtatggtatggcgtgcagacaccaaggct
aacgtacctggtggcgcatcctttaaagaccacgacaccggcgtttctccggtcttcgctggcgg
tgttgagtatgcgatcactcctgaaatcgctacccgtctggaataccagtggaccaacaacatcg
gtgacgcacacaccatcggcactcgtccggacaacggcatgctgagcctgggtgtttcctaccgt
ttcggtcagggcgaagtagctccagtagttgctccggctccagctccggcaccggaagtacagac
caagcacttcactctgaagtctgacgttctgttcaccttcaacaaagcaaccctgaaaccggaag
gtcaggctgctctggatcagctgtacagccagctgagcaacctggatccgaaagacggttccgta
gttgttctgggttacactgaccgcatcggttctgacgcttataaccaggctctgtccgagcgtcg
tgctcagtccgttgttgattacctgatctctaaaggtatcccggcagacaaaatctccgcacgtg
gtatgggcgaatccaacccggttactggcaacacctgtgacaacgtgaaacagcgtgctgcactg
atcgattgcctggctccggatcgtcgcgtagagatcgaagttaaaggcatcaaagacgttgtaac
tcagccgcaggcttaa
E. coli OmpX
Sequence ID Number 23
MKLSRIALATMLVAAPLAAANAATSTVTGGYAQSDAQGQMNKMGGFNLKYRYEEDNSPLGVIGSF
TYTEKSRTASSGDYNKNQYYGITAGPAYRINDWASIYGVVGVGYGKFQTTEYPTYKHDTSDYGFS
YGAGLQFNPMENVALDFSYEQSRIRSVDVGTWIAGVGYRF.
*In bold A. baumannii signal peptide
Nucleotide sequence from Sequence ID Number 23
atgaaattgagtcgtattgcacttgctactatgcttgttgctgctccattagctgctgctaatgc
tgcgacttctaccgtaactggcggttacgcacaaagcgacgctcagggccaaatgaacaaaatgg
gggtttcaacctgaaataccgctatgaagaagacaacagcccgctgggtgtgatcggttctttca
cttacaccgagaaaagccgtactgcaagctctggtgactacaacaaaaaccagtactacggcatc
actgctggtccggcttaccgcattaacgactgggcaagcatctacggtgtagtgggtgtgggtta
tggtaaattccagaccactgaatacccgacctacaaacacgacaccagcgactacggtttctcct
acggtgctggtttgcagttcaacccgatggaaaacgttgctctggacttctcttacgagcagagc
cgtattcgtagcgttgacgtaggcacctggattgccggtgttggttaccgcttctaa
E. coli FyuA
Sequence ID Number 24
MKLSRIALATMLVAAPLAAANANAQTSQQDESTLVVTASKQSSRSASANNVSSTVVSAPELSDAG
VTASDKLPRVLPGLNIENSGNMLFSTISLRGVSSAQDFYNPAVTLYVDGVPQLSTNTIQALTDVQ
SVELLRGPQGTLYGKSAQGGIINIVTQQPDSTPRGYIEGGVSSRDSYRSKFNLSGPIQDGLLYGS
VTLLRQVDDGDMINPATGSDDLGGTRASIGNVKLRLAPDDQPWEMGFAASRECTRATQDAYVGWN
DIKGRKLSISDGSPDPYMRRCTDSQTLSGKYTTDDWVFNLISAWQQQHYSRTFPSGSLIVNMPQR
WNQDVQELRAATLGDARTVDMVFGLYRQNTREKLNSAYDMPTMPYLSSTGYTTAETLAAYSDLTW
HLTDRFDIGGGVRFSHDKSSTQYHGSMLGNPFGDQGKSNDDQVLGQLSAGYMLTDDWRVYTRVAQ
GYKPSGYNIVPTAGLDAKPFVAEKSINYELGTRYETADVTLQAATFYTHTKDMQLYSGPVGMQTL
SNAGKADATGVELEAKWRFAPGWSWDINGNVIRSEFTNDSELYHGNRVPFVPRYGAGSSVNGVID
TRYGALMPRLAVNLVGPHYFDGDNQLRQGTYATLDSSLGWQATERMNISVYVDNLFDRRYRTYGY
MNGSSAVAQVNMGRTVGINTRIDFF.
*In bold A. baumannii signal peptide
Nucleotide sequence from Sequence ID Number 24
atgaaattgagtcgtattgcacttgctactatgcttgttgctgctccattagctgctgctaatgc
tcagacttcacagcaagacgaaagcacgctggtggttaccgccagtaaacaatcttcccgctcgg
catcagccaacaacgtctcatctactgttgtcagcgcgccggaattaagcgacgccggcgtcacc
gccagcgacaaactccccagagtcttgcccgggctcaatattgaaaatagcggcaacatgctttt
ttcgacgatctcgctacgcggcgtctcttcagcgcaggacttctataaccccgccgtcaccctgt
atgtcgatggcgtccctcagctttccaccaacaccatccaggcgcttaccgatgtgcaaagcgtg
gagttgctgcgaggcccacagggaacgttatatggcaaaagcgctcagggcgggatcatcaacat
cgtcacccagcagccggacagcacgccgcgcggctatattgaaggcggcgtcagtagccgcgaca
gttatcgaagtaagttcaacctgagcggccccattcaggatggcctgctgtacggcagcgtcacc
ctgttacgccaggttgatgacggcgacatgattaaccccgcgacgggaagcgatgacttaggcgg
cacccgcgccagcatagggaatgtgaaactgcgtctggcgccggacgatcagccctgggaaatgg
gctttgccgcctcacgcgaatgtacccgcgccacccaggatgcctatgtgggatggaatgatatt
aagggccgtaagctgtcgatcagcgatggttcaccagacccgtacatgcggcgctgcactgacag
ccagaccctgagtgggaaatacaccaccgatgactgggttttcaacctgatcagcgcctggcagc
agcagcattattcgcgcaccttcccttccggttcgttaatcgtcaatatgcctcagcgctggaat
caggatgtgcaggagctgcgcgccgcaaccctgggcgatgcgcgtaccgttgatatggtgtttgg
gctgtaccggcagaacacccgcgagaagttaaattcagcctacgacatgccgacaatgccttatt
taagcagtaccggctataccaccgctgaaacgctggccgcatacagtgacctgacctggcattta
accgatcgttttgatatcggcggcggcgtgcgcttctcgcatgataaatccagtacacaatatca
cggcagcatgctcggcaacccgtttggcgaccagggtaagagcaatgacgatcaggtgctcgggc
agctatccgcaggctatatgctgaccgatgactggagagtgtatacccgtgtagcccagggatat
aaaccttccgggtacaacatcgtgcctactgcgggtcttgatgccaaaccgttcgtcgccgagaa
atccatcaactatgaacttggcacccgctacgaaaccgctgacgtcacgctgcaagccgcgacgt
tttatacccacaccaaagacatgcagctttactctggcccggtcgggatgcagacattaagcaat
gcgggtaaagccgacgccaccggcgttgagcttgaagcgaagtggcggtttgcgccaggctggtc
atgggatatcaatggcaacgtgatccgttccgaattcaccaatgacagtgagttgtatcacggta
accgggtgccgttcgtaccacgttatggcgcgggaagcagcgtgaacggcgtgattgatacgcgc
tatggcgcactgatgccccgactggcggttaatctggtcgggccgcattatttcgatggcgacaa
ccagttgcggcaaggcacctatgccaccctggacagcagcctgggctggcaggcgactgaacgga
tgaacatttccgtctatgtcgataacctgttcgaccgtcgttaccgtacctatggctacatgaac
ggcagcagcgccgtcgcgcaggtcaatatgggtcgcaccgtcggtatcaatacgcgaattgattt
cttctga
E. coli Hma
Sequence ID Number 25
MKLSRIALATMLVAAPLAAANATSSETKISNEETLVVTTNRSASNLWESPATIQVIDQQTLQNST
NASIADNLQDIPGVEITDNSLAGRKQIRIRGEASSRVLILIDGQEVTYQRAGDNYGVGLLIDESA
LERVEVVKGPYSVLYGSQAIGGIVNFITKKGGDKLASGVVKAVYNSATAGWEESIAVQGSIGGFD
YRINGSYSDQGNRDTPDGRLPNTNYRNNSQGVWLGYNSGNHRFGLSLDRYRLATQTYYEDPDGSY
EAFSVKIPKLEREKVGVFYDTDVDGDYLKKIHFDAYEQTIQRQFANEVKTTQPVPSPMIQALTVH
NKTDTHDKQYTQAVTLQSHFSLPANNELVTGAQYKQDRVSQRSGGMTSSKSLTGFINKETRTRSY
YESEQSTVSLFAQNDWRFADHWTWTMGVRQYWLSSKLTRGDGVSYTAGIISDTSLARESASDHEM
VTSTSLRYSGFDNLELRAAFAQGYVFPTLSQLFMQTSAGGSVTYGNPDLKAEHSNNFELGARYNG
NQWLIDSAVYYSEAKDYIASLICDGSIVCNGNTNSSRSSYYYYDNIDRAKTWGLEISAEYNGWVF
SPYISGNLIRRQYETSTLKTTNTGEPAINGRIGLKHTLVMGQANIISDVFIRAASSAKDDSNGTE
TNVPGWATLNFAVNTEFGNEDQYRINLALNNLTDKRYRTAHETIPAAGFNAAIGFVWNF.
*In bold A. baumannii signal peptide
Nucleotide sequence from Sequence ID Number 25
atgaaattgagtcgtattgcacttgctactatgcttgttgctgctccattagctgctgctaatgc
tacttcttcagaaacaaaaatcagcaacgaagagacgctcgtcgtgaccacgaatcgttcggcaa
gcaacctttgggaaagcccggcgactatacaggttattgaccaacaaacattgcagaactccacc
aatgcctccatagccgataatttgcaggacatccccggagtagagataacagacaactccttggc
aggccgtaaacaaatccgcattcgtggcgaagcatcctcccgtgttttaattctcattgatggtc
aggaggtaacttatcagcgcgccggagataattatggtgtgggactgttgatagatgagtctgcg
ctggagcgtgttgaggtagtgaaaggtccatattccgtactgtacggttcacaggcaattggcgg
tattgttaacttcataaccaaaaagggaggtgacaaacttgcatctggagttgtgaaagctgttt
ataattccgcaacagcaggctgggaagaatcaatcgcggtccaggggagcatcggtggatttgat
tatcgcatcaacggtagttattctgatcagggcaatcgtgatacgccggatggacgtctgccgaa
taccaactatcgtaacaatagtcagggtgtatggttgggttataactccggaaaccatcgttttg
gcctctcgcttgatcgctacagactcgcgacgcaaacttactatgaggatccagacggaagctat
gaggcatttagtgtcaaaatacctaaacttgaacgagagaaagttggggtattctatgacacaga
cgtggacggtgactatctaaaaaaaattcatttcgacgcgtatgagcagaccatccagcgccaat
ttgccaacgaagtaaaaacgacacagcctgttcccagtccgatgattcaggctctgaccgttcat
aacaagactgacacccatgataagcaatacactcaggcggtcacattgcagagtcacttttcgct
gcctgctaataatgaacttgttaccggtgcacagtacaaacaagacagggtcagccaaaggtccg
gtggcatgacctcaagcaaatctctgaccggcttcattaataaggaaacacgaactcgctcctat
tatgagtcagagcaaagtacagtctcactattcgcacaaaatgactggcgattcgccgatcactg
gacatggacaatgggagttcgccaatactggctttcttcaaagttgacgcgtggtgacggagtat
catataccgcaggcattataagcgatacctctcttgccagagagtctgcgagtgatcacgaaatg
gtaacatctacaagcctgcgctattcaggtttcgataacttggagttacgcgctgcgttcgcgca
aggctacgtatttcccacactctcccagctttttatgcagacatctgcgggcggcagtgtcacat
acggaaatcctgatcttaaggctgaacactccaataactttgaattaggtgcacgatataatggt
aatcagtggctgattgacagcgcagtttactactcagaagctaaagattatattgcaagtctgat
ctgtgatggcagtatagtttgcaatggtaacaccaactcctcccgtagtagctactattattatg
acaatattgatcgggcaaaaacatggggactggaaataagcgcggaatataatggctgggttttc
tcgccatatatcagtggcaatttaattcgtcggcaatatgaaacttcaacattaaaaacaactaa
tacaggagaaccagcgataaacggacgtatagggctgaaacatactcttgtgatgggtcaggcca
acataatctctgatgtttttattcgtgctgcctctagtgcaaaagatgacagtaacggtaccgaa
acaaatgttccgggctgggccactctcaactttgcagtaaatacagaattcggtaacgaggatca
gtaccggattaacctagcactcaataacctgacagacaaacgctaccgtacagcacatgaaacta
ttcctgcagcaggttttaatgcagctataggttttgtatggaatttctga
E. coli IutA
Sequence ID Number 26
MKLSRIALATMLVAAPLAAANAQQNDDNEIIVSASRSNRTVAEMAQTTWVIENAELEQQIQGGKE
LKDALAQLIPGLDVSSQSRTNYGMNMRGRPLVVLIDGVRLNSSRSDSRQLDSVDPFNIDHIEVIS
GATALYGGGSTGGLINIVTKKGQPETMMEFEAGTKSGENSSKDHDERIAGAVSGGNDHISGRLSV
AYQKFGGWFDGNGDATLLDNTQTGLQHSNRLDIMGTGTLNIDESRQLQLITQYYKSQGDDNYGLN
LGKGFSAISGSSTPYVSKGLNSDRIPGTERHLISLQYSDSDFLRQELVGQVYYRDESLRFYPFPT
VNANKQATAFSSSQQDTDQYGMKLTLNSQLMDGWQITWGLDAEHERFTSNQMFFDLAQASASGGL
NNHKIYTTGRYPSYDITNLAAFLQSSYDINDIFTVSGGVRYQYTENRVDDFIDYTQQQKIAAGKA
ISADAIPGGSVDYDNFLFNAGLLMHITERQQAWFNFSQGVALPDPGKYYGRGIYGAAVNGHLPLT
KSVNVSDSKLEGVKVDSYELGWRFTGDNLRTQIAAYYSLSNKSVERNKDLTISVKDDRRRIYGVE
GAVDYLIPDTDWSTGVNFNVLKTESKVNGQWQKYDVKESSPSKATAYINWAPEPWSLRVQSTTSF
DVSDAEGNDINGYTTVDFISSWQLPVGTLSFSVENLFDRDYTTVWGQRAPLYYSPGYGPASLYDY
KGRGRTFGLNYSVLF.
*In bold A. baumannii signal peptide
Nucleotide sequence from Sequence ID Number 26
atgaaattgagtcgtattgcacttgctactatgcttgttgctgctccattagctgctgctaatgc
tcagcaaaacgatgataatgagatcatagtgtctgccagccgcagcaatcgaactgtagcggaga
tggcgcaaaccacctgggttatcgaaaatgccgaactggagcagcagattcagggcggtaaagag
ctgaaagacgcactggctcagttaatccccggccttgatgtcagcagccagagccgaaccaacta
cggtatgaacatgcgtggccgcccgctggttgtcctgattgacggtgtgcgcctcaactcttcac
gttccgacagccgacaactggactctgtcgatccttttaatatcgaccatattgaagtgatctcc
ggcgcgacggccctgtacggtgggggagtaccggagggttgatcaacatcgtgaccaaaaaaggc
cagccggaaaccatgatggagtttgaggctggcacaaaaagtggctttaacagcagtaaagatca
cgatgagcgcattgccggtgctgtctccggcggaaatgaccatatctccggacgtctttccgtgg
catatcagaaatttggcggctggtttgacggtaacggcgatgccaccctgcttgataacacccag
accggcctgcagcactccaatcggctggacatcatgggaaccggtacgctgaacatcgatgaatc
ccggcagcttcaactgataacgcagtactataaaagtcagggggacgacaattacgggcttaatc
tcgggaaaggcttttccgccatcagcgggagcagcacaccatacgtcagtaaggggctgaattct
gaccgcattcccggcactgagcggcatttgatcagcctgcagtactctgacagtgatttcctgag
acaggaactggtcggtcaggtttactaccgcgatgagtcgttgcggttctacccgttcccgacgg
taaatgcgaataaacaggcgacggctttctcctcgtcacagcaggataccgaccagtacggcatg
aaactgactctgaacagccaacttatggacggctggcaaatcacctgggggctggatgctgagca
tgagcgctttacctccaaccagatgttcttcgatctggctcaggcaagtgcttccggagggctga
acaaccataagatttacaccaccgggcgctatccgtcatatgacatcaccaatctggcggccttc
ctgcaatccagctatgacattaatgatatttttaccgttagcggtggcgtacgctatcagtatac
tgagaacagggtagatgatttcatcgactacacgcagcaacagaagattgctgccgggaaggcga
tatctgccgacgccattcctggtggttcggtagattacgataactttctgttcaatgctggtctg
ctgatgcacatcaccgaacgtcagcaggcatggttcaatttttcccagggggtggcattgccgga
tccggggaaatattatggtcgcggcatctatggtgcagcagtgaacggccatcttcccctgacaa
agagcgtgaacgtcagcgacagtaagctggaaggcgtgaaagtcgattcttatgaactgggctgg
cgctttaccggtgacaacctgcggactcaaatcgcggcatattactcgctttccaataagagcgt
ggaaaggaataaagatctgaccatcagtgtgaaggacgacaggcgccgtatttacggcgtggaag
gtgcggtggactacctgatcccggatactgactggagtaccggtgtgaacttcaatgtgctgaaa
accgagtcgaaagtgaacggtcaatggcaaaaatatgacgtgaaggaatcaagtccatcgaaagc
gacagcttacattaactgggcgccggaaccgtggagtctgcgtgtacagagcaccacttctttcg
acgtaagcgatgcagagggtaacgatattaatggttacactaccgtcgattttatcagtagttgg
cagcttccggtgggaacactcagcttcagcgttgagaacctcttcgaccgtgactataccactgt
ctggggacagcgtgcacctctgtactacagcccgggttacggccctgcttcactgtacgactaca
aaggccggggccgaacctttggtctgaactactcagtgctgttctga
A. pleuropneumoniae OmpA
Sequence ID Number 27
MKLSRIALATMLVAAPLAAANAAAPQQNTFYAGAKAGWASFHDGIEQLDSAKNTDRGTKYGINRN
SVTYGVFGGYQILNQDKLGLAAELGYDYFGRVRGSEKPNGKADKKTFRHAAHGATIALKPSYEVL
PDLDVYGKVGIALVNNTYKTFNAAQEKVKTRRFQSSLILGAGVEYAILPELAARVEYQWLNNAGK
ASYSTLNRMGATDYRSDISSVSAGLSYRFGQGAVPVAAPAVETKNFAFSSDVLFAFGKSNLKPAA
ATALDAMQTEINNAGLSNAAIQVNGYTDRIGKEASNLKLSQRRAETVANYIVSKGALAANVTAVG
YGEANPVTGATCDKVKGRKALIACLAPDRRVEVQVQGTKEVTM.
*In bold A. baumannii signal peptide
Nucleotide sequence from Sequence ID Number 27
atgaaattgagtcgtattgcacttgctactatgcttgttgctgctccattagctgctgctaatgc
tgcagcgccacaacaaaatactttctacgcaggtgcgaaagcaggttgggcgtcattccatgatg
gtatcgaacaattagattcagctaaaaacacagatcgcggtacaaaatacggtatcaaccgtaat
tcagtaacttacggcgtattcggcggttaccaaattttaaaccaagacaaattaggtttagcggc
tgaattaggttatgactatttcggtcgtgtgcgcggttctgaaaaaccaaacggtaaagcggaca
agaaaactttccgtcacgctgcacacggtgcgacaatcgcattaaaacctagctacgaagtatta
cctgacttagacgtttacggtaaagtaggtatcgcattagtaaacaatacatataaaacattcaa
tgcagcacaagagaaagtgaaaactcgtcgtttccaaagttctttaattttaggtgcgggtgttg
agtacgcaattcttcctgaattagcggcacgtgttgaataccaatggttaaacaacgcaggtaaa
gcaagctactctactttaaatcgtatgggtgcaactgactaccgttcggatatcagttccgtatc
tgcaggtttaagctaccgtttcggtcaaggtgcggtaccggttgcagctccggcagttgaaacta
aaaacttcgcattcagctctgacgtattattcgcattcggtaaatcaaacttaaaaccggctgcg
gcaacagcattagatgcaatgcaaaccgaaatcaataacgcaggtttatcaaatgctgcgatcca
agtaaacggttacacggaccgtatcggtaaagaagcttcaaacttaaaactttcacaacgtcgtg
cggaaacagtagctaactacatcgtttctaaaggtgctctggcagctaacgtaactgcagtaggt
tacggtgaagcaaaccctgtaaccggcgcaacatgtgacaaagttaaaggtcgtaaagcattaat
cgcttgcttagcaccggatcgtcgtgttgaagttcaagttcaaggtactaaagaagtaactatgt
aa
A. pleuropneumoniae OmpW
Sequence ID Number 28
MKLSRIALATMLVAAPLAAANAAHQAGDVIFRAGAIGVIANSSSDYQTGADVNLDVNNNIQLGLT
GTYMLSDNLGLELLAATPFSHKITGKLGATDLGEVAKVKHLPPSLYLQYYFFDSNATVRPYVGAG
LNYTRFFSAESLKPQLVQNLRVKKHSVAPIANLGVDVKLTDNLSFNAAAWYTRIKTTADYDVPGL
GHVSTPITLDPVVLFSGISYKF.
*In bold A. baumannii signal peptide
Nucleotide sequence from Sequence ID Number 28
atgaaattgagtcgtattgcacttgctactatgcttgttgctgctccattagctgctgctaatgc
tgcacatcaagcgggcgatgtgattttccgtgccggtgcgatcggtgtgattgcaaattcaagtt
cggattatcaaaccggggcggacgtaaacttagatgtaaataataatattcagcttggtttaacc
ggtacctatatgttaagtgataatttaggtcttgaattattagcggcaacaccgttttctcacaa
aatcaccggtaagttaggtgcaacagatttaggcgaagtggcaaaagtaaaacatcttccgccga
gcctttacttacaatattatttctttgattctaatgcgacagttcgtccatacgttggtgccggt
ttaaactatactcgctttttcagtgctgaaagtttaaaaccgcaattagtacaaaacttacgtgt
taaaaaacattccgtcgcaccgattgcgaatttaggtgttgatgtgaaattaacggataatctat
cattcaatgcggcagcttggtacacacgtattaaaactactgccgattatgatgttccgggatta
ggtcatgtaagtacaccgattactttagatcctgttgtattattctcaggtattagctacaaatt
ctaa
A. pleuropneumoniae TbpA
Sequence ID Number 29
MKLSRIALATMLVAAPLAAANAAEQAVQLNDVYVTGTKKKAHKKENEVTGLGKVVKTPDTLSKEQ
VLGIRDLTRYDPGISVVEQGRGATTGYSIRGVDRNRVGLALDGLPQIQSYVSQYSRSSSGAINEI
EYENLRSIQISKGASSSEFGSGSLGGSVQFRTKEVSDIIKPGQSWGLDTKSAYSSKNQQWLNSLA
FAGTHNGFDALVIYTHRDGKETKAHKNAESRSQNITRVGVETNELDTSNRYTATTNNQHTYGWFL
IKDECPTLDCTPKQMARVTKDTPSFRSYPEYTPEEKQAYENQKHITERLNAQDYTGEYRALPDPL
KYKSDSWLVKLGYTFSPKHYVAGTYEHSKQRYDTRDMTYTAYWQPSDLLRTGRNWYPMNNAKGLY
RDNALDGVAIDYFTEDGVKSSKGLRWAKARFIDEWHTRDRLGALYRYTNQDGNRLIDRLSLSFDQ
QKINLSTRLRENNCSEYPTIDKNCRATLDKLWSSTKNEQSSYEEKHDTIQLSLDKTVQTGLGKHQ
LNMLLGSDRFNSTLKRHEILSEFSVGSWGLVRDIGYRNGSYNNPYVYELKDQAIYSKNECDYSGT
IAGRADCATSKIKGHNHYIALRDNFAITKYLDIGLGYRFDKHKFRSTHRWANQGDYKNSAWNIGI
VAKPTSFLSLSYRASSGFRVPSFQELFGLRYDGAMKGSSDAYQKTEKLSPEKSLNQEVAATFKGD
FGVVEVSYFKNDYKQLIAPAERMHQTQSMINYFNVQDIKLDGINLIGKLDWNGVFDKIPEGIYTT
LAYSKMRVKEVKNYQGYMNIRSPLLDTIQPARYVVGVGYDQPDEKWGVNLTMTHSSGKNPNELRG
NEQVGFANYERTATKKRTRSWHTFDLTGYITPWKHTTVRAGVYNLMNYRYTTWESVRQSSLNAIH
QHTNVKDYARYAAPGRNYVVSFEMKF.
*In bold A. baumannii signal peptide
Nucleotide sequence from Sequence ID Number 29
atgaaattgagtcgtattgcacttgctactatgcttgttgctgctccattagctgctgctaatgc
tgcagaacaagcggtgcaattgaacgatgtttatgtcacaggaacaaaaaagaaagcacataaaa
aagagaacgaagtcacaggcttagggaaagtagttaaaacaccagatactcttagtaaggagcaa
gtgttaggaatacgagatctgactcgttacgaccccggtatttctgtcgtagaacaagggagagg
tgcgactacaggctactcaattcgtggggtagatcgtaaccgtgttggcttggcattagatggtt
taccacagattcagtcctatgtaagccagtattcacgttcctcaagcggtgccattaatgaaata
gaatacgaaaatctgcgttcgatccaaattagtaaaggggctagttcttctgagtttggtagtgg
ctcactaggcggttcggtgcaattccgtaccaaagaggtaagcgacattattaagccagggcaat
cttggggattagataccaaaagtgcctacagtagcaaaaatcaacaatggttaaactcacttgct
tttgctggtactcacaatggctttgatgctcttgtgatttacactcaccgtgatggtaaggaaac
gaaagctcataaaaatgcagagagtcgttctcagaatatcacccgagtaggagtggaaaccaacg
agcttgatacctcaaatagatatactgcgacgacgaataatcaacatacttatggctggtttttg
attaaagatgaatgtccaacgttagattgtacgccgaaacagatggctagggtgacaaaagatac
gccatctttccgttcttaccctgaatatactcctgaggaaaaacaggcttatgagaaccaaaaac
atattacagagcgtctaaatgctcaggattacactggtgaatatagagctttacctgatccgctt
aaatataaatctgattcttggctggttaaattaggatacacattctctccgaaacattatgtcgc
tggtacttatgaacatagcaaacagcgttacgacacccgagatatgacctataccgcttattggc
aaccatcggatttacttagaactggtagaaattggtatccaatgaataatgctaaaggattatat
cgtgataatgctttagatggtgttgctattgactactttacggaagatggtgtgaaatcctcaaa
aggtttacgttgggcaaaagctcgttttattgacgagtggcacactcgtgatcgcttaggtgctt
tatatcgttatactaatcaagatggaaatcgtctgattgatagactatccttgagtttcgatcag
caaaaaattaatttatctacccgcttgagagaaaacaactgttcggaatatccaaccatagataa
gaattgccgtgcaactcttgataaactttggtcttctactaaaaatgagcaaagttcttatgaag
aaaaacacgacactattcagctctcgttagataaaaccgtacaaacgggattgggtaaacatcaa
ttaaatatgttattaggttcagaccgtttcaattccaccttaaaacgccatgaaattttgagtga
attttctgtagggagttgggggcttgttagagacattggctatcgaaatggatcttacaataatc
cttatgtgtatgagctaaaagatcaggcaatttatagtaaaaatgaatgtgattatagtggcact
attgcaggtagggctgattgtgctacaagtaaaatcaaagggcataatcactacatcgctctgag
agataattttgccataaccaagtatttggatattggtttgggttaccgtttcgataagcataaat
tccgtagcactcatcgctgggcaaatcaaggcgattataaaaatagtgcgtggaatattggcata
gtcgcaaaaccaacgtcattcctatcgctctcttatcgagcatcatctggctttagagtgccaag
tttccaagagctatttggcttacgttatgatggtgcaatgaaaggctccagcgatgcttaccaaa
aaacagagaagttatctcctgaaaaatccttaaaccaagaggttgctgcgactttcaaaggtgat
tttggtgtcgttgaagtcagttatttcaaaaatgactataagcagttaattgctccagcagaaag
aatgcaccaaactcaatcaatgattaactattttaatgtgcaagatattaaattggacggcatta
atcttattggtaagctagattggaatggggtatttgataaaattcctgagggcatttacacaaca
ttggcttatagcaaaatgcgagtaaaagaggtgaaaaactatcaagggtatatgaatattcgttc
tccattgttagataccattcagcctgctcgctatgttgtaggagtggggtacgatcagccagatg
aaaaatggggcgtgaatctaacaatgacacactccagtggaaaaaatccaaatgagttaagaggt
aatgaacaagtcggttttgccaattatgagcgaactgccacgaagaaaagaacacgttcttggca
tacctttgacttaacgggatatatcaccccttggaaacatacaacggtacgagctggcgtatata
acctgatgaattatcgttacaccacttgggaatccgtacgtcaatcttcgcttaatgcaattcat
cagcatactaacgtaaaagactatgcaaggtatgcagcgcccggtagaaattatgttgtttcatt
cgaaatgaaattctaa
A. pleuropneumoniae ApfA
Sequence ID Number 30
MKLSRIALATMLVAAPLAAANAFTLIELMIVIAIIAILATVAIPSYNSYTQKAALSELLAASASY
KTDVEICIYNTGDSKNCSGGQNGVRKMTELRQAKYLNAITVEGGTITVTGKGNLQEYGYTMTPIH
NGSTISWETKCKGEDLSLFPANFCAIN.
*In bold A. baumannii signal peptide
Nucleotide sequence from Sequence ID Number 30
atgaaattgagtcgtattgcacttgctactatgcttgttgctgctccattagctgctgctaatgc
ttttactttaattgaattgatgatcgtgattgcgattattgccattttagctacggttgcaattc
cgtcatataacagttatacccaaaaagcggcgctttcggagctattggcggcatcggcttcttat
aaaacggatgtcgagatctgcatatataacaccggagattctaaaaactgtagcggcggtcaaaa
cggtgtcagaaaaatgacggagcttagacaggctaaatatttaaatgccattacggtggaaggcg
gaacgattacggtaacggggaaggggaatctgcaggaatacggttatacgatgacaccgattcat
aacggtagcactatttcttgggaaacgaaatgtaaaggggaggacttaagtttatttccggcaaa
tttctgtgcgataaattag
K. pneumoniae OmpA
Sequence ID Number 31
GGKLGWSQYHDTGFYGNGFQNNNGPTRNDQLGAGAFGGYQVNPYLGFEMGYDWLGRMAYKGSVDN
GAFKAQGVQLTAKLGYPITDDLDIYTRLGGMVWRADSKGNYASTGVSRSEHDTGVSPVFAGGVEW
AVTRDIATRLEYQWVNNIGDAGTVGTRPDNGMLSLGVSYRFGQEDAAPVVAPAPAPAPEVATKHF
TLKSDVLFNFNKATLKPEGQQALDQLYTQLSNMDPKDGSAVVLGYTDRIGSEAYNQQLSEKRAQS
VVDYLVAKGIPAGKISARGMGESNPVTGNTCDNVKARAALIDCLAPDRRVEIEVKGYKEVVTQPQ
A
Nucleotide sequence from Sequence ID Number 31
ggtggtaaactgggttggtcccagtatcacgacaccggtttctacggtaacggtttccagaacaa
caacggtccgacccgtaacgatcagcttggtgctggtgcgttcggtggttaccaggttaacccgt
acctcggtttcgaaatgggttatgactggctgggccgtatggcatataaaggcagcgttgacaac
ggtgctttcaaagctcagggcgttcagctgaccgctaaactgggttacccgatcactgacgatct
ggacatctacacccgtctgggcggcatggtttggcgcgctgactccaaaggcaactacgcttcta
ccggcgtttcccgtagcgaacacgacactggcgtttccccagtatttgctggcggcgtagagtgg
gctgttactcgtgacatcgctacccgtctggaataccagtgggttaacaacatcggcgacgcggg
cactgtgggtacccgtcctgataacggcatgctgagcctgggcgtttcctaccgcttcggtcagg
aagatgctgcaccggttgttgctccggctccggctccggctccggaagtggctaccaagcacttc
accctgaagtctgacgttctgttcaacttcaacaaagctaccctgaaaccggaaggtcagcaggc
tctggatcagctgtacactcagctgagcaacatggaCccgaaagacggttccgctgttgttctgg
gctacaccgaccgcatcggttccgaagcttacaaccagcagctgtctgagaaacgtgctcagtcc
gttgttgactacctggttgctaaaggcatcccggctggcaaaatctccgctcgcggcatgggtga
atccaacccggttactggcaacacctgtgacaacgtgaaagctcgcgctgccctgatcgattgcc
tggctccggatcgtcgtgtagagatcgaagttaaaggctacaaagaagttgtaactcagccgcag
gcttaa
K. pneumoniae OmpK36
Sequence ID Number 32
AEIYNKDGNKLDLYGKIDGLHYFSDDKSVDGDQTYMRVGVKGETQINDQLTGYGQWEYNVQANNT
ESSSDQAWTRLAFAGLKFGDAGSFDYGRNYGVVYDVTSWTDVLPEFGGDTYGSDNFLQSRANGVA
TYRNSDFFGLVDGLNFALQYQGKNGSVSGEGALSPTNNGRTALKQNGDGYGTSLTYDIYDGISAG
FAYSNSKRLGDQNSKLALGRGDNAETYTGGLKYDANNIYLATQYTQTYNATRAGSLGFANKAQNF
EVVAQYQFDFGLRPSVAYLQSKGKDLEGYGDQDILKYVDVGATYYFNKNMSTYVDYKINLLDDNS
FTHNAGISTDDVVALGLVYQF.
Nucleotide sequence from Sequence ID Number 32
gctgaaatttataacaaagacggcaacaaattagacctgtacggtaaaattgacggtctgcacta
cttctctgacgacaagagcgtcgacggcgaccagacctacatgcgtgtaggcgtgaaaggcgaaa
cccagatcaacgaccagctgaccggttacggccagtgggaatacaacgttcaggcgaacaacact
gaaagctccagcgatcaggcatggactcgtctggcattcgcaggcctgaaatttggcgacgcggg
ctctttcgactacggtcgtaactacggcgtagtatacgacgtaacgtcctggaccgacgttctgc
cggaattcggcggcgacacctacggttctgacaacttcctgcagtcccgtgctaacggcgttgca
acctaccgtaactctgatttcttcggtctggttgacggcctgaactttgctctgcagtatcaggg
taaaaacggcagcgtcagcggcgaaggcgctctgtctcctaccaacaacggtcgtaccgccttga
aacagaacggcgacggttacggtacttctctgacctatgacatctatgatggcatcagcgctggt
ttcgcatactctaactccaaacgtcttggcgaccagaacagcaagctggcactgggtcgtggcga
caacgctgaaacctacaccggcggtctgaaatacgacgcgaacaacatctacctggccactcagt
acacccagacctacaacgcgacccgcgccggttccctgggctttgctaacaaagcgcagaacttc
gaagtggttgctcagtaccagttcgacttcggtctgcgtccgtccgtggcttacctgcagtctaa
aggtaaggatctggaaggctacggcgaccaggacatcctgaaatatgttgacgttggcgcgacct
actacttcaacaaaaacatgtccacctatgttgactacaaaatcaacctgctggacgacaatagc
ttcacccacaacgccggtatctctaccgacgacgtggttgcactgggcctggtttaccagttcta
a
P. aeruginosa OprF
Sequence ID Number 33
QGQNSVEIEAFGKRYFTDSVRNMKNADLYGGSIGYFLTDDVELALSYGEYHDVRGTYETGNKKVH
GNLTSLDAIYHFGTPGVGLRPYVSAGLAHQNITNINSDSQGRQQMTMANIGAGLKYYFTENFFAK
ASLDGQYGLEKRDNGHQGEWMAGLGVGFNFGGSKAAPAPEPVADVCSDSDNDGVCDNVDKCPDTP
ANVTVDANGCPAVAEVVRVQLDVKFDFDKSKVKENSYADIKNLADFMKQYPSTSTTVEGHTDSVG
TDAYNQKLSERRANAVRDVLVNEYGVEGGRVNAVGYGESRPVADNATAEGRAINRRVEAEVEAEA
K.
Nucleotide sequence from Sequence ID Number 33
cagggccagaactcggtagagatcgaagccttcggcaagcgctacttcaccgacagcgttcgcaa
catgaagaacgcggacctgtacggcggctcgatcggttacttcctgaccgacgacgtcgagctgg
cgctgtcctacggtgagtaccatgacgttcgtggcacctacgaaaccggcaacaagaaggtccac
ggcaacctgacctccctggacgccattaccacttcggtaccccgggcgtaggtctgcgtccgtac
gtgtcggctggtctggctcaccagaacatcaccaacatcaacagcgacagccaaggccgtcagca
gatgaccatggccaacatcggcgctggtctgaagtactacttcaccgagaacttcttcgccaagg
ccagcctcgacggccagtacggtctggagaagcgtgacaacggtcaccagggcgagtggatggct
ggcctgggcgtcggcttcaacttcggtggttcgaaagccgctccggctccggaaccggttgccga
cgtttgctccgactccgacaacgacggcgtttgcgacaacgtcgacaagtgcccggataccccgg
ccaacgtcaccgttgacgccaacggctgcccggctgtcgccgaagtcgtacgcgtacagctggac
gtgaagttcgacttcgacaagtccaaggtcaaagagaacagctacgctgacatcaagaacctggc
tgacttcatgaagcagtacccgtccacttccaccaccgttgaaggtcacaccgactccgtcggca
ccgacgcttacaaccagaagctgtccgagcgtcgtgccaacgccgttcgtgacgtactggtcaac
gagtacggtgtagaaggtggtcgcgtgaacgctgttggttacggcgagtcccgcccggttgccga
caacgccaccgctgaaggccgcgctatcaaccgtcgcgttgaagccgaagtagaagctgaagcca
agtaa
P. aeruginosa OprI::PcrV fusion protein
Sequence ID Number 34
SHSKETEARLTATEDAAARAQARADEAYRKADEALGAAQKAQQTADEANERALRMLEKASRKGGG
GSGGGGSGGGGSSAAPASAEQEELLALLRSERIVLAHAGQPLSEAQVLKALAWLLAANPSAPPGQ
GLEVLREVLQARRQPGAQWDLREFLVSAYFSLHGRLDEDVIGVYKDVLQTQDGKRKALLDELKAL
TAELKVYSVIQSQINAALSAKQGIRIDAGGIDLVDPTLYGYAVGDPRWKDSPEYALLSNLDTFSG
KLSIKDFLSGSPKQSGELKGLSDEYPFEKDNNPVGNFATTVSDRSRPLNDKVNEKTTLLNDTSSR
YNSAVEALNRFIQKYDSVLRDILSAI.
Nucleotide sequence from Sequence ID Number 34
agccactccaaagaaaccgaagctcgtctgaccgctaccgaagacgcagctgctcgtgctcaggc
tcgcgctgacgaagcctatcgcaaggctgacgaagctctgggcgctgctcagaaagctcagcaga
ctgctgacgaggctaacgagcgtgccctgcgcatgctggaaaaagccagccgcaagggcggcgg
cggcagcggcggcggcggcagcggcggcggcggcagctcggcggcgcctgccagtgccgagcagg
aggaactgctggccctgttgcgcagcgagcggatcgtgctggcccacgccggccagccgctgagc
gaggcgcaagtgctcaaggcgctcgcctggttgctcgcggccaatccgtccgcgcctccggggca
gggcctcgaggtactccgcgaagtcctgcaggcacgtcggcagcccggtgcgcagtgggatctgc
gcgagttcctggtgtcggcctatttcagcctgcacgggcgtctcgacgaggatgtcatcggtgtc
tacaaggatgtcctgcagacccaggacggcaagcgcaaggcgctgctcgacgagctgaaggcgct
gaccgcggagttgaaggtctacagcgtgatccagtcgcagatcaacgccgcgctgtcggccaagc
agggcatcaggatcgacgctggcggtatcgatctggtcgaccccacgctatatggctatgccgtc
ggcgatcccaggtggaaggacagccccgagtatgcgctgctgagcaatctggataccttcagcgg
caagctgtcgatcaaggattttctcagcggctcgccgaagcagagcggggaactcaagggcctca
gcgatgagtaccccttcgagaaggacaacaacccggtcggcaatttcgccaccacggtgagcgac
cgctcgcgtccgctgaacgacaaggtcaacgagaagaccaccctgctcaacgacaccagctcccg
ctacaactcggcggtcgaggcgctcaaccgcttcatccagaaatacgacagcgtcctgcgcgaca
ttctcagcgcgatctag
P. aeruginosa OprI
Sequence ID Number 35
SHSKETEARLTATEDAAARAQARADEAYRKADEALGAAQKAQQTADEANERALRMLEKASRK.
Nucleotide sequence from Sequence ID Number 35
agccactccaaagaaaccgaagctcgtctgaccgctaccgaagacgcagctgctcgtgctcaggc
tcgcgctgacgaagcctatcgcaaggctgacgaagctctgggcgctgctcagaaagctcagcaga
ctgctgacgaggctaacgagcgtgccctgcgcatgctggaaaaagccagccgcaatag
E. coli OmpA
Sequence ID Number 36
APKDNTWYTGAKLGWSQYHDTGFIPNNGPTHENQLGAGAFGGYQVNPYVGFEMGYDWLGRMPYKG
DNINGAYKAQGVQLTAKLGYPITDDLDIYTRLGGMVWRADTKANVPGGASFKDHDTGVSPVFAGG
VEYAITPEIATRLEYQWTNNIGDAHTIGTRPDNGMLSLGVSYRFGQGEVAPVVAPAPAPAPEVQT
KHFTLKSDVLFTENKATLKPEGQAALDQLYSQLSNLDPKDGSVVVLGYTDRIGSDAYNQALSERR
AQSVVDYLISKGIPADKISARGMGESNPVTGNTCDNVKQRAALIDCLAPDRRVEIEVKGIKDVVT
QPQA.
Nucleotide sequence from Sequence ID Number 36
gctccgaaagataacacctggtacactggtgctaaactgggctggtcccagtaccatgacactgg
ttttattcctaacaatggtccgacccacgaaaaccaactgggtgcaggtgcttttggtggttacc
aggttaacccgtatgttggctttgaaatgggttacgactggttaggtcgtatgccgtacaaaggc
gacaacatcaacggcgcatacaaagctcagggcgttcagctgaccgctaaactgggttacccaat
cactgacgatctggacatctacactcgtctgggtggtatggtatggcgtgcagacaccaaggcta
acgtacctggtggcgcatcctttaaagaccacgacaccggcgtttctccggtcttcgctggcggt
gttgagtatgcgatcactcctgaaatcgctacccgtctggaataccagtggaccaacaacatcgg
tgacgcacacaccatcggcactcgtccggacaacggcatgctgagcctgggtgtttcctaccgtt
tcggtcagggcgaagtagctccagtagttgctccggctccagctccggcaccggaagtacagacc
aagcacttcactctgaagtctgacgttctgttcaccttcaacaaagcaaccctgaaaccggaagg
tcaggctgctctggatcagctgtacagccagctgagcaacctggatccgaaagacggttccgtag
ttgttctgggttacactgaccgcatcggttctgacgcttataaccaggctctgtccgagcgtcgt
gctcagtccgttgttgattacctgatctctaaaggtatcccggcagacaaaatctccgcacgtgg
tatgggcgaatccaacccggttactggcaacacctgtgacaacgtgaaacagcgtgctgcactga
tcgattgcctggctccggatcgtcgcgtagagatcgaagttaaaggcatcaaagacgttgtaact
cagccgcaggcttaa
E. coli OmpX
Sequence ID Number 37
ATSTVTGGYAQSDAQGQMNKMGGFNLKYRYEEDNSPLGVIGSFTYTEKSRTASSGDYNKNQYYGI
TAGPAYRINDWASIYGVVGVGYGKFQTTEYPTYKHDTSDYGFSYGAGLQFNPMENVALDFSYEQS
RIRSVDVGTWIAGVGYRF.
Nucleotide sequence from Sequence ID Number 37
gcgacttctaccgtaactggcggttacgcacaaagcgacgctcagggccaaatgaacaaaatggg
cggtttcaacctgaaataccgctatgaagaagacaacagcccgctgggtgtgatcggttctttca
cttacaccgagaaaagccgtactgcaagctctggtgactacaacaaaaaccagtactacggcatc
actgctggtccggcttaccgcattaacgactgggcaagcatctacggtgtagtgggtgtgggtta
tggtaaattccagaccactgaatacccgacctacaaacacgacaccagcgactacggtttctcct
acggtgctggtttgcagttcaacccgatggaaaacgttgctctggacttctcttacgagcagagc
cgtattcgtagcgttgacgtaggcacctggattgccggtgttggttaccgcttctaa
E. coli FyuA
Sequence ID Number 38
NAQTSQQDESTLVVTASKQSSRSASANNVSSTVVSAPELSDAGVTASDKLPRVLPGLNIENSGNM
LFSTISLRGVSSAQDFYNPAVTLYVDGVPQLSTNTIQALTDVQSVELLRGPQGTLYGKSAQGGII
NIVTQQPDSTPRGYIEGGVSSRDSYRSKFNLSGPIQDGLLYGSVTLLRQVDDGDMINPATGSDDL
GGTRASIGNVKLRLAPDDQPWEMGFAASRECTRATQDAYVGWNDIKGRKLSISDGSPDPYMRRCT
DSQTLSGKYTTDDWVFNLISAWQQQHYSRTFPSGSLIVNMPQRWNQDVQELRAATLGDARTVDMV
FGLYRQNTREKLNSAYDMPTMPYLSSTGYTTAETLAAYSDLTWHLTDRFDIGGGVRFSHDKSSTQ
YHGSMLGNPFGDQGKSNDDQVLGQLSAGYMLTDDWRVYTRVAQGYKPSGYNIVPTAGLDAKPFVA
EKSINYELGTRYETADVTLQAATFYTHTKDMQLYSGPVGMQTLSNAGKADATGVELEAKWRFAPG
WSWDINGNVIRSEFTNDSELYHGNRVPFVPRYGAGSSVNGVIDTRYGALMPRLAVNLVGPHYFDG
DNQLRQGTYATLDSSLGWQATERMNISVYVDNLFDRRYRTYGYMNGSSAVAQVNMGRTVGINTRI
DFF.
Nucleotide sequence from Sequence ID Number 38
cagacttcacagcaagacgaaagcacgctggtggttaccgccagtaaacaatcttcccgctcggc
atcagccaacaacgtctcatctactgttgtcagcgcgccggaattaagcgacgccggcgtcaccg
ccagcgacaaactccccagagtcttgcccgggctcaatattgaaaatagcggcaacatgcttttt
tcgacgatctcgctacgcggcgtctcttcagcgcaggacttctataaccccgccgtcaccctgta
tgtcgatggcgtccctcagctttccaccaacaccatccaggcgcttaccgatgtgcaaagcgtgg
agttgctgcgaggcccacagggaacgttatatggcaaaagcgctcaggggggatcatcaacatcg
tcacccagcagccggacagcacgccgcgcggctatattgaaggcggcgtcagtagccgcgacagt
tatcgaagtaagttcaacctgagcggccccattcaggatggcctgctgtacggcagcgtcaccct
gttacgccaggttgatgacggcgacatgattaaccccgcgacgggaagcgatgacttaggcggca
cccgcgccagcatagggaatgtgaaactgcgtctggcgccggacgatcagccctgggaaatgggc
tttgccgcctcacgcgaatgtacccgcgccacccaggatgcctatgtgggatggaatgatattaa
gggccgtaagctgtcgatcagcgatggttcaccagacccgtacatgcggcgctgcactgacagcc
agaccctgagtgggaaatacaccaccgatgactgggttttcaacctgatcagcgcctggcagcag
cagcattattcgcgcaccttcccttccggttcgttaatcgtcaatatgcctcagcgctggaatca
ggatgtgcaggagctgcgcgccgcaaccctgggcgatgcgcgtaccgttgatatggtgtttgggc
tgtaccggcagaacacccgcgagaagttaaattcagcctacgacatgccgacaatgccttattta
agcagtaccggctataccaccgctgaaacgctggccgcatacagtgacctgacctggcatttaac
cgatcgttttgatatcggcggcggcgtgcgcttctcgcatgataaatccagtacacaatatcacg
gcagcatgctcggcaacccgtttggcgaccagggtaagagcaatgacgatcaggtgctcgggcag
ctatccgcaggctatatgctgaccgatgactggagagtgtatacccgtgtagcccagggatataa
accttccgggtacaacatcgtgcctactgcgggtcttgatgccaaaccgttcgtcgccgagaaat
ccatcaactatgaacttggcacccgctacgaaaccgctgacgtcacgctgcaagccgcgacgttt
tatacccacaccaaagacatgcagctttactctggcccggtcgggatgcagacattaagcaatgc
gggtaaagccgacgccaccggcgttgagcttgaagcgaagtggcggtttgcgccaggctggtcat
gggatatcaatggcaacgtgatccgttccgaattcaccaatgacagtgagttgtatcacggtaac
cgggtgccgttcgtaccacgttatggcgcgggaagcagcgtgaacggcgtgattgatacgcgcta
tggcgcactgatgccccgactggcggttaatctggtcgggccgcattatttcgatggcgacaacc
agttgcggcaaggcacctatgccaccctggacagcagcctgggctggcaggcgactgaacggatg
aacatttccgtctatgtcgataacctgttcgaccgtcgttaccgtacctatggctacatgaacgg
cagcagcgccgtcgcgcaggtcaatatgggtcgcaccgtcggtatcaatacgcgaattgatttct
tctga
E. coli Hma
Sequence ID Number 39
TSSETKISNEETLVVTTNRSASNLWESPATIQVIDQQTLQNSTNASIADNLQDIPGVEITDNSLA
GRKQIRIRGEASSRVLILIDGQEVTYQRAGDNYGVGLLIDESALERVEVVKGPYSVLYGSQAIGG
IVNFITKKGGDKLASGVVKAVYNSATAGWEESIAVQGSIGGFDYRINGSYSDQGNRDTPDGRLPN
TNYRNNSQGVWLGYNSGNHRFGLSLDRYRLATQTYYEDPDGSYEAFSVKIPKLEREKVGVFYDTD
VDGDYLKKIHFDAYEQTIQRQFANEVKTTQPVPSPMIQALTVHNKTDTHDKQYTQAVTLQSHFSL
PANNELVTGAQYKQDRVSQRSGGMTSSKSLTGFINKETRTRSYYESEQSTVSLFAQNDWRFADHW
TWTMGVRQYWLSSKLTRGDGVSYTAGIISDTSLARESASDHEMVTSTSLRYSGFDNLELRAAFAQ
GYVFPTLSQLFMQTSAGGSVTYGNPDLKAEHSNNFELGARYNGNQWLIDSAVYYSEAKDYIASLI
CDGSIVCNGNTNSSRSSYYYYDNIDRAKTWGLEISAEYNGWVFSPYISGNLIRRQYETSTLKTTN
TGEPAINGRIGLKHTLVMGQANIISDVFIRAASSAKDDSNGTETNVPGWATLNFAVNTEFGNEDQ
YRINLALNNLTDKRYRTAHETIPAAGFNAAIGFVWNF.
Nucleotide sequence from Sequence ID Number 39
acttcttcagaaacaaaaatcagcaacgaagagacgctcgtcgtgaccacgaatcgttcggcaag
caacctttgggaaagcccggcgactatacaggttattgaccaacaaacattgcagaactccacca
atgcctccatagccgataatttgcaggacatccccggagtagagataacagacaactccttggca
ggccgtaaacaaatccgcattcgtggcgaagcatcctcccgtgttttaattctcattgatggtca
ggaggtaacttatcagcgcgccggagataattatggtgtgggactgttgatagatgagtctgcgc
tggagcgtgttgaggtagtgaaaggtccatattccgtactgtacggttcacaggcaattggcggt
attgttaacttcataaccaaaaagggaggtgacaaacttgcatctggagttgtgaaagctgttta
taattccgcaacagcaggctgggaagaatcaatcgcggtccaggggagcatcggtggatttgatt
atcgcatcaacggtagttattctgatcagggcaatcgtgatacgccggatggacgtctgccgaat
accaactatcgtaacaatagtcagggtgtatggttgggttataactccggaaaccatcgttttgg
cctctcgcttgatcgctacagactcgcgacgcaaacttactatgaggatccagacggaagctatg
aggcatttagtgtcaaaatacctaaacttgaacgagagaaagttggggtattctatgacacagac
gtggacggtgactatctaaaaaaaattcatttcgacgcgtatgagcagaccatccagcgccaatt
tgccaacgaagtaaaaacgacacagcctgttcccagtccgatgattcaggctctgaccgttcata
acaagactgacacccatgataagcaatacactcaggcggtcacattgcagagtcacttttcgctg
cctgctaataatgaacttgttaccggtgcacagtacaaacaagacagggtcagccaaaggtccgg
tggcatgacctcaagcaaatctctgaccggcttcattaataaggaaacacgaactcgctcctatt
atgagtcagagcaaagtacagtctcactattcgcacaaaatgactggcgattcgccgatcactgg
acatggacaatgggagttcgccaatactggctttcttcaaagttgacgcgtggtgacggagtatc
atataccgcaggcattataagcgatacctctcttgccagagagtctgcgagtgatcacgaaatgg
taacatctacaagcctgcgctattcaggtttcgataacttggagttacgcgctgcgttcgcgcaa
ggctacgtatttcccacactctcccagctttttatgcagacatctgcgggcggcagtgtcacata
cggaaatcctgatcttaaggctgaacactccaataactttgaattaggtgcacgatataatggta
atcagtggctgattgacagcgcagtttactactcagaagctaaagattatattgcaagtctgatc
tgtgatggcagtatagtttgcaatggtaacaccaactcctcccgtagtagctactattattatga
caatattgatcgggcaaaaacatggggactggaaataagcgcggaatataatggctgggttttct
cgccatatatcagtggcaatttaattcgtcggcaatatgaaacttcaacattaaaaacaactaat
acaggagaaccagcgataaacggacgtatagggctgaaacatactcttgtgatgggtcaggccaa
cataatctctgatgtttttattcgtgctgcctctagtgcaaaagatgacagtaacggtaccgaaa
caaatgttccgggctgggccactctcaactttgcagtaaatacagaattcggtaacgaggatcag
taccggattaacctagcactcaataacctgacagacaaacgctaccgtacagcacatgaaactat
tcctgcagcaggttttaatgcagctataggttttgtatggaatttctga
E. coli IutA
Sequence ID Number 40
QQNDDNEIIVSASRSNRTVAEMAQTTWVIENAELEQQIQGGKELKDALAQLIPGLDVSSQSRTNY
GMNMRGRPLVVLIDGVRLNSSRSDSRQLDSVDPFNIDHIEVISGATALYGGGSTGGLINIVTKKG
QPETMMEFEAGTKSGFNSSKDHDERIAGAVSGGNDHISGRLSVAYQKFGGWFDGNGDATLLDNTQ
TGLQHSNRLDIMGTGTLNIDESRQLQLITQYYKSQGDDNYGLNLGKGFSAISGSSTPYVSKGLNS
DRIPGTERHLISLQYSDSDFLRQELVGQVYYRDESLRFYPFPTVNANKQATAFSSSQQDTDQYGM
KLTLNSQLMDGWQITWGLDAEHERFTSNQMFFDLAQASASGGLNNHKIYTTGRYPSYDITNLAAF
LQSSYDINDIFTVSGGVRYQYTENRVDDFIDYTQQQKIAAGKAISADAIPGGSVDYDNFLFNAGL
LMHITERQQAWFNFSQGVALPDPGKYYGRGIYGAAVNGHLPLTKSVNVSDSKLEGVKVDSYELGW
RFTGDNLRTQIAAYYSLSNKSVERNKDLTISVKDDRRRIYGVEGAVDYLIPDTDWSTGVNFNVLK
TESKVNGQWQKYDVKESSPSKATAYINWAPEPWSLRVQSTTSFDVSDAEGNDINGYTTVDFISSW
QLPVGTLSFSVENLFDRDYTTVWGQRAPLYYSPGYGPASLYDYKGRGRTFGLNYSVLF.
Nucleotide sequence from Sequence ID Number 40
cagcaaaacgatgataatgagatcatagtgtctgccagccgcagcaatcgaactgtagcggagat
ggcgcaaaccacctgggttatcgaaaatgccgaactggagcagcagattcagggcggtaaagagc
tgaaagacgcactggctcagttaatccccggccttgatgtcagcagccagagccgaaccaactac
ggtatgaacatgcgtggccgcccgctggttgtcctgattgacggtgtgcgcctcaactcttcacg
ttccgacagccgacaactggactctgtcgatccttttaatatcgaccatattgaagtgatctccg
gcgcgacggccctgtacggtggcgggagtaccggagggttgatcaacatcgtgaccaaaaaaggc
cagccggaaaccatgatggagtttgaggctggcacaaaaagtggctttaacagcagtaaagatca
cgatgagcgcattgccggtgctgtctccggcggaaatgaccatatctccggacgtctttccgtgg
catatcagaaatttggcggctggtttgacggtaacggcgatgccaccctgcttgataacacccag
accggcctgcagcactccaatcggctggacatcatgggaaccggtacgctgaacatcgatgaatc
ccggcagcttcaactgataacgcagtactataaaagtcagggggacgacaattacgggcttaatc
tcgggaaaggcttttccgccatcagcgggagcagcacaccatacgtcagtaaggggctgaattct
gaccgcattcccggcactgagcggcatttgatcagcctgcagtactctgacagtgatttcctgag
acaggaactggtcggtcaggtttactaccgcgatgagtcgttgcggttctacccgttcccgacgg
taaatgcgaataaacaggcgacggctttctcctcgtcacagcaggataccgaccagtacggcatg
aaactgactctgaacagccaacttatggacggctggcaaatcacctgggggctggatgctgagca
tgagcgctttacctccaaccagatgttcttcgatctggctcaggcaagtgcttccggagggctga
acaaccataagatttacaccaccgggcgctatccgtcatatgacatcaccaatctggcggccttc
ctgcaatccagctatgacattaatgatatttttaccgttagcggtggcgtacgctatcagtatac
tgagaacagggtagatgatttcatcgactacacgcagcaacagaagattgctgccgggaaggcga
tatctgccgacgccattcctggtggttcggtagattacgataactttctgttcaatgctggtctg
ctgatgcacatcaccgaacgtcagcaggcatggttcaatttttcccagggggtggcattgccgga
tccggggaaatattatggtcgcggcatctatggtgcagcagtgaacggccatcttcccctgacaa
agagcgtgaacgtcagcgacagtaagctggaaggcgtgaaagtcgattcttatgaactgggctgg
cgctttaccggtgacaacctgcggactcaaatcgcggcatattactcgctttccaataagagcgt
ggaaaggaataaagatctgaccatcagtgtgaaggacgacaggcgccgtatttacggcgtggaag
gtgcggtggactacctgatcccggatactgactggagtaccggtgtgaacttcaatgtgctgaaa
accgagtcgaaagtgaacggtcaatggcaaaaatatgacgtgaaggaatcaagtccatcgaaagc
gacagcttacattaactgggcgccggaaccgtggagtctgcgtgtacagagcaccacttctttcg
acgtaagcgatgcagagggtaacgatattaatggttacactaccgtcgattttatcagtagttgg
cagcttccggtgggaacactcagcttcagcgttgagaacctcttcgaccgtgactataccactgt
ctggggacagcgtgcacctctgtactacagcccgggttacggccctgcttcactgtacgactaca
aaggccggggccgaacctttggtctgaactactcagtgctgttctga
A. pleuropneumoniae OmpA
Sequence ID Number 41
AAPQQNTFYAGAKAGWASFHDGIEQLDSAKNTDRGTKYGINRNSVTYGVFGGYQILNQDKLGLAA
ELGYDYFGRVRGSEKPNGKADKKTFRHAAHGATIALKPSYEVLPDLDVYGKVGIALVNNTYKTFN
AAQEKVKTRRFQSSLILGAGVEYAILPELAARVEYQWLNNAGKASYSTLNRMGATDYRSDISSVS
AGLSYRFGQGAVPVAAPAVETKNFAFSSDVLFAFGKSNLKPAAATALDAMQTEINNAGLSNAAIQ
VNGYTDRIGKEASNLKLSQRRAETVANYIVSKGALAANVTAVGYGEANPVTGATCDKVKGRKALI
ACLAPDRRVEVQVQGTKEVTM.
Nucleotide sequence from Sequence ID Number 41
Gcagcgccacaacaaaatactttctacgcaggtgcgaaagcaggttgggcgtcattccatgatgg
tatcgaacaattagattcagctaaaaacacagatcgcggtacaaaatacggtatcaaccgtaatt
cagtaacttacggcgtattcggcggttaccaaattttaaaccaagacaaattaggtttagcggct
gaattaggttatgactatttcggtcgtgtgcgcggttctgaaaaaccaaacggtaaagcggacaa
gaaaactttccgtcacgctgcacacggtgcgacaatcgcattaaaacctagctacgaagtattac
ctgacttagacgtttacggtaaagtaggtatcgcattagtaaacaatacatataaaacattcaat
gcagcacaagagaaagtgaaaactcgtcgtttccaaagttctttaattttaggtgcgggtgttga
gtacgcaattcttcctgaattagcggcacgtgttgaataccaatggttaaacaacgcaggtaaag
caagctactctactttaaatcgtatgggtgcaactgactaccgttcggatatcagttccgtatct
gcaggtttaagctaccgtttcggtcaaggtgcggtaccggttgcagctccggcagttgaaactaa
aaacttcgcattcagctctgacgtattattcgcattcggtaaatcaaacttaaaaccggctgcgg
caacagcattagatgcaatgcaaaccgaaatcaataacgcaggtttatcaaatgctgcgatccaa
gtaaacggttacacggaccgtatcggtaaagaagcttcaaacttaaaactttcacaacgtcgtgc
ggaaacagtagctaactacatcgtttctaaaggtgctctggcagctaacgtaactgcagtaggtt
acggtgaagcaaaccctgtaaccggcgcaacatgtgacaaagttaaaggtcgtaaagcattaatc
gcttgcttagcaccggatcgtcgtgttgaagttcaagttcaaggtactaaagaagtaactatgta
a
A. pleuropneumoniae OmpW
Sequence ID Number 42
AHQAGDVIFRAGAIGVIANSSSDYQTGADVNLDVNNNIQLGLTGTYMLSDNLGLELLAATPFSHK
ITGKLGATDLGEVAKVKHLPPSLYLQYYFFDSNATVRPYVGAGLNYTRFFSAESLKPQLVQNLRV
KKHSVAPIANLGVDVKLTDNLSFNAAAWYTRIKTTADYDVPGLGHVSTPITLDPVVLFSGISYKF
.
Nucleotide sequence from Sequence ID Number 42
gcacatcaagcgggcgatgtgattttccgtgccggtgcgatcggtgtgattgcaaattcaagttc
ggattatcaaaccggggcggacgtaaacttagatgtaaataataatattcagcttggtttaaccg
gtacctatatgttaagtgataatttaggtcttgaattattagcggcaacaccgttttctcacaaa
atcaccggtaagttaggtgcaacagatttaggcgaagtggcaaaagtaaaacatcttccgccgag
cctttacttacaatattatttctttgattctaatgcgacagttcgtccatacgttggtgccggtt
taaactatactcgctttttcagtgctgaaagtttaaaaccgcaattagtacaaaacttacgtgtt
aaaaaacattccgtcgcaccgattgcgaatttaggtgttgatgtgaaattaacggataatctatc
attcaatgcggcagcttggtacacacgtattaaaactactgccgattatgatgttccgggattag
gtcatgtaagtacaccgattactttagatcctgttgtattattctcaggtattagctacaaattc
taa
A. pleuropneumoniae TbpA
Sequence ID Number 43
AEQAVQLNDVYVTGTKKKAHKKENEVTGLGKVVKTPDTLSKEQVLGIRDLTRYDPGISVVEQGRG
ATTGYSIRGVDRNRVGLALDGLPQIQSYVSQYSRSSSGAINEIEYENLRSIQISKGASSSEFGSG
SLGGSVQFRTKEVSDIIKPGQSWGLDTKSAYSSKNQQWLNSLAFAGTHNGFDALVIYTHRDGKET
KAHKNAESRSQNITRVGVETNELDTSNRYTATTNNQHTYGWFLIKDECPTLDCTPKQMARVTKDT
PSFRSYPEYTPEEKQAYENQKHITERLNAQDYTGEYRALPDPLKYKSDSWLVKLGYTFSPKHYVA
GTYEHSKQRYDTRDMTYTAYWQPSDLLRTGRNWYPMNNAKGLYRDNALDGVAIDYFTEDGVKSSK
GLRWAKARFIDEWHTRDRLGALYRYTNQDGNRLIDRLSLSFDQQKINLSTRLRENNCSEYPTIDK
NCRATLDKLWSSTKNEQSSYEEKHDTIQLSLDKTVQTGLGKHQLNMLLGSDRFNSTLKRHEILSE
FSVGSWGLVRDIGYRNGSYNNPYVYELKDQAIYSKNECDYSGTIAGRADCATSKIKGHNHYIALR
DNFAITKYLDIGLGYRFDKHKFRSTHRWANQGDYKNSAWNIGIVAKPTSFLSLSYRASSGFRVPS
FQELFGLRYDGAMKGSSDAYQKTEKLSPEKSLNQEVAATFKGDFGVVEVSYFKNDYKQLIAPAER
MHQTQSMINYFNVQDIKLDGINLIGKLDWNGVFDKIPEGIYTTLAYSKMRVKEVKNYQGYMNIRS
PLLDTIQPARYVVGVGYDQPDEKWGVNLTMTHSSGKNPNELRGNEQVGFANYERTATKKRTRSWH
TFDLTGYITPWKHTTVRAGVYNLMNYRYTTWESVRQSSLNAIHQHTNVKDYARYAAPGRNYVVSF
EMKF.
Nucleotide sequence from Sequence ID Number 43
gcagaacaagcggtgcaattgaacgatgtttatgtcacaggaacaaaaaagaaagcacataaaaa
agagaacgaagtcacaggcttagggaaagtagttaaaacaccagatactcttagtaaggagcaag
tgttaggaatacgagatctgactcgttacgaccccggtatttctgtcgtagaacaagggagaggt
gcgactacaggctactcaattcgtggggtagatcgtaaccgtgttggcttggcattagatggttt
accacagattcagtcctatgtaagccagtattcacgttcctcaagcggtgccattaatgaaatag
aatacgaaaatctgcgttcgatccaaattagtaaaggggctagttcttctgagtttggtagtggc
tcactaggcggttcggtgcaattccgtaccaaagaggtaagcgacattattaagccagggcaatc
ttggggattagataccaaaagtgcctacagtagcaaaaatcaacaatggttaaactcacttgctt
ttgctggtactcacaatggctttgatgctcttgtgatttacactcaccgtgatggtaaggaaacg
aaagctcataaaaatgcagagagtcgttctcagaatatcacccgagtaggagtggaaaccaacga
gcttgatacctcaaatagatatactgcgacgacgaataatcaacatacttatggctggtttttga
ttaaagatgaatgtccaacgttagattgtacgccgaaacagatggctagggtgacaaaagatacg
ccatctttccgttcttaccctgaatatactcctgaggaaaaacaggcttatgagaaccaaaaaca
tattacagagcgtctaaatgctcaggattacactggtgaatatagagctttacctgatccgctta
aatataaatctgattcttggctggttaaattaggatacacattctctccgaaacattatgtcgct
ggtacttatgaacatagcaaacagcgttacgacacccgagatatgacctataccgcttattggca
accatcggatttacttagaactggtagaaattggtatccaatgaataatgctaaaggattatatc
gtgataatgctttagatggtgttgctattgactactttacggaagatggtgtgaaatcctcaaaa
ggtttacgttgggcaaaagctcgttttattgacgagtggcacactcgtgatcgcttaggtgcttt
atatcgttatactaatcaagatggaaatcgtctgattgatagactatccttgagtttcgatcagc
aaaaaattaatttatctacccgcttgagagaaaacaactgttcggaatatccaaccatagataag
aattgccgtgcaactcttgataaactttggtcttctactaaaaatgagcaaagttcttatgaaga
aaaacacgacactattcagctctcgttagataaaaccgtacaaacgggattgggtaaacatcaat
taaatatgttattaggttcagaccgtttcaattccaccttaaaacgccatgaaattttgagtgaa
ttttctgtagggagttgggggcttgttagagacattggctatcgaaatggatcttacaataatcc
ttatgtgtatgagctaaaagatcaggcaatttatagtaaaaatgaatgtgattatagtggcacta
ttgcaggtagggctgattgtgctacaagtaaaatcaaagggcataatcactacatcgctctgaga
gataattttgccataaccaagtatttggatattggtttgggttaccgtttcgataagcataaatt
ccgtagcactcatcgctgggcaaatcaaggcgattataaaaatagtgcgtggaatattggcatag
tcgcaaaaccaacgtcattcctatcgctctcttatcgagcatcatctggctttagagtgccaagt
ttccaagagctatttggcttacgttatgatggtgcaatgaaaggctccagcgatgcttaccaaaa
aacagagaagttatctcctgaaaaatccttaaaccaagaggttgctgcgactttcaaaggtgatt
ttggtgtcgttgaagtcagttatttcaaaaatgactataagcagttaattgctccagcagaaaga
atgcaccaaactcaatcaatgattaactattttaatgtgcaagatattaaattggacggcattaa
tcttattggtaagctagattggaatggggtatttgataaaattcctgagggcatttacacaacat
tggcttatagcaaaatgcgagtaaaagaggtgaaaaactatcaagggtatatgaatattcgttct
ccattgttagataccattcagcctgctcgctatgttgtaggagtggggtacgatcagccagatga
aaaatggggcgtgaatctaacaatgacacactccagtggaaaaaatccaaatgagttaagaggta
atgaacaagtcggttttgccaattatgagcgaactgccacgaagaaaagaacacgttcttggcat
acctttgacttaacgggatatatcaccccttggaaacatacaacggtacgagctggcgtatataa
cctgatgaattatcgttacaccacttgggaatccgtacgtcaatcttcgcttaatgcaattcatc
agcatactaacgtaaaagactatgcaaggtatgcagcgcccggtagaaattatgttgtttcattc
gaaatgaaattctaa
A. pleuropneumoniae ApfA
Sequence ID Number 44
FTLIELMIVIAIIAILATVAIPSYNSYTQKAALSELLAASASYKTDVEICIYNTGDSKNCSGGQN
GVRKMTELRQAKYLNAITVEGGTITVTGKGNLQEYGYTMTPIHNGSTISWETKCKGEDLSLFPAN
FCAIN.
Nucleotide sequence from Sequence ID Number 44
tttactttaattgaattgatgatcgtgattgcgattattgccattttagctacggttgcaattcc
gtcatataacagttatacccaaaaagcggcgctttcggagctattggcggcatcggcttcttata
aaacggatgtcgagatctgcatatataacaccggagattctaaaaactgtagcggcggtcaaaac
ggtgtcagaaaaatgacggagcttagacaggctaaatatttaaatgccattacggtggaaggcgg
aacgattacggtaacggggaaggggaatctgcaggaatacggttatacgatgacaccgattcata
acggtagcactatttcttgggaaacgaaatgtaaaggggaggacttaagtttatttccggcaaat
ttctgtgcgataaattag
P. aeruginosa PcrV
Sequence ID Number 45
AAPASAEQEELLALLRSERIVLAHAGQPLSEAQVLKALAWLLAANPSAPPGQGLEVLREVLQARR
QPGAQWDLREFLVSAYFSLHGRLDEDVIGVYKDVLQTQDGKRKALLDELKALTAELKVYSVIQSQ
INAALSAKQGIRIDAGGIDLVDPTLYGYAVGDPRWKDSPEYALLSNLDTFSGKLSIKDFLSGSPK
QSGELKGLSDEYPFEKDNNPVGNFATTVSDRSRPLNDKVNEKTTLLNDTSSRYNSAVEALNRFIQ
KYDSVLRDILSAI.
Nucleotide sequence from Sequence ID Number 45
tcggcggcgcctgccagtgccgagcaggaggaactgctggccctgttgcgcagcgagcggatcgt
gctggcccacgccggccagccgctgagcgaggcgcaagtgctcaaggcgctcgcctggttgctcg
cggccaatccgtccgcgcctccggggcagggcctcgaggtactccgcgaagtcctgcaggcacgt
cggcagcccggtgcgcagtgggatctgcgcgagttcctggtgtcggcctatttcagcctgcacgg
gcgtctcgacgaggatgtcatcggtgtctacaaggatgtcctgcagacccaggacggcaagcgca
aggcgctgctcgacgagctcaaggcgctgaccgcggagttgaaggtctacagcgtgatccagtcg
cagatcaacgccgcgctgtcggccaagcagggcatcaggatcgacgctggcggtatcgatctggt
cgaccccacgctatatggctatgccgtcggcgatcccaggtggaaggacagccccgagtatgcgc
tgctgagcaatctggataccttcagcggcaagctgtcgatcaaggattttctcagcggctcgccg
aagcagagcggggaactcaagggcctcagcgatgagtaccccttcgagaaggacaacaacccggt
cggcaatttcgccaccacggtgagcgaccgctcgcgtccgctgaacgacaaggtcaacgagaaga
ccaccctgctcaacgacaccagctcccgctacaactcggcggtcgaggcgctcaaccgcttcatc
cagaaatacgacagcgtcctgcgcgacattctcagcgcgatctag

In another embodiment of the first aspect of the invention, the heterologous antigens expressed at the Outer Membrane of the A. baumannii strain deficient in lipopolysaccharide (LPS) are at least derived from K. pneumoniae and are selected from the list consisting of Kp-OmpA and/or Kp-OmpK36, and/or any identical sequences thereto as taught in the present invention; and/or are derived from P. aeruginosa and are selected from the list consisting of Pa-OprF and/or Pa-OprI, and or the fusion protein Pa-OprI::PcrV, and/or any identical sequences thereto as taught in the present invention; and/or are derived from E. coli and are selected from the list consisting of Ec-OmpA and/or Ec-OmpX and/or Ec-FuyA and/or Ec-Hma and/or Ec-IutA, and/or any identical sequences thereto as taught in the present invention; and/or are derived from A. pleuropneumoniae and are selected from the list Ap-OmpA and/or Ap-OmpW and/or Ap-TbpA and/or Ap-ApfA, and/or any identical sequences thereto as taught in the present invention, and preferably the A. baumannii strain deficient in lipopolysaccharide (LPS) further comprises the expression of A. baumannii antigens Ab-OmpA and Ab-Omp22.

In another embodiment of the first aspect of the invention, the A. baumannii strain deficient in lipopolysaccharide (LPS) is characterized by the partial or complete inactivation of the genes selected from the list consisting of lpxA, lpxC, and lpxD.

A second aspect of the invention refers to a composition, hereinafter composition of the invention, comprising the A. baumannii strain deficient in lipopolysaccharide (LPS) expressing the one or multiple copies of antigenic outer-membrane heterologous proteins from one or more microorganisms at its Outer Membrane, as defined in the first aspect of the invention or in any of its preferred embodiments.

Preferably, the composition of the invention, is a pharmaceutical composition optionally comprising acceptable pharmaceutical vehicles, carriers and/or excipients. Still more preferably, the composition of the invention is a vaccine formulation optionally comprising an adjuvant. Preferably the composition or the vaccine formulation, comprises from about 10⁶ to about 10¹² A. baumannii strains deficient in lipopolysaccharide (LPS) expressing the one or multiple copies of antigenic outer-membrane heterologous proteins from one or more microorganisms at its Outer Membrane, as defined in the first aspect of the invention or in any of its preferred embodiments, preferably about 10⁹ of such strains. Also preferably, the adjuvant, if present in the vaccine formulation, is preferably Al(OH)3. Further preferably, the A. baumannii strains deficient in lipopolysaccharide (LPS) present in the composition, preferably in the vaccine formulation, are inactivated. Nothwithstanding the above, the dosing for obtaining an effective therapeutic quantity depends on a variety of factors such as for example, age, weight, sex, tolerante, . . . of the mammal. As used in this description, any therapeutic quantity should be effective and should thus refer to the quantity of inactive cells of the genus Acinetobacter that produces the desired effect, and in general are determined by the therapeutic effect that is desired.

The term “excipient” makes reference to a substance that helps in the absorption of the elements of the composition of the medicaments of the invention, stabilizing said elements, activating or helping the preparation of the medicament such that it provides consistency or flavours that make it more palatable. The excipients can maintain the ingredients together, like for example is the case with starches, sugars, cellulose, sweeteners, colouring agents, the function of protecting the medicament, for example isolating it form air and/or humidity, the function of filling the pill, capsule or any other form of presentation, for example, the case of dibasic calcium phosphate, the function for facilitating dissolution of the components and their absorption in the intestine, without excluding other types of excipients described in this paragraph.

The vehicle, in the same way as the excipient, is a substance that is used in the medicament to dilute any of the components of the present invention to a desired volume or weight. The pharmaceutically acceptable vehicle is an inert substance or of similar action to any of the elements of the present invention. The function of the vehicle is to facilitate the incorporation of other elements, permit better dosing and administration and give consistency and form to the medicament. When the form of presentation is liquid, the pharmaceutically acceptable vehicle is the diluent.

The adjuvants and pharmaceutically acceptable vehicle that can be used in the composition of the invention are those vehicles known by experts in the field. In this invention, the term “adjuvant” refers to any agent that does not pose antigenic activity in and of itself, that can be used to stimulate the immune system to increase the response to a vaccine. There are many adjuvants, for example but not limited to, aluminium phosphate, aluminium hydroxide, toll-like receptor agonists, cytokines, squaline, Freunds incomplete and complete adjuvants. In a preferred form of this aspect of the invention, the adjuvant is selected for a list that consists of aluminium phosphate, aluminium hydroxide, toll-like receptor agonists, cytokines, squaline, saponins, Freunds incomplete and complete adjuvants.

In the context of the present invention, the term “vaccine” refers to an antigenic preparation employed for inducing an immune response to a disease. They are prepared from antigens that, once inside the host, provoke an immune response through the production of antibodies, and generate immunologic memory producing transient or permanent immunity.

Manufacturing of the vaccine formulation of the present invention can be achieved, but without being limited to, via a two-step fermentation process and subsequent heat-inactivation. The vaccine formulation shall be preferably in the form of a sterile, lyophilized preparation of the vaccine. Lyophilization shall be applied to the final product, once it has been formulated with excipients and the adjuvant. The vaccine formulation based on the LPS-null A. baumannii cell technology is preferably not filtered or autoclaved in order to obtain sterility. The cells are usually inactivated in the last step of manufacturing of the drug substance after fermentation, before formulating the final product. The inactivation method shall be preferably based on a mild heat-inactivation protocol (75° C. for 30 min) in order to preserve the potency of the whole-cell, multi-antigen active ingredient.

A third aspect refers to the A. baumannii strain deficient in lipopolysaccharide (LPS) expressing the one or multiple copies of antigenic outer-membrane heterologous proteins from one or more microorganisms at its Outer Membrane, as defined in the first aspect of the invention or in any of its preferred embodiments or to the composition of the second aspect of the invention, for use as a medicament or for use in therapy. More preferably, for use as a vaccine for inducing an immune response to a disease caused by any microorganisms originally expressing the one or multiple copies of the antigenic Outer Membrane proteins.

The term “medicament” as used in this report, makes reference to any substance used for the prevention, alleviation, treatment or cure of a disease in man or animals.

In a fourth aspect, the A. baumannii strain deficient in lipopolysaccharide (LPS) of the first aspect of the invention or of any of its preferred embodiments or the composition of the second aspect of the invention or of any of its preferred embodiments, is used for delivering bacterial Outer Membrane antigens to a subject, preferably a human subject, in need thereof for immunizing said subject against any infectious disease comprising, carrying or expressing said antigens. In a preferred embodiment, the A. baumannii strain deficient in lipopolysaccharide (LPS) of the first aspect of the invention or of any of its preferred embodiments or the composition of the second aspect of the invention or of any of its preferred embodiments, is use for delivering bacterial Outer Membrane antigens for inducing an immunological response against K. pneumoniae, P. aeruginosa, E. coli, and/or A. pleuropneumoniae and optionally A. baumannii in a subject in need thereof. In a preferred embodiment, the A. baumannii strain deficient in lipopolysaccharide (LPS) of the first aspect of the invention or any of its preferred embodiments or the composition of the second aspect of the invention or any of its preferred embodiments, is used for delivering bacterial Outer Membrane antigens for the prevention, improvement or treatment of an infection caused by K. pneumoniae, P. aeruginosa, E. coli, and/or A. pleuropneumoniae and optionally A. baumannii. Preferably, for any of these purposes the heterologous antigens expressed at the Outer Membrane of the A. baumannii strain deficient in lipopolysaccharide (LPS) are at least derived from any of those shown throughout the present invention for K. pneumoniae, P. aeruginosa, E. coli, and A. pleuropneumoniae, such as, but not limited to, for K. pneumoniae those selected from the list consisting of Kp-OmpA and/or Kp-OmpK36; for P. aeruginosa those selected from the list consisting of Pa-OprF and/or Pa-OprI, and or the fusion protein Pa-OprI::PcrV; for E. coli those selected from the list consisting of Ec-OmpA and/or Ec-OmpX and/or Ec-FuyA and/or Ec-Hma and/or Ec-IutA; and for A. pleuropneumoniae those selected from the list Ap-OmpA and/or Ap-OmpW and/or Ap-TbpA and/or Ap-ApfA; and preferably the A. baumannii strain deficient in lipopolysaccharide (LPS) further comprises the expression of A. baumannii antigens Ab-OmpA and/or Ab-Omp22.

The medicaments and compositions of the invention can be used alone or in combination with other medicaments or active ingredients or compositions for the treatment of diseases produced by organisms from the genus Acinetobacter.

As used here, the term “active ingredient” refers to any component that potentially provides pharmacological activity or other different effect in the diagnosis, cure, alleviation, treatment or prevention of a disease or that affects the structure or function of the human or animal body. The term includes those components that promote a chemical change in the elaboration of the drug and are present in the same and a modified form that provides specific activity or the effect.

It is understood by “an infection caused by K. pneumoniae, P. aeruginosa, E. coli, and/or A. pleuropneumoniae and optionally A. baumannii” are those diseases in which the causal agent of the pathology is any of K. pneumoniae, P. aeruginosa, E. coli, and/or A. pleuropneumoniae and/or A. baumannii. The genus Acinetobacter produces diverse pathologies for example but not limited to, bacteremia, meningitis, urinary tract infections, skin and soft tissue infections, surgical site infections and pneumonia. For these reasons, one of the more preferred forms, diseases produced by organisms of the genus Acinetobacter are selected from a list that consists of bacteremia, meningitis, urinary tract infections, skin and soft tissue infections, surgical site infections and pneumonia.

A fifth aspect of the invention refers to an antibody or an active fragment thereof obtainable by immunization of a mammal with the composition of the first or second aspect of the invention, preferably said antibody or active fragment consists of a composition in which preferably said composition is a pharmaceutical composition and said pharmaceutical composition is used as a therapy, particularly for the treatment of infections caused caused by K. pneumoniae, P. aeruginosa, E. coli, and/or A. pleuropneumoniae and optionally A. baumannii.

### Method for Preparing an LPS-Null Acinetobacter Cell Able to Present Heterologous Outer-Membrane Antigens Following the Invention

This specific section constitutes a non-limiting example of a method for preparing an A. baumannii strain in accordance with the first aspect of the invention or to any of its preferred embodiments.

First, A. baumannii cells are transformed with a vector, preferably a suicide vector, comprising sequences for promoting recombination at the aimed target locus into the A. baumannii genome, being such locus any locus of the A. baumannii genome comprising sequences suitable to undergo recombination. For example, a suitable locus for integration of an insert by recombination might be: cysI, trpE, lpxA, lpxC, lpxD, lpxB, lpxK, lpxL, lpxM, and/or Tn5/Tn7 sites. In a preferred embodiment of this invention, the suitable locus is cysI. The cysI locus contains the cysI ORF, encoding gene, which comprises a 1,644-nt ORF encoding a 547-amino-acid protein that is essential for the biosynthesis of the amino-acid cysteine. In another preferred embodiment, the suitable locus is lpxA, which contains the ORF encoding the acyltransferase LpxA, the first enzyme in the lipopolysaccharide biosynthesis pathway.

The sequences of the vector for promoting recombination are flanking an expression construct comprising at least one or more transcription promoter sequences, one or more ORFs encoding OMP proteins heterologous for A. baumannii, and one or more transcription termination sequences. The promoter sequence might be any known sequence in the state of the art able to promote transcription in an A. baumannii cell. These promoter sequences are routinely used in bacteriology research. In a preferred embodiment of this invention, the promoter sequence used is a promoter sequence located upstream of the A. baumannii ORF encoding the Outer Membrane protein OmpA. The expression construct might comprise several ORFs in tandem, each one flanked by a promoter and a termination, transcriptional termination, sequence upstream and downstream of the ORF, respectively, therefore built for allowing independent expression of each ORF, controlled by a specific promoter. Alternatively, the expression construct might have one or several ORFs within an operon-like structure with polycistronic expression controlled by a common promoter. The ORFs of the expression vector encode OMP proteins or peptides selected by their immunogenic properties, derived from known humans or animal pathogens distinct from A. baumannii and whose expression in the A. baumannii cell is intended for raising an immune response against pathogens distinct from A. baumannii in a human or animal vaccinated with the A. baumannii cell. The technology of this invention is based on using at the upstream part of each ORF a signal sequence for post-translational processing of the encoded protein, in particular for promoting the location of the encoded proteins at the outer-membrane of the A. baumannii cells. The nature of the signal sequences able to promote location of the expressed proteins at the outer-membrane of the A. baumannii cells are well-known in the art. In a preferred embodiment of this invention, the signal sequence of the outer membrane protein OmpA of A. baumannii is used to promote location of the encoded proteins at the outer-membrane of A. baumannii. It is also well known in the art that any OMP protein with a transmembrane domain, even from any other bacterial cell distinct from A. baumannii, when expressed as a fusion protein comprising the A. baumannii signal sequence at the N-terminus will be processed by the A. baumannii cell for integration into the external membrane of the A. baumannii cell. Therefore, the technology developed in this invention can be applied to any OMP protein from a bacterial cell, and particularly from a Gram-negative bacterial cell with well-known OMP transmembrane domains, independently from the nature of the remaining domains of the selected OMP protein for expression in the A. baumannii cell.

The heterologous OMP proteins selected for expression in the A. baumannii cells as described before must therefore be OMP proteins derived from any known bacterial pathogen with OMP transmembrane domains for insertion into the bacterial external membrane. The nature of these OMP proteins is well known in the art. The outer membrane protects Gram-negative bacteria against a harsh environment. At the same time, the embedded proteins fulfil a number of tasks that are crucial to the bacterial cell, such as solute and protein translocation, as well as signal transduction. Unlike membrane proteins from all other sources, integral OMP proteins do not consist of transmembrane alpha-helices, but instead fold into antiparallel beta-barrels. They include the OmpA membrane domain, the OmpX protein, phospholipase A, general porins, substrate-specific porins, and iron siderophore transporters. It is also well known in the art that the location of these OMP proteins at the external surface of bacterial pathogens confer them immunogenic properties. It is well known in the art that the sera from humans infected by bacterial pathogens recognize predominantly OMP proteins on a crude protein extract from the bacterial cells, indicating the presence of a significant titer of serum antibodies raised against the bacterial OMP proteins.

In a particular embodiment of this invention, the sequence of the heterologous OMP protein derived from a pathogen distinct from A. baumannii can be altered by inserting peptides from the same or other pathogen with the intention of adding extra antigenic sequences at domains of the OMP protein where expression and final location in the external membrane of A. baumannii as taught in this invention is not preclude by the insertion of the extra antigenic sequences in the final fusion protein based on the initial OMP.

In a preferred embodiment of this invention, the heterologous OMPs selected for expression into the A. baumannii cells derived from the most worrisome Gram-negative bacteria, and in particular from K. pneumoniae, P. aeruginosa and/or E. coli. In another preferred embodiment of this invention the heterologous OMPs were selected from Actinobacillus pleuropneumoniae, the causative agent of porcine pleuropneumonia. In another preferred embodiment of this invention, the OMP proteins derived from K. pneumoniae are the OMP proteins OmpA and/or OmpK36 from K. pneumoniae. In another preferred embodiment of this invention, the OMP proteins derived from P. aeruginosa are the OMP protein OprF and/or a fusion between the OMP protein OprI from P. aeruginosa and the protein PcrV of the Type 3 secretion system from P. aeruginosa. In yet another preferred embodiment of this invention, the OMP proteins derived from E. coli are the outer membrane receptors involved in iron acquisition FuyA, HmA, and IutA or the OMPs OmpA and OmpX. And in another preferred embodiment of this invention, the OMP proteins derived from Actinobacillus pleuropneumoniae are the OMPs OmpA and OmpW, the transferrin binding protein A, TpbA, and/or the Type IV fimbrial subunit protein ApfA.

The transcription termination sequences of the expression construct might be any transcription termination sequence able to allow transcription termination in A. baumannii, well known in the art.

Second, once the A. baumannii cells are transformed with the vector, preferably a suicide vector, recombination between specific selected sequences of the target loci in the host and homologous sequences in the vector leads to the production of recombinant cells where the sequences of the vector have integrated into the targeted loci on the A. baumannii chromosome. This recombination event can be assisted by strategies well-known in the art, like inducing double strand breaks at the insertion site with genome-editing tools, like those based on the CRISPR methodology. In a preferred embodiment of this invention, a first recombination event leads to the insertion of the vector sequences comprising selectable markers and the expression constructs. The selectable markers are used for selection of cells where such first recombination event has occurred. A second recombination event might lead to recombinant cells where only the expression construct but not the sequences encoding the selectable markers remain integrated at the insertion site. By using as a selection tool the lack of the selectable markers, A. baumannii cells where such second recombination event has occurred can be selected. Once selected, common techniques like PCR and DNA sequencing are used to check whether the integration of the vector sequences into the targeted loci has occurred as expected.

Third, expression of the proteins encoded by the expression construct in the recombinant A. baumannii cells can be analyzed by basic proteomic techniques like Western-Blot or ELISA. Location of the encoded proteins at the Outer Membrane of the recombinant A. baumannii cells can be analyzed by common proteomic methods previously described in the art such as the methods described in the examples.

Fourth, once the expression of the heterologous proteins analyzed in the recombinant A. baumannii cells is considered satisfactory, loss of the LPS is induced in the A. baumannii cells.

All steps executed until this point, i.e. transformation of A. baumannii cells with the expression construct and integration of the expression construct into the chromosome, have been executed on a wild-type A. baumannii cell. It is well known in the art that any strain, clone or variant of A. baumannii can be transformed by the techniques described in the invention thus far. In addition, it is known in the art that viable A. baumannii mutants unable to synthesize the Lipid A, i.e. the core component of the LPS, can be obtained. In a preferred embodiment of this invention, the A. baumannii cells are grown on plates with the antibiotic colistin and colistin-resistant mutants are selected. It is well known in the art that loss of the LPS make A. baumannii cells resistant to colistin. Since other mutations not affecting the LPS synthesis can also lead to colistin resistance, once the colistin-resistant mutants have been isolated, specific PCR reactions and DNA sequencing are used to look for mutations in the LPS-synthesis genes, including the genes selected from the list consisting of lpxA, lpxB, lpxC, lpxD, lpxK, lpxL and/or lpxM. Once the mutations in the LPS-synthesis genes are confirmed, the absence of the LPS can be checked by techniques well known in the art, like a quantitative chromogenic LAL-based assay to detect the presence of the endotoxin, i.e. LPS, in bacterial cultures, or confirmed lack of detection by anti-LPS antibodies of a crude extract from the bacterial cells. As taught in the examples, loss of LPS can be induced and LPS-null A. baumannii mutants can be obtained independently from the A. baumannii strain, clone or variant used. As taught in the examples, a variety of A. baumannii strains have been used in our laboratories to induce loss of the LPS and a variety of mutations in the LPS-synthesis genes have been found responsible for the loss of the LPS.

In addition to the method described above, loss of LPS can be induced by alternative methods like site-directed mutagenesis of a LPS-synthesis gene leading to partial or complete inactivation of LPS-synthesis genes, including the genes selected from the list consisting of lpxA, lpxB, lpxC, lpxD, lpxK, lpxL and/or lpxM;

In a particular embodiment of this invention, when the targeted locus for insertion of the recombination events described above is a locus comprising an ORF encoding an LPS-synthesis gene, including genes selected from the list consisting of lpxA, lpxB, lpxC, lpxD, lpxK, lpxL and/or lpxM, and the recombination leads to partial or complete inactivation of such ORF, the resulting recombinant A. baumannii cells are LPS-negative cells due to partial or complete inactivation of LPS-synthesis genes, including the genes selected from the list consisting of lpxA, lpxB, lpxC, lpxD, lpxK, lpxL and/or lpxM.

It is understood that “infection” in the present invention is that pathology generated by the invasion or colonization of any host tissue by any organisms of the genus Acinetobacter, preferably A. baumannii or by any organisms from any other bacterial species targeted by the vaccine candidates, including the bacterial species K. pneumoniae, P. aeruginosa, E. coli and A. pleuropneumoniae.

The term “antigen” in the invention refers to a molecule (generally a protein or polysaccharide) that can induce the formation of antibodies. There are many different types of molecules that can act as antigens, such as proteins, peptides, polysaccharides, and more rarely other molecules such as nucleic acids.

The following examples merely illustrate but do not limit the scope of the present invention.

### EXAMPLES

### Abbreviatures

cysI: gene coding for the sulfite reductase involved in the biosynthesis of the amino-acid L-cysteine.

trpE: gene coding for the anthranilate synthase (subunit I) involved in the biosynthesis of the amino-acid tryptophan.

lpxA: gene coding for the Acyl-[acyl-carrier-protein]-UDP-N-acetylglucosamine O-acyltransferase involved in the biosynthesis of lipid A, a phosphorylated glycolipid that anchors the lipopolysaccharide to the outer membrane of the cell.

lpxC: gene coding for the DP-3-O-acyl-N-acetylglucosamine deacetylase that catalyzes the hydrolysis of UDP-3-O-myristoyl-N-acetylglucosamine to form UDP-3-O-myristoylglucosamine and acetate, the committed step in lipid A biosynthesis.

lpxD: gene coding for the DP-3-O-acylglucosamine N-acyltransferase involved in the biosynthesis of lipid A that catalyzes the N-acylation of UDP-3-O-acylglucosamine using 3-hydroxyacyl-ACP as the acyl donor.

lpxB: gene coding for the Lipid-A-disaccharide synthase involved in the condensation of UDP-2,3-diacylglucosamine and 2,3-diacylglucosamine-1-phosphate to form lipid A disaccharide, a precursor of lipid A.

lpxK: gene coding for the Tetraacyldisaccharide 4′-kinase that transfers the gamma-phosphate of ATP to the 4′-position of a tetraacyldisaccharide 1-phosphate intermediate (termed DS-1-P) to form tetraacyldisaccharide 1,4′-bis-phosphate (lipid IVA). This protein is involved in step 6 of the subpathway that synthesizes lipid IV(A) from (3R)-3-hydroxytetradecanoyl-[acyl-carrier-protein] and UDP-N-acetyl-alpha-D-glucosamine.

lpxL: gene coding for the Lipid A biosynthesis lauroyltransferase that catalyzes the transfer of laurate from lauroyl-acyl carrier protein (ACP) to Kdo₂-lipid IV(A) to form Kdo₂-(lauroyl)-lipid IV(A).

lpxM: gene coding for the Lipid A biosynthesis myristoyltransferase involved in step 4 of the subpathway that synthesizes KDO(2)-lipid A from CMP-3-deoxy-D-manno-octulosonate and lipid IV(A).

Tn5/Tn7 sites: regions of the A. baumannii genome preferred by the transposon Tn5 and Tn7 to be inserted.

### Example 1. Method for Selection of LPS-Null Mutants of A. Baumannii that can be Applied to any A. Baumannii Strain and Results in Different Selected Mutations of the LPS-Synthesis Genes

As shown in Moffatt et al. 2010, Garcia-Quintanilla et al. 2014, Pulido et al. 2018, and Carretero-Ledesma et al 2018, among others, colistin-resistant variants of a given strain of A. baumannii can be isolated by direct plating of these parent strains onto Mueller-Hinton agar containing 10 mcg/ml of colistin sulfate; colistin-resistant colonies can be identified (at a frequency of between 1 in 10⁸ and 1 in 10⁹) following a single round of selection. As also shown in Moffat et al. 2010, Garcia-Quintanilla et al. 2014, Pulido et al. 2018, and Carretero-Ledesma et al 2018, among others, the colistin-resistant phenotype of the selected colonies is frequently acquired by LPS-loss due to mutations in any of the LPS synthesis genes. Table 1 shows examples of colistin-resistant mutants selected by the authors of this invention by using the colistin-plating method commented above. In Table 1 examples are provided of LPS-null colistin-resistant derivatives from three different unrelated clinical isolates of A. baumannii, specifically an old clinical isolate now available at the ATCC as strain ATCC19606 and used as reference A. baumannii strain in the experimentation on animal models elsewhere, and clinical isolates from an outbreak in the Hospital Virgen del Rocio of Seville in 2002, named IB001 and Ab283. Different LPS-null derivatives are shown and the specific mutation found in the LPS biosynthesis gene is indicated.

In addition, Table 1 shows one LPS-null derivative of Ab283 obtained by direct mutagenesis of the LPS synthesis gene lpxA. This derivative was named K1 or K1-Vax. For building K1-Vax Acinetobacter baumannii Ab283 cells were transformed with a suicide vector (pVXD-40::K1-Vax) comprising sequences for promoting recombination at the lpxA target locus into the A. baumannii genome. The pVXD-40::K1-Vax plasmid was constructed by cloning the expression construct flanked by homologous regions adjacent to the Ab283 lpxA gene (2000 bp upstream and 2000 bp downstream of the lpxA gene). The suicide plasmid (non-replicative plasmid in A. baumannii) has as selection marker a tretracycline resistance cassette, which induces resistance to tetracycline as well as the gene sacB as counter-selection marker. The pVXD-40::K1-Vax suicide plasmid was introduced into the Ab283 cells by electroporation, matting or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected on tetracycline agar plates. Common techniques like PCR and DNA sequencing were used to check whether the integration of the plasmid into the targeted loci has occurred as expected. The second recombination event was induced by growing the selected tetracycline-resistant Ab283 cells in presence of sucrose leading to recombinant cells where the vector backbone upstream and downstream of the homologous regions was removed and only the expression construct remained into the recombinant Aab283 cells. The Ab283 cells where such second recombination event has occurred was obtained by selecting the recombinant Ab283 cells resistant to sucrose and susceptible to tetracycline. Once selected, common techniques like PCR and DNA sequencing were used to check whether the integration of the expression construct into the targeted loci has occurred as expected. The second recombination also led to complete inactivation of lpxA which is essential for the biosynthesis of the LPS in A. baumannii. Therefore, the second recombination led directly to a to a recombinant LPS-null Ab283 cell.

### Example 2. Construction and Validation of LPS-Null A. Baumannii Strains Expressing OMP Proteins from Other Bacterial Species

Drug-substances based on LPS-null A. baumannii cells expressing OMP proteins from other bacterial species, to be used as vaccine candidates against infections by different bacterial pathogens can be constructed by genome editing of A. baumannii using allelic exchange technology for recombinant strain production. Briefly, we selected an A. baumannii carrier strain. In particular, we screened for a pan-sensitive clinical isolate of A. baumannii that can live without LPS as taught in EP2942389A1. The final strain selected in the screening process was named Ab283. The insertion of the expression construct into the A. baumannii genome can be assessed by recombination by using different strategies well known in the art such as suicide plasmids or linear DNA fragments. For selection of LPS-negative cells we plated the strains on colistin plates (Ab283 was grown in presence of colistin and selection for colistin-resistant mutant was done) and selected for colistin-resistant mutants. Then, we screened by PCR for large insertions or deletions at the lpx genes.

Preparation of a First Drug-Substance, KapaVax2 by Insertion of an Expression Construct at the cysI Locus.

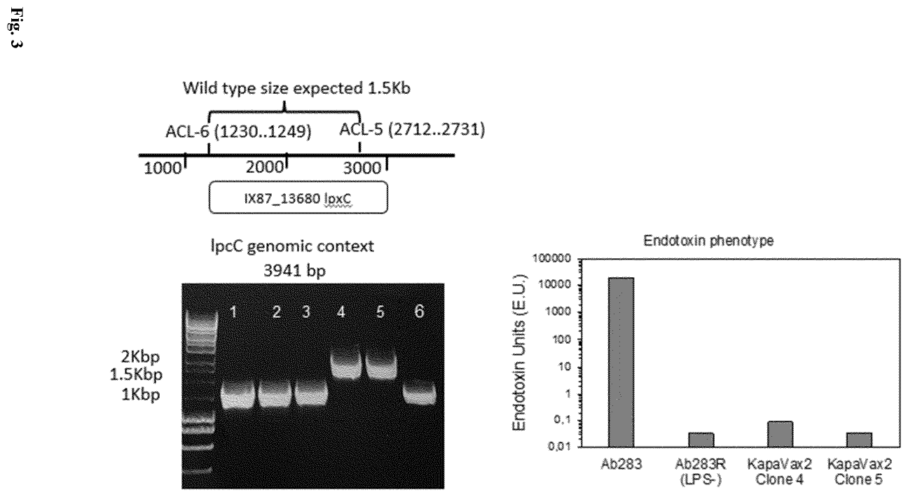

For building of KapaVax2 at the cysI locus, after having inserted the constructs at the cysI locus in the Ab283 wild-type strain, and having proof of expression and localization of the heterologous antigens at the outer-membrane, we selected an LPS-negative derivative after plating on colistin and selection of an LPS-negative mutant with a ISAba insertion of 1 Kb at the lpxC ORF (see FIG. 3).

This candidate KapaVax2 includes both selected K. pneumoniae antigens (OmpA and OmpK36) and three P. aeruginosa antigens (OprF, OprI and PcrV). Each antigen was expressed from an operon-like construct under the control of the A. baumannii OmpA promoter. PcrV was fused to the N-terminus of OprI as a fusion protein. Expression and location at the outer membrane of each antigen was confirmed by Western Blot and ELISA (see FIG. 8). Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::KapaVax2) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequences derived from the outer-membrane proteins OmpA and OmpK36 from K. pneumoniae and the sequences derived from the outer-membrane proteins OprF and OprI from P. aeruginosa and the protein PcrV of the Type 3 secretion system from P. aeruginosa. The oprI and pcrV coding sequences were combined in one coding sequence to produce the new chimeric gene oprI::pcrV. The four coding sequences were expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::KapaVax2 plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::KapaVax2 suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected.

Preparation of a Second Drug Substance, K-Vax by Insertion of an Expression Construct at the cysI Locus.

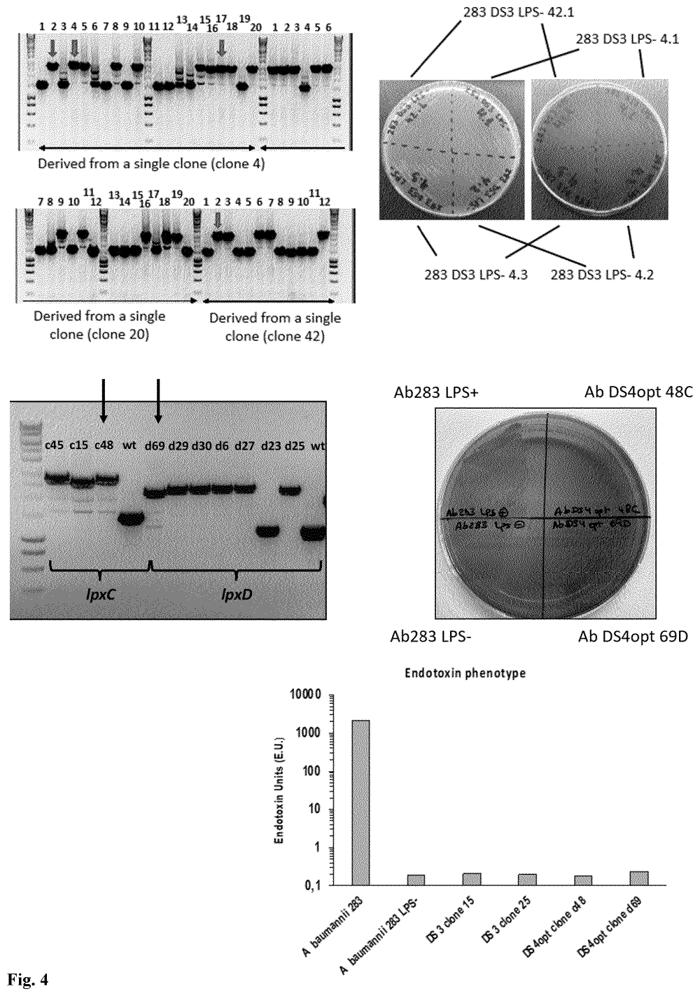

For building of K-Vax at the cysI locus, after having inserted the constructs at the cysI locus in the Ab283 wild-type strain, and having proof of expression and localization of the heterologous antigens at the outer-membrane, we selected an LPS-negative derivative after plating in colistin and selection of an LPS-negative mutant with a ISAba insertion of 1Kb at the lpxC ORF (see FIG. 4).

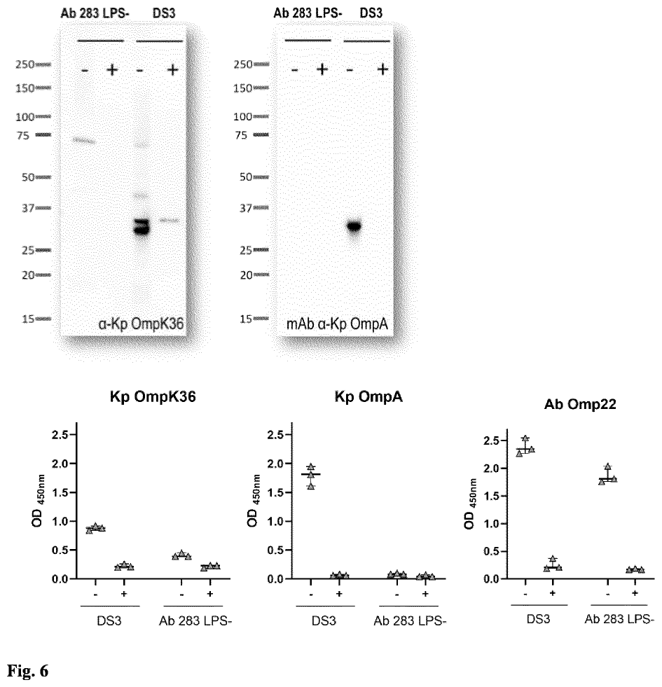

This candidate K-Vax includes the selected K. pneumoniae antigens (OmpA and OmpK36) Each antigen is expressed from an operon-like construct under the control of the A. baumannii OmpA promoter. Expression and location at the outer membrane of each antigen was confirmed by Western Blot and ELISA (see FIG. 6). Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::K-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprised the sequences derived from the outer-membrane proteins OmpA and OmpK36 from K. pneumoniae. Both coding sequences were expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::K-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::K-Vax suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

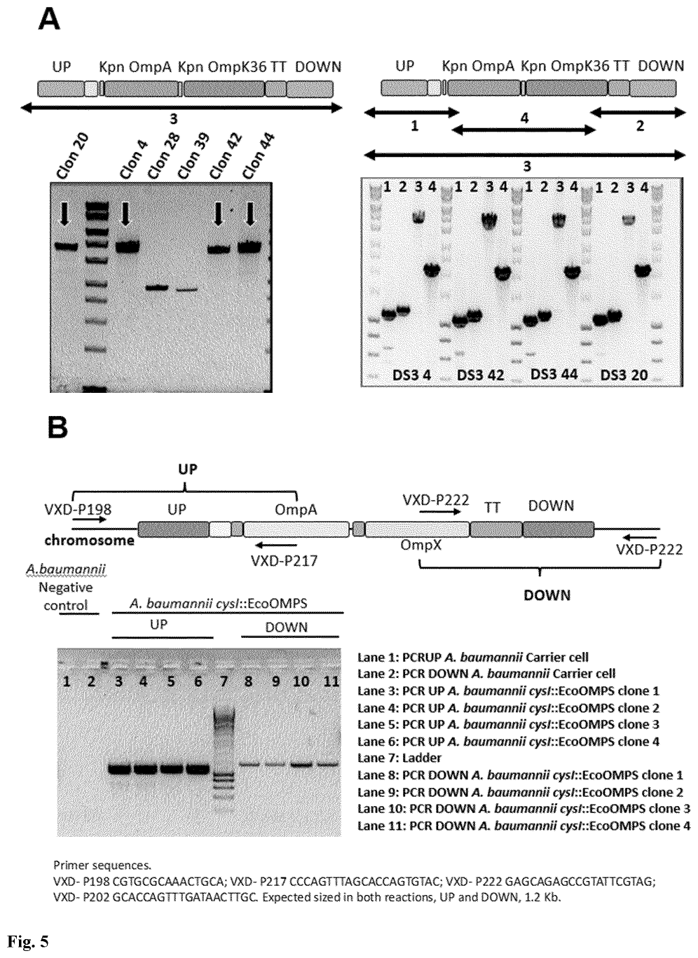

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected (see FIG. 5).

Preparation of a Third Drug Substance, P-Vax by Insertion of an Expression Construct at the cysI Locus.

For building of P-Vax at the cysI locus, after having inserted the constructs at the cysI locus in the Ab283 wild-type strain and having proof of expression and localization of the heterologous antigens at the outer-membrane, we selected an LPS-negative derivative after plating in colistin and selection of an LPS-negative mutant with a ISAba insertion of 1Kb at the lpxD ORF (see FIG. 4).

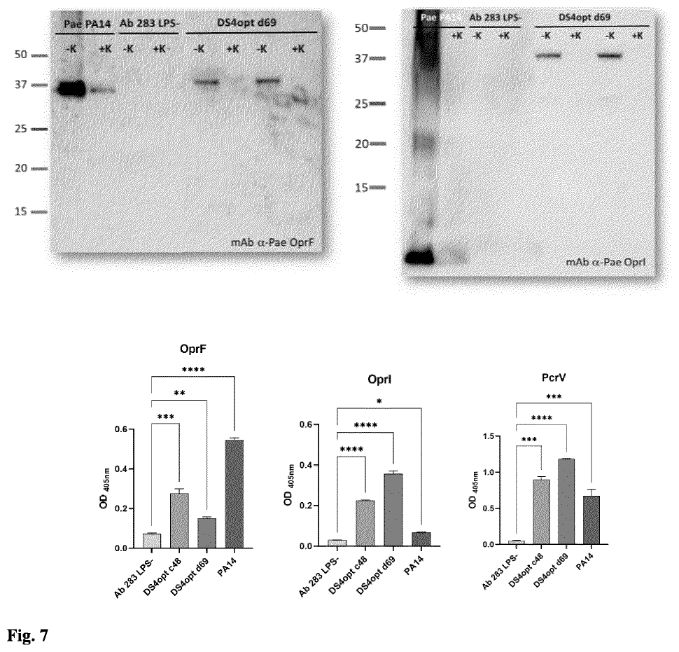

This candidate P-Vax includes three P. aeruginosa antigens (OprF, OprI and PcrV). Each antigen is expressed from an operon-like construct under the control of the A. baumannii OmpA promoter. PcrV was fused to the N-terminus of OprI as a fusion protein. Expression and location at the outer membrane of each antigen was confirmed by Western Blot and ELISA (see FIG. 7). Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::P-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequences derived from the OMPs OprF and OprI from P. aeruginosa and the protein PcrV of the Type 3 secretion system from P. aeruginosa. The oprI and pcrV coding sequences were combined in one coding sequence to produce the new chimeric gene oprI::pcrV. Both coding sequences were expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::P-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::P-Vax suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected.

Preparation of a Fourth Drug Substance, K1-Vax by Insertion of an Expression Construct at the cysI Locus.

This candidate includes the selected OMP from K. pneumoniae OmpA under the control of the A. baumannii OmpA promoter. Expression and location at the outer membrane of the OMP antigen from K. pneumoniae was confirmed by Western Blot and ELISA. Western blot analysis of whole cell lysates and outer membrane extracts with antigen-specific antibodies has been also used to confirm expression and localization of the heterologous antigens in the A. baumannii cell.

For building K1-Vax at lpxA locus, Acinetobacter baumannii Ab283 cells were transformed with a suicide vector (pVXD-40::K1-Vax) comprising sequences for promoting recombination at the lpxA target locus into the A. baumannii genome. LpxA protein is essential for the biosynthesis of the LPS in A. baumannii.

The procedure involved the design of a suicide plasmid that contains the expression construct flanked by homologous regions adjacent of the target gene and selection and counter-selection markers. The ORF included into the expression construct comprise the sequence derived from the outer-membrane protein OmpA from K. pneumoniae controlled by the A. baumannii OmpA promoter. The signal sequence of the outer-membrane protein OmpA from A. baumannii was used to promote location of the OmpA from K. pneumoniae at the outer-membrane of A. baumannii.

The pVXD-40::K1-Vax plasmid was constructed by cloning the expression construct flanked by homologous regions adjacent to the Ab283 lpxA gene (2000 bp upstream and 2000 bp downstream of the lpxA gene). The suicide plasmid (non-replicative plasmid in A. baumannii) has as selection marker a tretracycline resistance cassette, which induces resistance to tetracycline as well as the gene sacB as counter-selection marker.

The pVXD-40::K1-Vax suicide plasmid was introduced into the Ab283 cells by electroporation, matting or chemical transformation and the two-step allelic exchange follows. The first recombination event leads to the insertion of the vector when recombination between the upstream region cloned into the plasmid and the upstream region of the Ab283 genome occurred. Successfully recombinant cells with the plasmid integrated into the genome are selected in tetracycline agar plates. Common techniques like PCR and DNA sequencing are used to check whether the integration of the plasmid into the targeted loci has occurred as expected.

The second recombination event was induced by growing the selected tetracycline-resistant Ab283 cells in presence of sucrose leading to recombinant cells where the vector backbone upstream and downstream of the homologous regions are removed and only the expression construct remains into the recombinant Aab283 cells. The Ab283 cells where such second recombination event has occurred can be obtained by selecting the recombinant Ab283 cells resistant to sucrose and susceptible to tetracycline. Once selected, common techniques like PCR and DNA sequencing are used to check whether the integration of the expression construct into the targeted loci has occurred as expected.

The second recombination also led to complete inactivation of lpxA which is essential for the biosynthesis of the LPS in A. baumannii. Therefore, the second recombination led directly to a to a recombinant LPS-null Ab283 cell. Therefore, no subsequent step of LPS-null mutant selection on colistin plates was needed.

Expression of the heterologous protein OmpA from K. pneumoniae encoded by the expression construct in the recombinant Ab283 cells was analyzed by basic proteomic techniques like Western-Blot confirming its expression. Location of the K. pneumoniae OmpA protein at the outer-membrane of the recombinant A. baumannii cells was analyzed by Western-Blot when samples are treated with proteinase K.

Preparation of a Fifth Drug Substance, Eco1-Vax by Insertion of an Expression Construct at the cysI Locus.

For building of Eco1-Vax at the cysI locus, after having inserted the constructs at the cysI locus in the Ab283 wild-type strain, and having proof of expression and localization of the heterologous antigens at the outer-membrane, we selected an LPS-negative derivative after plating in colistin and selection of an LPS-negative mutant with a ISAba insertion of 1Kb at the lpxC ORF.

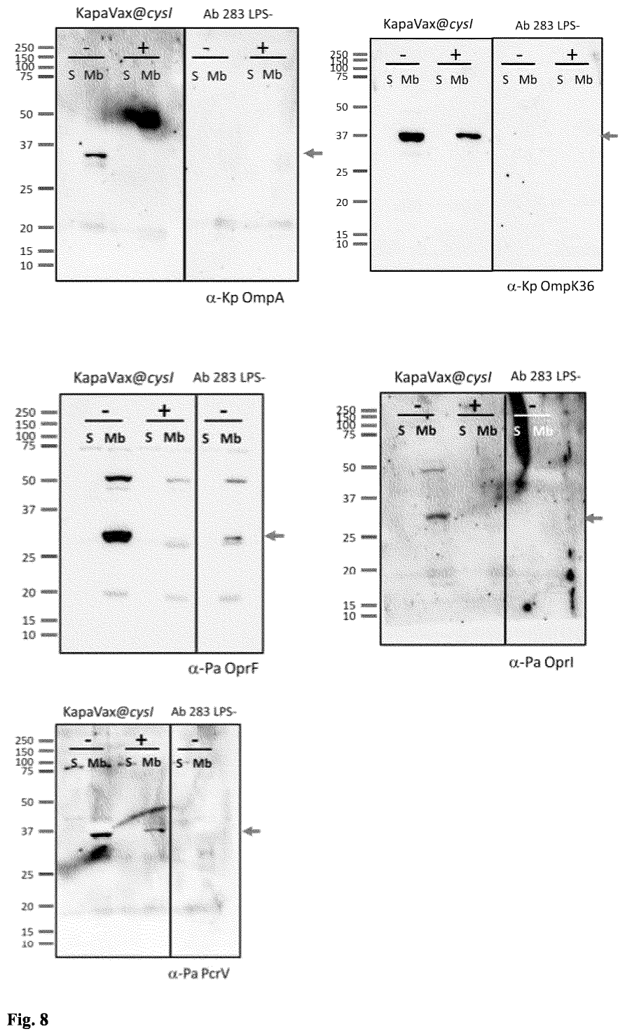

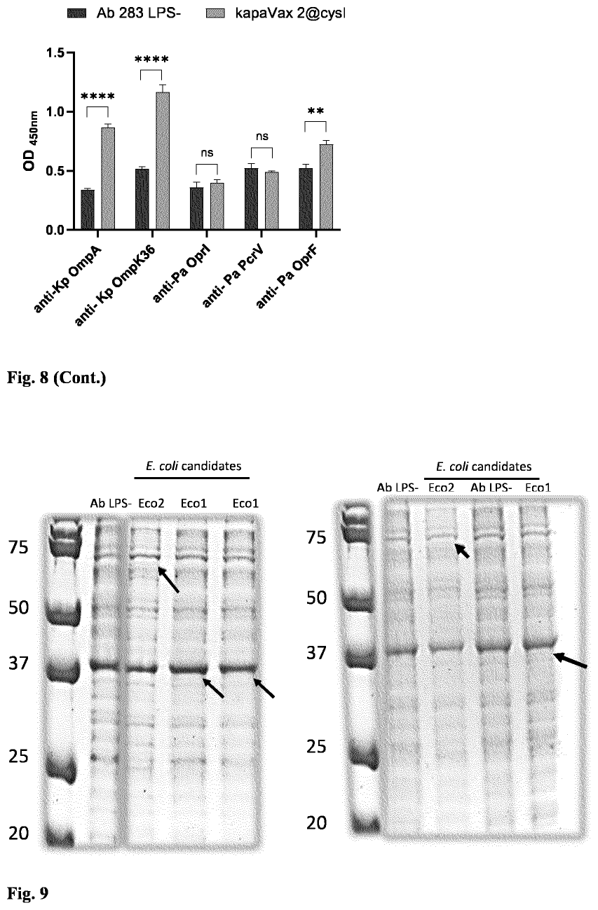

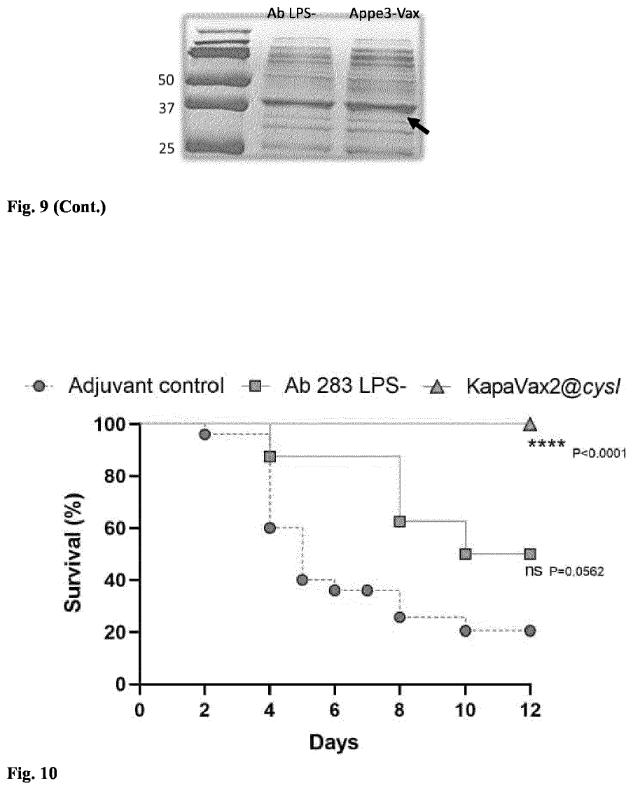

This candidate Eco1-Vax includes two E. coli antigens (OmpA and OmpX). Each antigen is expressed from an operon-like construct under the control of the A. baumannii OmpA promoter. Expression and location at the outer membrane of each antigen was confirmed by basic proteomic techniques including Western Blot and ELISA (see FIG. 9). Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::Eco1-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequences derived from the OMPs OmpA and OmpX from E. coli. Both coding sequences were expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::Eco1-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::Eco1-Vax suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected (see FIG. 5).

Preparation of a Sixth Drug Substance, Eco2-Vax by Insertion of an Expression Construct at the cysI Locus.

For building of Eco2-Vax at the cysI locus, after having inserted the constructs at the cysI locus in the Ab283 wild-type strain, and having proof of expression and localization of the heterologous antigens at the outer-membrane, we selected an LPS-negative derivative after plating in colistin and selection of an LPS-negative mutant with a ISAba insertion of 1Kb at the lpxC ORF.

This candidate Eco2-Vax includes three E. coli antigens (FuyA, HmA, IutA). Each antigen is expressed from an operon-like construct under the control of the A. baumannii OmpA promoter. Expression and location at the outer membrane of each antigen was confirmed by basic proteomic techniques including Western Blot and ELISA (see FIG. 9). Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::Eco2-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequences derived from the OMPs FuyA, HmA, and IutA from E. coli. The three coding sequences were expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::Eco2-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::Eco2-Vax suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected.

Preparation of a Seventh Drug Substance, Eco3-Vax by Insertion of an Expression Construct at the cysI Locus.

For building of Eco3-Vax at the cysI locus, after having inserted the constructs at the cysI locus in the Ab283 wild-type strain, and having proof of expression and localization of the heterologous antigens at the outer-membrane, we selected an LPS-negative derivative after plating in colistin and selection of an LPS-negative mutant with a ISAba insertion of 1Kb at the lpxC ORF.

This candidate Eco3-Vax includes three E. coli antigens (FuyA, HmA, OmpA). Each antigen is expressed from an operon-like construct under the control of the A. baumannii OmpA promoter. Expression and location at the outer membrane of each antigen was confirmed by Western Blot and ELISA. Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::Eco3-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequences derived from the OMPs FuyA, HmA, and OmpA from E. coli. The three coding sequences were expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::Eco3-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::Eco3-Vax suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected.

Preparation of an Eight Drug Substance, Appel-Vax by Insertion of an Expression Construct at the cysI Locus.

For building of Appel-Vax at the cysI locus, after having inserted the constructs at the cysI locus in the Ab283 wild-type strain, and having proof of expression and localization of the heterologous antigens at the outer-membrane, we selected an LPS-negative derivative after plating in colistin and selection of an LPS-negative mutant with a ISAba insertion of 1Kb at the lpxC ORF.

This candidate Appel-Vax includes one Actinobacillus pleuropneumoniae antigen (TpbA). The OMP antigen is expressed from an operon-like construct under the control of the A. baumannii OmpA promoter. Expression and location at the outer membrane of the OMP antigen was confirmed by Western Blot and ELISA. Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::Appel-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequence derived from the OMPs TpbA from A. pleuropneumoniae. The coding sequence was expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::Appel-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::Appel-Vax suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected.

Preparation of a Ninth Drug Substance, Appe2-Vax by Insertion of an Expression Construct at the cysI Locus.

For building of Appe2-Vax at the cysI locus, after having inserted the constructs at the cysI locus in the Ab283 wild-type strain, and having proof of expression and localization of the heterologous antigens at the outer-membrane, we selected an LPS-negative derivative after plating in colistin and selection of an LPS-negative mutant with a ISAba insertion of 1Kb at the lpxC ORF.

This candidate Appe2-Vax includes one Actinobacillus pleuropneumoniae antigen (ApfA). The OMP antigen is expressed from an operon-like construct under the control of the A. baumannii OmpA promoter. Expression and location at the outer membrane of each antigen was confirmed by Western Blot and ELISA. Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::Appe2-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequence derived from the OMPs ApfA from A. pleuropneumoniae. The coding sequence was expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::Appe2-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::Appe2-Vax suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected.

Preparation of a Tenth Drug Substance, Appe3-Vax by Insertion of an Expression Construct at the cysI Locus.

For building of Appe3-Vax at the cysI locus, after having inserted the constructs at the cysI locus in the Ab283 wild-type strain and having proof of expression and localization of the heterologous antigens at the outer-membrane, we selected an LPS-negative derivative after plating in colistin and selection of an LPS-negative mutant with a ISAba insertion of 1Kb at the lpxC ORF.

This candidate Appe3-Vax includes one Actinobacillus pleuropneumoniae antigen (OmpA). The OMP antigen is expressed from an operon-like construct under the control of the A. baumannii OmpA promoter. Expression and location at the outer membrane of each antigen was confirmed by basic proteomic techniques including Western Blot and ELISA (see FIG. 9). Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::Appe3-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequence derived from the OMPs OmpA from A. pleuropneumoniae. The coding sequence was expressed from an operon-like construct under the control of the A. baumannii OmpA promoter. The expression construct contained the signal sequence of the outer-membrane protein OmpA from A. baumannii upstream of each coding sequence in order to promote location of each antigen at the outer-membrane of A. baumannii. The expression construct also contains the transcription termination sequence of the protein OmpA from A. baumannii.

The pVXD-50::Appe3-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::Appe3-Vax suicide plasmid was introduced into the Ab283 cell by electroporation, mating or chemical transformation and a two-step allelic exchange followed. The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected.

Preparation of an Eleventh Drug Substance, Appe4-Vax by Insertion of an Expression Construct at the cysI Locus.

For building of Appe4-Vax at the cysI locus, after having inserted the constructs at the cysI locus in the Ab283 wild-type strain and having proof of expression and localization of the heterologous antigens at the outer-membrane, we selected an LPS-negative derivative after plating in colistin and selection of an LPS-negative mutant with a ISAba insertion of 1Kb at the lpxC ORF.

This candidate Appe4-Vax includes one Actinobacillus pleuropneumoniae antigen (OmpW). The OMP antigen is expressed from an operon-like construct under the control of the A. baumannii OmpA promoter. Expression and location at the outer membrane of each antigen was confirmed by Western Blot and ELISA. Western blot analysis of whole cell lysates and outer membrane extracts with antigen-specific antibodies has been also used to confirm expression and localization of the heterologous antigens in the A. baumannii cell.

Previously to the LPS-null mutant selection, for integration of the expression constructs at the cysI locus of A. baumannii Ab283, A. baumannii cells were transformed with a suicide vector (pVXD-50::Appe4-Vax) comprising sequences for promoting recombination at the cysI target locus into the A. baumannii genome. The procedure involved the design of a suicide plasmid that contains the expression construct flanked by homologous regions adjacent of the target gene and selection and counter-selection markers. The ORFs included into the expression construct comprise the sequence derived from the OMPs OmpW from A. pleuropneumoniae. The coding sequence was expressed from an operon-like construct under the control of the A. baumannii OmpA promoter. The expression construct contained the signal sequence of the outer-membrane protein OmpA from A. baumannii upstream of each coding sequence to promote location of each antigen at the outer-membrane of A. baumannii. The expression construct also contains the transcription termination sequence of the protein OmpA from A. baumannii.

The pVXD-50::Appe4-Vax plasmid was constructed by cloning the expression construct flanked by homologous regions adjacent to the Ab283 cysI gene (600 bp upstream and 600 bp downstream of the cysI gene). The suicide plasmid (non-replicative plasmid in A. baumannii) has as selection marker a kanamycin resistant gene, which induce resistance to kanamycin as well as the gene sacB as counter-selection marker.

The pVXD-50::Appe4-Vax suicide plasmid was introduced into the Ab283 cell by electroporation, mating or chemical transformation and a two-step allelic exchange followed.

The first recombination event led to the insertion of the vector when recombination between the upstream region cloned into the plasmid and the upstream region of the Ab283 genome occurred. Successfully recombinant cells with the plasmid integrated into the genome were selected in kanamycin agar plates. Common techniques like PCR and DNA sequencing were used to check whether the integration of the plasmid into the targeted loci has occurred as expected.

The second recombination event was induced by growing the selected kanamycin-resistant Ab283 cells in presence of sucrose leading to recombinant cells where the vector backbone upstream and downstream of the homologous regions were removed and only the expression construct remained into the recombinant Ab283 cells. The Ab283 cells where such second recombination event has occurred were obtained by selecting the recombinant Ab283 cells resistant to sucrose and susceptible to kanamycin. Once selected, common techniques like PCR and DNA sequencing were used to check whether the integration of the expression construct into the targeted loci has occurred as expected.

### Example 2. Protocol for OMP Sensitivity to Proteinase K

Objective: to test the location of the heterologous OMP antigens from other bacterial pathogens in the outer membrane of the LPS deficient Acinetobacter baumannii.

Brief description: A. baumannii LPS-whole cells, expressing OMP antigens from other bacterial pathogens or the parental LPS-version of the A. baumannii strain without expressing then OMP antigens, are treated with proteinase K. Membrane preparations from samples treated or not treated (control) are run into 12% acrylamide gels. Consecutively, Western-Blot is carried out using polyclonal or monoclonal antibodies raised against the specific heterologous OMP proteins. Proteins located at the membrane surface are degraded upon proteinase K treatment. This part of the protocol has been also performed with lysates of the whole cell and the results show that the polyclonal antibodies recognize cytosolic proteins due to cross-reaction in both samples, treated and not treated with proteinase K. Whereas OMPs with expected location at the outer-membrane are only detected in those samples that have not been treated.

### Methodology:

### Proteinase K Treatment:

1. Preinoculum from a single colony is grown o/n at 30° C. with shaking.
2. Cultures are diluted to OD 0.05 and then grown at 30° C. with shaking until OD 2.
3. 25 ml of culture is concentrated by centrifugation and supernatant is discarded.
4. Pellet is resuspended in 10 ml of PBS, with proteinase K 250 μg/ml in treated samples.
5. Incubation at 37° C. shaking for 2 hours.
6. Pellet is concentrated by centrifugation and then resuspended in 10 ml of PBS or PBS+3 mM PMFS (protease inhibitor) to stop the reaction in samples treated with proteinase K.
7. Incubation at room temperature for 10 min.

### Membrane Preparation

8. Sonication as follows: 10 seconds at 20% amplitude, 30 seconds' pause. This cycle is repeated 7 times.
9. Ultracentrifugation at 4000 rpm, 8° C. for 1 hour.
10. Discard supernatant and resuspend in 250 μl PBS triton 1%.
11. Incubate at 4° C. o/n for solubilization of membranes.

### SDS-PAGE and WB

12. Sample preparation: 25 μl of membrane preparation+25 μl loading buffer 4×+50 μl of milliQ Water.
13. Boil at 98° C. for 20 minutes.
14. Load 10 μl on 12% acrylamide gels
15. Run at 170V for 1 hour.
16. Wet transference to Nitrocellulose membrane at 360 mA for 1:30 hours
17. Ponceau staining for loading control.
18. Blocking with PBS tween 1% milk 5% for 1 hour
19. O/N incubation at 4° C. with primary antibody at 1:5000 in PBS tween 1% milk 5%
20. Wash membranes with PBS tween 1%. Repeat wash 2 times more.
21. Incubation with 1:5000 secondary antibody in PBS tween 1% milk 5% at room temperature for 1 hour.
22. Wash membranes with PBS tween 1%. Repeat wash 5 times more.
23. Treatment with chemiluminescence reagents for 5 min and visualization.

Examples of the final results with some of the drug-substance delivered with the technology of the invention are shown in FIGS. 6, 7, and 8. The technique was used to confirm outer-membrane location of all heterologous antigens expressed for construction of the drug-substances mentioned in Example 1.

### Example 3. Efficacy of the Drug Substances Created in A. Baumannii for Protection Against Infections Caused by Other Bacterial Pathogens

Vaccine candidates were prepared from the different drug substances by fermentation of the drug-substance cells at laboratory scale and subsequent heat inactivation.

For in vivo proof-of-concept studies, we employed a murine sepsis model that was previously developed to test in vivo potency of our monovalent vaccine against A. baumannii, AcinetoVax, as described in Garcia-Quintanilla et al. 2014. Briefly, 6-8 week old C57BL/6 animals (8-12 animals/group) were immunized by IM injection of 2 doses of inactivated drug-substance cells formulated with 0.83 mg/dose of Aluminum Hydroxide as adjuvant. The dose were administered 2-weeks apart. Seven days after the second immunization, the animals were challenged by intraperitoneal injection of an inoculum of a reference strain of the bacterial pathogen to be tested. The inoculum was equivalent to the minimum lethal dose (MLD), previously characterized for each strain in this model. We tested the in vivo efficacy in independent challenge experiments against each of the target pathogens. The animals were monitored for 7-12 days to determine survival. After exitus or at the end of the follow-up period the bacterial load in lungs, spleen and kidneys were determined as a quantitative value of bacterial spread, as well as in the blood to evaluate the bacteremia associated to the experimental sepsis. A separate group of animals (8-10 animals/group) were sacrificed 12 (or 18) hours after infection to evaluate the bacterial load in the different organs and serum levels of TNF-α, IL-1β and IL-6 to characterize the inflammatory response developed during infection in vaccinated animals versus unvaccinated control animals.

The primary variable for evaluation of the efficacy of each candidate was survival in the post-challenge period in the vaccinated mice compared to control mice inoculated with the vaccine vehicle (only adjuvant). As secondary variable, comparison of protection between the drug substance “LPS-null Acinetobacter baumannii Ab283 with expression of the heterologous OMP antigens at the outer-membrane” and the drug-substance “LPS-null Acinetobacter baumannii Ab283 carrier cell, without the heterologous OMP antigens on the external membrane” was done for evaluation of the contribution of the heterologous OMP antigens to protection against their target pathogen.

### Example of Protection of KapaVax Against a Lethal Sepsis Produced by Klebsiella pneumoniae

KapaVax2 (KapaVax) is made of LPS-null A. baumannii Ab283 cells expressing the OMP antigens from K. pneumoniae OmpA and OmpK36 and P. aeruginosa OprF and fusion protein OprI::PcrV.

As shown in FIG. 10, there was significant protection of mice vaccinated with KapaVax2 (KapaVax) compared with mice vaccinated with the adjuvant alone (vehicle) against lethal sepsis produced by the hypervirulent, hypercapsulated K. pneumoniae clinical isolate ATCC43816. In addition, mice vaccinated with the carrier cell alone (LPS-null A. baumannii Ab283) showed partial cross-protection. The protection by KapaVax was significantly higher than that obtained with Ab283 LPS-, therefore the presence of the heterologous OMP antigens was required for increasing protection against the target pathogen K. pneumoniae.

### Example of Protection of KapaVax Against a Lethal Sepsis Produced by Pseudomonas aeruginosa

KapaVax2 (KapaVax) is made of LPS-null A. baumannii Ab283 cells expressing the OMP antigens from K. pneumoniae OmpA and OmpK36 and P. aeruginosa OprF and fusion protein OprI::PcrV.

As shown in FIG. 11, there was significant protection of mice vaccinated with KapaVax2 (KapaVax) compared with mice vaccinated with the adjuvant alone (vehicle) against lethal sepsis produced by the P. aeruginosa clinical isolate PA14.

### Example of Protection of K-Vax Against a Lethal Sepsis Produced by Klebsiella pneumoniae

K-Vax is made of LPS-null A. baumannii Ab283 cells expressing the OMP antigens from K. pneumoniae OmpA and OmpK36.

As shown in FIG. 12, there was significant protection of mice vaccinated with K-Vax (DS3) compared with mice vaccinated with the adjuvant alone (vehicle) against lethal sepsis produced by the hypervirulent, hypercapsulated K. pneumoniae clinical isolate ATCC43816. In addition, mice vaccinated with the carrier cell alone (LPS-null A. baumannii Ab283) showed partial cross-protection. The protection by K-Vax was significantly higher than that obtained with Ab283 LPS-, therefore the presence of the heterologous OMP antigens was required for increasing protection against the target pathogen K. pneumoniae.

### Example of Protection of P-Vax Against a Lethal Sepsis Produced by Pseudomonas aeruginosa

P-Vax is made of LPS-null A. baumannii Ab283 cells expressing the OMP antigens from P. aeruginosa OprF and fusion protein OprI::PcrV.

As shown in FIG. 13, there was significant protection of mice vaccinated with P-Vax (DS4) compared with mice vaccinated with the adjuvant alone (vehicle) against lethal sepsis produced by the virulent P. aeruginosa clinical isolate PA14. In addition, mice vaccinated with the carrier cell alone (LPS-null A. baumannii Ab283) showed partial cross-protection. The protection by P-Vax was significantly higher than that obtained with Ab283 LPS-, therefore the presence of the heterologous OMP antigens was required for increasing protection against the target pathogen P. aeruginosa.

### Example 4. The Drug-Substances Constructed in LPS-Null A. Baumannii Expressing OMP Antigens from Other Bacterial Pathogens Conserved the Capacity of LPS-Null A. Baumannii Cells of Protecting Against Infections Produced by A. Baumannii

### Protection of AcinetoVax Against Lethal Sepsis Caused by Diverse A. Baumannii Clinical Isolates

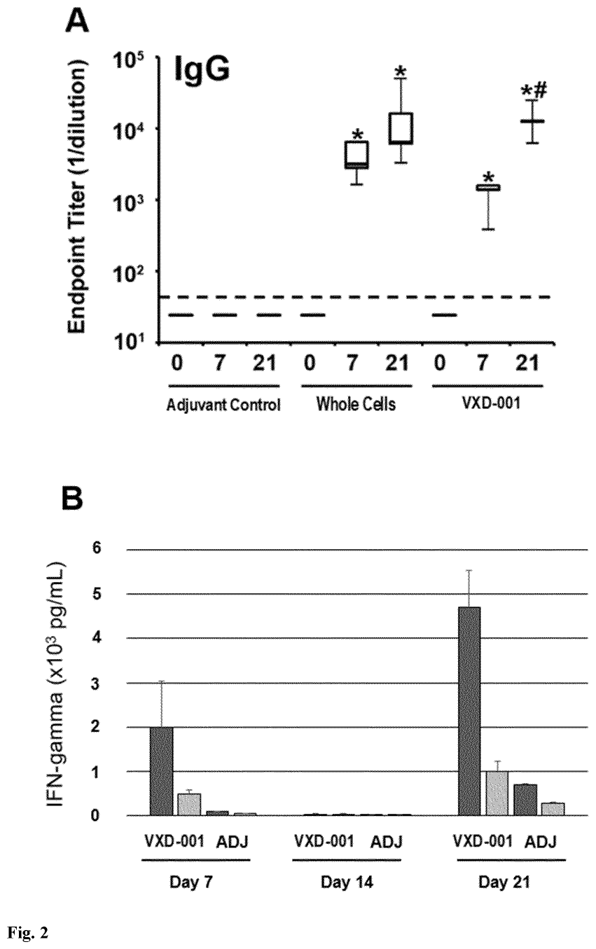

Vaccination with VXD001 (AcinetoVax, a vaccine based on inactivated whole-cells of an LPS-deficient A. baumannii strain characterized by the complete inactivation of lpxC as taught in EP2942389A1 plus the adjuvant aluminum hydroxide) led to protection against lethal sepsis caused by diverse A. baumannii clinical isolates. Protection was supported by a rapid humoral response established after the 1st dose, and detected T cell-mediated response (Thl, Th2, Thl7) established after the 1st dose and significantly boosted after the 2nd dose (see FIG. 2).

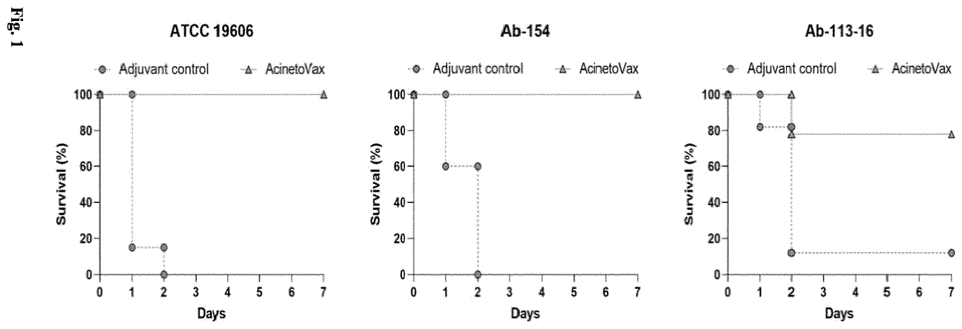

As shown in FIG. 1, VXD001 (AcinetoVax) protects against infection with A. baumannii clinical isolates. The same sepsis model described in Example 3 was used. Mice were infected with the indicated strains of A. baumannii (ATCC 19606, Ab-154, and Ab-113-16) 7 days after the second immunization with AcinetoVax (day 21) and survival was monitored for 7 days. ATCC19606 is a urine infection clinical isolate used as reference strain in sepsis and pneumonia models of infection in mice. Ab-154 is a clinical isolate sensitive to carbapenems, from an outbreak at the Hospital Virgen del Rocio of Seville in 2002. Ab-113-16 is a pan-resistant clinical isolate from a patient who died as a consequence of the 2002 outbreak.

### Protection of KapaVax Against Lethal Sepsis Caused by A. Baumannii

AcinetoVax is made of LPS-null A. baumannii AB-001 cells. As described above, the immunity raised by AcinetoVax is able to prevent infections by A. baumannii. With the LPS being eliminated from the outer-membrane, the authors found that the immunodominant OMP proteins responsible for the protective immunity raised by AcinetoVax are the A. baumannii proteins OmpA and Omp22. The authors also found by mass-spectrometry analysis of the external membrane of AcinetoVax and other A. baumannii LPS-strains created in different genetic backgrounds, that OmpA and Omp22 are the most abundant OMP proteins in the outer-membrane. Once the drug substances based on LPS-A. baumannii cells expressing heterologous OMP antigens from other bacterial pathogens at the external membrane were constructed, the question was whether the presence of the heterologous OMP antigens would affect the immunity raised against A. baumannii.

As shown in FIG. 14, the presence of the OMP antigens from K. pneumoniae OmpA and OmpK36 and from P. aeruginosa OprF and fusion OprI::PcrV in the drug-substance KapaVax did not affect the capacity of the vaccine of protecting mice against a lethal sepsis produced by A. baumannii, relative to that previously described by AcinetoVax. The result was consistent with the finding that, although built on a different strain of A. baumannii (Ab283) than that of AcinetoVax (AB001), mass-spectrometry analysis of the external membrane of KapaVax showed that A. baumannii OmpA and Omp22 were still the most abundant proteins in the outer-membrane. The same result was found for all the drug-substances constructed as shown in Example 1.

### Example 5. Immunogenicity Analyses Showed that Vaccination with Drug-Substances Constructed in LPS-Null A. Baumannii Expressing OMP Antigens from Other Bacterial Pathogens Raised Specific Immunity Against the Other Bacterial Pathogens and Also Against A. Baumannii

### Example of KapaVax Raising Specific Immunity Against the Challenge Strain of K. pneumoniae and Against the K. Pneumoniae Antigens Included in KapaVax

KapaVax2 (KapaVax) is made of LPS-null A. baumannii Ab283 cells expressing the OMP antigens from K. pneumoniae OmpA and OmpK36 and P. aeruginosa OprF and fusion protein OprI::PcrV.

Immunogenicity was determined by immunizing C57BL/6 mice (8-10 mice/group) by IM injection with 2 doses on days 0 and 14. KapaVax candidates were adjuvanted with aluminum hydroxide.

The primary variable for evaluation of the immunogenicity was total IgG against the vaccine cells, whole-cells of K. pneumoniae and P. aeruginosa (the target pathogens), against whole-cells of A. baumannii and against each of the antigens measured by indirect ELISA. All assays included control mice immunized with vehicle alone at the same timepoints.

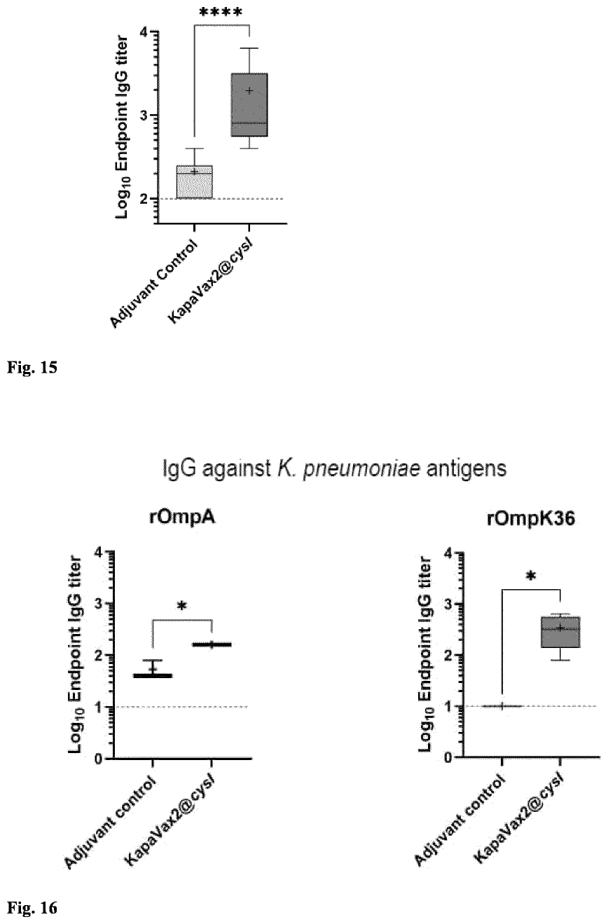

As shown in FIGS. 15 and 16, KapaVax raised specific immunity against the challenge strain of K. pneumoniae ATCC43816 and against the K. pneumoniae antigens included in KapaVax, i.e. K. pneumoniae OMPs OmpA and OmpK36. Immunogenicity data supported contribution of K. pneumoniae antigens & requirement for full protection against K. pneumoniae ATCC43186 as described in Example 3.

### Example of KapaVax Raising Specific Immunity Against the Challenge Strain of P. aeruginosa and Against the K. Pneumoniae Antigens Included in KapaVax

KapaVax2 (KapaVax) is made of LPS-null A. baumannii Ab283 cells expressing the OMP antigens from K. pneumoniae OmpA and OmpK36 and P. aeruginosa OprF and fusion protein OprI::PcrV.

FIG. 17 shows IgG levels against P. aeruginosa cells. ELISA recognition of P. aeruginosa PA14 cells by antisera from animals immunized with KapaVax (N=5) or vehicle alone (N=5), sampled at day 21 (7 days after 2nd immunization). Statistical significance: ns p>0,05; *p<0.05; ** p<0.005; ***p<0.001. Red dashed line indicates detection limit.

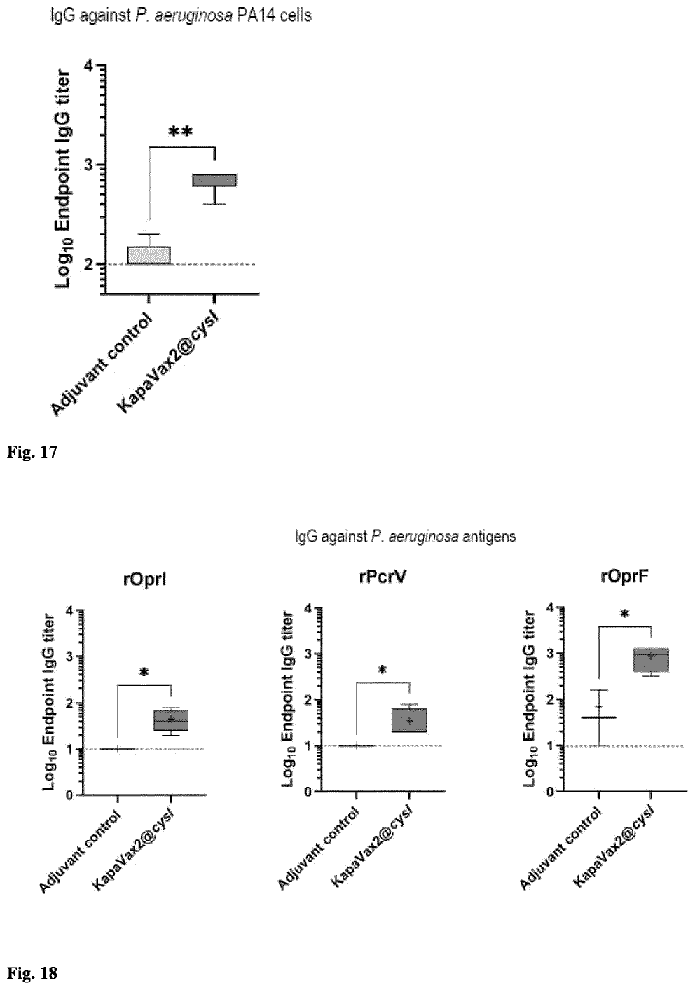

FIG. 18 shows IgG levels against P. aeruginosa antigens OprF, OprI, and PcrV. ELISA recognition of recombinant proteins by antisera from animals immunized with 2 doses of KapaVax (N=4) or vehicle alone (N=4), sampled at day 21 (7 days after 2nd immunization). Statistical significance: ns p>0,05; *p<0.05; ** p<0.005; ***p<0.001. Red dashed line indicates detection limit.

### Example of KapaVax Raising Specific Immunity Against A. Baumannii

KapaVax2 (KapaVax) is made of LPS-null A. baumannii Ab283 cells expressing the OMP antigens from K. pneumoniae OmpA and OmpK36 and P. aeruginosa OprF and fusion protein OprI::PcrV

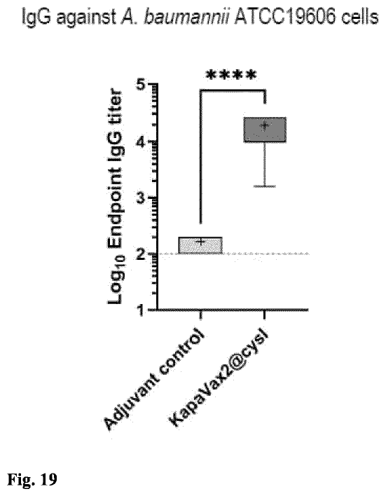

FIG. 19 shows IgG levels against A. baumannii cells. ELISA recognition of A. baumannii ATCC19606 cells by antisera from animals immunized with 2 doses of KapaVax (N=9) or vehicle alone (N=9), sampled at day 21 (7 days after 2nd immunization). Boxes show IQR, horizontal line and cross show median and mean, respectively. Error bars extend to the CI95%.

Statistical analysis by Unpaired 2-tailed Mann-Whitney test (ns p>0,05; *p<0.05; ** p<0.005; ***p<0.001). Red dashed line indicates detection limit.

### Example 6. Detection of a Panel of Global Isolates by ELISA Supports Global Strain Coverage

One of the many sought features of this invention was to overcome one of the major caveats of bacterial vaccines, typically based on specific sugar antigens from the LPS or the capsular polysaccharide. These vaccines, which are the predominant type of vaccines in the market or clinical development, raises specific immunity against a limited number of bacterial strains (serotypes), while others escape from the immunity raised by the vaccine. The technology of this invention, as has been shown extensively in the previous examples, is able to raise immunity against different bacterial OMP antigens. The OMPs are very well conserved proteins among all bacterial strains of a specific bacterial species, and even across bacterial species. The selection of the vaccine alleles for all drug substances shown in Example 1 (see also the sequences of the vaccine alleles, SEQ ID No. 2 to 16, excluding the N-terminal signal peptide) has been made based on the most prevalence highly-conserved OMP alleles among all circulating strains of each pathogen. The hypothesis though is that the immunity raised by the vaccines made with the present invention will have a high coverage among all diversity of circulating strains for each pathogen, i.e. will be universal. The following example, made with anti-sera raised by KapaVax, is shown to illustrate that the authors are obtaining results supporting this hypothesis. KapaVax2 (KapaVax) is made of LPS-null A. baumannii Ab283 cells expressing the OMP antigens from K. pneumoniae OmpA and OmpK36 and P. aeruginosa OprF and fusion protein OprI::PcrV.

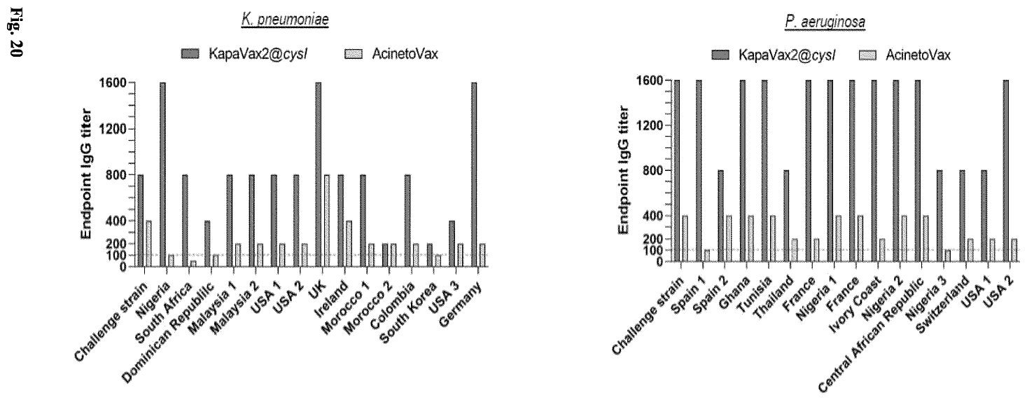

FIG. 20 shows that KapaVax2 antisera shows specific recognition of 93% of a panel of 15 diverse global clinical isolates of K. pneumoniae (73% with higher or similar ELISA titers to that against the challenge strain K. pneumoniae ATCC43816). In addition, KapaVax2 antisera shows specific recognition of 100% of a panel of 15 diverse global clinical isolates of P. aeruginosa (93% with higher or similar ELISA titers to that against the challenge strain PA14).

The information about the diversity of the strain panel used in FIG. 20, for confirmation of the universality of the vaccine anti-sera is shown in Tables 2 and 3. The strain panel is covering all the major types of K. pneumoniae and P. aeruginosa clinical isolates currently circulating on a global scale.

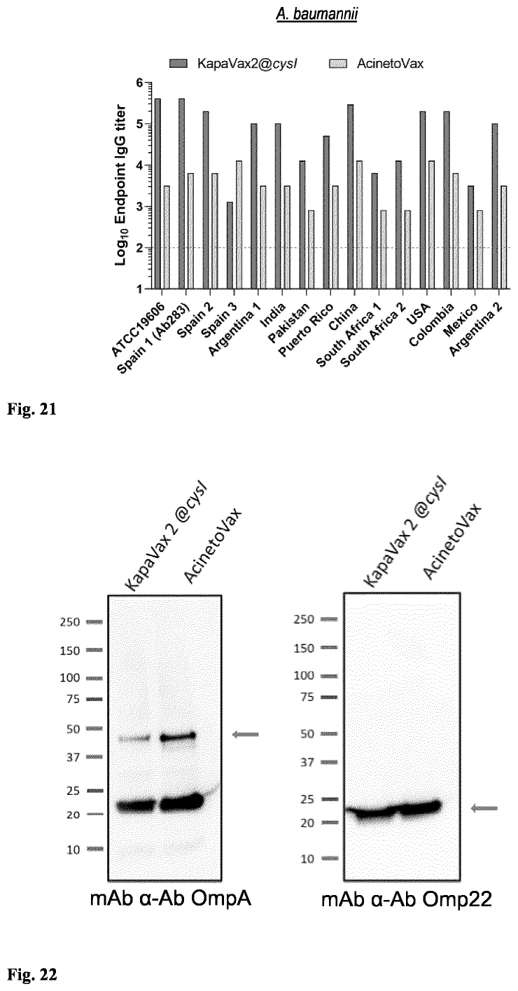

FIG. 21 also shows that the universality shown by KapaVax anti-sera against K. pneumoniae and P. aeruginosa is also true for the third pathogen targeted by the multi-pathogen vaccine made by applying the present invention, i.e. A. baumannii. The information about the diversity of the strain panel used in FIG. 21, for confirmation of the universality of the vaccine anti-sera is shown in Table 4.

TABLE 2
Table 2. Collection of K. pneumoniae clinical isolates.

| | Isolate | | | |
|---|---|---|---|---|
| Number of | geographical | | | |
| isolate | origin | ST | K_type | O_type |
| #1 | Nigeria | ST15 | K-112 | O1 |
| #2 | Morocco | ST307 | K-102 | O2 |
| #3 | South Africa | ST1401 | K-49 | OL101 |
| #4 | Morocco | ST405 | K-151 | O4 |
| #5 | South Africa | Pending for sequencing | | |
| #6 | Malaysia | ST17 | K-25 | O5 |
| #7 | Korea, South | ST14 | K-2 | O1 |
| #8 | Malaysia | ST11 | K-24 | O2 |
| #9 | Thailand | ST1107 | K-3 | O2 |
| #10 | Malaysia | ST45 | K-62 | O2 |
| #11 | France | ST461 | K-10 | O1 |
| #12 | Ireland | ST20 | K-28 | O1 |
| #13 | United Kingdom | ST661 | K-3 | O2 |
| #14 | Germany | ST661 | K-24 | O2 |
| #15 | United States | ST36 | K-27 | O2 |
| #16 | United States | ST1263 | K-10 | O3/O3a |
| #17 | United States | ST27 | K-38 | O1 |
| #18 | Colombia | ST268 | K-20 | O2 |
| #19 | Dominican | ST147 | K-10 | O3/O3a |
| | Republic | | | |
| #20 | Canada | ST147 | K-64 | O2 |
| #21 | South Africa | ST307 | K-102 | O2 |
| ATCC43816 | N/A | ST493 | K-2 | O1 |

The figure shows the country of origin of the clinical isolate, sequence type, the type of K locus (capsule) of each bacterial strain and the O type (O polysaccharide serotype).

TABLE 3
Table 3. Collection of P. aeruginosa clinical isolates.

| | Isolate geographical | | |
|---|---|---|---|
| Number of isolate | origin | ST | O serotype |
| Challenge strain | PA14 | 253 | O10 |
| #1 | Ivory Coast | 235 | O11 |
| #2 | Nigeria | 235 | O11 |
| #3 | Central African Republic | 235 | O11 |
| #4 | Switzerland | 235 | O11 |
| #5 | Nigeria | 235 | O11 |
| #6 | Switzerland | 235 | O11 |
| #7 | Switzerland | 235 | O11 |
| #8 | France | 235 | O11 |
| #9 | Spain | 235 | O11 |
| #10 | France | 111 | O12 |
| #11 | Nigeria | 111 | O12 |
| #12 | France | 111 | O12 |
| #13 | France | 111 | O12 |
| #14 | Spain | 111 | O12 |
| #15 | Spain | 175 | O4 |
| #16 | Spain | 175 | O4 |
| #17 | Spain | 274 | O3 |
| #18 | Spain* | 1089 | O3 |
| #19 | England* | 146 | O6 |
| #20 | Ghana | 233 | O6 |
| #21 | Tunisia | 654 | O4 |
| #22 | Thailand | 260 | O6 |
| #23/#24/#28 | USA | 298 | O11 |
| #25/#26/#27 | USA | 446 | O11 |

The figure shows the country of origin of the clinical isolate, sequence type, and the O type (O polysaccharide serotype) of each bacterial strain.

TABLE 4
Table 4. Collection of A. baumannii clinical isolates.

| | Isolate geographical | | | |
|---|---|---|---|---|
| Number of isolate | origin | ST | K locus | OC locus |
| Challenge strain | ATCC19606 | 52 | KL3 | OCL1 |
| #1 | USA | 1 | KL4 | OCL2 |
| #2 | Argentina | 1 | KL1 | OCL1 |
| #3 | India | 1 | KL15 | OCL3 |
| #4 | Pakistan | 1 | KL125 | OCL2 |
| #5 | Puerto Rico | 1 | KL1 | OCL1 |
| #6 | Australia | 2 | KL2 | OCL1 |
| #7 | China | 2 | KL34 | OCL1 |
| #8 | South Korea | 2 | KL2 | OCL1 |
| #9 | South Africa | 2 | KL2 | OCL1 |
| #10 | USA | 780 | KL1 | OCL1 |
| #11 | South Africa | 3 | KL1 | OCL1 |
| #12 | USA | 79 | KL124 | OCL10 |
| #13 | Argentina | 79 | KL22 | OCL10 |
| #14 | Colombia | 79 | KL12 | OCL10 |
| #15 | Mexico | 422 | KL7 | OCL10 |
| #16 | Singapore | 25 | KL10 | OCL7 |
| #17 | Argentina | 25 | KL14 | OCL7 |
| #18 | Mexico | 25 | KL7 | OCL7 |
| #19 | Spain | 2 | KL2 | OCL1 |
| #20 | Spain | 2 | KL2 | OCL1 |
| #21 | Spain | 745 | KL2 | OCL1 |
| #22 | Spain | 3 | KL1 | OCL1 |
| #23 | Spain | 79 | KL3 | OCL10 |
| #24 | Spain | 79 | KL3 | OCL10 |

The figure shows the country of origin of the clinical isolate, sequence type, the type of K locus (capsule) of each bacterial strain and the OC type.

### Example 7. Exposure of A. Baumannii Immunodominant Antigens OmpA and Omp22 at the Outer-Membrane of KapaVax2

KapaVax2 (KapaVax) is made of LPS-null A. baumannii Ab283 cells expressing the OMP antigens from K. pneumoniae OmpA and OmpK36 and P. aeruginosa OprF and fusion protein OprI::PcrV. Immunodominant OMPs from A. baumannii OmpA and Omp22 are detected by Western-Blot from crude extracts made from vaccine batches of KapaVax

FIG. 22 shows Expression of A. baumannii antigens in KapaVax2 and AcinetoVax vaccine batches: total lysate preparations from vaccine batches (2×10¹⁰ cells/ml). Samples were run on gels with 4-16% acrylamide gradient. Panels show the signal obtained with monoclonal antibodies raised against OmpA (left) and Omp22 (right).

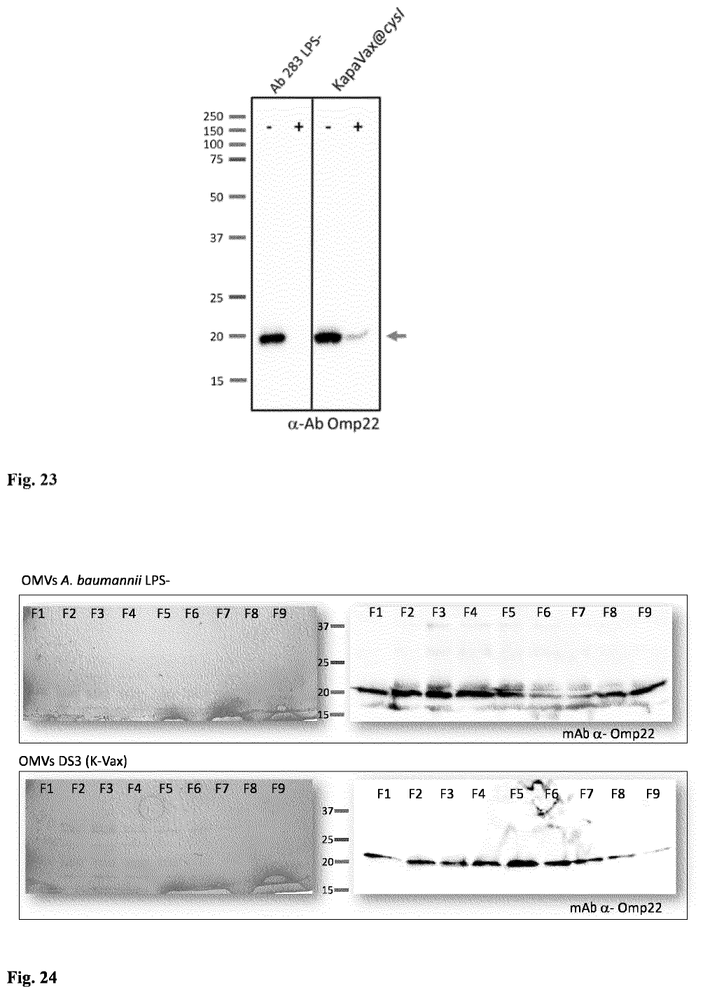

FIG. 23 shows Surface exposure of A. baumannii Omp22 at the outer-membrane of KapaVax and the carrier cell Ab283 LPS-. Cultures were washed after growth, resuspended in PBS and treated with 0.5 mg/ml of Proteinase K for 1 h at 37° C. Western-Blot with a monoclonal antibody raised against Ab-Omp22. Treatment with Proteinase K is indicated by +(treated) or—(untreated) in each sample). Mb: Outer-Membrane extract; S: extract supernatant. Protocol described in Example 2.

### Example 8. Purification of Outer-Membrane Vesicles is Also Possible from LPS-Null Cells of A. Baumannii, and Contains the OMP Heterologous Antigens from Other Bacterial Pathogens

As taught by the authors in EP2942389A1 and shown here, outer-membrane vesicles (OMVs) can be purified from cultures of A. baumannii LPS-null derivatives and used alternatively as drug-substances instead of the whole-cells. The authors have found that when heterologous OMP antigens from other bacterial pathogens are expressed on the external membrane of the LPS-null A. baumannii cell, the capacity to release OMVs is not impaired. The authors have purified OMVs from the cells of the drug-substances described in Example 1.

The protocol used for OMV purification is as follows: OMVs are obtained from 200 ml overnight culture grown in Müeller Hilton II at 30° C. and 150 rpm shaking. After growth, cultures are centrifuged at 10.000×g for 15 min at 4° C., supernatant is saved and pellet is discarded. This step is repeated 3 times. Concentration of OMVs is carried out in a first place by filtration using 0.22 micron filters. Filtrated OMVs are resuspended into 10-15 ml of solvent-resistant buffer. In second place, OMVs are ultracentrifuged at 100.000×g for 6 hours at 4° C. Supernatant form ultracentrifugation is carefully discarded and pellet is resuspended into 2 ml of sterile PBS. Purification of OMVs is carried out by size exclusion chromatography (SEC), fractions are collected and the presence of OMV in each fraction is evaluated by Western-Blot.

As example, the results of OMVs purification from DS3 (K-Vax) and the carrier cell LPS-null A. baumannii Ab283 are shown in FIG. 24. K-Vax is an LPS-null A. baumannii Ab283 cell expressing the OMPs OmpA and OmpK36 from K. pneumoniae at the outer-membrane.

### REFERENCES

1. Aguilera-Herce J, Garcia-Quintanilla M, Romero-Flores R, McConnell M J, Ramos-Morales F. A live Salmonella vaccine delivering PcrV through the type III secretion system protects against Pseudomonas aeruginosa. mSphere 2019; 4:e00116-19.
2. Ali, S, Yu X Q, Robbie G J, Shoemaker K. et al. Phase 1 study of MEDI3902, an investigational anti-Pseudomonas aeruginosa PcrV and Psl bispecific human monoclonal antibody, in healthy adults. Clin. Microbiol. Infect. 2019, 25(5):629.e1-629.e6
3. Antenucci F, Ovsepian A, Wrobel A, Winther-Larsen H, and Bojesen, A. Design and Characterization of a Novel Tool for the Antigenic Enrichment of Actinobacillus pleuropneumoniae Outer Membrane. Pathogens. 2020, 9: 1014.
4. Babu L, Uppalapati S R, Sripathy M H, Reddy P N. Evaluation of Recombinant Multi-Epitope Outer Membrane Protein-Based Klebsiella pneumoniae Subunit Vaccine in Mouse Model. Front. Microbiol. 2017; 8:1-12.
5. Carretero-Ledesma M, Garcia-Quintanilla M, Martin-Pena R, Pulido M R, Pachón J, McConnell M J. Phenotypic changes associated with Colistin resistance due to Lipopolysaccharide loss in Acinetobacter baumannii. Virulence. 2018, 9:930-42.
6. Chen X, Shao Z, Wu L, He B, Yang H, Chen J, Jin E, Huang Q, Lei L, Xu J, Li, H, Zhang H, Wan, Y, Liu W, and Zhou R. Involvement of the Actinobacillus pleuropneumoniae ompW Gene in Confrontation of Environmental Pressure. Front. Vet Sci. 2022, 9: 846322.
7. Cote-Sierra J, Bredan A, Toldos C M, et al. Bacterial lipoprotein-based vaccines induce tumor necrosis factor-dependent type 1 protective immunity against Leishmania major. Infection and Immunity 2002; 70(1):240-8.
8. Finke M, Duchene M, Eckhardt A, Domdey H. Protection against Experimental Pseudomonas aeruginosa Infection by Recombinant P. aeruginosa Lipoprotein I Expressed in Escherichia coli. Infection and Immunity. 1990; 58(7):2241-4.
9. Finke M, Muth G, Reichhelm T, Thoma M, Duchene M et al. Protection of Immunosuppressed Mice against Infection with Pseudomonas aeruginosa by Recombinant P. aeruginosa Lipoprotein I and Lipoprotein I-Specific Monoclonal Antibodies. Infection and Immunity. 1991; 59:1251-1254.
10. Forsyth V S, Himpsl S D, Smith S N, Sarkissian C A, Mike L A, Stocki J A, Sintsova A, Alteri C J, Mobley HLT. Optimization of an Experimental Vaccine To Prevent Escherichia coli Urinary Tract Infection. mBIO 2020 11(2):e00555-20.
11. Francois, B, Luyt C E, Dugard A, Wolff M. et al. Safety and pharmacokinetics of an anti-PcrV PEGylated monoclonal antibody fragment in mechanically ventilated patients colonized with Pseudomonas aeruginosa: a randomized, double-blind, placebo-controlled trial. Critical Care Medicine. 2012; 40, 2320-2326.
12. García-Quintanilla M, Pulido M R, Pachón J, McConnell M J. Immunization with lipopolysaccharide-deficient whole cells provides protective immunity in an experimental mouse model of Acinetobacter baumannii infection. PLoS One 2014 9(12):e114410.
13. Gartner T, Baeten M, Otieno S, Revets H, Baetselier P et al. Mucosal prime-boost vaccination for tuberculosis based on TLR triggering OprI lipoprotein from Pseudomonas aeruginosa fused to mycolyl-transferase Ag85A. Immunology letters. 2007; 111:26-35.
14. Gu H, Liao Y, Zhang J, Wang Y, Liu Z, Cheng P, Wang X, Zou Q, and Gu J. Rational Design and Evaluation of an Artificial Escherichia coli K1 Protein Vaccine Candidate Based on the Structure of OmpA. Front. Cell. Infect. Microbiol. 2018, 8:172.
15. Guilleland Jr, Parker M G, Matthews J M et al. Use of a Purified Outer Membrane Protein F (Porin) Preparation of Pseudomonas aeruginosa as a Protective Vaccine in Mice. Infection and Immunity. 1984; 44:49-54.
16. Habibi, M, Reza M, Karam A, Bouzari S. Evaluation of prevalence, immunogenicity and efficacy of FyuA iron receptor in uropathogenic Escherichia coli isolates as a vaccine target against urinary tract infection. Microb. Pathog. 2017, 110:477-83.
17. Hagan E C and Mobley HLT. Heme acquisition is facilitated by a novel receptor Hma and required by uropathogenic Escherichia coli for kidney infection. Mol. Microbiol. 2009, 71: 79-91.
18. Hamaoka S, Naito Y, Katoh H, Shimizu M, Kinoshita M et al. Efficacy comparison of adjuvants in PcrV vaccine against Pseudomonas aeruginosa pneumonia. Microbiology and Immunology. 2017; 61:64-67.
19. Hancock V, Ferrieres L, Klemm P. The ferric yersiniabactin uptake receptor FyuA is required for efficient biofilm formation by urinary tract infectious Escherichia coli in human urine. Microbiology (Reading) 2008, 154:167-175.
20. Hassan R, El-naggar W, El-aziz A M, Saaban M, Kenawy H I, Ali Y M. Immunization with outer membrane proteins (OprF and OprI) and flagellin B protects mice from pulmonary infection with mucoid and nonmucoid Pseudomonas aeruginosa. J. Microbiol. Immunol. Infect. 2018; 51(3):312-320.
21. Hussein K E, Bahey-el-din M, Sheweita S A. Immunization with the outer membrane proteins OmpK17 and OmpK36 elicits protection against Klebsiella pneumoniae in the murine infection model. Microbial Pathogenesis 2018; 119:12-8.
22. Juncker A S, Willenbrock H, von Heijne G., Brunak S, Nielsen H, and Krogh A. Prediction of lipoprotein signal peptides in Gram-negative bacteria. Protein Sci. 2003 12: 1652-62.
23. Koebnik R, Locher K P, and Van Gelder, P. Structure and function of bacterial outer membrane proteins: barrels in a nutshell. Mol. Microbiol. 2000 37:239-53.
24. Kurupati P, Ramachandran N P, Poh C L. Protective Efficacy of DNA Vaccines Encoding Outer Membrane Protein A and OmpK36 of Klebsiella pneumoniae in Mice. Clin. Vaccine Immunol. 2011; 18(1):82-8.
25. Kurupati P, Teh B K, Kumarasinghe G, Poh C L. Identification of vaccine candidate antigens of an ESBL producing Klebsiella pneumoniae clinical strain by immunoproteome analysis. Proteomics. 2006; 836-44.
26. Maisnier-Patin K, Malissard M, Jeannin P, Haeuw J F, Corbiere J C, Hoeffel G, Gauchat J F, Nguyen T, Saez J M, and Delneste Y. The outer membrane protein X from Escherichia coli exhibits immune properties. Vaccine 2003, 21:3765-74.
27. Mellata, M, Mitchell N M, Schodel F, Curtiss R, and Pierd G B. Novel vaccine antigen combinations elicit protective immune responses against Escherichia coli sepsis. Vaccine. 2016 34: 656-662.
28. Meynet E, Laurin D, Luc J, Camara B, Toussaint B, Le A. Killed but metabolically active Pseudomonas aeruginosa-based vaccine induces protective humoral- and cell-mediated immunity against Pseudomonas aeruginosa pulmonary infections. Vaccine. 2018; 36(14):1893-1900.
29. Milla C E, Chmiel J F, Accurso F J, VanDevanter D R, Konstan M W, Yarranton G, et al. Anti-PcrV antibody in cystic fibrosis: a novel approach targeting Pseudomonas aeruginosa airway infection. Pediatric Pulmonology 2014; 49:650-8.
30. Moffatt J H, Harper M, Harrison P, Hale JDF, Vinogradov E, Seemann T, Henry R, Crane B, St Michael F, Cox A D, Adler B, Nation R L, Li J, and Boyce J D. Colistin resistance in Acinetobacter baumannii is mediated by complete loss of lipopolysaccharide production. Antimicrob Agents Chemother. 2010, 54(12):4971-7.
31. Moriyama K, Wiener-Kronish J P, Sawa T. Protective effects of affinity-purified antibody and truncated vaccines against Pseudomonas aeruginosa V-antigen in neutropenic mice. Microbiol. Immunol. 2009; 53: 587-594.
32. Pulido M R, Garcia-Quintanilla M, Pach6n J, McConnell M J. Immunization with lipopolysaccharide-free outer membrane complexes protects against Acinetobacter baumannii infection. Vaccine 2018, 36:4153-56.
33. Rau H, Revets H, Cornelis P, Titzmann A, Ruggli N et al. Efficacy and functionality of lipoprotein OprI from Pseudomonas aeruginosa as adjuvant for a subunit vaccine against classical swine fever. Vaccine. 2006; 24:4757-4768.
34. Sawa T, Ito E, Nguyen V H, Haight M. Anti-PcrV antibody strategies against virulent Pseudomonas aeruginosa. Hum. Vaccin. Immunother. 2014; 10:2843-52.
35. Sawa T, Yahr T L, Ohara M, Kurahashi K, Gropper M A, Wiener-Kronish J P, Frank D W. Active and passive immunization with the Pseudomonas V antigen protects against type III intoxication and lung injury. Nat. Med. 1999; 5:392-398.
36. Smithers L, Olatunji S, and Caffrey M. Bacterial Lipoprotein Posttranslational Modifications. New Insights and Opportunities for Antibiotic and Vaccine Development. Front. Microbiol 2021 12:788445.
37. Vogt J, and Schulz G E. The structure of the outer membrane protein OmpX from Escherichia coli reveals possible mechanisms of virulence. Structure 1999, 7:1301-9.
38. von Specht B U, Licking H C, Schmitt A, Hungerert K D, Domdey H. Safety and immunogenicity of a Pseudomonas aeruginosa outer membrane protein I vaccine in human volunteers. Vaccine. 1996; 14(12):1111-7.
39. Wang Y. The function of OmpA in Escherichia coli. Biochem. Biophys. Res. Commun.
   2002, 292:396-401.
40. Zhang Y, Tennent J M, Ingham A, Beddome G, Prideaux C, and Michalski W P. Identification of type 4 fimbriae in Actinobacillus pleuropneumoniae. FEMS Microbiol. Lett. 2000, 189: 15-18.

1. A composition, comprising:
a) an A. baumannii strain deficient in lipopolysaccharide (LPS) characterized by the partial or complete inactivation of one or various cellular nucleic acid molecules that encode endogenous LPS biosynthesis genes; wherein the A. baumannii strain deficient in lipopolysaccharide (LPS) is characterized by the partial or complete inactivation of the genes selected from the list consisting of lpxA, lpxB, lpxC, lpxD, lpxK, lpxL and/or lpxM; and wherein the A. baumannii strain deficient in lipopolysaccharide (LPS) expresses one or more heterologous antigens or proteins with targeted location at the Outer Membrane; and/or
b) an outer membrane vesicle (OMV) derived from an A. baumannii strain deficient in lipopolysaccharide (LPS) as defined in paragraph a) above;
wherein the one or more heterologous antigens with targeted location at the outer membrane are characterized by comprising:
I) at the N-terminus of the one or more heterologous antigens, a signal sequence derived from an outer membrane protein (OMP) which is processed by A. baumannii to direct the location of the expressed protein to the external membrane of A. baumannii;
II) OMP transmembrane domains typical of integral OMP proteins or bacterial lipoproteins which allow insertion of the expressed protein at the external membrane of A. baumannii; and
III) immunogenic domains;
and wherein the strain is modified to express the one or more heterologous antigens with targeted location at the outer membrane by insertion of an expression construct comprising at least one or more transcription promoter sequences, one or more ORFs (Open Reading Frames) encoding the one or more heterologous antigens, and one or more transcription termination sequences at a locus in the A. baumannii chromosome that can incorporate an insert by recombination, selected from the list consisting of: cysI, trpE, lpxA, lpxC, lpxD, lpxB, lpxK, lpxL, lpxM, and/or the Tn5/Tn7 sites.

2. The composition according to claim 1, wherein the signal sequence is the outer-membrane protein OmpA of A. baumannii (SEQ ID No. 1) or any signal sequence that has at least 90%, 91%, 92%, 93%, 94%, 95%, 96%, 97%, 98%, or 99% identity with the full/length sequence of SEQ ID NO 1 with the proviso that these identical sequences can be processed by A. baumannii cells to promote the location of the expressed protein in the outer-membrane of A. baumannii.

3. The composition according to claim 1, wherein the locus in the A. baumannii chromosome that can incorporate an insert by recombination is cysI.

4. The composition according to claim 1, a wherein the heterologous antigens or proteins expressed at the Outer Membrane of the A. baumannii strain deficient in lipopolysaccharide (LPS) are at least derived from K. pneumoniae, P. aeruginosa, E. coli, and/or A. pleuropneumoniae.

5. The composition according to claim 4, wherein the heterologous antigens or proteins expressed at the Outer Membrane of the A. baumannii strain deficient in lipopolysaccharide (LPS) are at least derived from K. pneumoniae and are selected from the list consisting of Kp-OmpA (SEQ ID No 31 or 17) and Kp-Ompk36 (SEQ ID No. 32 or 18), or any sequence that has at least 90%, 91%, 92%, 93%, 94%, 95%, 96%, 97%, 98%, or 99% identity with the full/length sequence of any of SEQ ID No. 31, 32, 17 or 18 that when expressed or exposed in one or multiple copies in the outer-membrane of A. baumannii by using a signal sequence that can be processed by A. baumannii cells to promote the location of the expressed protein in the outer-membrane of A. baumannii, and upon being inoculated in a subject in need thereof, produces immunization not only against A. baumannii infection but also against infections caused by K. pneumoniae.

6. The composition according to claim 4, wherein the heterologous antigens expressed at the Outer Membrane of the Acinetobacter baumannii strain deficient in lipopolysaccharide (LPS) are at least derived from P. aeruginosa and are selected from the list consisting of Pa-OprF (SEQ ID NO. 33 or 19), Pa-OprI (SEQ ID NO. 35 or 21), Pa-PcrV (SEQ ID NO 45), or Pa-OprI:PcrV (SEQ ID NO 34 or 20), or any sequence that has at least 90%, 91%, 92%, 93%, 94%, 95%, 96%, 97%, 98%, or 99% identity with the full/length sequence of any of SEQ ID No. 33 to 35, 19 to 21 or 45 that when expressed or exposed in one or multiple copies in the outer-membrane of A. baumannii by using a signal sequence that can be processed by A. baumannii cells to promote the location of the expressed protein in the outer-membrane of A. baumannii, and upon being inoculated in a subject in need thereof, produces immunization not only against A. baumannii infection but also against infections caused by P. aeruginosa.

7. The composition according to claim 4, wherein the heterologous antigens expressed at the Outer Membrane of the Acinetobacter baumannii strain deficient in lipopolysaccharide (LPS) are at least derived from E. coli and are selected from the list consisting of Ec-OmpA (SEQ ID NO. 36 or 22), Ec-OmpX (SEQ ID NO. 37 or 23), Ec-FuyA (SEQ ID NO 38 or 24), Ec-Hma (SEQ ID NO 39 or 25) or Ec-IutA (SEQ ID NO 40 or 26), including any sequence that has at least 90%, 91%, 92%, 93%, 94%, 95%, 96%, 97%, 98%, or 99% identity with the full/length sequence of any of SEQ ID No: 22 to 26 or 36 to 40 that when expressed or exposed in one or multiple copies in the outer-membrane of A. baumannii by using a signal sequence that can be processed by A. baumannii cells to promote the location of the expressed protein in the outer-membrane of A. baumannii, and upon being inoculated in a subject in need thereof, produces immunization not only against A. baumannii infection but also against infections caused by E. coli.

8. The composition according to claim 4, wherein the heterologous antigens expressed at the Outer Membrane of the Acinetobacter baumannii strain deficient in lipopolysaccharide (LPS) are at least derived from A. pleuropneumoniae and are selected from the list consisting of Ap-OmpA (SEQ ID NO. 27 or 41), Ap-OmpW (SEQ ID NO. 28 or 42), Ap-TbpA (SEQ ID NO 29 or 43), or Ap-ApfA (SEQ ID NO 30 or 44), including any sequence that has at least 90%, 91%, 92%, 93%, 94%, 95%, 96%, 97%, 98%, or 99% identity with the full/length sequence of any of SEQ ID: 27 to 30 or 41 to 44 that when expressed or exposed in one or multiple copies in the outer-membrane of A. baumannii by using a signal sequence that can be processed by A. baumannii cells to promote the location of the expressed protein in the outer-membrane of A. baumannii, and upon being inoculated in a subject in need thereof, produces immunization not only against A. baumannii infection but also against infections caused by A. pleuropneumoniae.

9. The composition according to claim 4, wherein the heterologous antigens expressed at the Outer Membrane of the A. baumannii strain deficient in lipopolysaccharide (LPS) are at least derived from K. pneumoniae and are selected from the list consisting of Kp-OmpA and/or Kp-OmpK36; and/or are derived from P. aeruginosa and are selected from the list consisting of Pa-OprF and/or Pa-OprI, and or the fusion protein Pa-OprI::PcrV; and/or are derived from E. coli and are selected from the list consisting of Ec-OmpA and/or Ec-OmpX and/or Ec-FuyA and/or Ec-Hma and/or Ec-IutA-a er any identical sequences thereto in accordance with claim 7; and/or are derived from A. pleuropneumoniae and are selected from the list Ap-OmpA and/or Ap-OmpW and/or Ap-TbpA and/or Ap-ApfA.

10. The composition according to claim 1, wherein the A. baumannii strain deficient in lipopolysaccharide (LPS) further comprises the expression of A. baumannii antigens Ab-OmpA and Ab-Omp22.

11. The composition according to claim 1, wherein the A. baumannii strain deficient in lipopolysaccharide (LPS) is characterized by the partial or complete inactivation of the genes selected from the list consisting of lpxA, lpxB, lpxC.

12. A vaccine, comprising the composition according to claim 1.

13. A method, comprising delivering bacterial Outer Membrane antigens to a subject in need thereof by immunizing the subject with the vaccine of claim 12.

14. A method, comprising delivering bacterial Outer Membrane antigens for inducing an immunological protective response at least against K. pneumoniae, P. aeruginosa, E. coli, and/or A. pleuropneumoniae and optionally A. baumannii to a subject by immunizing the subject with the vaccine of claim 12.

15. A vaccine composition, comprising an A. baumannii strain deficient in lipopolysaccharide (LPS) characterized by the partial or complete inactivation of one or various cellular nucleic acid molecules that encode endogenous LPS biosynthesis genes: wherein the A. baumannii strain deficient in lipopolysaccharide (LPS) is characterized by the partial or complete inactivation of the genes selected from the list consisting of lpxA, lpxB, lpxC, lpxD, lpxK, lpxL and/or lpxM; and wherein the A. baumannii strain deficient in lipopolysaccharide (LPS) expresses one or more heterologous antigens or proteins with targeted location at the Outer Membrane.

16. The vaccine according to claim 15, wherein the vaccine comprises from about 10⁶ to about 10¹² A. baumannii strains deficient in lipopolysaccharide (LPS).

17. The vaccine according to claim 15, further comprising an adjuvant.

18. The vaccine according to claim 17, wherein the adjuvant comprises Al(OH)₃.


SEQUENCE LISTING (90 sequences)

SEQ ID NO: 1 - protein, 22 aa, synthetic construct
MKLSRIALATMLVAAPLAAANA

SEQ ID NO: 2 - protein, 344 aa, Klebsiella pneumoniae
MKAIFVLNAAPKDNTWYAGGKLGWSQYHDTGFYGNGFQNNNGPTRNDQLGAGAFGGYQVNPYLGFEMGYDWLGRMAYKGSVDNGAFKAQGVQLTAKLGYPITDDLDIYTRLGGMVWRADSKGNYASTGVSRSEHDTGVSPVFAGGVEWAVTRDIATRLEYQWVNNIGDAGTVGTRPDNGMLSLGVSYRFGQEDAAPVVAPAPAPAPEVATKHFTLKSDVLFNFNKATLKPEGQQALDQLYTQLSNMDPKDGSAVVLGYTDRIGSEAYNQQLSEKRAQSVVDYLVAKGIPAGKISARGMGESNPVTGNTCDNVKARAALIDCLAPDRRVEIEVKGYKEVVTQPQA

SEQ ID NO: 3 - protein, 367 aa, Klebsiella singaporensis
MKVKVLSLLVPALLVAGAANAAEIYNKDGNKLDLYGKIDGLHYFSDDKSVDGDQTYMRVGVKGETQINDQLTGYGQWEYNVQANNTESSSDQAWTRLAFAGLKFGDAGSFDYGRNYGVVYDVTSWTDVLPEFGGDTYGSDNFLQSRANGVATYRNSDFFGLVDGLNFALQYQGKNGSVSGEGALSPTNNGRTALKQNGDGYGTSLTYDIYDGISAGFAYSNSKRLGDQNSKLALGRGDNAETYTGGLKYDANNIYLATQYTQTYNATRAGSLGFANKAQNFEVVAQYQFDFGLRPSVAYLQSKGKDLEGYGDQDILKYVDVGATYYFNKNMSTYVDYKINLLDDNSFTHNAGISTDDVVALGLVYQF

SEQ ID NO: 4 - protein, 350 aa, Pseudomonas aeruginosa
MKLKNTLGVVIGSLVAASAMNAFAQGQNSVEIEAFGKRYFTDSVRNMKNADLYGGSIGYFLTDDVELALSYGEYHDVRGTYETGNKKVHGNLTSLDAIYHFGTPGVGLRPYVSAGLAHQNITNINSDSQGRQQMTMANIGAGLKYYFTENFFAKASLDGQYGLEKRDNGHQGEWMAGLGVGFNFGGSKAAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 5 - protein, 372 aa, Pseudomonas aeruginosa
MNNVLKFSALALAAVLATGCSSHSKETEARLTATEDAAARAQARADEAYRKADEALGAAQKAQQTADEANERALRMLEKASRKGGGGSGGGGSGGGGSSAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLRDILSAI

SEQ ID NO: 6 - protein, 83 aa, Pseudomonas aeruginosa
MNNVLKFSALALAAVLATGCSSHSKETEARLTATEDAAARAQARADEAYRKADEALGAAQKAQQTADEANERALRMLEKASRK

SEQ ID NO: 7 - protein, 294 aa, Pseudomonas aeruginosa
MEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLRDILSAI

SEQ ID NO: 8 - protein, 350 aa, Escherichia coli
MKKTAIAIAVALAGFATVAQAAPKDNTWYTGAKLGWSQYHDTGFIPNNGPTHENQLGAGAFGGYQVNPYVGFEMGYDWLGRMPYKGDNINGAYKAQGVQLTAKLGYPITDDLDIYTRLGGMVWRADTKANVPGGASFKDHDTGVSPVFAGGVEYAITPEIATRLEYQWTNNIGDAHTIGTRPDNGMLSLGVSYRFGQGEVAPVVAPAPAPAPEVQTKHFTLKSDVLFTFNKATLKPEGQAALDQLYSQLSNLDPKDGSVVVLGYTDRIGSDAYNQALSERRAQSVVDYLISKGIPADKISARGMGESNPVTGNTCDNVKQRAALIDCLAPDRRVEIEVKGIKDVVTQPQA

SEQ ID NO: 9 - protein, 171 aa, Escherichia coli
MKKIACLSALAAVLAFTAGTSVAATSTVTGGYAQSDAQGQMNKMGGFNLKYRYEEDNSPLGVIGSFTYTEKSRTASSGDYNKNQYYGITAGPAYRINDWASIYGVVGVGYGKFQTTEYPTYKHDTSDYGFSYGAGLQFNPMENVALDFSYEQSRIRSVDVGTWIAGVGYRF

SEQ ID NO: 10 - protein, 673 aa, Escherichia coli
MKMTRLYPLALGGLLLPAIANAQTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMPQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVGMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 11 - protein, 715 aa, Escherichia coli
MLYNIPCRIYILSTLSLCISGIVSTATATSSETKISNEETLVVTTNRSASNLWESPATIQVIDQQTLQNSTNASIADNLQDIPGVEITDNSLAGRKQIRIRGEASSRVLILIDGQEVTYQRAGDNYGVGLLIDESALERVEVVKGPYSVLYGSQAIGGIVNFITKKGGDKLASGVVKAVYNSATAGWEESIAVQGSIGGFDYRINGSYSDQGNRDTPDGRLPNTNYRNNSQGVWLGYNSGNHRFGLSLDRYRLATQTYYEDPDGSYEAFSVKIPKLEREKVGVFYDTDVDGDYLKKIHFDAYEQTIQRQFANEVKTTQPVPSPMIQALTVHNKTDTHDKQYTQAVTLQSHFSLPANNELVTGAQYKQDRVSQRSGGMTSSKSLTGFINKETRTRSYYESEQSTVSLFAQNDWRFADHWTWTMGVRQYWLSSKLTRGDGVSYTAGIISDTSLARESASDHEMVTSTSLRYSGFDNLELRAAFAQGYVFPTLSQLFMQTSAGGSVTYGNPDLKAEHSNNFELGARYNGNQWLIDSAVYYSEAKDYIASLICDGSIVCNGNTNSSRSSYYYYDNIDRAKTWGLEISAEYNGWVFSPYISGNLIRRQYETSTLKTTNTGEPAINGRIGLKHTLVMGQANIISDVFIRAASSAKDDSNGTETNVPGWATLNFAVNTEFGNEDQYRINLALNNLTDKRYRTAHETIPAAGFNAAIGFVWNF

SEQ ID NO: 12 - protein, 726 aa, Escherichia coli
MPRALGPLLLVVLSPAVAQQNDDNEIIVSASRSNRTVAEMAQTTWVIENAELEQQIQGGKELKDALAQLIPGLDVSSQSRTNYGMNMRGRPLVVLIDGVRLNSSRSDSRQLDSVDPFNIDHIEVISGATALYGGGSTGGLINIVTKKGQPETMMEFEAGTKSGFNSSKDHDERIAGAVSGGNDHISGRLSVAYQKFGGWFDGNGDATLLDNTQTGLQHSNRLDIMGTGTLNIDESRQLQLITQYYKSQGDDNYGLNLGKGFSAISGSSTPYVSKGLNSDRIPGTERHLISLQYSDSDFLRQELVGQVYYRDESLRFYPFPTVNANKQATAFSSSQQDTDQYGMKLTLNSQLMDGWQITWGLDAEHERFTSNQMFFDLAQASASGGLNNHKIYTTGRYPSYDITNLAAFLQSSYDINDIFTVSGGVRYQYTENRVDDFIDYTQQQKIAAGKAISADAIPGGSVDYDNFLFNAGLLMHITERQQAWFNFSQGVALPDPGKYYGRGIYGAAVNGHLPLTKSVNVSDSKLEGVKVDSYELGWRFTGDNLRTQIAAYYSLSNKSVERNKDLTISVKDDRRRIYGVEGAVDYLIPDTDWSTGVNFNVLKTESKVNGQWQKYDVKESSPSKATAYINWAPEPWSLRVQSTTSFDVSDAEGNDINGYTTVDFISSWQLPVGTLSFSVENLFDRDYTTVWGQRAPLYYSPGYGPASLYDYKGRGRTFGLNYSVLF

SEQ ID NO: 13 - protein, 364 aa, Actinobacillus pleuropneumoniae
MKKSLVALTVLSAAAVAQAAPQQNTFYAGAKAGWASFHDGIEQLDSAKNTDRGTKYGINRNSVTYGVFGGYQILNQDKLGLAAELGYDYFGRVRGSEKPNGKADKKTFRHAAHGATIALKPSYEVLPDLDVYGKVGIALVNNTYKTFNAAQEKVKTRRFQSSLILGAGVEYAILPELAARVEYQWLNNAGKASYSTLNRMGATDYRSDISSVSAGLSYRFGQGAVPVAAPAVETKNFAFSSDVLFAFGKSNLKPAAATALDAMQTEINNAGLSNAAIQVNGYTDRIGKEASNLKLSQRRAETVANYIVSKGALAANVTAVGYGEANPVTGATCDKVKGRKALIACLAPDRRVEVQVQGTKEVTM

SEQ ID NO: 14 - protein, 215 aa, Actinobacillus pleuropneumoniae
MKKAVLAAVLGGALLADSAMAHQAGDVIFRAGAIGVIANSSSDYQTGADVNLDVNNNIQLGLTGTYMLSDNLGLELLAATPFSHKITGKLGATDLGEVAKVKHLPPSLYLQYYFFDSNATVRPYVGAGLNYTRFFSAESLKPQLVQNLRVKKHSVAPIANLGVDVKLTDNLSFNAAAWYTRIKTTADYDVPGLGHVSTPITLDPVVLFSGISYKF

SEQ ID NO: 15 - protein, 935 aa, Actinobacillus pleuropneumoniae
MKNKLNLISLALLSLFAVQSYAEQAVQLNDVYVTGTKKKAHKKENEVTGLGKVVKTPDTLSKEQVLGIRDLTRYDPGISVVEQGRGATTGYSIRGVDRNRVGLALDGLPQIQSYVSQYSRSSSGAINEIEYENLRSIQISKGASSSEFGSGSLGGSVQFRTKEVSDIIKPGQSWGLDTKSAYSSKNQQWLNSLAFAGTHNGFDALVIYTHRDGKETKAHKNAESRSQNITRVGVETNELDTSNRYTATTNNQHTYGWFLIKDECPTLDCTPKQMARVTKDTPSFRSYPEYTPEEKQAYENQKHITERLNAQDYTGEYRALPDPLKYKSDSWLVKLGYTFSPKHYVAGTYEHSKQRYDTRDMTYTAYWQPSDLLRTGRNWYPMNNAKGLYRDNALDGVAIDYFTEDGVKSSKGLRWAKARFIDEWHTRDRLGALYRYTNQDGNRLIDRLSLSFDQQKINLSTRLRENNCSEYPTIDKNCRATLDKLWSSTKNEQSSYEEKHDTIQLSLDKTVQTGLGKHQLNMLLGSDRFNSTLKRHEILSEFSVGSWGLVRDIGYRNGSYNNPYVYELKDQAIYSKNECDYSGTIAGRADCATSKIKGHNHYIALRDNFAITKYLDIGLGYRFDKHKFRSTHRWANQGDYKNSAWNIGIVAKPTSFLSLSYRASSGFRVPSFQELFGLRYDGAMKGSSDAYQKTEKLSPEKSLNQEVAATFKGDFGVVEVSYFKNDYKQLIAPAERMHQTQSMINYFNVQDIKLDGINLIGKLDWNGVFDKIPEGIYTTLAYSKMRVKEVKNYQGYMNIRSPLLDTIQPARYVVGVGYDQPDEKWGVNLTMTHSSGKNPNELRGNEQVGFANYERTATKKRTRSWHTFDLTGYITPWKHTTVRAGVYNLMNYRYTTWESVRQSSLNAIHQHTNVKDYARYAAPGRNYVVSFEMKF

SEQ ID NO: 16 - protein, 148 aa, Actinobacillus pleuropneumoniae
MQKLSLIRPLTNAFTLIELMIVIAIIAILATVAIPSYNSYTQKAALSELLAASASYKTDVEICIYNTGDSKNCSGGQNGVRKMTELRQAKYLNAITVEGGTITVTGKGNLQEYGYTMTPIHNGSTISWETKCKGEDLSLFPANFCAIN

SEQ ID NO: 17 - protein, 347 aa, Klebsiella pneumoniae
KLSRIALATMLVAAPLAAANAGGKLGWSQYHDTGFYGNGFQNNNGPTRNDQLGAGAFGGYQVNPYLGFEMGYDWLGRMAYKGSVDNGAFKAQGVQLTAKLGYPITDDLDIYTRLGGMVWRADSKGNYASTGVSRSEHDTGVSPVFAGGVEWAVTRDIATRLEYQWVNNIGDAGTVGTRPDNGMLSLGVSYRFGQEDAAPVVAPAPAPAPEVATKHFTLKSDVLFNFNKATLKPEGQQALDQLYTQLSNMDPKDGSAVVLGYTDRIGSEAYNQQLSEKRAQSVVDYLVAKGIPAGKISARGMGESNPVTGNTCDNVKARAALIDCLAPDRRVEIEVKGYKEVVTQPQA

SEQ ID NO: 18 - protein, 368 aa, Klebsiella pneumoniae
MKLSRIALATMLVAAPLAAANAAEIYNKDGNKLDLYGKIDGLHYFSDDKSVDGDQTYMRVGVKGETQINDQLTGYGQWEYNVQANNTESSSDQAWTRLAFAGLKFGDAGSFDYGRNYGVVYDVTSWTDVLPEFGGDTYGSDNFLQSRANGVATYRNSDFFGLVDGLNFALQYQGKNGSVSGEGALSPTNNGRTALKQNGDGYGTSLTYDIYDGISAGFAYSNSKRLGDQNSKLALGRGDNAETYTGGLKYDANNIYLATQYTQTYNATRAGSLGFANKAQNFEVVAQYQFDFGLRPSVAYLQSKGKDLEGYGDQDILKYVDVGATYYFNKNMSTYVDYKINLLDDNSFTHNAGISTDDVVALGLVYQF

SEQ ID NO: 19 - protein, 348 aa, Pseudomonas aeruginosa
MKLSRIALATMLVAAPLAAANAQGQNSVEIEAFGKRYFTDSVRNMKNADLYGGSIGYFLTDDVELALSYGEYHDVRGTYETGNKKVHGNLTSLDAIYHFGTPGVGLRPYVSAGLAHQNITNINSDSQGRQQMTMANIGAGLKYYFTENFFAKASLDGQYGLEKRDNGHQGEWMAGLGVGFNFGGSKAAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 20 - protein, 373 aa, Pseudomonas aeruginosa
MKLSRIALATMLVAAPLAAANASHSKETEARLTATEDAAARAQARADEAYRKADEALGAAQKAQQTADEANERALRMLEKASRKGGGGSGGGGSGGGGSSAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLRDILSAI

SEQ ID NO: 21 - protein, 84 aa, Pseudomonas aeruginosa
MKLSRIALATMLVAAPLAAANASHSKETEARLTATEDAAARAQARADEAYRKADEALGAAQKAQQTADEANERALRMLEKASRK

SEQ ID NO: 22 - protein, 351 aa, Escherichia coli
MKLSRIALATMLVAAPLAAANAAPKDNTWYTGAKLGWSQYHDTGFIPNNGPTHENQLGAGAFGGYQVNPYVGFEMGYDWLGRMPYKGDNINGAYKAQGVQLTAKLGYPITDDLDIYTRLGGMVWRADTKANVPGGASFKDHDTGVSPVFAGGVEYAITPEIATRLEYQWTNNIGDAHTIGTRPDNGMLSLGVSYRFGQGEVAPVVAPAPAPAPEVQTKHFTLKSDVLFTFNKATLKPEGQAALDQLYSQLSNLDPKDGSVVVLGYTDRIGSDAYNQALSERRAQSVVDYLISKGIPADKISARGMGESNPVTGNTCDNVKQRAALIDCLAPDRRVEIEVKGIKDVVTQPQA

SEQ ID NO: 23 - protein, 170 aa, Escherichia coli
MKLSRIALATMLVAAPLAAANAATSTVTGGYAQSDAQGQMNKMGGFNLKYRYEEDNSPLGVIGSFTYTEKSRTASSGDYNKNQYYGITAGPAYRINDWASIYGVVGVGYGKFQTTEYPTYKHDTSDYGFSYGAGLQFNPMENVALDFSYEQSRIRSVDVGTWIAGVGYRF

SEQ ID NO: 24 - protein, 675 aa, Escherichia coli
MKLSRIALATMLVAAPLAAANANAQTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMPQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVGMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 25 - protein, 709 aa, Escherichia coli
MKLSRIALATMLVAAPLAAANATSSETKISNEETLVVTTNRSASNLWESPATIQVIDQQTLQNSTNASIADNLQDIPGVEITDNSLAGRKQIRIRGEASSRVLILIDGQEVTYQRAGDNYGVGLLIDESALERVEVVKGPYSVLYGSQAIGGIVNFITKKGGDKLASGVVKAVYNSATAGWEESIAVQGSIGGFDYRINGSYSDQGNRDTPDGRLPNTNYRNNSQGVWLGYNSGNHRFGLSLDRYRLATQTYYEDPDGSYEAFSVKIPKLEREKVGVFYDTDVDGDYLKKIHFDAYEQTIQRQFANEVKTTQPVPSPMIQALTVHNKTDTHDKQYTQAVTLQSHFSLPANNELVTGAQYKQDRVSQRSGGMTSSKSLTGFINKETRTRSYYESEQSTVSLFAQNDWRFADHWTWTMGVRQYWLSSKLTRGDGVSYTAGIISDTSLARESASDHEMVTSTSLRYSGFDNLELRAAFAQGYVFPTLSQLFMQTSAGGSVTYGNPDLKAEHSNNFELGARYNGNQWLIDSAVYYSEAKDYIASLICDGSIVCNGNTNSSRSSYYYYDNIDRAKTWGLEISAEYNGWVFSPYISGNLIRRQYETSTLKTTNTGEPAINGRIGLKHTLVMGQANIISDVFIRAASSAKDDSNGTETNVPGWATLNFAVNTEFGNEDQYRINLALNNLTDKRYRTAHETIPAAGFNAAIGFVWNF

SEQ ID NO: 26 - protein, 730 aa, Escherichia coli
MKLSRIALATMLVAAPLAAANAQQNDDNEIIVSASRSNRTVAEMAQTTWVIENAELEQQIQGGKELKDALAQLIPGLDVSSQSRTNYGMNMRGRPLVVLIDGVRLNSSRSDSRQLDSVDPFNIDHIEVISGATALYGGGSTGGLINIVTKKGQPETMMEFEAGTKSGFNSSKDHDERIAGAVSGGNDHISGRLSVAYQKFGGWFDGNGDATLLDNTQTGLQHSNRLDIMGTGTLNIDESRQLQLITQYYKSQGDDNYGLNLGKGFSAISGSSTPYVSKGLNSDRIPGTERHLISLQYSDSDFLRQELVGQVYYRDESLRFYPFPTVNANKQATAFSSSQQDTDQYGMKLTLNSQLMDGWQITWGLDAEHERFTSNQMFFDLAQASASGGLNNHKIYTTGRYPSYDITNLAAFLQSSYDINDIFTVSGGVRYQYTENRVDDFIDYTQQQKIAAGKAISADAIPGGSVDYDNFLFNAGLLMHITERQQAWFNFSQGVALPDPGKYYGRGIYGAAVNGHLPLTKSVNVSDSKLEGVKVDSYELGWRFTGDNLRTQIAAYYSLSNKSVERNKDLTISVKDDRRRIYGVEGAVDYLIPDTDWSTGVNFNVLKTESKVNGQWQKYDVKESSPSKATAYINWAPEPWSLRVQSTTSFDVSDAEGNDINGYTTVDFISSWQLPVGTLSFSVENLFDRDYTTVWGQRAPLYYSPGYGPASLYDYKGRGRTFGLNYSVLF

SEQ ID NO: 27 - protein, 368 aa, Actinobacillus pleuropneumoniae
MKLSRIALATMLVAAPLAAANAAAPQQNTFYAGAKAGWASFHDGIEQLDSAKNTDRGTKYGINRNSVTYGVFGGYQILNQDKLGLAAELGYDYFGRVRGSEKPNGKADKKTFRHAAHGATIALKPSYEVLPDLDVYGKVGIALVNNTYKTFNAAQEKVKTRRFQSSLILGAGVEYAILPELAARVEYQWLNNAGKASYSTLNRMGATDYRSDISSVSAGLSYRFGQGAVPVAAPAVETKNFAFSSDVLFAFGKSNLKPAAATALDAMQTEINNAGLSNAAIQVNGYTDRIGKEASNLKLSQRRAETVANYIVSKGALAANVTAVGYGEANPVTGATCDKVKGRKALIACLAPDRRVEVQVQGTKEVTM

SEQ ID NO: 28 - protein, 217 aa, Actinobacillus pleuropneumoniae
MKLSRIALATMLVAAPLAAANAAHQAGDVIFRAGAIGVIANSSSDYQTGADVNLDVNNNIQLGLTGTYMLSDNLGLELLAATPFSHKITGKLGATDLGEVAKVKHLPPSLYLQYYFFDSNATVRPYVGAGLNYTRFFSAESLKPQLVQNLRVKKHSVAPIANLGVDVKLTDNLSFNAAAWYTRIKTTADYDVPGLGHVSTPITLDPVVLFSGISYKF

SEQ ID NO: 29 - protein, 936 aa, Actinobacillus pleuropneumoniae
MKLSRIALATMLVAAPLAAANAAEQAVQLNDVYVTGTKKKAHKKENEVTGLGKVVKTPDTLSKEQVLGIRDLTRYDPGISVVEQGRGATTGYSIRGVDRNRVGLALDGLPQIQSYVSQYSRSSSGAINEIEYENLRSIQISKGASSSEFGSGSLGGSVQFRTKEVSDIIKPGQSWGLDTKSAYSSKNQQWLNSLAFAGTHNGFDALVIYTHRDGKETKAHKNAESRSQNITRVGVETNELDTSNRYTATTNNQHTYGWFLIKDECPTLDCTPKQMARVTKDTPSFRSYPEYTPEEKQAYENQKHITERLNAQDYTGEYRALPDPLKYKSDSWLVKLGYTFSPKHYVAGTYEHSKQRYDTRDMTYTAYWQPSDLLRTGRNWYPMNNAKGLYRDNALDGVAIDYFTEDGVKSSKGLRWAKARFIDEWHTRDRLGALYRYTNQDGNRLIDRLSLSFDQQKINLSTRLRENNCSEYPTIDKNCRATLDKLWSSTKNEQSSYEEKHDTIQLSLDKTVQTGLGKHQLNMLLGSDRFNSTLKRHEILSEFSVGSWGLVRDIGYRNGSYNNPYVYELKDQAIYSKNECDYSGTIAGRADCATSKIKGHNHYIALRDNFAITKYLDIGLGYRFDKHKFRSTHRWANQGDYKNSAWNIGIVAKPTSFLSLSYRASSGFRVPSFQELFGLRYDGAMKGSSDAYQKTEKLSPEKSLNQEVAATFKGDFGVVEVSYFKNDYKQLIAPAERMHQTQSMINYFNVQDIKLDGINLIGKLDWNGVFDKIPEGIYTTLAYSKMRVKEVKNYQGYMNIRSPLLDTIQPARYVVGVGYDQPDEKWGVNLTMTHSSGKNPNELRGNEQVGFANYERTATKKRTRSWHTFDLTGYITPWKHTTVRAGVYNLMNYRYTTWESVRQSSLNAIHQHTNVKDYARYAAPGRNYVVSFEMKF

SEQ ID NO: 30 - protein, 157 aa, Actinobacillus pleuropneumoniae
MKLSRIALATMLVAAPLAAANAFTLIELMIVIAIIAILATVAIPSYNSYTQKAALSELLAASASYKTDVEICIYNTGDSKNCSGGQNGVRKMTELRQAKYLNAITVEGGTITVTGKGNLQEYGYTMTPIHNGSTISWETKCKGEDLSLFPANFCAIN

SEQ ID NO: 31 - protein, 326 aa, Klebsiella pneumoniae
GGKLGWSQYHDTGFYGNGFQNNNGPTRNDQLGAGAFGGYQVNPYLGFEMGYDWLGRMAYKGSVDNGAFKAQGVQLTAKLGYPITDDLDIYTRLGGMVWRADSKGNYASTGVSRSEHDTGVSPVFAGGVEWAVTRDIATRLEYQWVNNIGDAGTVGTRPDNGMLSLGVSYRFGQEDAAPVVAPAPAPAPEVATKHFTLKSDVLFNFNKATLKPEGQQALDQLYTQLSNMDPKDGSAVVLGYTDRIGSEAYNQQLSEKRAQSVVDYLVAKGIPAGKISARGMGESNPVTGNTCDNVKARAALIDCLAPDRRVEIEVKGYKEVVTQPQA

SEQ ID NO: 32 - protein, 346 aa, Klebsiella pneumoniae
AEIYNKDGNKLDLYGKIDGLHYFSDDKSVDGDQTYMRVGVKGETQINDQLTGYGQWEYNVQANNTESSSDQAWTRLAFAGLKFGDAGSFDYGRNYGVVYDVTSWTDVLPEFGGDTYGSDNFLQSRANGVATYRNSDFFGLVDGLNFALQYQGKNGSVSGEGALSPTNNGRTALKQNGDGYGTSLTYDIYDGISAGFAYSNSKRLGDQNSKLALGRGDNAETYTGGLKYDANNIYLATQYTQTYNATRAGSLGFANKAQNFEVVAQYQFDFGLRPSVAYLQSKGKDLEGYGDQDILKYVDVGATYYFNKNMSTYVDYKINLLDDNSFTHNAGISTDDVVALGLVYQF

SEQ ID NO: 33 - protein, 326 aa, Pseudomonas aeruginosa
QGQNSVEIEAFGKRYFTDSVRNMKNADLYGGSIGYFLTDDVELALSYGEYHDVRGTYETGNKKVHGNLTSLDAIYHFGTPGVGLRPYVSAGLAHQNITNINSDSQGRQQMTMANIGAGLKYYFTENFFAKASLDGQYGLEKRDNGHQGEWMAGLGVGFNFGGSKAAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 34 - protein, 351 aa, Pseudomonas aeruginosa
SHSKETEARLTATEDAAARAQARADEAYRKADEALGAAQKAQQTADEANERALRMLEKASRKGGGGSGGGGSGGGGSSAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLRDILSAI

SEQ ID NO: 35 - protein, 62 aa, Pseudomonas aeruginosa
SHSKETEARLTATEDAAARAQARADEAYRKADEALGAAQKAQQTADEANERALRMLEKASRK

SEQ ID NO: 36 - protein, 329 aa, Escherichia coli
APKDNTWYTGAKLGWSQYHDTGFIPNNGPTHENQLGAGAFGGYQVNPYVGFEMGYDWLGRMPYKGDNINGAYKAQGVQLTAKLGYPITDDLDIYTRLGGMVWRADTKANVPGGASFKDHDTGVSPVFAGGVEYAITPEIATRLEYQWTNNIGDAHTIGTRPDNGMLSLGVSYRFGQGEVAPVVAPAPAPAPEVQTKHFTLKSDVLFTFNKATLKPEGQAALDQLYSQLSNLDPKDGSVVVLGYTDRIGSDAYNQALSERRAQSVVDYLISKGIPADKISARGMGESNPVTGNTCDNVKQRAALIDCLAPDRRVEIEVKGIKDVVTQPQA

SEQ ID NO: 37 - protein, 148 aa, Escherichia coli
ATSTVTGGYAQSDAQGQMNKMGGFNLKYRYEEDNSPLGVIGSFTYTEKSRTASSGDYNKNQYYGITAGPAYRINDWASIYGVVGVGYGKFQTTEYPTYKHDTSDYGFSYGAGLQFNPMENVALDFSYEQSRIRSVDVGTWIAGVGYRF

SEQ ID NO: 38 - protein, 653 aa, Escherichia coli
NAQTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMPQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVGMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 39 - protein, 687 aa, Escherichia coli
TSSETKISNEETLVVTTNRSASNLWESPATIQVIDQQTLQNSTNASIADNLQDIPGVEITDNSLAGRKQIRIRGEASSRVLILIDGQEVTYQRAGDNYGVGLLIDESALERVEVVKGPYSVLYGSQAIGGIVNFITKKGGDKLASGVVKAVYNSATAGWEESIAVQGSIGGFDYRINGSYSDQGNRDTPDGRLPNTNYRNNSQGVWLGYNSGNHRFGLSLDRYRLATQTYYEDPDGSYEAFSVKIPKLEREKVGVFYDTDVDGDYLKKIHFDAYEQTIQRQFANEVKTTQPVPSPMIQALTVHNKTDTHDKQYTQAVTLQSHFSLPANNELVTGAQYKQDRVSQRSGGMTSSKSLTGFINKETRTRSYYESEQSTVSLFAQNDWRFADHWTWTMGVRQYWLSSKLTRGDGVSYTAGIISDTSLARESASDHEMVTSTSLRYSGFDNLELRAAFAQGYVFPTLSQLFMQTSAGGSVTYGNPDLKAEHSNNFELGARYNGNQWLIDSAVYYSEAKDYIASLICDGSIVCNGNTNSSRSSYYYYDNIDRAKTWGLEISAEYNGWVFSPYISGNLIRRQYETSTLKTTNTGEPAINGRIGLKHTLVMGQANIISDVFIRAASSAKDDSNGTETNVPGWATLNFAVNTEFGNEDQYRINLALNNLTDKRYRTAHETIPAAGFNAAIGFVWNF

SEQ ID NO: 40 - protein, 708 aa, Escherichia coli
QQNDDNEIIVSASRSNRTVAEMAQTTWVIENAELEQQIQGGKELKDALAQLIPGLDVSSQSRTNYGMNMRGRPLVVLIDGVRLNSSRSDSRQLDSVDPFNIDHIEVISGATALYGGGSTGGLINIVTKKGQPETMMEFEAGTKSGFNSSKDHDERIAGAVSGGNDHISGRLSVAYQKFGGWFDGNGDATLLDNTQTGLQHSNRLDIMGTGTLNIDESRQLQLITQYYKSQGDDNYGLNLGKGFSAISGSSTPYVSKGLNSDRIPGTERHLISLQYSDSDFLRQELVGQVYYRDESLRFYPFPTVNANKQATAFSSSQQDTDQYGMKLTLNSQLMDGWQITWGLDAEHERFTSNQMFFDLAQASASGGLNNHKIYTTGRYPSYDITNLAAFLQSSYDINDIFTVSGGVRYQYTENRVDDFIDYTQQQKIAAGKAISADAIPGGSVDYDNFLFNAGLLMHITERQQAWFNFSQGVALPDPGKYYGRGIYGAAVNGHLPLTKSVNVSDSKLEGVKVDSYELGWRFTGDNLRTQIAAYYSLSNKSVERNKDLTISVKDDRRRIYGVEGAVDYLIPDTDWSTGVNFNVLKTESKVNGQWQKYDVKESSPSKATAYINWAPEPWSLRVQSTTSFDVSDAEGNDINGYTTVDFISSWQLPVGTLSFSVENLFDRDYTTVWGQRAPLYYSPGYGPASLYDYKGRGRTFGLNYSVLF

SEQ ID NO: 41 - protein, 346 aa, Actinobacillus pleuropneumoniae
AAPQQNTFYAGAKAGWASFHDGIEQLDSAKNTDRGTKYGINRNSVTYGVFGGYQILNQDKLGLAAELGYDYFGRVRGSEKPNGKADKKTFRHAAHGATIALKPSYEVLPDLDVYGKVGIALVNNTYKTFNAAQEKVKTRRFQSSLILGAGVEYAILPELAARVEYQWLNNAGKASYSTLNRMGATDYRSDISSVSAGLSYRFGQGAVPVAAPAVETKNFAFSSDVLFAFGKSNLKPAAATALDAMQTEINNAGLSNAAIQVNGYTDRIGKEASNLKLSQRRAETVANYIVSKGALAANVTAVGYGEANPVTGATCDKVKGRKALIACLAPDRRVEVQVQGTKEVTM

SEQ ID NO: 42 - protein, 195 aa, Actinobacillus pleuropneumoniae
AHQAGDVIFRAGAIGVIANSSSDYQTGADVNLDVNNNIQLGLTGTYMLSDNLGLELLAATPFSHKITGKLGATDLGEVAKVKHLPPSLYLQYYFFDSNATVRPYVGAGLNYTRFFSAESLKPQLVQNLRVKKHSVAPIANLGVDVKLTDNLSFNAAAWYTRIKTTADYDVPGLGHVSTPITLDPVVLFSGISYKF

SEQ ID NO: 43 - protein, 914 aa, Actinobacillus pleuropneumoniae
AEQAVQLNDVYVTGTKKKAHKKENEVTGLGKVVKTPDTLSKEQVLGIRDLTRYDPGISVVEQGRGATTGYSIRGVDRNRVGLALDGLPQIQSYVSQYSRSSSGAINEIEYENLRSIQISKGASSSEFGSGSLGGSVQFRTKEVSDIIKPGQSWGLDTKSAYSSKNQQWLNSLAFAGTHNGFDALVIYTHRDGKETKAHKNAESRSQNITRVGVETNELDTSNRYTATTNNQHTYGWFLIKDECPTLDCTPKQMARVTKDTPSFRSYPEYTPEEKQAYENQKHITERLNAQDYTGEYRALPDPLKYKSDSWLVKLGYTFSPKHYVAGTYEHSKQRYDTRDMTYTAYWQPSDLLRTGRNWYPMNNAKGLYRDNALDGVAIDYFTEDGVKSSKGLRWAKARFIDEWHTRDRLGALYRYTNQDGNRLIDRLSLSFDQQKINLSTRLRENNCSEYPTIDKNCRATLDKLWSSTKNEQSSYEEKHDTIQLSLDKTVQTGLGKHQLNMLLGSDRFNSTLKRHEILSEFSVGSWGLVRDIGYRNGSYNNPYVYELKDQAIYSKNECDYSGTIAGRADCATSKIKGHNHYIALRDNFAITKYLDIGLGYRFDKHKFRSTHRWANQGDYKNSAWNIGIVAKPTSFLSLSYRASSGFRVPSFQELFGLRYDGAMKGSSDAYQKTEKLSPEKSLNQEVAATFKGDFGVVEVSYFKNDYKQLIAPAERMHQTQSMINYFNVQDIKLDGINLIGKLDWNGVFDKIPEGIYTTLAYSKMRVKEVKNYQGYMNIRSPLLDTIQPARYVVGVGYDQPDEKWGVNLTMTHSSGKNPNELRGNEQVGFANYERTATKKRTRSWHTFDLTGYITPWKHTTVRAGVYNLMNYRYTTWESVRQSSLNAIHQHTNVKDYARYAAPGRNYVVSFEMKF

SEQ ID NO: 44 - protein, 135 aa, Actinobacillus pleuropneumoniae
FTLIELMIVIAIIAILATVAIPSYNSYTQKAALSELLAASASYKTDVEICIYNTGDSKNCSGGQNGVRKMTELRQAKYLNAITVEGGTITVTGKGNLQEYGYTMTPIHNGSTISWETKCKGEDLSLFPANFCAIN

SEQ ID NO: 45 - protein, 273 aa, Pseudomonas aeruginosa
AAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLRDILSAI

SEQ ID NO: 46 - DNA, 66 bp, synthetic construct
  source 1..66: mol_type = other DNA
atgaaattgagtcgtattgcacttgctactatgcttgttgctgctccattagctgctgctaatgct

SEQ ID NO: 47 - DNA, 1035 bp, Klebsiella pneumoniae
  source 1..1035: mol_type = genomic DNA
atgaaagcaattttcgtactgaatgcggctccgaaagataacacctggtatgcaggtggtaaactgggttggtcccagtatcacgacaccggtttctacggtaacggtttccagaacaacaacggtccgacccgtaacgatcagcttggtgctggtgcgttcggtggttaccaggttaacccgtacctcggtttcgaaatgggttatgactggctgggccgtatggcatataaaggcagcgttgacaacggtgctttcaaagctcagggcgttcagctgaccgctaaactgggttacccgatcactgacgatctggacatctacacccgtctgggcggcatggtttggcgcgctgactccaaaggcaactacgcttctaccggcgtttcccgtagcgaacacgacactggcgtttccccagtatttgctggcggcgtagagtgggctgttactcgtgacatcgctacccgtctggaataccagtgggttaacaacatcggcgacgcgggcactgtgggtacccgtcctgataacggcatgctgagcctgggcgtttcctaccgcttcggtcaggaagatgctgcaccggttgttgctccggctccggctccggctccggaagtggctaccaagcacttcaccctgaagtctgacgttctgttcaacttcaacaaagctaccctgaaaccggaaggtcagcaggctctggatcagctgtacactcagctgagcaacatggacccgaaagacggttccgctgttgttctgggctacaccgaccgcatcggttccgaagcttacaaccagcagctgtctgagaaacgtgctcagtccgttgttgactacctggttgctaaaggcatcccggctggcaaaatctccgctcgcggcatgggtgaatccaacccggttactggcaacacctgtgacaacgtgaaagctcgcgctgccctgatcgattgcctggctccggatcgtcgtgtagagatcgaagttaaaggctacaaagaagttgtaactcagccgcaggcttaa

SEQ ID NO: 48 - DNA, 1104 bp, Klebsiella pneumoniae
  source 1..1104: mol_type = other DNA
atgaaagttaaagtactgtccctcctggtaccggctctgctggtagcaggcgcagcaaatgcggctgaaatttataacaaagacggcaacaaattagacctgtacggtaaaattgacggtctgcactacttctctgacgacaagagcgtcgacggcgaccagacctacatgcgtgtaggcgtgaaaggcgaaacccagatcaacgaccagctgaccggttacggccagtgggaatacaacgttcaggcgaacaacactgaaagctccagcgatcaggcatggactcgtctggcattcgcaggcctgaaatttggcgacgcgggctctttcgactacggtcgtaactacggcgtagtatacgacgtaacgtcctggaccgacgttctgccggaattcggcggcgacacctacggttctgacaacttcctgcagtcccgtgctaacggcgttgcaacctaccgtaactctgatttcttcggtctggttgacggcctgaactttgctctgcagtatcagggtaaaaacggcagcgtcagcggcgaaggcgctctgtctcctaccaacaacggtcgtaccgccttgaaacagaacggcgacggttacggtacttctctgacctatgacatctatgatggcatcagcgctggtttcgcatactctaactccaaacgtcttggcgaccagaacagcaagctggcactgggtcgtggcgacaacgctgaaacctacaccggcggtctgaaatacgacgcgaacaacatctacctggccactcagtacacccagacctacaacgcgacccgcgccggttccctgggctttgctaacaaagcgcagaacttcgaagtggttgctcagtaccagttcgacttcggtctgcgtccgtccgtggcttacctgcagtctaaaggtaaggatctggaaggctacggcgaccaggacatcctgaaatatgttgacgttggcgcgacctactacttcaacaaaaacatgtccacctatgttgactacaaaatcaacctgctggacgacaatagcttcacccacaacgccggtatctctaccgacgacgtggttgcactgggcctggtttaccagttctaa

SEQ ID NO: 49 - DNA, 1053 bp, Pseudomonas aeruginosa
  source 1..1053: mol_type = other DNA
atgaaactgaagaacaccttaggcgttgtcatcggctcgctggttgccgcttcggcaatgaacgcctttgcccagggccagaactcggtagagatcgaagccttcggcaagcgctacttcaccgacagcgttcgcaacatgaagaacgcggacctgtacggcggctcgatcggttacttcctgaccgacgacgtcgagctggcgctgtcctacggtgagtaccatgacgttcgtggcacctacgaaaccggcaacaagaaggtccacggcaacctgacctccctggacgccatctaccacttcggtaccccgggcgtaggtctgcgtccgtacgtgtcggctggtctggctcaccagaacatcaccaacatcaacagcgacagccaaggccgtcagcagatgaccatggccaacatcggcgctggtctgaagtactacttcaccgagaacttcttcgccaaggccagcctcgacggccagtacggtctggagaagcgtgacaacggtcaccagggcgagtggatggctggcctgggcgtcggcttcaacttcggtggttcgaaagccgctccggctccggaaccggttgccgacgtttgctccgactccgacaacgacggcgtttgcgacaacgtcgacaagtgcccggataccccggccaacgtcaccgttgacgccaacggctgcccggctgtcgccgaagtcgtacgcgtacagctggacgtgaagttcgacttcgacaagtccaaggtcaaagagaacagctacgctgacatcaagaacctggctgacttcatgaagcagtacccgtccacttccaccaccgttgaaggtcacaccgactccgtcggcaccgacgcttacaaccagaagctgtccgagcgtcgtgccaacgccgttcgtgacgtactggtcaacgagtacggtgtagaaggtggtcgcgtgaacgctgttggttacggcgagtcccgcccggttgccgacaacgccaccgctgaaggccgcgctatcaaccgtcgcgttgaagccgaagtagaagctgaagccaagtaa

SEQ ID NO: 50 - DNA, 864 bp, Pseudomonas aeruginosa
  source 1..864: mol_type = other DNA
atgaacaacgttctgaaattctctgctctggctctggctgctgttctggccaccggttgcagcagccactccaaagaaaccgaagctcgtctgaccgctaccgaagacgcagctgctcgtgctcaggctcgcgctgacgaagcctatcgcaaggctgacgaagctctgggcgctgctcagaaagctcagcagactgctgacgaggctaacgagcgtgccctgcgcatgctggaaaaagccagccgcaagggcggcggcggcagcggcggcggcggcagcggcggcggcggcagctcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgcgagttcctggtgtcggcctatttcagcctgcacgggcgtctcgacgaggatgtcatcggtgtctacaaggatgtcctgcagacccaggacggcaagcgcaaggcgctgctcgacgagctgaaggcgctgaccgcggagttgaaggtctacagcgtgatccagtcgcagatcaacgccgcgctgtcggccaagcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggatttt

SEQ ID NO: 51 - DNA, 251 bp, Pseudomonas aeruginosa
  source 1..251: mol_type = other DNA
atgaacaacgttctgaaattctctgctctggctctggctgctgttctggccaccggttgcagcagccactccaaagaaaccgaagctcgtctgaccgctaccgaagacgcagctgctcgtgctcaggctcgcgctgacgaagcctatcgcaaggctgacgaagctctgggcgctgctcagaaagctcagcagactgctgacgaggctaacgagcgtgccctgcgcatgctggaaaaagccagccgcaatag

SEQ ID NO: 52 - DNA, 885 bp, Pseudomonas aeruginosa
  source 1..885: mol_type = other DNA
atggaagtcagaaaccttaatgccgctcgcgagctgttcctggacgagctcctggccgcgtcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgcgagttcctggtgtcggcctatttcagcctgcacgggcgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaagcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgcgcgacattctcagcgcgatctag

SEQ ID NO: 53 - DNA, 1053 bp, Escherichia coli
  source 1..1053: mol_type = other DNA
atgaaaaagacagctatcgcgattgcagtggcactggctggtttcgctaccgtagcgcaggccgctccgaaagataacacctggtacactggtgctaaactgggctggtcccagtaccatgacactggttttattcctaacaatggtccgacccacgaaaaccaactgggtgcaggtgcttttggtggttaccaggttaacccgtatgttggctttgaaatgggttacgactggttaggtcgtatgccgtacaaaggcgacaacatcaacggcgcatacaaagctcagggcgttcagctgaccgctaaactgggttacccaatcactgacgatctggacatctacactcgtctgggtggtatggtatggcgtgcagacaccaaggctaacgtacctggtggcgcatcctttaaagaccacgacaccggcgtttctccggtcttcgctggcggtgttgagtatgcgatcactcctgaaatcgctacccgtctggaataccagtggaccaacaacatcggtgacgcacacaccatcggcactcgtccggacaacggcatgctgagcctgggtgtttcctaccgtttcggtcagggcgaagtagctccagtagttgctccggctccagctccggcaccggaagtacagaccaagcacttcactctgaagtctgacgttctgttcaccttcaacaaagcaaccctgaaaccggaaggtcaggctgctctggatcagctgtacagccagctgagcaacctggatccgaaagacggttccgtagttgttctgggttacactgaccgcatcggttctgacgcttataaccaggctctgtccgagcgtcgtgctcagtccgttgttgattacctgatctctaaaggtatcccggcagacaaaatctccgcacgtggtatgggcgaatccaacccggttactggcaacacctgtgacaacgtgaaacagcgtgctgcactgatcgattgcctggctccggatcgtcgcgtagagatcgaagttaaaggcatcaaagacgttgtaactcagccgcaggcttaa

SEQ ID NO: 54 - DNA, 516 bp, Escherichia coli
  source 1..516: mol_type = other DNA
atgaaaaaaattgcatgtctttcagcactggccgcagttctggctttcaccgcaggtacttccgtagctgcgacttctaccgtaactggcggttacgcacaaagcgacgctcagggccaaatgaacaaaatgggcggtttcaacctgaaataccgctatgaagaagacaacagcccgctgggtgtgatcggttctttcacttacaccgagaaaagccgtactgcaagctctggtgactacaacaaaaaccagtactacggcatcactgctggtccggcttaccgcattaacgactgggcaagcatctacggtgtagtgggtgtgggttatggtaaattccagaccactgaatacccgacctacaaacacgacaccagcgactacggtttctcctacggtgctggtttgcagttcaacccgatggaaaacgttgctctggacttctcttacgagcagagccgtattcgtagcgttgacgtaggcacctggattgccggtgttggttaccgcttctaa

SEQ ID NO: 55 - DNA, 2022 bp, Escherichia coli
  source 1..2022: mol_type = other DNA
atgaaaatgacacggctttatcctctggccttggggggattattgctccccgccattgctaatgcccagacttcacagcaagacgaaagcacgctggtggttaccgccagtaaacaatcttcccgctcggcatcagccaacaacgtctcatctactgttgtcagcgcgccggaattaagcgacgccggcgtcaccgccagcgacaaactccccagagtcttgcccgggctcaatattgaaaatagcggcaacatgcttttttcgacgatctcgctacgcggcgtctcttcagcgcaggacttctataaccccgccgtcaccctgtatgtcgatggcgtccctcagctttccaccaacaccatccaggcgcttaccgatgtgcaaagcgtggagttgctgcgaggcccacagggaacgttatatggcaaaagcgctcagggcgggatcatcaacatcgtcacccagcagccggacagcacgccgcgcggctatattgaaggcggcgtcagtagccgcgacagttatcgaagtaagttcaacctgagcggccccattcaggatggcctgctgtacggcagcgtcaccctgttacgccaggttgatgacggcgacatgattaaccccgcgacgggaagcgatgacttaggcggcacccgcgccagcatagggaatgtgaaactgcgtctggcgccggacgatcagccctgggaaatgggctttgccgcctcacgcgaatgtacccgcgccacccaggatgcctatgtgggatggaatgatattaagggccgtaagctgtcgatcagcgatggttcaccagacccgtacatgcggcgctgcactgacagccagaccctgagtgggaaatacaccaccgatgactgggttttcaacctgatcagcgcctggcagcagcagcattattcgcgcaccttcccttccggttcgttaatcgtcaatatgcctcagcgctggaatcaggatgtgcaggagctgcgcgccgcaaccctgggcgatgcgcgtaccgttgatatggtgtttgggctgtaccggcagaacacccgcgagaagttaaattcagcctacgacatgccgacaatgccttatttaagcagtaccggctataccaccgctgaaacgctggccgcatacagtgacctgacctggcatttaaccgatcgttttgatatcggcggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcgggtcttgatgccaaaccgttcgtcgccgagaaatccatcaactatgaacttggcacccgctacgaaaccgctgacgtcacgctgcaagccgcgacgttttatacccacaccaaagacatgcagctttactctggcccggtcgggatgcagacattaagcaatgcgggtaaagccgacgccaccggcgttgagcttgaagcgaagtggcggtttgcgccaggctggtcatgggatatcaatggcaacgtgatccgttccgaattcaccaatgacagtgagttgtatcacggtaaccgggtgccgttcgtaccacgttatggcgcgggaagcagcgtgaacggcgtgattgatacgcgctatggcgcactgatgccccgactggcggttaatctggtcgggccgcattatttcgatggcgacaaccagttgcggcaaggcacctatgccaccctggacagcagcctgggctggcaggcgactgaacggatgaacatttccgtctatgtcgataacctgttcgaccgtcgttaccgtacctatggctacatgaacggcagcagcgccgtcgcgcaggtcaatatgggtcgcaccgtcggtatcaatacgcgaattgatttcttctga

SEQ ID NO: 56 - DNA, 2148 bp, Escherichia coli
  source 1..2148: mol_type = other DNA
atgttatataatataccttgtcgaatttatatcctttccactctgtcattatgcatttctgggatagtttctactgcaaccgcaacttcttcagaaacaaaaatcagcaacgaagagacgctcgtcgtgaccacgaatcgttcggcaagcaacctttgggaaagcccggcgactatacaggttattgaccaacaaacattgcagaactccaccaatgcctccatagccgataatttgcaggacatccccggagtagagataacagacaactccttggcaggccgtaaacaaatccgcattcgtggcgaagcatcctcccgtgttttaattctcattgatggtcaggaggtaacttatcagcgcgccggagataattatggtgtgggactgttgatagatgagtctgcgctggagcgtgttgaggtagtgaaaggtccatattccgtactgtacggttcacaggcaattggcggtattgttaacttcataaccaaaaagggaggtgacaaacttgcatctggagttgtgaaagctgtttataattccgcaacagcaggctgggaagaatcaatcgcggtccaggggagcatcggtggatttgattatcgcatcaacggtagttattctgatcagggcaatcgtgatacgccggatggacgtctgccgaataccaactatcgtaacaatagtcagggtgtatggttgggttataactccggaaaccatcgttttggcctctcgcttgatcgctacagactcgcgacgcaaacttactatgaggatccagacggaagctatgaggcatttagtgtcaaaatacctaaacttgaacgagagaaagttggggtattctatgacacagacgtggacggtgactatctaaaaaaaattcatttcgacgcgtatgagcagaccatccagcgccaatttgccaacgaagtaaaaacgacacagcctgttcccagtccgatgattcaggctctgaccgttcataacaagactgacacccatgataagcaatacactcaggcggtcacattgcagagtcacttttcgctgcctgctaataatgaacttgttaccggtgcacagtacaaacaagacagggtcagccaaaggtccggtggcatgacctcaagcaaatctctgaccggcttcattaataaggaaacacgaactcgctcctattatgagtcagagcaaagtacagtctcactattcgcacaaaatgactggcgattcgccgatcactggacatggacaatgggagttcgccaatactggctttcttcaaagttgacgcgtggtgacggagtatcatataccgcaggcattataagcgatacctctcttgccagagagtctgcgagtgatcacgaaatggtaacatctacaagcctgcgctattcaggtttcgataacttggagttacgcgctgcgttcgcgcaaggctacgtatttcccacactctcccagctttttatgcagacatctgcgggcggcagtgtcacatacggaaatcctgatcttaaggctgaacactccaataactttgaattaggtgcacgatataatggtaatcagtggctgattgacagcgcagtttactactcagaagctaaagattatattgcaagtctgatctgtgatggcagtatagtttgcaatggtaacaccaactcctcccgtagtagctactattattatgacaatattgatcgggcaaaaacatggggactggaaataagcgcggaatataatggctgggttttctcgccatatatcagtggcaatttaattcgtcggcaatatgaaacttcaacattaaaaacaactaatacaggagaaccagcgataaacggacgtatagggctgaaacatactcttgtgatgggtcaggccaacataatctctgatgtttttattcgtgctgcctctagtgcaaaagatgacagtaacggtaccgaaacaaatgttccgggctgggccactctcaactttgcagtaaatacagaattcggtaacgaggatcagtaccggattaacctagcactcaataacctgacagacaaacgctaccgtacagcacatgaaactattcctgcagcaggttttaatgcagctataggttttgtatggaatttctga

SEQ ID NO: 57 - DNA, 2181 bp, Escherichia coli
  source 1..2181: mol_type = other DNA
atgccccgggctcttggtccgctgcttcttgtcgtgctgtcaccagctgtcgcccagcaaaacgatgataatgagatcatagtgtctgccagccgcagcaatcgaactgtagcggagatggcgcaaaccacctgggttatcgaaaatgccgaactggagcagcagattcagggcggtaaagagctgaaagacgcactggctcagttaatccccggccttgatgtcagcagccagagccgaaccaactacggtatgaacatgcgtggccgcccgctggttgtcctgattgacggtgtgcgcctcaactcttcacgttccgacagccgacaactggactctgtcgatccttttaatatcgaccatattgaagtgatctccggcgcgacggccctgtacggtggcgggagtaccggagggttgatcaacatcgtgaccaaaaaaggccagccggaaaccatgatggagtttgaggctggcacaaaaagtggctttaacagcagtaaagatcacgatgagcgcattgccggtgctgtctccggcggaaatgaccatatctccggacgtctttccgtggcatatcagaaatttggcggctggtttgacggtaacggcgatgccaccctgcttgataacacccagaccggcctgcagcactccaatcggctggacatcatgggaaccggtacgctgaacatcgatgaatcccggcagcttcaactgataacgcagtactataaaagtcagggggacgacaattacgggcttaatctcgggaaaggcttttccgccatcagcgggagcagcacaccatacgtcagtaaggggctgaattctgaccgcattcccggcactgagcggcatttgatcagcctgcagtactctgacagtgatttcctgagacaggaactggtcggtcaggtttactaccgcgatgagtcgttgcggttctacccgttcccgacggtaaatgcgaataaacaggcgacggctttctcctcgtcacagcaggataccgaccagtacggcatgaaactgactctgaacagccaacttatggacggctggcaaatcacctgggggctggatgctgagcatgagcgctttacctccaaccagatgttcttcgatctggctcaggcaagtgcttccggagggctgaacaaccataagatttacaccaccgggcgctatccgtcatatgacatcaccaatctggcggccttcctgcaatccagctatgacattaatgatatttttaccgttagcggtggcgtacgctatcagtatactgagaacagggtagatgatttcatcgactacacgcagcaacagaagattgctgccgggaaggcgatatctgccgacgccattcctggtggttcggtagattacgataactttctgttcaatgctggtctgctgatgcacatcaccgaacgtcagcaggcatggttcaatttttcccagggggtggcattgccggatccggggaaatattatggtcgcggcatctatggtgcagcagtgaacggccatcttcccctgacaaagagcgtgaacgtcagcgacagtaagctggaaggcgtgaaagtcgattcttatgaactgggctggcgctttaccggtgacaacctgcggactcaaatcgcggcatattactcgctttccaataagagcgtggaaaggaataaagatctgaccatcagtgtgaaggacgacaggcgccgtatttacggcgtggaaggtgcggtggactacctgatcccggatactgactggagtaccggtgtgaacttcaatgtgctgaaaaccgagtcgaaagtgaacggtcaatggcaaaaatatgacgtgaaggaatcaagtccatcgaaagcgacagcttacattaactgggcgccggaaccgtggagtctgcgtgtacagagcaccacttctttcgacgtaagcgatgcagagggtaacgatattaatggttacactaccgtcgattttatcagtagttggcagcttccggtgggaacactcagcttcagcgttgagaacctcttcgaccgtgactataccactgtctggggacagcgtgcacctctgtactacagcccgggttacggccctgcttcactgtacgactacaaaggccggggccgaacctttggtctgaactactcagtgctgttctga

SEQ ID NO: 58 - DNA, 1095 bp, Actinobacillus pleuropneumoniae
  source 1..1095: mol_type = other DNA
atgaaaaaatcattagttgctttaacagtattatcggctgcagcggtagctcaagcagcgccacaacaaaatactttctacgcaggtgcgaaagcaggttgggcgtcattccatgatggtatcgaacaattagattcagctaaaaacacagatcgcggtacaaaatacggtatcaaccgtaattcagtaacttacggcgtattcggcggttaccaaattttaaaccaagacaaattaggtttagcggctgaattaggttatgactatttcggtcgtgtgcgcggttctgaaaaaccaaacggtaaagcggacaagaaaactttccgtcacgctgcacacggtgcgacaatcgcattaaaacctagctacgaagtattacctgacttagacgtttacggtaaagtaggtatcgcattagtaaacaatacatataaaacattcaatgcagcacaagagaaagtgaaaactcgtcgtttccaaagttctttaattttaggtgcgggtgttgagtacgcaattcttcctgaattagcggcacgtgttgaataccaatggttaaacaacgcaggtaaagcaagctactctactttaaatcgtatgggtgcaactgactaccgttcggatatcagttccgtatctgcaggtttaagctaccgtttcggtcaaggtgcggtaccggttgcagctccggcagttgaaactaaaaacttcgcattcagctctgacgtattattcgcattcggtaaatcaaacttaaaaccggctgcggcaacagcattagatgcaatgcaaaccgaaatcaataacgcaggtttatcaaatgctgcgatccaagtaaacggttacacggaccgtatcggtaaagaagcttcaaacttaaaactttcacaacgtcgtgcggaaacagtagctaactacatcgtttctaaaggtgctctggcagctaacgtaactgcagtaggttacggtgaagcaaaccctgtaaccggcgcaacatgtgacaaagttaaaggtcgtaaagcattaatcgcttgcttagcaccggatcgtcgtgttgaagttcaagttcaaggtactaaagaagtaactatgtaa

SEQ ID NO: 59 - DNA, 648 bp, Actinobacillus pleuropneumoniae
  source 1..648: mol_type = other DNA
atgaaaaaagcagtattagcggcagtattaggcggtgcgttattagcggattcggcaatggcacatcaagcgggcgatgtgattttccgtgccggtgcgatcggtgtgattgcaaattcaagttcggattatcaaaccggggcggacgtaaacttagatgtaaataataatattcagcttggtttaaccggtacctatatgttaagtgataatttaggtcttgaattattagcggcaacaccgttttctcacaaaatcaccggtaagttaggtgcaacagatttaggcgaagtggcaaaagtaaaacatcttccgccgagcctttacttacaatattatttctttgattctaatgcgacagttcgtccatacgttggtgccggtttaaactatactcgctttttcagtgctgaaagtttaaaaccgcaattagtacaaaacttacgtgttaaaaaacattccgtcgcaccgattgcgaatttaggtgttgatgtgaaattaacggataatctatcattcaatgcggcagcttggtacacacgtattaaaactactgccgattatgatgttccgggattaggtcatgtaagtacaccgattactttagatcctgttgtattattctcaggtattagctacaaattctaa

SEQ ID NO: 60 - DNA, 2808 bp, Actinobacillus pleuropneumoniae
  source 1..2808: mol_type = other DNA
atgaaaaataaattaaatctgattagccttgctctgcttagcctctttgccgtacaaagctatgcagaacaagcggtgcaattgaacgatgtttatgtcacaggaacaaaaaagaaagcacataaaaaagagaacgaagtcacaggcttagggaaagtagttaaaacaccagatactcttagtaaggagcaagtgttaggaatacgagatctgactcgttacgaccccggtatttctgtcgtagaacaagggagaggtgcgactacaggctactcaattcgtggggtagatcgtaaccgtgttggcttggcattagatggtttaccacagattcagtcctatgtaagccagtattcacgttcctcaagcggtgccattaatgaaatagaatacgaaaatctgcgttcgatccaaattagtaaaggggctagttcttctgagtttggtagtggctcactaggcggttcggtgcaattccgtaccaaagaggtaagcgacattattaagccagggcaatcttggggattagataccaaaagtgcctacagtagcaaaaatcaacaatggttaaactcacttgcttttgctggtactcacaatggctttgatgctcttgtgatttacactcaccgtgatggtaaggaaacgaaagctcataaaaatgcagagagtcgttctcagaatatcacccgagtaggagtggaaaccaacgagcttgatacctcaaatagatatactgcgacgacgaataatcaacatacttatggctggtttttgattaaagatgaatgtccaacgttagattgtacgccgaaacagatggctagggtgacaaaagatacgccatctttccgttcttaccctgaatatactcctgaggaaaaacaggcttatgagaaccaaaaacatattacagagcgtctaaatgctcaggattacactggtgaatatagagctttacctgatccgcttaaatataaatctgattcttggctggttaaattaggatacacattctctccgaaacattatgtcgctggtacttatgaacatagcaaacagcgttacgacacccgagatatgacctataccgcttattggcaaccatcggatttacttagaactggtagaaattggtatccaatgaataatgctaaaggattatatcgtgataatgctttagatggtgttgctattgactactttacggaagatggtgtgaaatcctcaaaaggtttacgttgggcaaaagctcgttttattgacgagtggcacactcgtgatcgcttaggtgctttatatcgttatactaatcaagatggaaatcgtctgattgatagactatccttgagtttcgatcagcaaaaaattaatttatctacccgcttgagagaaaacaactgttcggaatatccaaccatagataagaattgccgtgcaactcttgataaactttggtcttctactaaaaatgagcaaagttcttatgaagaaaaacacgacactattcagctctcgttagataaaaccgtacaaacgggattgggtaaacatcaattaaatatgttattaggttcagaccgtttcaattccaccttaaaacgccatgaaattttgagtgaattttctgtagggagttgggggcttgttagagacattggctatcgaaatggatcttacaataatccttatgtgtatgagctaaaagatcaggcaatttatagtaaaaatgaatgtgattatagtggcactattgcaggtagggctgattgtgctacaagtaaaatcaaagggcataatcactacatcgctctgagagataattttgccataaccaagtatttggatattggtttgggttaccgtttcgataagcataaattccgtagcactcatcgctgggcaaatcaaggcgattataaaaatagtgcgtggaatattggcatagtcgcaaaaccaacgtcattcctatcgctctcttatcgagcatcatctggctttagagtgccaagtttccaagagctatttggcttacgttatgatggtgcaatgaaaggctccagcgatgcttaccaaaaaacagagaagttatctcctgaaaaatccttaaaccaagaggttgctgcgactttcaaaggtgattttggtgtcgttgaagtcagttatttcaaaaatgactataagcagttaattgctccagcagaaagaatgcaccaaactcaatcaatgattaactattttaatgtgcaagatattaaattggacggcattaatcttattggtaagctagattggaatggggtatttgataaaattcctgagggcatttacacaacattggcttatagcaaaatgcgagtaaaagaggtgaaaaactatcaagggtatatgaatattcgttctccattgttagataccattcagcctgctcgctatgttgtaggagtggggtacgatcagccagatgaaaaatggggcgtgaatctaacaatgacacactccagtggaaaaaatccaaatgagttaagaggtaatgaacaagtcggttttgccaattatgagcgaactgccacgaagaaaagaacacgttcttggcatacctttgacttaacgggatatatcaccccttggaaacatacaacggtacgagctggcgtatataacctgatgaattatcgttacaccacttgggaatccgtacgtcaatcttcgcttaatgcaattcatcagcatactaacgtaaaagactatgcaaggtatgcagcgcccggtagaaattatgttgtttcattcgaaatgaaattctaa

SEQ ID NO: 61 - DNA, 447 bp, Actinobacillus pleuropneumoniae
  source 1..447: mol_type = other DNA
atgcaaaaactaagtcttattcgaccgcttactaacgcgtttactttaattgaattgatgatcgtgattgcgattattgccattttagctacggttgcaattccgtcatataacagttatacccaaaaagcggcgctttcggagctattggcggcatcggcttcttataaaacggatgtcgagatctgcatatataacaccggagattctaaaaactgtagcggcggtcaaaacggtgtcagaaaaatgacggagcttagacaggctaaatatttaaatgccattacggtggaaggcggaacgattacggtaacggggaaggggaatctgcaggaatacggttatacgatgacaccgattcataacggtagcactatttcttgggaaacgaaatgtaaaggggaggacttaagtttatttccggcaaatttctgtgcgataaattag

SEQ ID NO: 62 - DNA, 1047 bp, Acinetobacter baumannii
  source 1..1047: mol_type = other DNA
atgaaattgagtcgtattgcacttgctactatgcttgttgctgctccattagctgctgctaatgctggtggtaaactgggttggtcccagtatcacgacaccggtttctacggtaacggtttccagaacaacaacggtccgacccgtaacgatcagcttggtgctggtgcgttcggtggttaccaggttaacccgtacctcggtttcgaaatgggttatgactggctgggccgtatggcatataaaggcagcgttgacaacggtgctttcaaagctcagggcgttcagctgaccgctaaactgggttacccgatcactgacgatctggacatctacacccgtctgggcggcatggtttggcgcgctgactccaaaggcaactacgcttctaccggcgtttcccgtagcgaacacgacactggcgtttccccagtatttgctggcggcgtagagtgggctgttactcgtgacatcgctacccgtctggaataccagtgggttaacaacatcggcgacgcgggcactgtgggtacccgtcctgataacggcatgctgagcctgggcgtttcctaccgcttcggtcaggaagatgctgcaccggttgttgctccggctccggctccggctccggaagtggctaccaagcacttcaccctgaagtctgacgttctgttcaacttcaacaaagctaccctgaaaccggaaggtcagcaggctctggatcagctgtacactcagctgagcaacatggacccgaaagacggttccgctgttgttctgggctacaccgaccgcatcggttccgaagcttacaaccagcagctgtctgagaaacgtgctcagtccgttgttgactacctggttgctaaaggcatcccggctggcaaaatctccgctcgcggcatgggtgaatccaacccggttactggcaacacctgtgacaacgtgaaagctcgcgctgccctgatcgattgcctggctccggatcgtcgtgtagagatcgaagttaaaggctacaaagaagttgtaactcagccgcaggcttaa

SEQ ID NO: 63 - DNA, 1107 bp, Acinetobacter baumannii
  source 1..1107: mol_type = other DNA
atgaaattgagtcgtattgcacttgctactatgcttgttgctgctccattagctgctgctaatgctgctgaaatttataacaaagacggcaacaaattagacctgtacggtaaaattgacggtctgcactacttctctgacgacaagagcgtcgacggcgaccagacctacatgcgtgtaggcgtgaaaggcgaaacccagatcaacgaccagctgaccggttacggccagtgggaatacaacgttcaggcgaacaacactgaaagctccagcgatcaggcatggactcgtctggcattcgcaggcctgaaatttggcgacgcgggctctttcgactacggtcgtaactacggcgtagtatacgacgtaacgtcctggaccgacgttctgccggaattcggcggcgacacctacggttctgacaacttcctgcagtcccgtgctaacggcgttgcaacctaccgtaactctgatttcttcggtctggttgacggcctgaactttgctctgcagtatcagggtaaaaacggcagcgtcagcggcgaaggcgctctgtctcctaccaacaacggtcgtaccgccttgaaacagaacggcgacggttacggtacttctctgacctatgacatctatgatggcatcagcgctggtttcgcatactctaactccaaacgtcttggcgaccagaacagcaagctggcactgggtcgtggcgacaacgctgaaacctacaccggcggtctgaaatacgacgcgaacaacatctacctggccactcagtacacccagacctacaacgcgacccgcgccggttccctgggctttgctaacaaagcgcagaacttcgaagtggttgctcagtaccagttcgacttcggtctgcgtccgtccgtggcttacctgcagtctaaaggtaaggatctggaaggctacggcgaccaggacatcctgaaatatgttgacgttggcgcgacctactacttcaacaaaaacatgtccacctatgttgactacaaaatcaacctgctggacgacaatagcttcacccacaacgccggtatctctaccgacgacgtggttgcactgggcctggtttaccagttctaa

SEQ ID NO: 64 - DNA, 1047 bp, Acinetobacter baumannii
  source 1..1047: mol_type = other DNA
atgaaattgagtcgtattgcacttgctactatgcttgttgctgctccattagctgctgctaatgctcagggccagaactcggtagagatcgaagccttcggcaagcgctacttcaccgacagcgttcgcaacatgaagaacgcggacctgtacggcggctcgatcggttacttcctgaccgacgacgtcgagctggcgctgtcctacggtgagtaccatgacgttcgtggcacctacgaaaccggcaacaagaaggtccacggcaacctgacctccctggacgccatctaccacttcggtaccccgggcgtaggtctgcgtccgtacgtgtcggctggtctggctcaccagaacatcaccaacatcaacagcgacagccaaggccgtcagcagatgaccatggccaacatcggcgctggtctgaagtactacttcaccgagaacttcttcgccaaggccagcctcgacggccagtacggtctggagaagcgtgacaacggtcaccagggcgagtggatggctggcctgggcgtcggcttcaacttcggtggttcgaaagccgctccggctccggaaccggttgccgacgtttgctccgactccgacaacgacggcgtttgcgacaacgtcgacaagtgcccggataccccggccaacgtcaccgttgacgccaacggctgcccggctgtcgccgaagtcgtacgcgtacagctggacgtgaagttcgacttcgacaagtccaaggtcaaagagaacagctacgctgacatcaagaacctggctgacttcatgaagcagtacccgtccacttccaccaccgttgaaggtcacaccgactccgtcggcaccgacgcttacaaccagaagctgtccgagcgtcgtgccaacgccgttcgtgacgtactggtcaacgagtacggtgtagaaggtggtcgcgtgaacgctgttggttacggcgagtcccgcccggttgccgacaacgccaccgctgaaggccgcgctatcaaccgtcgcgttgaagccgaagtagaagctgaagccaagtaa

SEQ ID NO: 65 - DNA, 1122 bp, Acinetobacter baumannii
  source 1..1122: mol_type = other DNA
atgaaattgagtcgtattgcacttgctactatgcttgttgctgctccattagctgctgctaatgctagccactccaaagaaaccgaagctcgtctgaccgctaccgaagacgcagctgctcgtgctcaggctcgcgctgacgaagcctatcgcaaggctgacgaagctctgggcgctgctcagaaagctcagcagactgctgacgaggctaacgagcgtgccctgcgcatgctggaaaaagccagccgcaagggcggcggcggcagcggcggcggcggcagcggcggcggcggcagctcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgcgagttcctggtgtcggcctatttcagcctgcacgggcgtctcgacgaggatgtcatcggtgtctacaaggatgtcctgcagacccaggacggcaagcgcaaggcgctgctcgacgagctgaaggcgctgaccgcggagttgaaggtctacagcgtgatccagtcgcagatcaacgccgcgctgtcggccaagcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgcgcgacattctcagcgcgatctag

SEQ ID NO: 66 - DNA, 254 bp, Acinetobacter baumannii
  source 1..254: mol_type = other DNA
atgaaattgagtcgtattgcacttgctactatgcttgttgctgctccattagctgctgctaatgctagccactccaaagaaaccgaagctcgtctgaccgctaccgaagacgcagctgctcgtgctcaggctcgcgctgacgaagcctatcgcaaggctgacgaagctctgggcgctgctcagaaagctcagcagactgctgacgaggctaacgagcgtgccctgcgcatgctggaaaaagccagccgcaatag

SEQ ID NO: 67 - DNA, 1056 bp, Acinetobacter baumannii
  source 1..1056: mol_type = other DNA
atgaaattgagtcgtattgcacttgctactatgcttgttgctgctccattagctgctgctaatgctgctccgaaagataacacctggtacactggtgctaaactgggctggtcccagtaccatgacactggttttattcctaacaatggtccgacccacgaaaaccaactgggtgcaggtgcttttggtggttaccaggttaacccgtatgttggctttgaaatgggttacgactggttaggtcgtatgccgtacaaaggcgacaacatcaacggcgcatacaaagctcagggcgttcagctgaccgctaaactgggttacccaatcactgacgatctggacatctacactcgtctgggtggtatggtatggcgtgcagacaccaaggctaacgtacctggtggcgcatcctttaaagaccacgacaccggcgtttctccggtcttcgctggcggtgttgagtatgcgatcactcctgaaatcgctacccgtctggaataccagtggaccaacaacatcggtgacgcacacaccatcggcactcgtccggacaacggcatgctgagcctgggtgtttcctaccgtttcggtcagggcgaagtagctccagtagttgctccggctccagctccggcaccggaagtacagaccaagcacttcactctgaagtctgacgttctgttcaccttcaacaaagcaaccctgaaaccggaaggtcaggctgctctggatcagctgtacagccagctgagcaacctggatccgaaagacggttccgtagttgttctgggttacactgaccgcatcggttctgacgcttataaccaggctctgtccgagcgtcgtgctcagtccgttgttgattacctgatctctaaaggtatcccggcagacaaaatctccgcacgtggtatgggcgaatccaacccggttactggcaacacctgtgacaacgtgaaacagcgtgctgcactgatcgattgcctggctccggatcgtcgcgtagagatcgaagttaaaggcatcaaagacgttgtaactcagccgcaggcttaa

SEQ ID NO: 68 - DNA, 513 bp, Acinetobacter baumannii
  source 1..513: mol_type = other DNA
atgaaattgagtcgtattgcacttgctactatgcttgttgctgctccattagctgctgctaatgctgcgacttctaccgtaactggcggttacgcacaaagcgacgctcagggccaaatgaacaaaatgggcggtttcaacctgaaataccgctatgaagaagacaacagcccgctgggtgtgatcggttctttcacttacaccgagaaaagccgtactgcaagctctggtgactacaacaaaaaccagtactacggcatcactgctggtccggcttaccgcattaacgactgggcaagcatctacggtgtagtgggtgtgggttatggtaaattccagaccactgaatacccgacctacaaacacgacaccagcgactacggtttctcctacggtgctggtttgcagttcaacccgatggaaaacgttgctctggacttctcttacgagcagagccgtattcgtagcgttgacgtaggcacctggattgccggtgttggttaccgcttctaa

SEQ ID NO: 69 - DNA, 2022 bp, Acinetobacter baumannii
  source 1..2022: mol_type = other DNA
atgaaattgagtcgtattgcacttgctactatgcttgttgctgctccattagctgctgctaatgctcagacttcacagcaagacgaaagcacgctggtggttaccgccagtaaacaatcttcccgctcggcatcagccaacaacgtctcatctactgttgtcagcgcgccggaattaagcgacgccggcgtcaccgccagcgacaaactccccagagtcttgcccgggctcaatattgaaaatagcggcaacatgcttttttcgacgatctcgctacgcggcgtctcttcagcgcaggacttctataaccccgccgtcaccctgtatgtcgatggcgtccctcagctttccaccaacaccatccaggcgcttaccgatgtgcaaagcgtggagttgctgcgaggcccacagggaacgttatatggcaaaagcgctcagggcgggatcatcaacatcgtcacccagcagccggacagcacgccgcgcggctatattgaaggcggcgtcagtagccgcgacagttatcgaagtaagttcaacctgagcggccccattcaggatggcctgctgtacggcagcgtcaccctgttacgccaggttgatgacggcgacatgattaaccccgcgacgggaagcgatgacttaggcggcacccgcgccagcatagggaatgtgaaactgcgtctggcgccggacgatcagccctgggaaatgggctttgccgcctcacgcgaatgtacccgcgccacccaggatgcctatgtgggatggaatgatattaagggccgtaagctgtcgatcagcgatggttcaccagacccgtacatgcggcgctgcactgacagccagaccctgagtgggaaatacaccaccgatgactgggttttcaacctgatcagcgcctggcagcagcagcattattcgcgcaccttcccttccggttcgttaatcgtcaatatgcctcagcgctggaatcaggatgtgcaggagctgcgcgccgcaaccctgggcgatgcgcgtaccgttgatatggtgtttgggctgtaccggcagaacacccgcgagaagttaaattcagcctacgacatgccgacaatgccttatttaagcagtaccggctataccaccgctgaaacgctggccgcatacagtgacctgacctggcatttaaccgatcgttttgatatcggcggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcgggtcttgatgccaaaccgttcgtcgccgagaaatccatcaactatgaacttggcacccgctacgaaaccgctgacgtcacgctgcaagccgcgacgttttatacccacaccaaagacatgcagctttactctggcccggtcgggatgcagacattaagcaatgcgggtaaagccgacgccaccggcgttgagcttgaagcgaagtggcggtttgcgccaggctggtcatgggatatcaatggcaacgtgatccgttccgaattcaccaatgacagtgagttgtatcacggtaaccgggtgccgttcgtaccacgttatggcgcgggaagcagcgtgaacggcgtgattgatacgcgctatggcgcactgatgccccgactggcggttaatctggtcgggccgcattatttcgatggcgacaaccagttgcggcaaggcacctatgccaccctggacagcagcctgggctggcaggcgactgaacggatgaacatttccgtctatgtcgataacctgttcgaccgtcgttaccgtacctatggctacatgaacggcagcagcgccgtcgcgcaggtcaatatgggtcgcaccgtcggtatcaatacgcgaattgatttcttctga

SEQ ID NO: 70 - DNA, 2130 bp, Acinetobacter baumannii
  source 1..2130: mol_type = other DNA
atgaaattgagtcgtattgcacttgctactatgcttgttgctgctccattagctgctgctaatgctacttcttcagaaacaaaaatcagcaacgaagagacgctcgtcgtgaccacgaatcgttcggcaagcaacctttgggaaagcccggcgactatacaggttattgaccaacaaacattgcagaactccaccaatgcctccatagccgataatttgcaggacatccccggagtagagataacagacaactccttggcaggccgtaaacaaatccgcattcgtggcgaagcatcctcccgtgttttaattctcattgatggtcaggaggtaacttatcagcgcgccggagataattatggtgtgggactgttgatagatgagtctgcgctggagcgtgttgaggtagtgaaaggtccatattccgtactgtacggttcacaggcaattggcggtattgttaacttcataaccaaaaagggaggtgacaaacttgcatctggagttgtgaaagctgtttataattccgcaacagcaggctgggaagaatcaatcgcggtccaggggagcatcggtggatttgattatcgcatcaacggtagttattctgatcagggcaatcgtgatacgccggatggacgtctgccgaataccaactatcgtaacaatagtcagggtgtatggttgggttataactccggaaaccatcgttttggcctctcgcttgatcgctacagactcgcgacgcaaacttactatgaggatccagacggaagctatgaggcatttagtgtcaaaatacctaaacttgaacgagagaaagttggggtattctatgacacagacgtggacggtgactatctaaaaaaaattcatttcgacgcgtatgagcagaccatccagcgccaatttgccaacgaagtaaaaacgacacagcctgttcccagtccgatgattcaggctctgaccgttcataacaagactgacacccatgataagcaatacactcaggcggtcacattgcagagtcacttttcgctgcctgctaataatgaacttgttaccggtgcacagtacaaacaagacagggtcagccaaaggtccggtggcatgacctcaagcaaatctctgaccggcttcattaataaggaaacacgaactcgctcctattatgagtcagagcaaagtacagtctcactattcgcacaaaatgactggcgattcgccgatcactggacatggacaatgggagttcgccaatactggctttcttcaaagttgacgcgtggtgacggagtatcatataccgcaggcattataagcgatacctctcttgccagagagtctgcgagtgatcacgaaatggtaacatctacaagcctgcgctattcaggtttcgataacttggagttacgcgctgcgttcgcgcaaggctacgtatttcccacactctcccagctttttatgcagacatctgcgggcggcagtgtcacatacggaaatcctgatcttaaggctgaacactccaataactttgaattaggtgcacgatataatggtaatcagtggctgattgacagcgcagtttactactcagaagctaaagattatattgcaagtctgatctgtgatggcagtatagtttgcaatggtaacaccaactcctcccgtagtagctactattattatgacaatattgatcgggcaaaaacatggggactggaaataagcgcggaatataatggctgggttttctcgccatatatcagtggcaatttaattcgtcggcaatatgaaacttcaacattaaaaacaactaatacaggagaaccagcgataaacggacgtatagggctgaaacatactcttgtgatgggtcaggccaacataatctctgatgtttttattcgtgctgcctctagtgcaaaagatgacagtaacggtaccgaaacaaatgttccgggctgggccactctcaactttgcagtaaatacagaattcggtaacgaggatcagtaccggattaacctagcactcaataacctgacagacaaacgctaccgtacagcacatgaaactattcctgcagcaggttttaatgcagctataggttttgtatggaatttctga

SEQ ID NO: 71 - DNA, 2193 bp, Acinetobacter baumannii
  source 1..2193: mol_type = other DNA
atgaaattgagtcgtattgcacttgctactatgcttgttgctgctccattagctgctgctaatgctcagcaaaacgatgataatgagatcatagtgtctgccagccgcagcaatcgaactgtagcggagatggcgcaaaccacctgggttatcgaaaatgccgaactggagcagcagattcagggcggtaaagagctgaaagacgcactggctcagttaatccccggccttgatgtcagcagccagagccgaaccaactacggtatgaacatgcgtggccgcccgctggttgtcctgattgacggtgtgcgcctcaactcttcacgttccgacagccgacaactggactctgtcgatccttttaatatcgaccatattgaagtgatctccggcgcgacggccctgtacggtggcgggagtaccggagggttgatcaacatcgtgaccaaaaaaggccagccggaaaccatgatggagtttgaggctggcacaaaaagtggctttaacagcagtaaagatcacgatgagcgcattgccggtgctgtctccggcggaaatgaccatatctccggacgtctttccgtggcatatcagaaatttggcggctggtttgacggtaacggcgatgccaccctgcttgataacacccagaccggcctgcagcactccaatcggctggacatcatgggaaccggtacgctgaacatcgatgaatcccggcagcttcaactgataacgcagtactataaaagtcagggggacgacaattacgggcttaatctcgggaaaggcttttccgccatcagcgggagcagcacaccatacgtcagtaaggggctgaattctgaccgcattcccggcactgagcggcatttgatcagcctgcagtactctgacagtgatttcctgagacaggaactggtcggtcaggtttactaccgcgatgagtcgttgcggttctacccgttcccgacggtaaatgcgaataaacaggcgacggctttctcctcgtcacagcaggataccgaccagtacggcatgaaactgactctgaacagccaacttatggacggctggcaaatcacctgggggctggatgctgagcatgagcgctttacctccaaccagatgttcttcgatctggctcaggcaagtgcttccggagggctgaacaaccataagatttacaccaccgggcgctatccgtcatatgacatcaccaatctggcggccttcctgcaatccagctatgacattaatgatatttttaccgttagcggtggcgtacgctatcagtatactgagaacagggtagatgatttcatcgactacacgcagcaacagaagattgctgccgggaaggcgatatctgccgacgccattcctggtggttcggtagattacgataactttctgttcaatgctggtctgctgatgcacatcaccgaacgtcagcaggcatggttcaatttttcccagggggtggcattgccggatccggggaaatattatggtcgcggcatctatggtgcagcagtgaacggccatcttcccctgacaaagagcgtgaacgtcagcgacagtaagctggaaggcgtgaaagtcgattcttatgaactgggctggcgctttaccggtgacaacctgcggactcaaatcgcggcatattactcgctttccaataagagcgtggaaaggaataaagatctgaccatcagtgtgaaggacgacaggcgccgtatttacggcgtggaaggtgcggtggactacctgatcccggatactgactggagtaccggtgtgaacttcaatgtgctgaaaaccgagtcgaaagtgaacggtcaatggcaaaaatatgacgtgaaggaatcaagtccatcgaaagcgacagcttacattaactgggcgccggaaccgtggagtctgcgtgtacagagcaccacttctttcgacgtaagcgatgcagagggtaacgatattaatggttacactaccgtcgattttatcagtagttggcagcttccggtgggaacactcagcttcagcgttgagaacctcttcgaccgtgactataccactgtctggggacagcgtgcacctctgtactacagcccgggttacggccctgcttcactgtacgactacaaaggccggggccgaacctttggtctgaactactcagtgctgttctga

SEQ ID NO: 72 - DNA, 1107 bp, Acinetobacter baumannii
  source 1..1107: mol_type = other DNA
atgaaattgagtcgtattgcacttgctactatgcttgttgctgctccattagctgctgctaatgctgcagcgccacaacaaaatactttctacgcaggtgcgaaagcaggttgggcgtcattccatgatggtatcgaacaattagattcagctaaaaacacagatcgcggtacaaaatacggtatcaaccgtaattcagtaacttacggcgtattcggcggttaccaaattttaaaccaagacaaattaggtttagcggctgaattaggttatgactatttcggtcgtgtgcgcggttctgaaaaaccaaacggtaaagcggacaagaaaactttccgtcacgctgcacacggtgcgacaatcgcattaaaacctagctacgaagtattacctgacttagacgtttacggtaaagtaggtatcgcattagtaaacaatacatataaaacattcaatgcagcacaagagaaagtgaaaactcgtcgtttccaaagttctttaattttaggtgcgggtgttgagtacgcaattcttcctgaattagcggcacgtgttgaataccaatggttaaacaacgcaggtaaagcaagctactctactttaaatcgtatgggtgcaactgactaccgttcggatatcagttccgtatctgcaggtttaagctaccgtttcggtcaaggtgcggtaccggttgcagctccggcagttgaaactaaaaacttcgcattcagctctgacgtattattcgcattcggtaaatcaaacttaaaaccggctgcggcaacagcattagatgcaatgcaaaccgaaatcaataacgcaggtttatcaaatgctgcgatccaagtaaacggttacacggaccgtatcggtaaagaagcttcaaacttaaaactttcacaacgtcgtgcggaaacagtagctaactacatcgtttctaaaggtgctctggcagctaacgtaactgcagtaggttacggtgaagcaaaccctgtaaccggcgcaacatgtgacaaagttaaaggtcgtaaagcattaatcgcttgcttagcaccggatcgtcgtgttgaagttcaagttcaaggtactaaagaagtaactatgtaa

SEQ ID NO: 73 - DNA, 654 bp, Acinetobacter baumannii
  source 1..654: mol_type = other DNA
atgaaattgagtcgtattgcacttgctactatgcttgttgctgctccattagctgctgctaatgctgcacatcaagcgggcgatgtgattttccgtgccggtgcgatcggtgtgattgcaaattcaagttcggattatcaaaccggggcggacgtaaacttagatgtaaataataatattcagcttggtttaaccggtacctatatgttaagtgataatttaggtcttgaattattagcggcaacaccgttttctcacaaaatcaccggtaagttaggtgcaacagatttaggcgaagtggcaaaagtaaaacatcttccgccgagcctttacttacaatattatttctttgattctaatgcgacagttcgtccatacgttggtgccggtttaaactatactcgctttttcagtgctgaaagtttaaaaccgcaattagtacaaaacttacgtgttaaaaaacattccgtcgcaccgattgcgaatttaggtgttgatgtgaaattaacggataatctatcattcaatgcggcagcttggtacacacgtattaaaactactgccgattatgatgttccgggattaggtcatgtaagtacaccgattactttagatcctgttgtattattctcaggtattagctacaaattctaa

SEQ ID NO: 74 - DNA, 2811 bp, Acinetobacter baumannii
  source 1..2811: mol_type = other DNA
atgaaattgagtcgtattgcacttgctactatgcttgttgctgctccattagctgctgctaatgctgcagaacaagcggtgcaattgaacgatgtttatgtcacaggaacaaaaaagaaagcacataaaaaagagaacgaagtcacaggcttagggaaagtagttaaaacaccagatactcttagtaaggagcaagtgttaggaatacgagatctgactcgttacgaccccggtatttctgtcgtagaacaagggagaggtgcgactacaggctactcaattcgtggggtagatcgtaaccgtgttggcttggcattagatggtttaccacagattcagtcctatgtaagccagtattcacgttcctcaagcggtgccattaatgaaatagaatacgaaaatctgcgttcgatccaaattagtaaaggggctagttcttctgagtttggtagtggctcactaggcggttcggtgcaattccgtaccaaagaggtaagcgacattattaagccagggcaatcttggggattagataccaaaagtgcctacagtagcaaaaatcaacaatggttaaactcacttgcttttgctggtactcacaatggctttgatgctcttgtgatttacactcaccgtgatggtaaggaaacgaaagctcataaaaatgcagagagtcgttctcagaatatcacccgagtaggagtggaaaccaacgagcttgatacctcaaatagatatactgcgacgacgaataatcaacatacttatggctggtttttgattaaagatgaatgtccaacgttagattgtacgccgaaacagatggctagggtgacaaaagatacgccatctttccgttcttaccctgaatatactcctgaggaaaaacaggcttatgagaaccaaaaacatattacagagcgtctaaatgctcaggattacactggtgaatatagagctttacctgatccgcttaaatataaatctgattcttggctggttaaattaggatacacattctctccgaaacattatgtcgctggtacttatgaacatagcaaacagcgttacgacacccgagatatgacctataccgcttattggcaaccatcggatttacttagaactggtagaaattggtatccaatgaataatgctaaaggattatatcgtgataatgctttagatggtgttgctattgactactttacggaagatggtgtgaaatcctcaaaaggtttacgttgggcaaaagctcgttttattgacgagtggcacactcgtgatcgcttaggtgctttatatcgttatactaatcaagatggaaatcgtctgattgatagactatccttgagtttcgatcagcaaaaaattaatttatctacccgcttgagagaaaacaactgttcggaatatccaaccatagataagaattgccgtgcaactcttgataaactttggtcttctactaaaaatgagcaaagttcttatgaagaaaaacacgacactattcagctctcgttagataaaaccgtacaaacgggattgggtaaacatcaattaaatatgttattaggttcagaccgtttcaattccaccttaaaacgccatgaaattttgagtgaattttctgtagggagttgggggcttgttagagacattggctatcgaaatggatcttacaataatccttatgtgtatgagctaaaagatcaggcaatttatagtaaaaatgaatgtgattatagtggcactattgcaggtagggctgattgtgctacaagtaaaatcaaagggcataatcactacatcgctctgagagataattttgccataaccaagtatttggatattggtttgggttaccgtttcgataagcataaattccgtagcactcatcgctgggcaaatcaaggcgattataaaaatagtgcgtggaatattggcatagtcgcaaaaccaacgtcattcctatcgctctcttatcgagcatcatctggctttagagtgccaagtttccaagagctatttggcttacgttatgatggtgcaatgaaaggctccagcgatgcttaccaaaaaacagagaagttatctcctgaaaaatccttaaaccaagaggttgctgcgactttcaaaggtgattttggtgtcgttgaagtcagttatttcaaaaatgactataagcagttaattgctccagcagaaagaatgcaccaaactcaatcaatgattaactattttaatgtgcaagatattaaattggacggcattaatcttattggtaagctagattggaatggggtatttgataaaattcctgagggcatttacacaacattggcttatagcaaaatgcgagtaaaagaggtgaaaaactatcaagggtatatgaatattcgttctccattgttagataccattcagcctgctcgctatgttgtaggagtggggtacgatcagccagatgaaaaatggggcgtgaatctaacaatgacacactccagtggaaaaaatccaaatgagttaagaggtaatgaacaagtcggttttgccaattatgagcgaactgccacgaagaaaagaacacgttcttggcatacctttgacttaacgggatatatcaccccttggaaacatacaacggtacgagctggcgtatataacctgatgaattatcgttacaccacttgggaatccgtacgtcaatcttcgcttaatgcaattcatcagcatactaacgtaaaagactatgcaaggtatgcagcgcccggtagaaattatgttgtttcattcgaaatgaaattctaa

SEQ ID NO: 75 - DNA, 474 bp, Acinetobacter baumannii
  source 1..474: mol_type = other DNA
atgaaattgagtcgtattgcacttgctactatgcttgttgctgctccattagctgctgctaatgcttttactttaattgaattgatgatcgtgattgcgattattgccattttagctacggttgcaattccgtcatataacagttatacccaaaaagcggcgctttcggagctattggcggcatcggcttcttataaaacggatgtcgagatctgcatatataacaccggagattctaaaaactgtagcggcggtcaaaacggtgtcagaaaaatgacggagcttagacaggctaaatatttaaatgccattacggtggaaggcggaacgattacggtaacggggaaggggaatctgcaggaatacggttatacgatgacaccgattcataacggtagcactatttcttgggaaacgaaatgtaaaggggaggacttaagtttatttccggcaaatttctgtgcgataaattag

SEQ ID NO: 76 - DNA, 981 bp, Klebsiella pneumoniae
  source 1..981: mol_type = other DNA
ggtggtaaactgggttggtcccagtatcacgacaccggtttctacggtaacggtttccagaacaacaacggtccgacccgtaacgatcagcttggtgctggtgcgttcggtggttaccaggttaacccgtacctcggtttcgaaatgggttatgactggctgggccgtatggcatataaaggcagcgttgacaacggtgctttcaaagctcagggcgttcagctgaccgctaaactgggttacccgatcactgacgatctggacatctacacccgtctgggcggcatggtttggcgcgctgactccaaaggcaactacgcttctaccggcgtttcccgtagcgaacacgacactggcgtttccccagtatttgctggcggcgtagagtgggctgttactcgtgacatcgctacccgtctggaataccagtgggttaacaacatcggcgacgcgggcactgtgggtacccgtcctgataacggcatgctgagcctgggcgtttcctaccgcttcggtcaggaagatgctgcaccggttgttgctccggctccggctccggctccggaagtggctaccaagcacttcaccctgaagtctgacgttctgttcaacttcaacaaagctaccctgaaaccggaaggtcagcaggctctggatcagctgtacactcagctgagcaacatggacccgaaagacggttccgctgttgttctgggctacaccgaccgcatcggttccgaagcttacaaccagcagctgtctgagaaacgtgctcagtccgttgttgactacctggttgctaaaggcatcccggctggcaaaatctccgctcgcggcatgggtgaatccaacccggttactggcaacacctgtgacaacgtgaaagctcgcgctgccctgatcgattgcctggctccggatcgtcgtgtagagatcgaagttaaaggctacaaagaagttgtaactcagccgcaggcttaa

SEQ ID NO: 77 - DNA, 1041 bp, Klebsiella pneumoniae
  source 1..1041: mol_type = other DNA
gctgaaatttataacaaagacggcaacaaattagacctgtacggtaaaattgacggtctgcactacttctctgacgacaagagcgtcgacggcgaccagacctacatgcgtgtaggcgtgaaaggcgaaacccagatcaacgaccagctgaccggttacggccagtgggaatacaacgttcaggcgaacaacactgaaagctccagcgatcaggcatggactcgtctggcattcgcaggcctgaaatttggcgacgcgggctctttcgactacggtcgtaactacggcgtagtatacgacgtaacgtcctggaccgacgttctgccggaattcggcggcgacacctacggttctgacaacttcctgcagtcccgtgctaacggcgttgcaacctaccgtaactctgatttcttcggtctggttgacggcctgaactttgctctgcagtatcagggtaaaaacggcagcgtcagcggcgaaggcgctctgtctcctaccaacaacggtcgtaccgccttgaaacagaacggcgacggttacggtacttctctgacctatgacatctatgatggcatcagcgctggtttcgcatactctaactccaaacgtcttggcgaccagaacagcaagctggcactgggtcgtggcgacaacgctgaaacctacaccggcggtctgaaatacgacgcgaacaacatctacctggccactcagtacacccagacctacaacgcgacccgcgccggttccctgggctttgctaacaaagcgcagaacttcgaagtggttgctcagtaccagttcgacttcggtctgcgtccgtccgtggcttacctgcagtctaaaggtaaggatctggaaggctacggcgaccaggacatcctgaaatatgttgacgttggcgcgacctactacttcaacaaaaacatgtccacctatgttgactacaaaatcaacctgctggacgacaatagcttcacccacaacgccggtatctctaccgacgacgtggttgcactgggcctggtttaccagttctaa

SEQ ID NO: 78 - DNA, 981 bp, Pseudomonas aeruginosa
  source 1..981: mol_type = other DNA
cagggccagaactcggtagagatcgaagccttcggcaagcgctacttcaccgacagcgttcgcaacatgaagaacgcggacctgtacggcggctcgatcggttacttcctgaccgacgacgtcgagctggcgctgtcctacggtgagtaccatgacgttcgtggcacctacgaaaccggcaacaagaaggtccacggcaacctgacctccctggacgccatctaccacttcggtaccccgggcgtaggtctgcgtccgtacgtgtcggctggtctggctcaccagaacatcaccaacatcaacagcgacagccaaggccgtcagcagatgaccatggccaacatcggcgctggtctgaagtactacttcaccgagaacttcttcgccaaggccagcctcgacggccagtacggtctggagaagcgtgacaacggtcaccagggcgagtggatggctggcctgggcgtcggcttcaacttcggtggttcgaaagccgctccggctccggaaccggttgccgacgtttgctccgactccgacaacgacggcgtttgcgacaacgtcgacaagtgcccggataccccggccaacgtcaccgttgacgccaacggctgcccggctgtcgccgaagtcgtacgcgtacagctggacgtgaagttcgacttcgacaagtccaaggtcaaagagaacagctacgctgacatcaagaacctggctgacttcatgaagcagtacccgtccacttccaccaccgttgaaggtcacaccgactccgtcggcaccgacgcttacaaccagaagctgtccgagcgtcgtgccaacgccgttcgtgacgtactggtcaacgagtacggtgtagaaggtggtcgcgtgaacgctgttggttacggcgagtcccgcccggttgccgacaacgccaccgctgaaggccgcgctatcaaccgtcgcgttgaagccgaagtagaagctgaagccaagtaa

SEQ ID NO: 79 - DNA, 1056 bp, Pseudomonas aeruginosa
  source 1..1056: mol_type = other DNA
agccactccaaagaaaccgaagctcgtctgaccgctaccgaagacgcagctgctcgtgctcaggctcgcgctgacgaagcctatcgcaaggctgacgaagctctgggcgctgctcagaaagctcagcagactgctgacgaggctaacgagcgtgccctgcgcatgctggaaaaagccagccgcaagggcggcggcggcagcggcggcggcggcagcggcggcggcggcagctcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgcgagttcctggtgtcggcctatttcagcctgcacgggcgtctcgacgaggatgtcatcggtgtctacaaggatgtcctgcagacccaggacggcaagcgcaaggcgctgctcgacgagctgaaggcgctgaccgcggagttgaaggtctacagcgtgatccagtcgcagatcaacgccgcgctgtcggccaagcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgcgcgacattctcagcgcgatctag

SEQ ID NO: 80 - DNA, 188 bp, Pseudomonas aeruginosa
  source 1..188: mol_type = other DNA
agccactccaaagaaaccgaagctcgtctgaccgctaccgaagacgcagctgctcgtgctcaggctcgcgctgacgaagcctatcgcaaggctgacgaagctctgggcgctgctcagaaagctcagcagactgctgacgaggctaacgagcgtgccctgcgcatgctggaaaaagccagccgcaatag

SEQ ID NO: 81 - DNA, 990 bp, Escherichia coli
  source 1..990: mol_type = other DNA
gctccgaaagataacacctggtacactggtgctaaactgggctggtcccagtaccatgacactggttttattcctaacaatggtccgacccacgaaaaccaactgggtgcaggtgcttttggtggttaccaggttaacccgtatgttggctttgaaatgggttacgactggttaggtcgtatgccgtacaaaggcgacaacatcaacggcgcatacaaagctcagggcgttcagctgaccgctaaactgggttacccaatcactgacgatctggacatctacactcgtctgggtggtatggtatggcgtgcagacaccaaggctaacgtacctggtggcgcatcctttaaagaccacgacaccggcgtttctccggtcttcgctggcggtgttgagtatgcgatcactcctgaaatcgctacccgtctggaataccagtggaccaacaacatcggtgacgcacacaccatcggcactcgtccggacaacggcatgctgagcctgggtgtttcctaccgtttcggtcagggcgaagtagctccagtagttgctccggctccagctccggcaccggaagtacagaccaagcacttcactctgaagtctgacgttctgttcaccttcaacaaagcaaccctgaaaccggaaggtcaggctgctctggatcagctgtacagccagctgagcaacctggatccgaaagacggttccgtagttgttctgggttacactgaccgcatcggttctgacgcttataaccaggctctgtccgagcgtcgtgctcagtccgttgttgattacctgatctctaaaggtatcccggcagacaaaatctccgcacgtggtatgggcgaatccaacccggttactggcaacacctgtgacaacgtgaaacagcgtgctgcactgatcgattgcctggctccggatcgtcgcgtagagatcgaagttaaaggcatcaaagacgttgtaactcagccgcaggcttaa

SEQ ID NO: 82 - DNA, 447 bp, Escherichia coli
  source 1..447: mol_type = other DNA
gcgacttctaccgtaactggcggttacgcacaaagcgacgctcagggccaaatgaacaaaatgggcggtttcaacctgaaataccgctatgaagaagacaacagcccgctgggtgtgatcggttctttcacttacaccgagaaaagccgtactgcaagctctggtgactacaacaaaaaccagtactacggcatcactgctggtccggcttaccgcattaacgactgggcaagcatctacggtgtagtgggtgtgggttatggtaaattccagaccactgaatacccgacctacaaacacgacaccagcgactacggtttctcctacggtgctggtttgcagttcaacccgatggaaaacgttgctctggacttctcttacgagcagagccgtattcgtagcgttgacgtaggcacctggattgccggtgttggttaccgcttctaa

SEQ ID NO: 83 - DNA, 1956 bp, Escherichia coli
  source 1..1956: mol_type = other DNA
cagacttcacagcaagacgaaagcacgctggtggttaccgccagtaaacaatcttcccgctcggcatcagccaacaacgtctcatctactgttgtcagcgcgccggaattaagcgacgccggcgtcaccgccagcgacaaactccccagagtcttgcccgggctcaatattgaaaatagcggcaacatgcttttttcgacgatctcgctacgcggcgtctcttcagcgcaggacttctataaccccgccgtcaccctgtatgtcgatggcgtccctcagctttccaccaacaccatccaggcgcttaccgatgtgcaaagcgtggagttgctgcgaggcccacagggaacgttatatggcaaaagcgctcagggcgggatcatcaacatcgtcacccagcagccggacagcacgccgcgcggctatattgaaggcggcgtcagtagccgcgacagttatcgaagtaagttcaacctgagcggccccattcaggatggcctgctgtacggcagcgtcaccctgttacgccaggttgatgacggcgacatgattaaccccgcgacgggaagcgatgacttaggcggcacccgcgccagcatagggaatgtgaaactgcgtctggcgccggacgatcagccctgggaaatgggctttgccgcctcacgcgaatgtacccgcgccacccaggatgcctatgtgggatggaatgatattaagggccgtaagctgtcgatcagcgatggttcaccagacccgtacatgcggcgctgcactgacagccagaccctgagtgggaaatacaccaccgatgactgggttttcaacctgatcagcgcctggcagcagcagcattattcgcgcaccttcccttccggttcgttaatcgtcaatatgcctcagcgctggaatcaggatgtgcaggagctgcgcgccgcaaccctgggcgatgcgcgtaccgttgatatggtgtttgggctgtaccggcagaacacccgcgagaagttaaattcagcctacgacatgccgacaatgccttatttaagcagtaccggctataccaccgctgaaacgctggccgcatacagtgacctgacctggcatttaaccgatcgttttgatatcggcggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcgggtcttgatgccaaaccgttcgtcgccgagaaatccatcaactatgaacttggcacccgctacgaaaccgctgacgtcacgctgcaagccgcgacgttttatacccacaccaaagacatgcagctttactctggcccggtcgggatgcagacattaagcaatgcgggtaaagccgacgccaccggcgttgagcttgaagcgaagtggcggtttgcgccaggctggtcatgggatatcaatggcaacgtgatccgttccgaattcaccaatgacagtgagttgtatcacggtaaccgggtgccgttcgtaccacgttatggcgcgggaagcagcgtgaacggcgtgattgatacgcgctatggcgcactgatgccccgactggcggttaatctggtcgggccgcattatttcgatggcgacaaccagttgcggcaaggcacctatgccaccctggacagcagcctgggctggcaggcgactgaacggatgaacatttccgtctatgtcgataacctgttcgaccgtcgttaccgtacctatggctacatgaacggcagcagcgccgtcgcgcaggtcaatatgggtcgcaccgtcggtatcaatacgcgaattgatttcttctga

SEQ ID NO: 84 - DNA, 2064 bp, Escherichia coli
  source 1..2064: mol_type = other DNA
acttcttcagaaacaaaaatcagcaacgaagagacgctcgtcgtgaccacgaatcgttcggcaagcaacctttgggaaagcccggcgactatacaggttattgaccaacaaacattgcagaactccaccaatgcctccatagccgataatttgcaggacatccccggagtagagataacagacaactccttggcaggccgtaaacaaatccgcattcgtggcgaagcatcctcccgtgttttaattctcattgatggtcaggaggtaacttatcagcgcgccggagataattatggtgtgggactgttgatagatgagtctgcgctggagcgtgttgaggtagtgaaaggtccatattccgtactgtacggttcacaggcaattggcggtattgttaacttcataaccaaaaagggaggtgacaaacttgcatctggagttgtgaaagctgtttataattccgcaacagcaggctgggaagaatcaatcgcggtccaggggagcatcggtggatttgattatcgcatcaacggtagttattctgatcagggcaatcgtgatacgccggatggacgtctgccgaataccaactatcgtaacaatagtcagggtgtatggttgggttataactccggaaaccatcgttttggcctctcgcttgatcgctacagactcgcgacgcaaacttactatgaggatccagacggaagctatgaggcatttagtgtcaaaatacctaaacttgaacgagagaaagttggggtattctatgacacagacgtggacggtgactatctaaaaaaaattcatttcgacgcgtatgagcagaccatccagcgccaatttgccaacgaagtaaaaacgacacagcctgttcccagtccgatgattcaggctctgaccgttcataacaagactgacacccatgataagcaatacactcaggcggtcacattgcagagtcacttttcgctgcctgctaataatgaacttgttaccggtgcacagtacaaacaagacagggtcagccaaaggtccggtggcatgacctcaagcaaatctctgaccggcttcattaataaggaaacacgaactcgctcctattatgagtcagagcaaagtacagtctcactattcgcacaaaatgactggcgattcgccgatcactggacatggacaatgggagttcgccaatactggctttcttcaaagttgacgcgtggtgacggagtatcatataccgcaggcattataagcgatacctctcttgccagagagtctgcgagtgatcacgaaatggtaacatctacaagcctgcgctattcaggtttcgataacttggagttacgcgctgcgttcgcgcaaggctacgtatttcccacactctcccagctttttatgcagacatctgcgggcggcagtgtcacatacggaaatcctgatcttaaggctgaacactccaataactttgaattaggtgcacgatataatggtaatcagtggctgattgacagcgcagtttactactcagaagctaaagattatattgcaagtctgatctgtgatggcagtatagtttgcaatggtaacaccaactcctcccgtagtagctactattattatgacaatattgatcgggcaaaaacatggggactggaaataagcgcggaatataatggctgggttttctcgccatatatcagtggcaatttaattcgtcggcaatatgaaacttcaacattaaaaacaactaatacaggagaaccagcgataaacggacgtatagggctgaaacatactcttgtgatgggtcaggccaacataatctctgatgtttttattcgtgctgcctctagtgcaaaagatgacagtaacggtaccgaaacaaatgttccgggctgggccactctcaactttgcagtaaatacagaattcggtaacgaggatcagtaccggattaacctagcactcaataacctgacagacaaacgctaccgtacagcacatgaaactattcctgcagcaggttttaatgcagctataggttttgtatggaatttctga

SEQ ID NO: 85 - DNA, 2127 bp, Escherichia coli
  source 1..2127: mol_type = other DNA
cagcaaaacgatgataatgagatcatagtgtctgccagccgcagcaatcgaactgtagcggagatggcgcaaaccacctgggttatcgaaaatgccgaactggagcagcagattcagggcggtaaagagctgaaagacgcactggctcagttaatccccggccttgatgtcagcagccagagccgaaccaactacggtatgaacatgcgtggccgcccgctggttgtcctgattgacggtgtgcgcctcaactcttcacgttccgacagccgacaactggactctgtcgatccttttaatatcgaccatattgaagtgatctccggcgcgacggccctgtacggtggcgggagtaccggagggttgatcaacatcgtgaccaaaaaaggccagccggaaaccatgatggagtttgaggctggcacaaaaagtggctttaacagcagtaaagatcacgatgagcgcattgccggtgctgtctccggcggaaatgaccatatctccggacgtctttccgtggcatatcagaaatttggcggctggtttgacggtaacggcgatgccaccctgcttgataacacccagaccggcctgcagcactccaatcggctggacatcatgggaaccggtacgctgaacatcgatgaatcccggcagcttcaactgataacgcagtactataaaagtcagggggacgacaattacgggcttaatctcgggaaaggcttttccgccatcagcgggagcagcacaccatacgtcagtaaggggctgaattctgaccgcattcccggcactgagcggcatttgatcagcctgcagtactctgacagtgatttcctgagacaggaactggtcggtcaggtttactaccgcgatgagtcgttgcggttctacccgttcccgacggtaaatgcgaataaacaggcgacggctttctcctcgtcacagcaggataccgaccagtacggcatgaaactgactctgaacagccaacttatggacggctggcaaatcacctgggggctggatgctgagcatgagcgctttacctccaaccagatgttcttcgatctggctcaggcaagtgcttccggagggctgaacaaccataagatttacaccaccgggcgctatccgtcatatgacatcaccaatctggcggccttcctgcaatccagctatgacattaatgatatttttaccgttagcggtggcgtacgctatcagtatactgagaacagggtagatgatttcatcgactacacgcagcaacagaagattgctgccgggaaggcgatatctgccgacgccattcctggtggttcggtagattacgataactttctgttcaatgctggtctgctgatgcacatcaccgaacgtcagcaggcatggttcaatttttcccagggggtggcattgccggatccggggaaatattatggtcgcggcatctatggtgcagcagtgaacggccatcttcccctgacaaagagcgtgaacgtcagcgacagtaagctggaaggcgtgaaagtcgattcttatgaactgggctggcgctttaccggtgacaacctgcggactcaaatcgcggcatattactcgctttccaataagagcgtggaaaggaataaagatctgaccatcagtgtgaaggacgacaggcgccgtatttacggcgtggaaggtgcggtggactacctgatcccggatactgactggagtaccggtgtgaacttcaatgtgctgaaaaccgagtcgaaagtgaacggtcaatggcaaaaatatgacgtgaaggaatcaagtccatcgaaagcgacagcttacattaactgggcgccggaaccgtggagtctgcgtgtacagagcaccacttctttcgacgtaagcgatgcagagggtaacgatattaatggttacactaccgtcgattttatcagtagttggcagcttccggtgggaacactcagcttcagcgttgagaacctcttcgaccgtgactataccactgtctggggacagcgtgcacctctgtactacagcccgggttacggccctgcttcactgtacgactacaaaggccggggccgaacctttggtctgaactactcagtgctgttctga

SEQ ID NO: 86 - DNA, 1041 bp, Actinobacillus pleuropneumoniae
  source 1..1041: mol_type = other DNA
gcagcgccacaacaaaatactttctacgcaggtgcgaaagcaggttgggcgtcattccatgatggtatcgaacaattagattcagctaaaaacacagatcgcggtacaaaatacggtatcaaccgtaattcagtaacttacggcgtattcggcggttaccaaattttaaaccaagacaaattaggtttagcggctgaattaggttatgactatttcggtcgtgtgcgcggttctgaaaaaccaaacggtaaagcggacaagaaaactttccgtcacgctgcacacggtgcgacaatcgcattaaaacctagctacgaagtattacctgacttagacgtttacggtaaagtaggtatcgcattagtaaacaatacatataaaacattcaatgcagcacaagagaaagtgaaaactcgtcgtttccaaagttctttaattttaggtgcgggtgttgagtacgcaattcttcctgaattagcggcacgtgttgaataccaatggttaaacaacgcaggtaaagcaagctactctactttaaatcgtatgggtgcaactgactaccgttcggatatcagttccgtatctgcaggtttaagctaccgtttcggtcaaggtgcggtaccggttgcagctccggcagttgaaactaaaaacttcgcattcagctctgacgtattattcgcattcggtaaatcaaacttaaaaccggctgcggcaacagcattagatgcaatgcaaaccgaaatcaataacgcaggtttatcaaatgctgcgatccaagtaaacggttacacggaccgtatcggtaaagaagcttcaaacttaaaactttcacaacgtcgtgcggaaacagtagctaactacatcgtttctaaaggtgctctggcagctaacgtaactgcagtaggttacggtgaagcaaaccctgtaaccggcgcaacatgtgacaaagttaaaggtcgtaaagcattaatcgcttgcttagcaccggatcgtcgtgttgaagttcaagttcaaggtactaaagaagtaactatgtaa

SEQ ID NO: 87 - DNA, 588 bp, Actinobacillus pleuropneumoniae
  source 1..588: mol_type = other DNA
gcacatcaagcgggcgatgtgattttccgtgccggtgcgatcggtgtgattgcaaattcaagttcggattatcaaaccggggcggacgtaaacttagatgtaaataataatattcagcttggtttaaccggtacctatatgttaagtgataatttaggtcttgaattattagcggcaacaccgttttctcacaaaatcaccggtaagttaggtgcaacagatttaggcgaagtggcaaaagtaaaacatcttccgccgagcctttacttacaatattatttctttgattctaatgcgacagttcgtccatacgttggtgccggtttaaactatactcgctttttcagtgctgaaagtttaaaaccgcaattagtacaaaacttacgtgttaaaaaacattccgtcgcaccgattgcgaatttaggtgttgatgtgaaattaacggataatctatcattcaatgcggcagcttggtacacacgtattaaaactactgccgattatgatgttccgggattaggtcatgtaagtacaccgattactttagatcctgttgtattattctcaggtattagctacaaattctaa

SEQ ID NO: 88 - DNA, 2745 bp, Actinobacillus pleuropneumoniae
  source 1..2745: mol_type = other DNA
gcagaacaagcggtgcaattgaacgatgtttatgtcacaggaacaaaaaagaaagcacataaaaaagagaacgaagtcacaggcttagggaaagtagttaaaacaccagatactcttagtaaggagcaagtgttaggaatacgagatctgactcgttacgaccccggtatttctgtcgtagaacaagggagaggtgcgactacaggctactcaattcgtggggtagatcgtaaccgtgttggcttggcattagatggtttaccacagattcagtcctatgtaagccagtattcacgttcctcaagcggtgccattaatgaaatagaatacgaaaatctgcgttcgatccaaattagtaaaggggctagttcttctgagtttggtagtggctcactaggcggttcggtgcaattccgtaccaaagaggtaagcgacattattaagccagggcaatcttggggattagataccaaaagtgcctacagtagcaaaaatcaacaatggttaaactcacttgcttttgctggtactcacaatggctttgatgctcttgtgatttacactcaccgtgatggtaaggaaacgaaagctcataaaaatgcagagagtcgttctcagaatatcacccgagtaggagtggaaaccaacgagcttgatacctcaaatagatatactgcgacgacgaataatcaacatacttatggctggtttttgattaaagatgaatgtccaacgttagattgtacgccgaaacagatggctagggtgacaaaagatacgccatctttccgttcttaccctgaatatactcctgaggaaaaacaggcttatgagaaccaaaaacatattacagagcgtctaaatgctcaggattacactggtgaatatagagctttacctgatccgcttaaatataaatctgattcttggctggttaaattaggatacacattctctccgaaacattatgtcgctggtacttatgaacatagcaaacagcgttacgacacccgagatatgacctataccgcttattggcaaccatcggatttacttagaactggtagaaattggtatccaatgaataatgctaaaggattatatcgtgataatgctttagatggtgttgctattgactactttacggaagatggtgtgaaatcctcaaaaggtttacgttgggcaaaagctcgttttattgacgagtggcacactcgtgatcgcttaggtgctttatatcgttatactaatcaagatggaaatcgtctgattgatagactatccttgagtttcgatcagcaaaaaattaatttatctacccgcttgagagaaaacaactgttcggaatatccaaccatagataagaattgccgtgcaactcttgataaactttggtcttctactaaaaatgagcaaagttcttatgaagaaaaacacgacactattcagctctcgttagataaaaccgtacaaacgggattgggtaaacatcaattaaatatgttattaggttcagaccgtttcaattccaccttaaaacgccatgaaattttgagtgaattttctgtagggagttgggggcttgttagagacattggctatcgaaatggatcttacaataatccttatgtgtatgagctaaaagatcaggcaatttatagtaaaaatgaatgtgattatagtggcactattgcaggtagggctgattgtgctacaagtaaaatcaaagggcataatcactacatcgctctgagagataattttgccataaccaagtatttggatattggtttgggttaccgtttcgataagcataaattccgtagcactcatcgctgggcaaatcaaggcgattataaaaatagtgcgtggaatattggcatagtcgcaaaaccaacgtcattcctatcgctctcttatcgagcatcatctggctttagagtgccaagtttccaagagctatttggcttacgttatgatggtgcaatgaaaggctccagcgatgcttaccaaaaaacagagaagttatctcctgaaaaatccttaaaccaagaggttgctgcgactttcaaaggtgattttggtgtcgttgaagtcagttatttcaaaaatgactataagcagttaattgctccagcagaaagaatgcaccaaactcaatcaatgattaactattttaatgtgcaagatattaaattggacggcattaatcttattggtaagctagattggaatggggtatttgataaaattcctgagggcatttacacaacattggcttatagcaaaatgcgagtaaaagaggtgaaaaactatcaagggtatatgaatattcgttctccattgttagataccattcagcctgctcgctatgttgtaggagtggggtacgatcagccagatgaaaaatggggcgtgaatctaacaatgacacactccagtggaaaaaatccaaatgagttaagaggtaatgaacaagtcggttttgccaattatgagcgaactgccacgaagaaaagaacacgttcttggcatacctttgacttaacgggatatatcaccccttggaaacatacaacggtacgagctggcgtatataacctgatgaattatcgttacaccacttgggaatccgtacgtcaatcttcgcttaatgcaattcatcagcatactaacgtaaaagactatgcaaggtatgcagcgcccggtagaaattatgttgtttcattcgaaatgaaattctaa

SEQ ID NO: 89 - DNA, 408 bp, Actinobacillus pleuropneumoniae
  source 1..408: mol_type = other DNA
tttactttaattgaattgatgatcgtgattgcgattattgccattttagctacggttgcaattccgtcatataacagttatacccaaaaagcggcgctttcggagctattggcggcatcggcttcttataaaacggatgtcgagatctgcatatataacaccggagattctaaaaactgtagcggcggtcaaaacggtgtcagaaaaatgacggagcttagacaggctaaatatttaaatgccattacggtggaaggcggaacgattacggtaacggggaaggggaatctgcaggaatacggttatacgatgacaccgattcataacggtagcactatttcttgggaaacgaaatgtaaaggggaggacttaagtttatttccggcaaatttctgtgcgataaattag

SEQ ID NO: 90 - DNA, 825 bp, Pseudomonas aeruginosa
  source 1..825: mol_type = other DNA
tcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgcgagttcctggtgtcggcctatttcagcctgcacgggcgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaagcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgcgcgacattctcagcgcgatctag